SEQUENCE LISTING (3531 sequences)

SEQ ID NO: 1 - protein, 407 aa, Homo sapiens
MVLAAPLLLGFLLLALELRPRGEAAEGPAAAAAAAAAAAAAGVGGERSSRPAPSVAPEPDGCPVCVWRQHSRELRLESIKSQILSKLRLKEAPNISREVVKQLLPKAPPLQQILDLHDFQGDALQPEDFLEEDEYHATTETVISMAQETDPAVQTDGSPLCCHFHFSPKVMFTKVLKAQLWVYLRPVPRPATVYLQILRLKPLTGEGTAGGGGGGRRHIRIRSLKIELHSRSGHWQSIDFKQVLHSWFRQPQSNWGIEINAFDPSGTDLAVTSLGPGAEGLHPFMELRVLENTKRSRRNLGLDCDEHSSESRCCRYPLTVDFEAFGWDWIIAPKRYKANYCSGQCEYMFMQKYPHTHLVQQANPRGSAGPCCTPTKMSPINMLYFNDKQQIIYGKIPGMVVDRCGCS

SEQ ID NO: 2 - protein, 3 aa, Homo sapiens
MVL

SEQ ID NO: 3 - protein, 3 aa, Homo sapiens
VLA

SEQ ID NO: 4 - protein, 3 aa, Homo sapiens
LAA

SEQ ID NO: 5 - protein, 3 aa, Homo sapiens
AAP

SEQ ID NO: 6 - protein, 3 aa, Homo sapiens
APL

SEQ ID NO: 7 - protein, 3 aa, Homo sapiens
PLL

SEQ ID NO: 8 - protein, 3 aa, Homo sapiens
LLL

SEQ ID NO: 9 - protein, 3 aa, Homo sapiens
LLG

SEQ ID NO: 10 - protein, 3 aa, Homo sapiens
LGF

SEQ ID NO: 11 - protein, 3 aa, Homo sapiens
GFL

SEQ ID NO: 12 - protein, 3 aa, Homo sapiens
FLL

SEQ ID NO: 13 - protein, 3 aa, Homo sapiens
LLA

SEQ ID NO: 14 - protein, 3 aa, Homo sapiens
LAL

SEQ ID NO: 15 - protein, 3 aa, Homo sapiens
ALE

SEQ ID NO: 16 - protein, 3 aa, Homo sapiens
LEL

SEQ ID NO: 17 - protein, 3 aa, Homo sapiens
ELR

SEQ ID NO: 18 - protein, 3 aa, Homo sapiens
LRP

SEQ ID NO: 19 - protein, 3 aa, Homo sapiens
RPR

SEQ ID NO: 20 - protein, 3 aa, Homo sapiens
PRG

SEQ ID NO: 21 - protein, 3 aa, Homo sapiens
RGE

SEQ ID NO: 22 - protein, 3 aa, Homo sapiens
GEA

SEQ ID NO: 23 - protein, 3 aa, Homo sapiens
EAA

SEQ ID NO: 24 - protein, 3 aa, Homo sapiens
AAE

SEQ ID NO: 25 - protein, 3 aa, Homo sapiens
AEG

SEQ ID NO: 26 - protein, 3 aa, Homo sapiens
EGP

SEQ ID NO: 27 - protein, 3 aa, Homo sapiens
GPA

SEQ ID NO: 28 - protein, 3 aa, Homo sapiens
PAA

SEQ ID NO: 29 - protein, 3 aa, Homo sapiens
AAA

SEQ ID NO: 30 - protein, 3 aa, Homo sapiens
AAG

SEQ ID NO: 31 - protein, 3 aa, Homo sapiens
AGV

SEQ ID NO: 32 - protein, 3 aa, Homo sapiens
GVG

SEQ ID NO: 33 - protein, 3 aa, Homo sapiens
VGG

SEQ ID NO: 34 - protein, 3 aa, Homo sapiens
GGE

SEQ ID NO: 35 - protein, 3 aa, Homo sapiens
GER

SEQ ID NO: 36 - protein, 3 aa, Homo sapiens
ERS

SEQ ID NO: 37 - protein, 3 aa, Homo sapiens
RSS

SEQ ID NO: 38 - protein, 3 aa, Homo sapiens
SSR

SEQ ID NO: 39 - protein, 3 aa, Homo sapiens
SRP

SEQ ID NO: 40 - protein, 3 aa, Homo sapiens
RPA

SEQ ID NO: 41 - protein, 3 aa, Homo sapiens
PAP

SEQ ID NO: 42 - protein, 3 aa, Homo sapiens
APS

SEQ ID NO: 43 - protein, 3 aa, Homo sapiens
PSV

SEQ ID NO: 44 - protein, 3 aa, Homo sapiens
SVA

SEQ ID NO: 45 - protein, 3 aa, Homo sapiens
VAP

SEQ ID NO: 46 - protein, 3 aa, Homo sapiens
APE

SEQ ID NO: 47 - protein, 3 aa, Homo sapiens
PEP

SEQ ID NO: 48 - protein, 3 aa, Homo sapiens
EPD

SEQ ID NO: 49 - protein, 3 aa, Homo sapiens
PDG

SEQ ID NO: 50 - protein, 3 aa, Homo sapiens
DGC

SEQ ID NO: 51 - protein, 3 aa, Homo sapiens
GCP

SEQ ID NO: 52 - protein, 3 aa, Homo sapiens
CPV

SEQ ID NO: 53 - protein, 3 aa, Homo sapiens
PVC

SEQ ID NO: 54 - protein, 3 aa, Homo sapiens
VCV

SEQ ID NO: 55 - protein, 3 aa, Homo sapiens
CVW

SEQ ID NO: 56 - protein, 3 aa, Homo sapiens
VWR

SEQ ID NO: 57 - protein, 3 aa, Homo sapiens
WRQ

SEQ ID NO: 58 - protein, 3 aa, Homo sapiens
RQH

SEQ ID NO: 59 - protein, 3 aa, Homo sapiens
QHS

SEQ ID NO: 60 - protein, 3 aa, Homo sapiens
HSR

SEQ ID NO: 61 - protein, 3 aa, Homo sapiens
SRE

SEQ ID NO: 62 - protein, 3 aa, Homo sapiens
REL

SEQ ID NO: 63 - protein, 3 aa, Homo sapiens
LRL

SEQ ID NO: 64 - protein, 3 aa, Homo sapiens
RLE

SEQ ID NO: 65 - protein, 3 aa, Homo sapiens
LES

SEQ ID NO: 66 - protein, 3 aa, Homo sapiens
ESI

SEQ ID NO: 67 - protein, 3 aa, Homo sapiens
SIK

SEQ ID NO: 68 - protein, 3 aa, Homo sapiens
IKS

SEQ ID NO: 69 - protein, 3 aa, Homo sapiens
KSQ

SEQ ID NO: 70 - protein, 3 aa, Homo sapiens
SQI

SEQ ID NO: 71 - protein, 3 aa, Homo sapiens
QIL

SEQ ID NO: 72 - protein, 3 aa, Homo sapiens
ILS

SEQ ID NO: 73 - protein, 3 aa, Homo sapiens
LSK

SEQ ID NO: 74 - protein, 3 aa, Homo sapiens
SKL

SEQ ID NO: 75 - protein, 3 aa, Homo sapiens
KLR

SEQ ID NO: 76 - protein, 3 aa, Homo sapiens
RLK

SEQ ID NO: 77 - protein, 3 aa, Homo sapiens
LKE

SEQ ID NO: 78 - protein, 3 aa, Homo sapiens
KEA

SEQ ID NO: 79 - protein, 3 aa, Homo sapiens
EAP

SEQ ID NO: 80 - protein, 3 aa, Homo sapiens
APN

SEQ ID NO: 81 - protein, 3 aa, Homo sapiens
PNI

SEQ ID NO: 82 - protein, 3 aa, Homo sapiens
NIS

SEQ ID NO: 83 - protein, 3 aa, Homo sapiens
ISR

SEQ ID NO: 84 - protein, 3 aa, Homo sapiens
REV

SEQ ID NO: 85 - protein, 3 aa, Homo sapiens
EVV

SEQ ID NO: 86 - protein, 3 aa, Homo sapiens
VVK

SEQ ID NO: 87 - protein, 3 aa, Homo sapiens
VKQ

SEQ ID NO: 88 - protein, 3 aa, Homo sapiens
KQL

SEQ ID NO: 89 - protein, 3 aa, Homo sapiens
QLL

SEQ ID NO: 90 - protein, 3 aa, Homo sapiens
LLP

SEQ ID NO: 91 - protein, 3 aa, Homo sapiens
LPK

SEQ ID NO: 92 - protein, 3 aa, Homo sapiens
PKA

SEQ ID NO: 93 - protein, 3 aa, Homo sapiens
KAP

SEQ ID NO: 94 - protein, 3 aa, Homo sapiens
APP

SEQ ID NO: 95 - protein, 3 aa, Homo sapiens
PPL

SEQ ID NO: 96 - protein, 3 aa, Homo sapiens
PLQ

SEQ ID NO: 97 - protein, 3 aa, Homo sapiens
LQQ

SEQ ID NO: 98 - protein, 3 aa, Homo sapiens
QQI

SEQ ID NO: 99 - protein, 3 aa, Homo sapiens
ILD

SEQ ID NO: 100 - protein, 3 aa, Homo sapiens
LDL

SEQ ID NO: 101 - protein, 3 aa, Homo sapiens
DLH

SEQ ID NO: 102 - protein, 3 aa, Homo sapiens
LHD

SEQ ID NO: 103 - protein, 3 aa, Homo sapiens
HDF

SEQ ID NO: 104 - protein, 3 aa, Homo sapiens
DFQ

SEQ ID NO: 105 - protein, 3 aa, Homo sapiens
FQG

SEQ ID NO: 106 - protein, 3 aa, Homo sapiens
QGD

SEQ ID NO: 107 - protein, 3 aa, Homo sapiens
GDA

SEQ ID NO: 108 - protein, 3 aa, Homo sapiens
DAL

SEQ ID NO: 109 - protein, 3 aa, Homo sapiens
ALQ

SEQ ID NO: 110 - protein, 3 aa, Homo sapiens
LQP

SEQ ID NO: 111 - protein, 3 aa, Homo sapiens
QPE

SEQ ID NO: 112 - protein, 3 aa, Homo sapiens
PED

SEQ ID NO: 113 - protein, 3 aa, Homo sapiens
EDF

SEQ ID NO: 114 - protein, 3 aa, Homo sapiens
DFL

SEQ ID NO: 115 - protein, 3 aa, Homo sapiens
FLE

SEQ ID NO: 116 - protein, 3 aa, Homo sapiens
LEE

SEQ ID NO: 117 - protein, 3 aa, Homo sapiens
EED

SEQ ID NO: 118 - protein, 3 aa, Homo sapiens
EDE

SEQ ID NO: 119 - protein, 3 aa, Homo sapiens
DEY

SEQ ID NO: 120 - protein, 3 aa, Homo sapiens
EYH

SEQ ID NO: 121 - protein, 3 aa, Homo sapiens
YHA

SEQ ID NO: 122 - protein, 3 aa, Homo sapiens
HAT

SEQ ID NO: 123 - protein, 3 aa, Homo sapiens
ATT

SEQ ID NO: 124 - protein, 3 aa, Homo sapiens
TTE

SEQ ID NO: 125 - protein, 3 aa, Homo sapiens
TET

SEQ ID NO: 126 - protein, 3 aa, Homo sapiens
ETV

SEQ ID NO: 127 - protein, 3 aa, Homo sapiens
TVI

SEQ ID NO: 128 - protein, 3 aa, Homo sapiens
VIS

SEQ ID NO: 129 - protein, 3 aa, Homo sapiens
ISM

SEQ ID NO: 130 - protein, 3 aa, Homo sapiens
SMA

SEQ ID NO: 131 - protein, 3 aa, Homo sapiens
MAQ

SEQ ID NO: 132 - protein, 3 aa, Homo sapiens
AQE

SEQ ID NO: 133 - protein, 3 aa, Homo sapiens
QET

SEQ ID NO: 134 - protein, 3 aa, Homo sapiens
ETD

SEQ ID NO: 135 - protein, 3 aa, Homo sapiens
TDP

SEQ ID NO: 136 - protein, 3 aa, Homo sapiens
DPA

SEQ ID NO: 137 - protein, 3 aa, Homo sapiens
PAV

SEQ ID NO: 138 - protein, 3 aa, Homo sapiens
AVQ

SEQ ID NO: 139 - protein, 3 aa, Homo sapiens
VQT

SEQ ID NO: 140 - protein, 3 aa, Homo sapiens
QTD

SEQ ID NO: 141 - protein, 3 aa, Homo sapiens
TDG

SEQ ID NO: 142 - protein, 3 aa, Homo sapiens
DGS

SEQ ID NO: 143 - protein, 3 aa, Homo sapiens
GSP

SEQ ID NO: 144 - protein, 3 aa, Homo sapiens
SPL

SEQ ID NO: 145 - protein, 3 aa, Homo sapiens
PLC

SEQ ID NO: 146 - protein, 3 aa, Homo sapiens
LCC

SEQ ID NO: 147 - protein, 3 aa, Homo sapiens
CCH

SEQ ID NO: 148 - protein, 3 aa, Homo sapiens
CHF

SEQ ID NO: 149 - protein, 3 aa, Homo sapiens
HFH

SEQ ID NO: 150 - protein, 3 aa, Homo sapiens
FHF

SEQ ID NO: 151 - protein, 3 aa, Homo sapiens
HFS

SEQ ID NO: 152 - protein, 3 aa, Homo sapiens
FSP

SEQ ID NO: 153 - protein, 3 aa, Homo sapiens
SPK

SEQ ID NO: 154 - protein, 3 aa, Homo sapiens
PKV

SEQ ID NO: 155 - protein, 3 aa, Homo sapiens
KVM

SEQ ID NO: 156 - protein, 3 aa, Homo sapiens
VMF

SEQ ID NO: 157 - protein, 3 aa, Homo sapiens
MFT

SEQ ID NO: 158 - protein, 3 aa, Homo sapiens
FTK

SEQ ID NO: 159 - protein, 3 aa, Homo sapiens
TKV

SEQ ID NO: 160 - protein, 3 aa, Homo sapiens
KVL

SEQ ID NO: 161 - protein, 3 aa, Homo sapiens
VLK

SEQ ID NO: 162 - protein, 3 aa, Homo sapiens
LKA

SEQ ID NO: 163 - protein, 3 aa, Homo sapiens
KAQ

SEQ ID NO: 164 - protein, 3 aa, Homo sapiens
AQL

SEQ ID NO: 165 - protein, 3 aa, Homo sapiens
QLW

SEQ ID NO: 166 - protein, 3 aa, Homo sapiens
LWV

SEQ ID NO: 167 - protein, 3 aa, Homo sapiens
WVY

SEQ ID NO: 168 - protein, 3 aa, Homo sapiens
VYL

SEQ ID NO: 169 - protein, 3 aa, Homo sapiens
YLR

SEQ ID NO: 170 - protein, 3 aa, Homo sapiens
RPV

SEQ ID NO: 171 - protein, 3 aa, Homo sapiens
PVP

SEQ ID NO: 172 - protein, 3 aa, Homo sapiens
VPR

SEQ ID NO: 173 - protein, 3 aa, Homo sapiens
PRP

SEQ ID NO: 174 - protein, 3 aa, Homo sapiens
PAT

SEQ ID NO: 175 - protein, 3 aa, Homo sapiens
ATV

SEQ ID NO: 176 - protein, 3 aa, Homo sapiens
TVY

SEQ ID NO: 177 - protein, 3 aa, Homo sapiens
YLQ

SEQ ID NO: 178 - protein, 3 aa, Homo sapiens
LQI

SEQ ID NO: 179 - protein, 3 aa, Homo sapiens
ILR

SEQ ID NO: 180 - protein, 3 aa, Homo sapiens
LKP

SEQ ID NO: 181 - protein, 3 aa, Homo sapiens
KPL

SEQ ID NO: 182 - protein, 3 aa, Homo sapiens
PLT

SEQ ID NO: 183 - protein, 3 aa, Homo sapiens
LTG

SEQ ID NO: 184 - protein, 3 aa, Homo sapiens
TGE

SEQ ID NO: 185 - protein, 3 aa, Homo sapiens
GEG

SEQ ID NO: 186 - protein, 3 aa, Homo sapiens
EGT

SEQ ID NO: 187 - protein, 3 aa, Homo sapiens
GTA

SEQ ID NO: 188 - protein, 3 aa, Homo sapiens
TAG

SEQ ID NO: 189 - protein, 3 aa, Homo sapiens
AGG

SEQ ID NO: 190 - protein, 3 aa, Homo sapiens
GGG

SEQ ID NO: 191 - protein, 3 aa, Homo sapiens
GGR

SEQ ID NO: 192 - protein, 3 aa, Homo sapiens
GRR

SEQ ID NO: 193 - protein, 3 aa, Homo sapiens
RRH

SEQ ID NO: 194 - protein, 3 aa, Homo sapiens
RHI

SEQ ID NO: 195 - protein, 3 aa, Homo sapiens
HIR

SEQ ID NO: 196 - protein, 3 aa, Homo sapiens
IRI

SEQ ID NO: 197 - protein, 3 aa, Homo sapiens
RIR

SEQ ID NO: 198 - protein, 3 aa, Homo sapiens
IRS

SEQ ID NO: 199 - protein, 3 aa, Homo sapiens
RSL

SEQ ID NO: 200 - protein, 3 aa, Homo sapiens
SLK

SEQ ID NO: 201 - protein, 3 aa, Homo sapiens
LKI

SEQ ID NO: 202 - protein, 3 aa, Homo sapiens
KIE

SEQ ID NO: 203 - protein, 3 aa, Homo sapiens
IEL

SEQ ID NO: 204 - protein, 3 aa, Homo sapiens
ELH

SEQ ID NO: 205 - protein, 3 aa, Homo sapiens
LHS

SEQ ID NO: 206 - protein, 3 aa, Homo sapiens
SRS

SEQ ID NO: 207 - protein, 3 aa, Homo sapiens
RSG

SEQ ID NO: 208 - protein, 3 aa, Homo sapiens
SGH

SEQ ID NO: 209 - protein, 3 aa, Homo sapiens
GHW

SEQ ID NO: 210 - protein, 3 aa, Homo sapiens
HWQ

SEQ ID NO: 211 - protein, 3 aa, Homo sapiens
WQS

SEQ ID NO: 212 - protein, 3 aa, Homo sapiens
QSI

SEQ ID NO: 213 - protein, 3 aa, Homo sapiens
SID

SEQ ID NO: 214 - protein, 3 aa, Homo sapiens
IDF

SEQ ID NO: 215 - protein, 3 aa, Homo sapiens
DFK

SEQ ID NO: 216 - protein, 3 aa, Homo sapiens
FKQ

SEQ ID NO: 217 - protein, 3 aa, Homo sapiens
KQV

SEQ ID NO: 218 - protein, 3 aa, Homo sapiens
QVL

SEQ ID NO: 219 - protein, 3 aa, Homo sapiens
VLH

SEQ ID NO: 220 - protein, 3 aa, Homo sapiens
HSW

SEQ ID NO: 221 - protein, 3 aa, Homo sapiens
SWF

SEQ ID NO: 222 - protein, 3 aa, Homo sapiens
WFR

SEQ ID NO: 223 - protein, 3 aa, Homo sapiens
FRQ

SEQ ID NO: 224 - protein, 3 aa, Homo sapiens
RQP

SEQ ID NO: 225 - protein, 3 aa, Homo sapiens
QPQ

SEQ ID NO: 226 - protein, 3 aa, Homo sapiens
PQS

SEQ ID NO: 227 - protein, 3 aa, Homo sapiens
QSN

SEQ ID NO: 228 - protein, 3 aa, Homo sapiens
SNW

SEQ ID NO: 229 - protein, 3 aa, Homo sapiens
NWG

SEQ ID NO: 230 - protein, 3 aa, Homo sapiens
WGI

SEQ ID NO: 231 - protein, 3 aa, Homo sapiens
GIE

SEQ ID NO: 232 - protein, 3 aa, Homo sapiens
IEI

SEQ ID NO: 233 - protein, 3 aa, Homo sapiens
EIN

SEQ ID NO: 234 - protein, 3 aa, Homo sapiens
INA

SEQ ID NO: 235 - protein, 3 aa, Homo sapiens
NAF

SEQ ID NO: 236 - protein, 3 aa, Homo sapiens
AFD

SEQ ID NO: 237 - protein, 3 aa, Homo sapiens
FDP

SEQ ID NO: 238 - protein, 3 aa, Homo sapiens
DPS

SEQ ID NO: 239 - protein, 3 aa, Homo sapiens
PSG

SEQ ID NO: 240 - protein, 3 aa, Homo sapiens
SGT

SEQ ID NO: 241 - protein, 3 aa, Homo sapiens
GTD

SEQ ID NO: 242 - protein, 3 aa, Homo sapiens
TDL

SEQ ID NO: 243 - protein, 3 aa, Homo sapiens
DLA

SEQ ID NO: 244 - protein, 3 aa, Homo sapiens
LAV

SEQ ID NO: 245 - protein, 3 aa, Homo sapiens
AVT

SEQ ID NO: 246 - protein, 3 aa, Homo sapiens
VTS

SEQ ID NO: 247 - protein, 3 aa, Homo sapiens
TSL

SEQ ID NO: 248 - protein, 3 aa, Homo sapiens
SLG

SEQ ID NO: 249 - protein, 3 aa, Homo sapiens
LGP

SEQ ID NO: 250 - protein, 3 aa, Homo sapiens
GPG

SEQ ID NO: 251 - protein, 3 aa, Homo sapiens
PGA

SEQ ID NO: 252 - protein, 3 aa, Homo sapiens
GAE

SEQ ID NO: 253 - protein, 3 aa, Homo sapiens
EGL

SEQ ID NO: 254 - protein, 3 aa, Homo sapiens
GLH

SEQ ID NO: 255 - protein, 3 aa, Homo sapiens
LHP

SEQ ID NO: 256 - protein, 3 aa, Homo sapiens
HPF

SEQ ID NO: 257 - protein, 3 aa, Homo sapiens
PFM

SEQ ID NO: 258 - protein, 3 aa, Homo sapiens
FME

SEQ ID NO: 259 - protein, 3 aa, Homo sapiens
MEL

SEQ ID NO: 260 - protein, 3 aa, Homo sapiens
LRV

SEQ ID NO: 261 - protein, 3 aa, Homo sapiens
RVL

SEQ ID NO: 262 - protein, 3 aa, Homo sapiens
VLE

SEQ ID NO: 263 - protein, 3 aa, Homo sapiens
LEN

SEQ ID NO: 264 - protein, 3 aa, Homo sapiens
ENT

SEQ ID NO: 265 - protein, 3 aa, Homo sapiens
NTK

SEQ ID NO: 266 - protein, 3 aa, Homo sapiens
TKR

SEQ ID NO: 267 - protein, 3 aa, Homo sapiens
KRS

SEQ ID NO: 268 - protein, 3 aa, Homo sapiens
RSR

SEQ ID NO: 269 - protein, 3 aa, Homo sapiens
SRR

SEQ ID NO: 270 - protein, 3 aa, Homo sapiens
RRN

SEQ ID NO: 271 - protein, 3 aa, Homo sapiens
RNL

SEQ ID NO: 272 - protein, 3 aa, Homo sapiens
NLG

SEQ ID NO: 273 - protein, 3 aa, Homo sapiens
LGL

SEQ ID NO: 274 - protein, 3 aa, Homo sapiens
GLD

SEQ ID NO: 275 - protein, 3 aa, Homo sapiens
LDC

SEQ ID NO: 276 - protein, 3 aa, Homo sapiens
DCD

SEQ ID NO: 277 - protein, 3 aa, Homo sapiens
CDE

SEQ ID NO: 278 - protein, 3 aa, Homo sapiens
DEH

SEQ ID NO: 279 - protein, 3 aa, Homo sapiens
EHS

SEQ ID NO: 280 - protein, 3 aa, Homo sapiens
HSS

SEQ ID NO: 281 - protein, 3 aa, Homo sapiens
SSE

SEQ ID NO: 282 - protein, 3 aa, Homo sapiens
SES

SEQ ID NO: 283 - protein, 3 aa, Homo sapiens
ESR

SEQ ID NO: 284 - protein, 3 aa, Homo sapiens
SRC

SEQ ID NO: 285 - protein, 3 aa, Homo sapiens
RCC

SEQ ID NO: 286 - protein, 3 aa, Homo sapiens
CCR

SEQ ID NO: 287 - protein, 3 aa, Homo sapiens
CRY

SEQ ID NO: 288 - protein, 3 aa, Homo sapiens
RYP

SEQ ID NO: 289 - protein, 3 aa, Homo sapiens
YPL

SEQ ID NO: 290 - protein, 3 aa, Homo sapiens
LTV

SEQ ID NO: 291 - protein, 3 aa, Homo sapiens
TVD

SEQ ID NO: 292 - protein, 3 aa, Homo sapiens
VDF

SEQ ID NO: 293 - protein, 3 aa, Homo sapiens
DFE

SEQ ID NO: 294 - protein, 3 aa, Homo sapiens
FEA

SEQ ID NO: 295 - protein, 3 aa, Homo sapiens
EAF

SEQ ID NO: 296 - protein, 3 aa, Homo sapiens
AFG

SEQ ID NO: 297 - protein, 3 aa, Homo sapiens
FGW

SEQ ID NO: 298 - protein, 3 aa, Homo sapiens
GWD

SEQ ID NO: 299 - protein, 3 aa, Homo sapiens
WDW

SEQ ID NO: 300 - protein, 3 aa, Homo sapiens
DWI

SEQ ID NO: 301 - protein, 3 aa, Homo sapiens
WII

SEQ ID NO: 302 - protein, 3 aa, Homo sapiens
IIA

SEQ ID NO: 303 - protein, 3 aa, Homo sapiens
IAP

SEQ ID NO: 304 - protein, 3 aa, Homo sapiens
APK

SEQ ID NO: 305 - protein, 3 aa, Homo sapiens
PKR

SEQ ID NO: 306 - protein, 3 aa, Homo sapiens
KRY

SEQ ID NO: 307 - protein, 3 aa, Homo sapiens
RYK

SEQ ID NO: 308 - protein, 3 aa, Homo sapiens
YKA

SEQ ID NO: 309 - protein, 3 aa, Homo sapiens
KAN

SEQ ID NO: 310 - protein, 3 aa, Homo sapiens
ANY

SEQ ID NO: 311 - protein, 3 aa, Homo sapiens
NYC

SEQ ID NO: 312 - protein, 3 aa, Homo sapiens
YCS

SEQ ID NO: 313 - protein, 3 aa, Homo sapiens
CSG

SEQ ID NO: 314 - protein, 3 aa, Homo sapiens
SGQ

SEQ ID NO: 315 - protein, 3 aa, Homo sapiens
GQC

SEQ ID NO: 316 - protein, 3 aa, Homo sapiens
QCE

SEQ ID NO: 317 - protein, 3 aa, Homo sapiens
CEY

SEQ ID NO: 318 - protein, 3 aa, Homo sapiens
EYM

SEQ ID NO: 319 - protein, 3 aa, Homo sapiens
YMF

SEQ ID NO: 320 - protein, 3 aa, Homo sapiens
MFM

SEQ ID NO: 321 - protein, 3 aa, Homo sapiens
FMQ

SEQ ID NO: 322 - protein, 3 aa, Homo sapiens
MQK

SEQ ID NO: 323 - protein, 3 aa, Homo sapiens
QKY

SEQ ID NO: 324 - protein, 3 aa, Homo sapiens
KYP

SEQ ID NO: 325 - protein, 3 aa, Homo sapiens
YPH

SEQ ID NO: 326 - protein, 3 aa, Homo sapiens
PHT

SEQ ID NO: 327 - protein, 3 aa, Homo sapiens
HTH

SEQ ID NO: 328 - protein, 3 aa, Homo sapiens
THL

SEQ ID NO: 329 - protein, 3 aa, Homo sapiens
HLV

SEQ ID NO: 330 - protein, 3 aa, Homo sapiens
LVQ

SEQ ID NO: 331 - protein, 3 aa, Homo sapiens
VQQ

SEQ ID NO: 332 - protein, 3 aa, Homo sapiens
QQA

SEQ ID NO: 333 - protein, 3 aa, Homo sapiens
QAN

SEQ ID NO: 334 - protein, 3 aa, Homo sapiens
ANP

SEQ ID NO: 335 - protein, 3 aa, Homo sapiens
NPR

SEQ ID NO: 336 - protein, 3 aa, Homo sapiens
RGS

SEQ ID NO: 337 - protein, 3 aa, Homo sapiens
GSA

SEQ ID NO: 338 - protein, 3 aa, Homo sapiens
SAG

SEQ ID NO: 339 - protein, 3 aa, Homo sapiens
AGP

SEQ ID NO: 340 - protein, 3 aa, Homo sapiens
GPC

SEQ ID NO: 341 - protein, 3 aa, Homo sapiens
PCC

SEQ ID NO: 342 - protein, 3 aa, Homo sapiens
CCT

SEQ ID NO: 343 - protein, 3 aa, Homo sapiens
CTP

SEQ ID NO: 344 - protein, 3 aa, Homo sapiens
TPT

SEQ ID NO: 345 - protein, 3 aa, Homo sapiens
PTK

SEQ ID NO: 346 - protein, 3 aa, Homo sapiens
TKM

SEQ ID NO: 347 - protein, 3 aa, Homo sapiens
KMS

SEQ ID NO: 348 - protein, 3 aa, Homo sapiens
MSP

SEQ ID NO: 349 - protein, 3 aa, Homo sapiens
SPI

SEQ ID NO: 350 - protein, 3 aa, Homo sapiens
PIN

SEQ ID NO: 351 - protein, 3 aa, Homo sapiens
INM

SEQ ID NO: 352 - protein, 3 aa, Homo sapiens
NML

SEQ ID NO: 353 - protein, 3 aa, Homo sapiens
MLY

SEQ ID NO: 354 - protein, 3 aa, Homo sapiens
LYF

SEQ ID NO: 355 - protein, 3 aa, Homo sapiens
YFN

SEQ ID NO: 356 - protein, 3 aa, Homo sapiens
FND

SEQ ID NO: 357 - protein, 3 aa, Homo sapiens
NDK

SEQ ID NO: 358 - protein, 3 aa, Homo sapiens
DKQ

SEQ ID NO: 359 - protein, 3 aa, Homo sapiens
KQQ

SEQ ID NO: 360 - protein, 3 aa, Homo sapiens
QII

SEQ ID NO: 361 - protein, 3 aa, Homo sapiens
IIY

SEQ ID NO: 362 - protein, 3 aa, Homo sapiens
IYG

SEQ ID NO: 363 - protein, 3 aa, Homo sapiens
YGK

SEQ ID NO: 364 - protein, 3 aa, Homo sapiens
GKI

SEQ ID NO: 365 - protein, 3 aa, Homo sapiens
KIP

SEQ ID NO: 366 - protein, 3 aa, Homo sapiens
IPG

SEQ ID NO: 367 - protein, 3 aa, Homo sapiens
PGM

SEQ ID NO: 368 - protein, 3 aa, Homo sapiens
GMV

SEQ ID NO: 369 - protein, 3 aa, Homo sapiens
MVV

SEQ ID NO: 370 - protein, 3 aa, Homo sapiens
VVD

SEQ ID NO: 371 - protein, 3 aa, Homo sapiens
VDR

SEQ ID NO: 372 - protein, 3 aa, Homo sapiens
DRC

SEQ ID NO: 373 - protein, 3 aa, Homo sapiens
RCG

SEQ ID NO: 374 - protein, 3 aa, Homo sapiens
CGC

SEQ ID NO: 375 - protein, 3 aa, Homo sapiens
GCS

SEQ ID NO: 376 - protein, 4 aa, Homo sapiens
MVLA

SEQ ID NO: 377 - protein, 4 aa, Homo sapiens
VLAA

SEQ ID NO: 378 - protein, 4 aa, Homo sapiens
LAAP

SEQ ID NO: 379 - protein, 4 aa, Homo sapiens
AAPL

SEQ ID NO: 380 - protein, 4 aa, Homo sapiens
APLL

SEQ ID NO: 381 - protein, 4 aa, Homo sapiens
PLLL

SEQ ID NO: 382 - protein, 4 aa, Homo sapiens
LLLG

SEQ ID NO: 383 - protein, 4 aa, Homo sapiens
LLGF

SEQ ID NO: 384 - protein, 4 aa, Homo sapiens
LGFL

SEQ ID NO: 385 - protein, 4 aa, Homo sapiens
GFLL

SEQ ID NO: 386 - protein, 4 aa, Homo sapiens
FLLL

SEQ ID NO: 387 - protein, 4 aa, Homo sapiens
LLLA

SEQ ID NO: 388 - protein, 4 aa, Homo sapiens
LLAL

SEQ ID NO: 389 - protein, 4 aa, Homo sapiens
LALE

SEQ ID NO: 390 - protein, 4 aa, Homo sapiens
ALEL

SEQ ID NO: 391 - protein, 4 aa, Homo sapiens
LELR

SEQ ID NO: 392 - protein, 4 aa, Homo sapiens
ELRP

SEQ ID NO: 393 - protein, 4 aa, Homo sapiens
LRPR

SEQ ID NO: 394 - protein, 4 aa, Homo sapiens
RPRG

SEQ ID NO: 395 - protein, 4 aa, Homo sapiens
PRGE

SEQ ID NO: 396 - protein, 4 aa, Homo sapiens
RGEA

SEQ ID NO: 397 - protein, 4 aa, Homo sapiens
GEAA

SEQ ID NO: 398 - protein, 4 aa, Homo sapiens
EAAE

SEQ ID NO: 399 - protein, 4 aa, Homo sapiens
AAEG

SEQ ID NO: 400 - protein, 4 aa, Homo sapiens
AEGP

SEQ ID NO: 401 - protein, 4 aa, Homo sapiens
EGPA

SEQ ID NO: 402 - protein, 4 aa, Homo sapiens
GPAA

SEQ ID NO: 403 - protein, 4 aa, Homo sapiens
PAAA

SEQ ID NO: 404 - protein, 4 aa, Homo sapiens
AAAA

SEQ ID NO: 405 - protein, 4 aa, Homo sapiens
AAAG

SEQ ID NO: 406 - protein, 4 aa, Homo sapiens
AAGV

SEQ ID NO: 407 - protein, 4 aa, Homo sapiens
AGVG

SEQ ID NO: 408 - protein, 4 aa, Homo sapiens
GVGG

SEQ ID NO: 409 - protein, 4 aa, Homo sapiens
VGGE

SEQ ID NO: 410 - protein, 4 aa, Homo sapiens
GGER

SEQ ID NO: 411 - protein, 4 aa, Homo sapiens
GERS

SEQ ID NO: 412 - protein, 4 aa, Homo sapiens
ERSS

SEQ ID NO: 413 - protein, 4 aa, Homo sapiens
RSSR

SEQ ID NO: 414 - protein, 4 aa, Homo sapiens
SSRP

SEQ ID NO: 415 - protein, 4 aa, Homo sapiens
SRPA

SEQ ID NO: 416 - protein, 4 aa, Homo sapiens
RPAP

SEQ ID NO: 417 - protein, 4 aa, Homo sapiens
PAPS

SEQ ID NO: 418 - protein, 4 aa, Homo sapiens
APSV

SEQ ID NO: 419 - protein, 4 aa, Homo sapiens
PSVA

SEQ ID NO: 420 - protein, 4 aa, Homo sapiens
SVAP

SEQ ID NO: 421 - protein, 4 aa, Homo sapiens
VAPE

SEQ ID NO: 422 - protein, 4 aa, Homo sapiens
APEP

SEQ ID NO: 423 - protein, 4 aa, Homo sapiens
PEPD

SEQ ID NO: 424 - protein, 4 aa, Homo sapiens
EPDG

SEQ ID NO: 425 - protein, 4 aa, Homo sapiens
PDGC

SEQ ID NO: 426 - protein, 4 aa, Homo sapiens
DGCP

SEQ ID NO: 427 - protein, 4 aa, Homo sapiens
GCPV

SEQ ID NO: 428 - protein, 4 aa, Homo sapiens
CPVC

SEQ ID NO: 429 - protein, 4 aa, Homo sapiens
PVCV

SEQ ID NO: 430 - protein, 4 aa, Homo sapiens
VCVW

SEQ ID NO: 431 - protein, 4 aa, Homo sapiens
CVWR

SEQ ID NO: 432 - protein, 4 aa, Homo sapiens
VWRQ

SEQ ID NO: 433 - protein, 4 aa, Homo sapiens
WRQH

SEQ ID NO: 434 - protein, 4 aa, Homo sapiens
RQHS

SEQ ID NO: 435 - protein, 4 aa, Homo sapiens
QHSR

SEQ ID NO: 436 - protein, 4 aa, Homo sapiens
HSRE

SEQ ID NO: 437 - protein, 4 aa, Homo sapiens
SREL

SEQ ID NO: 438 - protein, 4 aa, Homo sapiens
RELR

SEQ ID NO: 439 - protein, 4 aa, Homo sapiens
ELRL

SEQ ID NO: 440 - protein, 4 aa, Homo sapiens
LRLE

SEQ ID NO: 441 - protein, 4 aa, Homo sapiens
RLES

SEQ ID NO: 442 - protein, 4 aa, Homo sapiens
LESI

SEQ ID NO: 443 - protein, 4 aa, Homo sapiens
ESIK

SEQ ID NO: 444 - protein, 4 aa, Homo sapiens
SIKS

SEQ ID NO: 445 - protein, 4 aa, Homo sapiens
IKSQ

SEQ ID NO: 446 - protein, 4 aa, Homo sapiens
KSQI

SEQ ID NO: 447 - protein, 4 aa, Homo sapiens
SQIL

SEQ ID NO: 448 - protein, 4 aa, Homo sapiens
QILS

SEQ ID NO: 449 - protein, 4 aa, Homo sapiens
ILSK

SEQ ID NO: 450 - protein, 4 aa, Homo sapiens
LSKL

SEQ ID NO: 451 - protein, 4 aa, Homo sapiens
SKLR

SEQ ID NO: 452 - protein, 4 aa, Homo sapiens
KLRL

SEQ ID NO: 453 - protein, 4 aa, Homo sapiens
LRLK

SEQ ID NO: 454 - protein, 4 aa, Homo sapiens
RLKE

SEQ ID NO: 455 - protein, 4 aa, Homo sapiens
LKEA

SEQ ID NO: 456 - protein, 4 aa, Homo sapiens
KEAP

SEQ ID NO: 457 - protein, 4 aa, Homo sapiens
EAPN

SEQ ID NO: 458 - protein, 4 aa, Homo sapiens
APNI

SEQ ID NO: 459 - protein, 4 aa, Homo sapiens
PNIS

SEQ ID NO: 460 - protein, 4 aa, Homo sapiens
NISR

SEQ ID NO: 461 - protein, 4 aa, Homo sapiens
ISRE

SEQ ID NO: 462 - protein, 4 aa, Homo sapiens
SREV

SEQ ID NO: 463 - protein, 4 aa, Homo sapiens
REVV

SEQ ID NO: 464 - protein, 4 aa, Homo sapiens
EVVK

SEQ ID NO: 465 - protein, 4 aa, Homo sapiens
VVKQ

SEQ ID NO: 466 - protein, 4 aa, Homo sapiens
VKQL

SEQ ID NO: 467 - protein, 4 aa, Homo sapiens
KQLL

SEQ ID NO: 468 - protein, 4 aa, Homo sapiens
QLLP

SEQ ID NO: 469 - protein, 4 aa, Homo sapiens
LLPK

SEQ ID NO: 470 - protein, 4 aa, Homo sapiens
LPKA

SEQ ID NO: 471 - protein, 4 aa, Homo sapiens
PKAP

SEQ ID NO: 472 - protein, 4 aa, Homo sapiens
KAPP

SEQ ID NO: 473 - protein, 4 aa, Homo sapiens
APPL

SEQ ID NO: 474 - protein, 4 aa, Homo sapiens
PPLQ

SEQ ID NO: 475 - protein, 4 aa, Homo sapiens
PLQQ

SEQ ID NO: 476 - protein, 4 aa, Homo sapiens
LQQI

SEQ ID NO: 477 - protein, 4 aa, Homo sapiens
QQIL

SEQ ID NO: 478 - protein, 4 aa, Homo sapiens
QILD

SEQ ID NO: 479 - protein, 4 aa, Homo sapiens
ILDL

SEQ ID NO: 480 - protein, 4 aa, Homo sapiens
LDLH

SEQ ID NO: 481 - protein, 4 aa, Homo sapiens
DLHD

SEQ ID NO: 482 - protein, 4 aa, Homo sapiens
LHDF

SEQ ID NO: 483 - protein, 4 aa, Homo sapiens
HDFQ

SEQ ID NO: 484 - protein, 4 aa, Homo sapiens
DFQG

SEQ ID NO: 485 - protein, 4 aa, Homo sapiens
FQGD

SEQ ID NO: 486 - protein, 4 aa, Homo sapiens
QGDA

SEQ ID NO: 487 - protein, 4 aa, Homo sapiens
GDAL

SEQ ID NO: 488 - protein, 4 aa, Homo sapiens
DALQ

SEQ ID NO: 489 - protein, 4 aa, Homo sapiens
ALQP

SEQ ID NO: 490 - protein, 4 aa, Homo sapiens
LQPE

SEQ ID NO: 491 - protein, 4 aa, Homo sapiens
QPED

SEQ ID NO: 492 - protein, 4 aa, Homo sapiens
PEDF

SEQ ID NO: 493 - protein, 4 aa, Homo sapiens
EDFL

SEQ ID NO: 494 - protein, 4 aa, Homo sapiens
DFLE

SEQ ID NO: 495 - protein, 4 aa, Homo sapiens
FLEE

SEQ ID NO: 496 - protein, 4 aa, Homo sapiens
LEED

SEQ ID NO: 497 - protein, 4 aa, Homo sapiens
EEDE

SEQ ID NO: 498 - protein, 4 aa, Homo sapiens
EDEY

SEQ ID NO: 499 - protein, 4 aa, Homo sapiens
DEYH

SEQ ID NO: 500 - protein, 4 aa, Homo sapiens
EYHA

SEQ ID NO: 501 - protein, 4 aa, Homo sapiens
YHAT

SEQ ID NO: 502 - protein, 4 aa, Homo sapiens
HATT

SEQ ID NO: 503 - protein, 4 aa, Homo sapiens
ATTE

SEQ ID NO: 504 - protein, 4 aa, Homo sapiens
TTET

SEQ ID NO: 505 - protein, 4 aa, Homo sapiens
TETV

SEQ ID NO: 506 - protein, 4 aa, Homo sapiens
ETVI

SEQ ID NO: 507 - protein, 4 aa, Homo sapiens
TVIS

SEQ ID NO: 508 - protein, 4 aa, Homo sapiens
VISM

SEQ ID NO: 509 - protein, 4 aa, Homo sapiens
ISMA

SEQ ID NO: 510 - protein, 4 aa, Homo sapiens
SMAQ

SEQ ID NO: 511 - protein, 4 aa, Homo sapiens
MAQE

SEQ ID NO: 512 - protein, 4 aa, Homo sapiens
AQET

SEQ ID NO: 513 - protein, 4 aa, Homo sapiens
QETD

SEQ ID NO: 514 - protein, 4 aa, Homo sapiens
ETDP

SEQ ID NO: 515 - protein, 4 aa, Homo sapiens
TDPA

SEQ ID NO: 516 - protein, 4 aa, Homo sapiens
DPAV

SEQ ID NO: 517 - protein, 4 aa, Homo sapiens
PAVQ

SEQ ID NO: 518 - protein, 4 aa, Homo sapiens
AVQT

SEQ ID NO: 519 - protein, 4 aa, Homo sapiens
VQTD

SEQ ID NO: 520 - protein, 4 aa, Homo sapiens
QTDG

SEQ ID NO: 521 - protein, 4 aa, Homo sapiens
TDGS

SEQ ID NO: 522 - protein, 4 aa, Homo sapiens
DGSP

SEQ ID NO: 523 - protein, 4 aa, Homo sapiens
GSPL

SEQ ID NO: 524 - protein, 4 aa, Homo sapiens
SPLC

SEQ ID NO: 525 - protein, 4 aa, Homo sapiens
PLCC

SEQ ID NO: 526 - protein, 4 aa, Homo sapiens
LCCH

SEQ ID NO: 527 - protein, 4 aa, Homo sapiens
CCHF

SEQ ID NO: 528 - protein, 4 aa, Homo sapiens
CHFH

SEQ ID NO: 529 - protein, 4 aa, Homo sapiens
HFHF

SEQ ID NO: 530 - protein, 4 aa, Homo sapiens
FHFS

SEQ ID NO: 531 - protein, 4 aa, Homo sapiens
HFSP

SEQ ID NO: 532 - protein, 4 aa, Homo sapiens
FSPK

SEQ ID NO: 533 - protein, 4 aa, Homo sapiens
SPKV

SEQ ID NO: 534 - protein, 4 aa, Homo sapiens
PKVM

SEQ ID NO: 535 - protein, 4 aa, Homo sapiens
KVMF

SEQ ID NO: 536 - protein, 4 aa, Homo sapiens
VMFT

SEQ ID NO: 537 - protein, 4 aa, Homo sapiens
MFTK

SEQ ID NO: 538 - protein, 4 aa, Homo sapiens
FTKV

SEQ ID NO: 539 - protein, 4 aa, Homo sapiens
TKVL

SEQ ID NO: 540 - protein, 4 aa, Homo sapiens
KVLK

SEQ ID NO: 541 - protein, 4 aa, Homo sapiens
VLKA

SEQ ID NO: 542 - protein, 4 aa, Homo sapiens
LKAQ

SEQ ID NO: 543 - protein, 4 aa, Homo sapiens
KAQL

SEQ ID NO: 544 - protein, 4 aa, Homo sapiens
AQLW

SEQ ID NO: 545 - protein, 4 aa, Homo sapiens
QLWV

SEQ ID NO: 546 - protein, 4 aa, Homo sapiens
LWVY

SEQ ID NO: 547 - protein, 4 aa, Homo sapiens
WVYL

SEQ ID NO: 548 - protein, 4 aa, Homo sapiens
VYLR

SEQ ID NO: 549 - protein, 4 aa, Homo sapiens
YLRP

SEQ ID NO: 550 - protein, 4 aa, Homo sapiens
LRPV

SEQ ID NO: 551 - protein, 4 aa, Homo sapiens
RPVP

SEQ ID NO: 552 - protein, 4 aa, Homo sapiens
PVPR

SEQ ID NO: 553 - protein, 4 aa, Homo sapiens
VPRP

SEQ ID NO: 554 - protein, 4 aa, Homo sapiens
PRPA

SEQ ID NO: 555 - protein, 4 aa, Homo sapiens
RPAT

SEQ ID NO: 556 - protein, 4 aa, Homo sapiens
PATV

SEQ ID NO: 557 - protein, 4 aa, Homo sapiens
ATVY

SEQ ID NO: 558 - protein, 4 aa, Homo sapiens
TVYL

SEQ ID NO: 559 - protein, 4 aa, Homo sapiens
VYLQ

SEQ ID NO: 560 - protein, 4 aa, Homo sapiens
YLQI

SEQ ID NO: 561 - protein, 4 aa, Homo sapiens
LQIL

SEQ ID NO: 562 - protein, 4 aa, Homo sapiens
QILR

SEQ ID NO: 563 - protein, 4 aa, Homo sapiens
ILRL

SEQ ID NO: 564 - protein, 4 aa, Homo sapiens
RLKP

SEQ ID NO: 565 - protein, 4 aa, Homo sapiens
LKPL

SEQ ID NO: 566 - protein, 4 aa, Homo sapiens
KPLT

SEQ ID NO: 567 - protein, 4 aa, Homo sapiens
PLTG

SEQ ID NO: 568 - protein, 4 aa, Homo sapiens
LTGE

SEQ ID NO: 569 - protein, 4 aa, Homo sapiens
TGEG

SEQ ID NO: 570 - protein, 4 aa, Homo sapiens
GEGT

SEQ ID NO: 571 - protein, 4 aa, Homo sapiens
EGTA

SEQ ID NO: 572 - protein, 4 aa, Homo sapiens
GTAG

SEQ ID NO: 573 - protein, 4 aa, Homo sapiens
TAGG

SEQ ID NO: 574 - protein, 4 aa, Homo sapiens
AGGG

SEQ ID NO: 575 - protein, 4 aa, Homo sapiens
GGGG

SEQ ID NO: 576 - protein, 4 aa, Homo sapiens
GGGR

SEQ ID NO: 577 - protein, 4 aa, Homo sapiens
GGRR

SEQ ID NO: 578 - protein, 4 aa, Homo sapiens
GRRH

SEQ ID NO: 579 - protein, 4 aa, Homo sapiens
RRHI

SEQ ID NO: 580 - protein, 4 aa, Homo sapiens
RHIR

SEQ ID NO: 581 - protein, 4 aa, Homo sapiens
HIRI

SEQ ID NO: 582 - protein, 4 aa, Homo sapiens
IRIR

SEQ ID NO: 583 - protein, 4 aa, Homo sapiens
RIRS

SEQ ID NO: 584 - protein, 4 aa, Homo sapiens
IRSL

SEQ ID NO: 585 - protein, 4 aa, Homo sapiens
RSLK

SEQ ID NO: 586 - protein, 4 aa, Homo sapiens
SLKI

SEQ ID NO: 587 - protein, 4 aa, Homo sapiens
LKIE

SEQ ID NO: 588 - protein, 4 aa, Homo sapiens
KIEL

SEQ ID NO: 589 - protein, 4 aa, Homo sapiens
IELH

SEQ ID NO: 590 - protein, 4 aa, Homo sapiens
ELHS

SEQ ID NO: 591 - protein, 4 aa, Homo sapiens
LHSR

SEQ ID NO: 592 - protein, 4 aa, Homo sapiens
HSRS

SEQ ID NO: 593 - protein, 4 aa, Homo sapiens
SRSG

SEQ ID NO: 594 - protein, 4 aa, Homo sapiens
RSGH

SEQ ID NO: 595 - protein, 4 aa, Homo sapiens
SGHW

SEQ ID NO: 596 - protein, 4 aa, Homo sapiens
GHWQ

SEQ ID NO: 597 - protein, 4 aa, Homo sapiens
HWQS

SEQ ID NO: 598 - protein, 4 aa, Homo sapiens
WQSI

SEQ ID NO: 599 - protein, 4 aa, Homo sapiens
QSID

SEQ ID NO: 600 - protein, 4 aa, Homo sapiens
SIDF

SEQ ID NO: 601 - protein, 4 aa, Homo sapiens
IDFK

SEQ ID NO: 602 - protein, 4 aa, Homo sapiens
DFKQ

SEQ ID NO: 603 - protein, 4 aa, Homo sapiens
FKQV

SEQ ID NO: 604 - protein, 4 aa, Homo sapiens
KQVL

SEQ ID NO: 605 - protein, 4 aa, Homo sapiens
QVLH

SEQ ID NO: 606 - protein, 4 aa, Homo sapiens
VLHS

SEQ ID NO: 607 - protein, 4 aa, Homo sapiens
LHSW

SEQ ID NO: 608 - protein, 4 aa, Homo sapiens
HSWF

SEQ ID NO: 609 - protein, 4 aa, Homo sapiens
SWFR

SEQ ID NO: 610 - protein, 4 aa, Homo sapiens
WFRQ

SEQ ID NO: 611 - protein, 4 aa, Homo sapiens
FRQP

SEQ ID NO: 612 - protein, 4 aa, Homo sapiens
RQPQ

SEQ ID NO: 613 - protein, 4 aa, Homo sapiens
QPQS

SEQ ID NO: 614 - protein, 4 aa, Homo sapiens
PQSN

SEQ ID NO: 615 - protein, 4 aa, Homo sapiens
QSNW

SEQ ID NO: 616 - protein, 4 aa, Homo sapiens
SNWG

SEQ ID NO: 617 - protein, 4 aa, Homo sapiens
NWGI

SEQ ID NO: 618 - protein, 4 aa, Homo sapiens
WGIE

SEQ ID NO: 619 - protein, 4 aa, Homo sapiens
GIEI

SEQ ID NO: 620 - protein, 4 aa, Homo sapiens
IEIN

SEQ ID NO: 621 - protein, 4 aa, Homo sapiens
EINA

SEQ ID NO: 622 - protein, 4 aa, Homo sapiens
INAF

SEQ ID NO: 623 - protein, 4 aa, Homo sapiens
NAFD

SEQ ID NO: 624 - protein, 4 aa, Homo sapiens
AFDP

SEQ ID NO: 625 - protein, 4 aa, Homo sapiens
FDPS

SEQ ID NO: 626 - protein, 4 aa, Homo sapiens
DPSG

SEQ ID NO: 627 - protein, 4 aa, Homo sapiens
PSGT

SEQ ID NO: 628 - protein, 4 aa, Homo sapiens
SGTD

SEQ ID NO: 629 - protein, 4 aa, Homo sapiens
GTDL

SEQ ID NO: 630 - protein, 4 aa, Homo sapiens
TDLA

SEQ ID NO: 631 - protein, 4 aa, Homo sapiens
DLAV

SEQ ID NO: 632 - protein, 4 aa, Homo sapiens
LAVT

SEQ ID NO: 633 - protein, 4 aa, Homo sapiens
AVTS

SEQ ID NO: 634 - protein, 4 aa, Homo sapiens
VTSL

SEQ ID NO: 635 - protein, 4 aa, Homo sapiens
TSLG

SEQ ID NO: 636 - protein, 4 aa, Homo sapiens
SLGP

SEQ ID NO: 637 - protein, 4 aa, Homo sapiens
LGPG

SEQ ID NO: 638 - protein, 4 aa, Homo sapiens
GPGA

SEQ ID NO: 639 - protein, 4 aa, Homo sapiens
PGAE

SEQ ID NO: 640 - protein, 4 aa, Homo sapiens
GAEG

SEQ ID NO: 641 - protein, 4 aa, Homo sapiens
AEGL

SEQ ID NO: 642 - protein, 4 aa, Homo sapiens
EGLH

SEQ ID NO: 643 - protein, 4 aa, Homo sapiens
GLHP

SEQ ID NO: 644 - protein, 4 aa, Homo sapiens
LHPF

SEQ ID NO: 645 - protein, 4 aa, Homo sapiens
HPFM

SEQ ID NO: 646 - protein, 4 aa, Homo sapiens
PFME

SEQ ID NO: 647 - protein, 4 aa, Homo sapiens
FMEL

SEQ ID NO: 648 - protein, 4 aa, Homo sapiens
MELR

SEQ ID NO: 649 - protein, 4 aa, Homo sapiens
ELRV

SEQ ID NO: 650 - protein, 4 aa, Homo sapiens
LRVL

SEQ ID NO: 651 - protein, 4 aa, Homo sapiens
RVLE

SEQ ID NO: 652 - protein, 4 aa, Homo sapiens
VLEN

SEQ ID NO: 653 - protein, 4 aa, Homo sapiens
LENT

SEQ ID NO: 654 - protein, 4 aa, Homo sapiens
ENTK

SEQ ID NO: 655 - protein, 4 aa, Homo sapiens
NTKR

SEQ ID NO: 656 - protein, 4 aa, Homo sapiens
TKRS

SEQ ID NO: 657 - protein, 4 aa, Homo sapiens
KRSR

SEQ ID NO: 658 - protein, 4 aa, Homo sapiens
RSRR

SEQ ID NO: 659 - protein, 4 aa, Homo sapiens
SRRN

SEQ ID NO: 660 - protein, 4 aa, Homo sapiens
RRNL

SEQ ID NO: 661 - protein, 4 aa, Homo sapiens
RNLG

SEQ ID NO: 662 - protein, 4 aa, Homo sapiens
NLGL

SEQ ID NO: 663 - protein, 4 aa, Homo sapiens
LGLD

SEQ ID NO: 664 - protein, 4 aa, Homo sapiens
GLDC

SEQ ID NO: 665 - protein, 4 aa, Homo sapiens
LDCD

SEQ ID NO: 666 - protein, 4 aa, Homo sapiens
DCDE

SEQ ID NO: 667 - protein, 4 aa, Homo sapiens
CDEH

SEQ ID NO: 668 - protein, 4 aa, Homo sapiens
DEHS

SEQ ID NO: 669 - protein, 4 aa, Homo sapiens
EHSS

SEQ ID NO: 670 - protein, 4 aa, Homo sapiens
HSSE

SEQ ID NO: 671 - protein, 4 aa, Homo sapiens
SSES

SEQ ID NO: 672 - protein, 4 aa, Homo sapiens
SESR

SEQ ID NO: 673 - protein, 4 aa, Homo sapiens
ESRC

SEQ ID NO: 674 - protein, 4 aa, Homo sapiens
SRCC

SEQ ID NO: 675 - protein, 4 aa, Homo sapiens
RCCR

SEQ ID NO: 676 - protein, 4 aa, Homo sapiens
CCRY

SEQ ID NO: 677 - protein, 4 aa, Homo sapiens
CRYP

SEQ ID NO: 678 - protein, 4 aa, Homo sapiens
RYPL

SEQ ID NO: 679 - protein, 4 aa, Homo sapiens
YPLT

SEQ ID NO: 680 - protein, 4 aa, Homo sapiens
PLTV

SEQ ID NO: 681 - protein, 4 aa, Homo sapiens
LTVD

SEQ ID NO: 682 - protein, 4 aa, Homo sapiens
TVDF

SEQ ID NO: 683 - protein, 4 aa, Homo sapiens
VDFE

SEQ ID NO: 684 - protein, 4 aa, Homo sapiens
DFEA

SEQ ID NO: 685 - protein, 4 aa, Homo sapiens
FEAF

SEQ ID NO: 686 - protein, 4 aa, Homo sapiens
EAFG

SEQ ID NO: 687 - protein, 4 aa, Homo sapiens
AFGW

SEQ ID NO: 688 - protein, 4 aa, Homo sapiens
FGWD

SEQ ID NO: 689 - protein, 4 aa, Homo sapiens
GWDW

SEQ ID NO: 690 - protein, 4 aa, Homo sapiens
WDWI

SEQ ID NO: 691 - protein, 4 aa, Homo sapiens
DWII

SEQ ID NO: 692 - protein, 4 aa, Homo sapiens
WIIA

SEQ ID NO: 693 - protein, 4 aa, Homo sapiens
IIAP

SEQ ID NO: 694 - protein, 4 aa, Homo sapiens
IAPK

SEQ ID NO: 695 - protein, 4 aa, Homo sapiens
APKR

SEQ ID NO: 696 - protein, 4 aa, Homo sapiens
PKRY

SEQ ID NO: 697 - protein, 4 aa, Homo sapiens
KRYK

SEQ ID NO: 698 - protein, 4 aa, Homo sapiens
RYKA

SEQ ID NO: 699 - protein, 4 aa, Homo sapiens
YKAN

SEQ ID NO: 700 - protein, 4 aa, Homo sapiens
KANY

SEQ ID NO: 701 - protein, 4 aa, Homo sapiens
ANYC

SEQ ID NO: 702 - protein, 4 aa, Homo sapiens
NYCS

SEQ ID NO: 703 - protein, 4 aa, Homo sapiens
YCSG

SEQ ID NO: 704 - protein, 4 aa, Homo sapiens
CSGQ

SEQ ID NO: 705 - protein, 4 aa, Homo sapiens
SGQC

SEQ ID NO: 706 - protein, 4 aa, Homo sapiens
GQCE

SEQ ID NO: 707 - protein, 4 aa, Homo sapiens
QCEY

SEQ ID NO: 708 - protein, 4 aa, Homo sapiens
CEYM

SEQ ID NO: 709 - protein, 4 aa, Homo sapiens
EYMF

SEQ ID NO: 710 - protein, 4 aa, Homo sapiens
YMFM

SEQ ID NO: 711 - protein, 4 aa, Homo sapiens
MFMQ

SEQ ID NO: 712 - protein, 4 aa, Homo sapiens
FMQK

SEQ ID NO: 713 - protein, 4 aa, Homo sapiens
MQKY

SEQ ID NO: 714 - protein, 4 aa, Homo sapiens
QKYP

SEQ ID NO: 715 - protein, 4 aa, Homo sapiens
KYPH

SEQ ID NO: 716 - protein, 4 aa, Homo sapiens
YPHT

SEQ ID NO: 717 - protein, 4 aa, Homo sapiens
PHTH

SEQ ID NO: 718 - protein, 4 aa, Homo sapiens
HTHL

SEQ ID NO: 719 - protein, 4 aa, Homo sapiens
THLV

SEQ ID NO: 720 - protein, 4 aa, Homo sapiens
HLVQ

SEQ ID NO: 721 - protein, 4 aa, Homo sapiens
LVQQ

SEQ ID NO: 722 - protein, 4 aa, Homo sapiens
VQQA

SEQ ID NO: 723 - protein, 4 aa, Homo sapiens
QQAN

SEQ ID NO: 724 - protein, 4 aa, Homo sapiens
QANP

SEQ ID NO: 725 - protein, 4 aa, Homo sapiens
ANPR

SEQ ID NO: 726 - protein, 4 aa, Homo sapiens
NPRG

SEQ ID NO: 727 - protein, 4 aa, Homo sapiens
PRGS

SEQ ID NO: 728 - protein, 4 aa, Homo sapiens
RGSA

SEQ ID NO: 729 - protein, 4 aa, Homo sapiens
GSAG

SEQ ID NO: 730 - protein, 4 aa, Homo sapiens
SAGP

SEQ ID NO: 731 - protein, 4 aa, Homo sapiens
AGPC

SEQ ID NO: 732 - protein, 4 aa, Homo sapiens
GPCC

SEQ ID NO: 733 - protein, 4 aa, Homo sapiens
PCCT

SEQ ID NO: 734 - protein, 4 aa, Homo sapiens
CCTP

SEQ ID NO: 735 - protein, 4 aa, Homo sapiens
CTPT

SEQ ID NO: 736 - protein, 4 aa, Homo sapiens
TPTK

SEQ ID NO: 737 - protein, 4 aa, Homo sapiens
PTKM

SEQ ID NO: 738 - protein, 4 aa, Homo sapiens
TKMS

SEQ ID NO: 739 - protein, 4 aa, Homo sapiens
KMSP

SEQ ID NO: 740 - protein, 4 aa, Homo sapiens
MSPI

SEQ ID NO: 741 - protein, 4 aa, Homo sapiens
SPIN

SEQ ID NO: 742 - protein, 4 aa, Homo sapiens
PINM

SEQ ID NO: 743 - protein, 4 aa, Homo sapiens
INML

SEQ ID NO: 744 - protein, 4 aa, Homo sapiens
NMLY

SEQ ID NO: 745 - protein, 4 aa, Homo sapiens
MLYF

SEQ ID NO: 746 - protein, 4 aa, Homo sapiens
LYFN

SEQ ID NO: 747 - protein, 4 aa, Homo sapiens
YFND

SEQ ID NO: 748 - protein, 4 aa, Homo sapiens
FNDK

SEQ ID NO: 749 - protein, 4 aa, Homo sapiens
NDKQ

SEQ ID NO: 750 - protein, 4 aa, Homo sapiens
DKQQ

SEQ ID NO: 751 - protein, 4 aa, Homo sapiens
KQQI

SEQ ID NO: 752 - protein, 4 aa, Homo sapiens
QQII

SEQ ID NO: 753 - protein, 4 aa, Homo sapiens
QIIY

SEQ ID NO: 754 - protein, 4 aa, Homo sapiens
IIYG

SEQ ID NO: 755 - protein, 4 aa, Homo sapiens
IYGK

SEQ ID NO: 756 - protein, 4 aa, Homo sapiens
YGKI

SEQ ID NO: 757 - protein, 4 aa, Homo sapiens
GKIP

SEQ ID NO: 758 - protein, 4 aa, Homo sapiens
KIPG

SEQ ID NO: 759 - protein, 4 aa, Homo sapiens
IPGM

SEQ ID NO: 760 - protein, 4 aa, Homo sapiens
PGMV

SEQ ID NO: 761 - protein, 4 aa, Homo sapiens
GMVV

SEQ ID NO: 762 - protein, 4 aa, Homo sapiens
MVVD

SEQ ID NO: 763 - protein, 4 aa, Homo sapiens
VVDR

SEQ ID NO: 764 - protein, 4 aa, Homo sapiens
VDRC

SEQ ID NO: 765 - protein, 4 aa, Homo sapiens
DRCG

SEQ ID NO: 766 - protein, 4 aa, Homo sapiens
RCGC

SEQ ID NO: 767 - protein, 4 aa, Homo sapiens
CGCS

SEQ ID NO: 768 - protein, 5 aa, Homo sapiens
MVLAA

SEQ ID NO: 769 - protein, 5 aa, Homo sapiens
VLAAP

SEQ ID NO: 770 - protein, 5 aa, Homo sapiens
LAAPL

SEQ ID NO: 771 - protein, 5 aa, Homo sapiens
AAPLL

SEQ ID NO: 772 - protein, 5 aa, Homo sapiens
APLLL

SEQ ID NO: 773 - protein, 5 aa, Homo sapiens
PLLLG

SEQ ID NO: 774 - protein, 5 aa, Homo sapiens
LLLGF

SEQ ID NO: 775 - protein, 5 aa, Homo sapiens
LLGFL

SEQ ID NO: 776 - protein, 5 aa, Homo sapiens
LGFLL

SEQ ID NO: 777 - protein, 5 aa, Homo sapiens
GFLLL

SEQ ID NO: 778 - protein, 5 aa, Homo sapiens
FLLLA

SEQ ID NO: 779 - protein, 5 aa, Homo sapiens
LLLAL

SEQ ID NO: 780 - protein, 5 aa, Homo sapiens
LLALE

SEQ ID NO: 781 - protein, 5 aa, Homo sapiens
LALEL

SEQ ID NO: 782 - protein, 5 aa, Homo sapiens
ALELR

SEQ ID NO: 783 - protein, 5 aa, Homo sapiens
LELRP

SEQ ID NO: 784 - protein, 5 aa, Homo sapiens
ELRPR

SEQ ID NO: 785 - protein, 5 aa, Homo sapiens
LRPRG

SEQ ID NO: 786 - protein, 5 aa, Homo sapiens
RPRGE

SEQ ID NO: 787 - protein, 5 aa, Homo sapiens
PRGEA

SEQ ID NO: 788 - protein, 5 aa, Homo sapiens
RGEAA

SEQ ID NO: 789 - protein, 5 aa, Homo sapiens
GEAAE

SEQ ID NO: 790 - protein, 5 aa, Homo sapiens
EAAEG

SEQ ID NO: 791 - protein, 5 aa, Homo sapiens
AAEGP

SEQ ID NO: 792 - protein, 5 aa, Homo sapiens
AEGPA

SEQ ID NO: 793 - protein, 5 aa, Homo sapiens
EGPAA

SEQ ID NO: 794 - protein, 5 aa, Homo sapiens
GPAAA

SEQ ID NO: 795 - protein, 5 aa, Homo sapiens
PAAAA

SEQ ID NO: 796 - protein, 5 aa, Homo sapiens
AAAAA

SEQ ID NO: 797 - protein, 5 aa, Homo sapiens
AAAAG

SEQ ID NO: 798 - protein, 5 aa, Homo sapiens
AAAGV

SEQ ID NO: 799 - protein, 5 aa, Homo sapiens
AAGVG

SEQ ID NO: 800 - protein, 5 aa, Homo sapiens
AGVGG

SEQ ID NO: 801 - protein, 5 aa, Homo sapiens
GVGGE

SEQ ID NO: 802 - protein, 5 aa, Homo sapiens
VGGER

SEQ ID NO: 803 - protein, 5 aa, Homo sapiens
GGERS

SEQ ID NO: 804 - protein, 5 aa, Homo sapiens
GERSS

SEQ ID NO: 805 - protein, 5 aa, Homo sapiens
ERSSR

SEQ ID NO: 806 - protein, 5 aa, Homo sapiens
RSSRP

SEQ ID NO: 807 - protein, 5 aa, Homo sapiens
SSRPA

SEQ ID NO: 808 - protein, 5 aa, Homo sapiens
SRPAP

SEQ ID NO: 809 - protein, 5 aa, Homo sapiens
RPAPS

SEQ ID NO: 810 - protein, 5 aa, Homo sapiens
PAPSV

SEQ ID NO: 811 - protein, 5 aa, Homo sapiens
APSVA

SEQ ID NO: 812 - protein, 5 aa, Homo sapiens
PSVAP

SEQ ID NO: 813 - protein, 5 aa, Homo sapiens
SVAPE

SEQ ID NO: 814 - protein, 5 aa, Homo sapiens
VAPEP

SEQ ID NO: 815 - protein, 5 aa, Homo sapiens
APEPD

SEQ ID NO: 816 - protein, 5 aa, Homo sapiens
PEPDG

SEQ ID NO: 817 - protein, 5 aa, Homo sapiens
EPDGC

SEQ ID NO: 818 - protein, 5 aa, Homo sapiens
PDGCP

SEQ ID NO: 819 - protein, 5 aa, Homo sapiens
DGCPV

SEQ ID NO: 820 - protein, 5 aa, Homo sapiens
GCPVC

SEQ ID NO: 821 - protein, 5 aa, Homo sapiens
CPVCV

SEQ ID NO: 822 - protein, 5 aa, Homo sapiens
PVCVW

SEQ ID NO: 823 - protein, 5 aa, Homo sapiens
VCVWR

SEQ ID NO: 824 - protein, 5 aa, Homo sapiens
CVWRQ

SEQ ID NO: 825 - protein, 5 aa, Homo sapiens
VWRQH

SEQ ID NO: 826 - protein, 5 aa, Homo sapiens
WRQHS

SEQ ID NO: 827 - protein, 5 aa, Homo sapiens
RQHSR

SEQ ID NO: 828 - protein, 5 aa, Homo sapiens
QHSRE

SEQ ID NO: 829 - protein, 5 aa, Homo sapiens
HSREL

SEQ ID NO: 830 - protein, 5 aa, Homo sapiens
SRELR

SEQ ID NO: 831 - protein, 5 aa, Homo sapiens
RELRL

SEQ ID NO: 832 - protein, 5 aa, Homo sapiens
ELRLE

SEQ ID NO: 833 - protein, 5 aa, Homo sapiens
LRLES

SEQ ID NO: 834 - protein, 5 aa, Homo sapiens
RLESI

SEQ ID NO: 835 - protein, 5 aa, Homo sapiens
LESIK

SEQ ID NO: 836 - protein, 5 aa, Homo sapiens
ESIKS

SEQ ID NO: 837 - protein, 5 aa, Homo sapiens
SIKSQ

SEQ ID NO: 838 - protein, 5 aa, Homo sapiens
IKSQI

SEQ ID NO: 839 - protein, 5 aa, Homo sapiens
KSQIL

SEQ ID NO: 840 - protein, 5 aa, Homo sapiens
SQILS

SEQ ID NO: 841 - protein, 5 aa, Homo sapiens
QILSK

SEQ ID NO: 842 - protein, 5 aa, Homo sapiens
ILSKL

SEQ ID NO: 843 - protein, 5 aa, Homo sapiens
LSKLR

SEQ ID NO: 844 - protein, 5 aa, Homo sapiens
SKLRL

SEQ ID NO: 845 - protein, 5 aa, Homo sapiens
KLRLK

SEQ ID NO: 846 - protein, 5 aa, Homo sapiens
LRLKE

SEQ ID NO: 847 - protein, 5 aa, Homo sapiens
RLKEA

SEQ ID NO: 848 - protein, 5 aa, Homo sapiens
LKEAP

SEQ ID NO: 849 - protein, 5 aa, Homo sapiens
KEAPN

SEQ ID NO: 850 - protein, 5 aa, Homo sapiens
EAPNI

SEQ ID NO: 851 - protein, 5 aa, Homo sapiens
APNIS

SEQ ID NO: 852 - protein, 5 aa, Homo sapiens
PNISR

SEQ ID NO: 853 - protein, 5 aa, Homo sapiens
NISRE

SEQ ID NO: 854 - protein, 5 aa, Homo sapiens
ISREV

SEQ ID NO: 855 - protein, 5 aa, Homo sapiens
SREVV

SEQ ID NO: 856 - protein, 5 aa, Homo sapiens
REVVK

SEQ ID NO: 857 - protein, 5 aa, Homo sapiens
EVVKQ

SEQ ID NO: 858 - protein, 5 aa, Homo sapiens
VVKQL

SEQ ID NO: 859 - protein, 5 aa, Homo sapiens
VKQLL

SEQ ID NO: 860 - protein, 5 aa, Homo sapiens
KQLLP

SEQ ID NO: 861 - protein, 5 aa, Homo sapiens
QLLPK

SEQ ID NO: 862 - protein, 5 aa, Homo sapiens
LLPKA

SEQ ID NO: 863 - protein, 5 aa, Homo sapiens
LPKAP

SEQ ID NO: 864 - protein, 5 aa, Homo sapiens
PKAPP

SEQ ID NO: 865 - protein, 5 aa, Homo sapiens
KAPPL

SEQ ID NO: 866 - protein, 5 aa, Homo sapiens
APPLQ

SEQ ID NO: 867 - protein, 5 aa, Homo sapiens
PPLQQ

SEQ ID NO: 868 - protein, 5 aa, Homo sapiens
PLQQI

SEQ ID NO: 869 - protein, 5 aa, Homo sapiens
LQQIL

SEQ ID NO: 870 - protein, 5 aa, Homo sapiens
QQILD

SEQ ID NO: 871 - protein, 5 aa, Homo sapiens
QILDL

SEQ ID NO: 872 - protein, 5 aa, Homo sapiens
ILDLH

SEQ ID NO: 873 - protein, 5 aa, Homo sapiens
LDLHD

SEQ ID NO: 874 - protein, 5 aa, Homo sapiens
DLHDF

SEQ ID NO: 875 - protein, 5 aa, Homo sapiens
LHDFQ

SEQ ID NO: 876 - protein, 5 aa, Homo sapiens
HDFQG

SEQ ID NO: 877 - protein, 5 aa, Homo sapiens
DFQGD

SEQ ID NO: 878 - protein, 5 aa, Homo sapiens
FQGDA

SEQ ID NO: 879 - protein, 5 aa, Homo sapiens
QGDAL

SEQ ID NO: 880 - protein, 5 aa, Homo sapiens
GDALQ

SEQ ID NO: 881 - protein, 5 aa, Homo sapiens
DALQP

SEQ ID NO: 882 - protein, 5 aa, Homo sapiens
ALQPE

SEQ ID NO: 883 - protein, 5 aa, Homo sapiens
LQPED

SEQ ID NO: 884 - protein, 5 aa, Homo sapiens
QPEDF

SEQ ID NO: 885 - protein, 5 aa, Homo sapiens
PEDFL

SEQ ID NO: 886 - protein, 5 aa, Homo sapiens
EDFLE

SEQ ID NO: 887 - protein, 5 aa, Homo sapiens
DFLEE

SEQ ID NO: 888 - protein, 5 aa, Homo sapiens
FLEED

SEQ ID NO: 889 - protein, 5 aa, Homo sapiens
LEEDE

SEQ ID NO: 890 - protein, 5 aa, Homo sapiens
EEDEY

SEQ ID NO: 891 - protein, 5 aa, Homo sapiens
EDEYH

SEQ ID NO: 892 - protein, 5 aa, Homo sapiens
DEYHA

SEQ ID NO: 893 - protein, 5 aa, Homo sapiens
EYHAT

SEQ ID NO: 894 - protein, 5 aa, Homo sapiens
YHATT

SEQ ID NO: 895 - protein, 5 aa, Homo sapiens
HATTE

SEQ ID NO: 896 - protein, 5 aa, Homo sapiens
ATTET

SEQ ID NO: 897 - protein, 5 aa, Homo sapiens
TTETV

SEQ ID NO: 898 - protein, 5 aa, Homo sapiens
TETVI

SEQ ID NO: 899 - protein, 5 aa, Homo sapiens
ETVIS

SEQ ID NO: 900 - protein, 5 aa, Homo sapiens
TVISM

SEQ ID NO: 901 - protein, 5 aa, Homo sapiens
VISMA

SEQ ID NO: 902 - protein, 5 aa, Homo sapiens
ISMAQ

SEQ ID NO: 903 - protein, 5 aa, Homo sapiens
SMAQE

SEQ ID NO: 904 - protein, 5 aa, Homo sapiens
MAQET

SEQ ID NO: 905 - protein, 5 aa, Homo sapiens
AQETD

SEQ ID NO: 906 - protein, 5 aa, Homo sapiens
QETDP

SEQ ID NO: 907 - protein, 5 aa, Homo sapiens
ETDPA

SEQ ID NO: 908 - protein, 5 aa, Homo sapiens
TDPAV

SEQ ID NO: 909 - protein, 5 aa, Homo sapiens
DPAVQ

SEQ ID NO: 910 - protein, 5 aa, Homo sapiens
PAVQT

SEQ ID NO: 911 - protein, 5 aa, Homo sapiens
AVQTD

SEQ ID NO: 912 - protein, 5 aa, Homo sapiens
VQTDG

SEQ ID NO: 913 - protein, 5 aa, Homo sapiens
QTDGS

SEQ ID NO: 914 - protein, 5 aa, Homo sapiens
TDGSP

SEQ ID NO: 915 - protein, 5 aa, Homo sapiens
DGSPL

SEQ ID NO: 916 - protein, 5 aa, Homo sapiens
GSPLC

SEQ ID NO: 917 - protein, 5 aa, Homo sapiens
SPLCC

SEQ ID NO: 918 - protein, 5 aa, Homo sapiens
PLCCH

SEQ ID NO: 919 - protein, 5 aa, Homo sapiens
LCCHF

SEQ ID NO: 920 - protein, 5 aa, Homo sapiens
CCHFH

SEQ ID NO: 921 - protein, 5 aa, Homo sapiens
CHFHF

SEQ ID NO: 922 - protein, 5 aa, Homo sapiens
HFHFS

SEQ ID NO: 923 - protein, 5 aa, Homo sapiens
FHFSP

SEQ ID NO: 924 - protein, 5 aa, Homo sapiens
HFSPK

SEQ ID NO: 925 - protein, 5 aa, Homo sapiens
FSPKV

SEQ ID NO: 926 - protein, 5 aa, Homo sapiens
SPKVM

SEQ ID NO: 927 - protein, 5 aa, Homo sapiens
PKVMF

SEQ ID NO: 928 - protein, 5 aa, Homo sapiens
KVMFT

SEQ ID NO: 929 - protein, 5 aa, Homo sapiens
VMFTK

SEQ ID NO: 930 - protein, 5 aa, Homo sapiens
MFTKV

SEQ ID NO: 931 - protein, 5 aa, Homo sapiens
FTKVL

SEQ ID NO: 932 - protein, 5 aa, Homo sapiens
TKVLK

SEQ ID NO: 933 - protein, 5 aa, Homo sapiens
KVLKA

SEQ ID NO: 934 - protein, 5 aa, Homo sapiens
VLKAQ

SEQ ID NO: 935 - protein, 5 aa, Homo sapiens
LKAQL

SEQ ID NO: 936 - protein, 5 aa, Homo sapiens
KAQLW

SEQ ID NO: 937 - protein, 5 aa, Homo sapiens
AQLWV

SEQ ID NO: 938 - protein, 5 aa, Homo sapiens
QLWVY

SEQ ID NO: 939 - protein, 5 aa, Homo sapiens
LWVYL

SEQ ID NO: 940 - protein, 5 aa, Homo sapiens
WVYLR

SEQ ID NO: 941 - protein, 5 aa, Homo sapiens
VYLRP

SEQ ID NO: 942 - protein, 5 aa, Homo sapiens
YLRPV

SEQ ID NO: 943 - protein, 5 aa, Homo sapiens
LRPVP

SEQ ID NO: 944 - protein, 5 aa, Homo sapiens
RPVPR

SEQ ID NO: 945 - protein, 5 aa, Homo sapiens
PVPRP

SEQ ID NO: 946 - protein, 5 aa, Homo sapiens
VPRPA

SEQ ID NO: 947 - protein, 5 aa, Homo sapiens
PRPAT

SEQ ID NO: 948 - protein, 5 aa, Homo sapiens
RPATV

SEQ ID NO: 949 - protein, 5 aa, Homo sapiens
PATVY

SEQ ID NO: 950 - protein, 5 aa, Homo sapiens
ATVYL

SEQ ID NO: 951 - protein, 5 aa, Homo sapiens
TVYLQ

SEQ ID NO: 952 - protein, 5 aa, Homo sapiens
VYLQI

SEQ ID NO: 953 - protein, 5 aa, Homo sapiens
YLQIL

SEQ ID NO: 954 - protein, 5 aa, Homo sapiens
LQILR

SEQ ID NO: 955 - protein, 5 aa, Homo sapiens
QILRL

SEQ ID NO: 956 - protein, 5 aa, Homo sapiens
ILRLK

SEQ ID NO: 957 - protein, 5 aa, Homo sapiens
LRLKP

SEQ ID NO: 958 - protein, 5 aa, Homo sapiens
RLKPL

SEQ ID NO: 959 - protein, 5 aa, Homo sapiens
LKPLT

SEQ ID NO: 960 - protein, 5 aa, Homo sapiens
KPLTG

SEQ ID NO: 961 - protein, 5 aa, Homo sapiens
PLTGE

SEQ ID NO: 962 - protein, 5 aa, Homo sapiens
LTGEG

SEQ ID NO: 963 - protein, 5 aa, Homo sapiens
TGEGT

SEQ ID NO: 964 - protein, 5 aa, Homo sapiens
GEGTA

SEQ ID NO: 965 - protein, 5 aa, Homo sapiens
EGTAG

SEQ ID NO: 966 - protein, 5 aa, Homo sapiens
GTAGG

SEQ ID NO: 967 - protein, 5 aa, Homo sapiens
TAGGG

SEQ ID NO: 968 - protein, 5 aa, Homo sapiens
AGGGG

SEQ ID NO: 969 - protein, 5 aa, Homo sapiens
GGGGG

SEQ ID NO: 970 - protein, 5 aa, Homo sapiens
GGGGR

SEQ ID NO: 971 - protein, 5 aa, Homo sapiens
GGGRR

SEQ ID NO: 972 - protein, 5 aa, Homo sapiens
GGRRH

SEQ ID NO: 973 - protein, 5 aa, Homo sapiens
GRRHI

SEQ ID NO: 974 - protein, 5 aa, Homo sapiens
RRHIR

SEQ ID NO: 975 - protein, 5 aa, Homo sapiens
RHIRI

SEQ ID NO: 976 - protein, 5 aa, Homo sapiens
HIRIR

SEQ ID NO: 977 - protein, 5 aa, Homo sapiens
IRIRS

SEQ ID NO: 978 - protein, 5 aa, Homo sapiens
RIRSL

SEQ ID NO: 979 - protein, 5 aa, Homo sapiens
IRSLK

SEQ ID NO: 980 - protein, 5 aa, Homo sapiens
RSLKI

SEQ ID NO: 981 - protein, 5 aa, Homo sapiens
SLKIE

SEQ ID NO: 982 - protein, 5 aa, Homo sapiens
LKIEL

SEQ ID NO: 983 - protein, 5 aa, Homo sapiens
KIELH

SEQ ID NO: 984 - protein, 5 aa, Homo sapiens
IELHS

SEQ ID NO: 985 - protein, 5 aa, Homo sapiens
ELHSR

SEQ ID NO: 986 - protein, 5 aa, Homo sapiens
LHSRS

SEQ ID NO: 987 - protein, 5 aa, Homo sapiens
HSRSG

SEQ ID NO: 988 - protein, 5 aa, Homo sapiens
SRSGH

SEQ ID NO: 989 - protein, 5 aa, Homo sapiens
RSGHW

SEQ ID NO: 990 - protein, 5 aa, Homo sapiens
SGHWQ

SEQ ID NO: 991 - protein, 5 aa, Homo sapiens
GHWQS

SEQ ID NO: 992 - protein, 5 aa, Homo sapiens
HWQSI

SEQ ID NO: 993 - protein, 5 aa, Homo sapiens
WQSID

SEQ ID NO: 994 - protein, 5 aa, Homo sapiens
QSIDF

SEQ ID NO: 995 - protein, 5 aa, Homo sapiens
SIDFK

SEQ ID NO: 996 - protein, 5 aa, Homo sapiens
IDFKQ

SEQ ID NO: 997 - protein, 5 aa, Homo sapiens
DFKQV

SEQ ID NO: 998 - protein, 5 aa, Homo sapiens
FKQVL

SEQ ID NO: 999 - protein, 5 aa, Homo sapiens
KQVLH

SEQ ID NO: 1000 - protein, 5 aa, Homo sapiens
QVLHS

SEQ ID NO: 1001 - protein, 5 aa, Homo sapiens
VLHSW

SEQ ID NO: 1002 - protein, 5 aa, Homo sapiens
LHSWF

SEQ ID NO: 1003 - protein, 5 aa, Homo sapiens
HSWFR

SEQ ID NO: 1004 - protein, 5 aa, Homo sapiens
SWFRQ

SEQ ID NO: 1005 - protein, 5 aa, Homo sapiens
WFRQP

SEQ ID NO: 1006 - protein, 5 aa, Homo sapiens
FRQPQ

SEQ ID NO: 1007 - protein, 5 aa, Homo sapiens
RQPQS

SEQ ID NO: 1008 - protein, 5 aa, Homo sapiens
QPQSN

SEQ ID NO: 1009 - protein, 5 aa, Homo sapiens
PQSNW

SEQ ID NO: 1010 - protein, 5 aa, Homo sapiens
QSNWG

SEQ ID NO: 1011 - protein, 5 aa, Homo sapiens
SNWGI

SEQ ID NO: 1012 - protein, 5 aa, Homo sapiens
NWGIE

SEQ ID NO: 1013 - protein, 5 aa, Homo sapiens
WGIEI

SEQ ID NO: 1014 - protein, 5 aa, Homo sapiens
GIEIN

SEQ ID NO: 1015 - protein, 5 aa, Homo sapiens
IEINA

SEQ ID NO: 1016 - protein, 5 aa, Homo sapiens
EINAF

SEQ ID NO: 1017 - protein, 5 aa, Homo sapiens
INAFD

SEQ ID NO: 1018 - protein, 5 aa, Homo sapiens
NAFDP

SEQ ID NO: 1019 - protein, 5 aa, Homo sapiens
AFDPS

SEQ ID NO: 1020 - protein, 5 aa, Homo sapiens
FDPSG

SEQ ID NO: 1021 - protein, 5 aa, Homo sapiens
DPSGT

SEQ ID NO: 1022 - protein, 5 aa, Homo sapiens
PSGTD

SEQ ID NO: 1023 - protein, 5 aa, Homo sapiens
SGTDL

SEQ ID NO: 1024 - protein, 5 aa, Homo sapiens
GTDLA

SEQ ID NO: 1025 - protein, 5 aa, Homo sapiens
TDLAV

SEQ ID NO: 1026 - protein, 5 aa, Homo sapiens
DLAVT

SEQ ID NO: 1027 - protein, 5 aa, Homo sapiens
LAVTS

SEQ ID NO: 1028 - protein, 5 aa, Homo sapiens
AVTSL

SEQ ID NO: 1029 - protein, 5 aa, Homo sapiens
VTSLG

SEQ ID NO: 1030 - protein, 5 aa, Homo sapiens
TSLGP

SEQ ID NO: 1031 - protein, 5 aa, Homo sapiens
SLGPG

SEQ ID NO: 1032 - protein, 5 aa, Homo sapiens
LGPGA

SEQ ID NO: 1033 - protein, 5 aa, Homo sapiens
GPGAE

SEQ ID NO: 1034 - protein, 5 aa, Homo sapiens
PGAEG

SEQ ID NO: 1035 - protein, 5 aa, Homo sapiens
GAEGL

SEQ ID NO: 1036 - protein, 5 aa, Homo sapiens
AEGLH

SEQ ID NO: 1037 - protein, 5 aa, Homo sapiens
EGLHP

SEQ ID NO: 1038 - protein, 5 aa, Homo sapiens
GLHPF

SEQ ID NO: 1039 - protein, 5 aa, Homo sapiens
LHPFM

SEQ ID NO: 1040 - protein, 5 aa, Homo sapiens
HPFME

SEQ ID NO: 1041 - protein, 5 aa, Homo sapiens
PFMEL

SEQ ID NO: 1042 - protein, 5 aa, Homo sapiens
FMELR

SEQ ID NO: 1043 - protein, 5 aa, Homo sapiens
MELRV

SEQ ID NO: 1044 - protein, 5 aa, Homo sapiens
ELRVL

SEQ ID NO: 1045 - protein, 5 aa, Homo sapiens
LRVLE

SEQ ID NO: 1046 - protein, 5 aa, Homo sapiens
RVLEN

SEQ ID NO: 1047 - protein, 5 aa, Homo sapiens
VLENT

SEQ ID NO: 1048 - protein, 5 aa, Homo sapiens
LENTK

SEQ ID NO: 1049 - protein, 5 aa, Homo sapiens
ENTKR

SEQ ID NO: 1050 - protein, 5 aa, Homo sapiens
NTKRS

SEQ ID NO: 1051 - protein, 5 aa, Homo sapiens
TKRSR

SEQ ID NO: 1052 - protein, 5 aa, Homo sapiens
KRSRR

SEQ ID NO: 1053 - protein, 5 aa, Homo sapiens
RSRRN

SEQ ID NO: 1054 - protein, 5 aa, Homo sapiens
SRRNL

SEQ ID NO: 1055 - protein, 5 aa, Homo sapiens
RRNLG

SEQ ID NO: 1056 - protein, 5 aa, Homo sapiens
RNLGL

SEQ ID NO: 1057 - protein, 5 aa, Homo sapiens
NLGLD

SEQ ID NO: 1058 - protein, 5 aa, Homo sapiens
LGLDC

SEQ ID NO: 1059 - protein, 5 aa, Homo sapiens
GLDCD

SEQ ID NO: 1060 - protein, 5 aa, Homo sapiens
LDCDE

SEQ ID NO: 1061 - protein, 5 aa, Homo sapiens
DCDEH

SEQ ID NO: 1062 - protein, 5 aa, Homo sapiens
CDEHS

SEQ ID NO: 1063 - protein, 5 aa, Homo sapiens
DEHSS

SEQ ID NO: 1064 - protein, 5 aa, Homo sapiens
EHSSE

SEQ ID NO: 1065 - protein, 5 aa, Homo sapiens
HSSES

SEQ ID NO: 1066 - protein, 5 aa, Homo sapiens
SSESR

SEQ ID NO: 1067 - protein, 5 aa, Homo sapiens
SESRC

SEQ ID NO: 1068 - protein, 5 aa, Homo sapiens
ESRCC

SEQ ID NO: 1069 - protein, 5 aa, Homo sapiens
SRCCR

SEQ ID NO: 1070 - protein, 5 aa, Homo sapiens
RCCRY

SEQ ID NO: 1071 - protein, 5 aa, Homo sapiens
CCRYP

SEQ ID NO: 1072 - protein, 5 aa, Homo sapiens
CRYPL

SEQ ID NO: 1073 - protein, 5 aa, Homo sapiens
RYPLT

SEQ ID NO: 1074 - protein, 5 aa, Homo sapiens
YPLTV

SEQ ID NO: 1075 - protein, 5 aa, Homo sapiens
PLTVD

SEQ ID NO: 1076 - protein, 5 aa, Homo sapiens
LTVDF

SEQ ID NO: 1077 - protein, 5 aa, Homo sapiens
TVDFE

SEQ ID NO: 1078 - protein, 5 aa, Homo sapiens
VDFEA

SEQ ID NO: 1079 - protein, 5 aa, Homo sapiens
DFEAF

SEQ ID NO: 1080 - protein, 5 aa, Homo sapiens
FEAFG

SEQ ID NO: 1081 - protein, 5 aa, Homo sapiens
EAFGW

SEQ ID NO: 1082 - protein, 5 aa, Homo sapiens
AFGWD

SEQ ID NO: 1083 - protein, 5 aa, Homo sapiens
FGWDW

SEQ ID NO: 1084 - protein, 5 aa, Homo sapiens
GWDWI

SEQ ID NO: 1085 - protein, 5 aa, Homo sapiens
WDWII

SEQ ID NO: 1086 - protein, 5 aa, Homo sapiens
DWIIA

SEQ ID NO: 1087 - protein, 5 aa, Homo sapiens
WIIAP

SEQ ID NO: 1088 - protein, 5 aa, Homo sapiens
IIAPK

SEQ ID NO: 1089 - protein, 5 aa, Homo sapiens
IAPKR

SEQ ID NO: 1090 - protein, 5 aa, Homo sapiens
APKRY

SEQ ID NO: 1091 - protein, 5 aa, Homo sapiens
PKRYK

SEQ ID NO: 1092 - protein, 5 aa, Homo sapiens
KRYKA

SEQ ID NO: 1093 - protein, 5 aa, Homo sapiens
RYKAN

SEQ ID NO: 1094 - protein, 5 aa, Homo sapiens
YKANY

SEQ ID NO: 1095 - protein, 5 aa, Homo sapiens
KANYC

SEQ ID NO: 1096 - protein, 5 aa, Homo sapiens
ANYCS

SEQ ID NO: 1097 - protein, 5 aa, Homo sapiens
NYCSG

SEQ ID NO: 1098 - protein, 5 aa, Homo sapiens
YCSGQ

SEQ ID NO: 1099 - protein, 5 aa, Homo sapiens
CSGQC

SEQ ID NO: 1100 - protein, 5 aa, Homo sapiens
SGQCE

SEQ ID NO: 1101 - protein, 5 aa, Homo sapiens
GQCEY

SEQ ID NO: 1102 - protein, 5 aa, Homo sapiens
QCEYM

SEQ ID NO: 1103 - protein, 5 aa, Homo sapiens
CEYMF

SEQ ID NO: 1104 - protein, 5 aa, Homo sapiens
EYMFM

SEQ ID NO: 1105 - protein, 5 aa, Homo sapiens
YMFMQ

SEQ ID NO: 1106 - protein, 5 aa, Homo sapiens
MFMQK

SEQ ID NO: 1107 - protein, 5 aa, Homo sapiens
FMQKY

SEQ ID NO: 1108 - protein, 5 aa, Homo sapiens
MQKYP

SEQ ID NO: 1109 - protein, 5 aa, Homo sapiens
QKYPH

SEQ ID NO: 1110 - protein, 5 aa, Homo sapiens
KYPHT

SEQ ID NO: 1111 - protein, 5 aa, Homo sapiens
YPHTH

SEQ ID NO: 1112 - protein, 5 aa, Homo sapiens
PHTHL

SEQ ID NO: 1113 - protein, 5 aa, Homo sapiens
HTHLV

SEQ ID NO: 1114 - protein, 5 aa, Homo sapiens
THLVQ

SEQ ID NO: 1115 - protein, 5 aa, Homo sapiens
HLVQQ

SEQ ID NO: 1116 - protein, 5 aa, Homo sapiens
LVQQA

SEQ ID NO: 1117 - protein, 5 aa, Homo sapiens
VQQAN

SEQ ID NO: 1118 - protein, 5 aa, Homo sapiens
QQANP

SEQ ID NO: 1119 - protein, 5 aa, Homo sapiens
QANPR

SEQ ID NO: 1120 - protein, 5 aa, Homo sapiens
ANPRG

SEQ ID NO: 1121 - protein, 5 aa, Homo sapiens
NPRGS

SEQ ID NO: 1122 - protein, 5 aa, Homo sapiens
PRGSA

SEQ ID NO: 1123 - protein, 5 aa, Homo sapiens
RGSAG

SEQ ID NO: 1124 - protein, 5 aa, Homo sapiens
GSAGP

SEQ ID NO: 1125 - protein, 5 aa, Homo sapiens
SAGPC

SEQ ID NO: 1126 - protein, 5 aa, Homo sapiens
AGPCC

SEQ ID NO: 1127 - protein, 5 aa, Homo sapiens
GPCCT

SEQ ID NO: 1128 - protein, 5 aa, Homo sapiens
PCCTP

SEQ ID NO: 1129 - protein, 5 aa, Homo sapiens
CCTPT

SEQ ID NO: 1130 - protein, 5 aa, Homo sapiens
CTPTK

SEQ ID NO: 1131 - protein, 5 aa, Homo sapiens
TPTKM

SEQ ID NO: 1132 - protein, 5 aa, Homo sapiens
PTKMS

SEQ ID NO: 1133 - protein, 5 aa, Homo sapiens
TKMSP

SEQ ID NO: 1134 - protein, 5 aa, Homo sapiens
KMSPI

SEQ ID NO: 1135 - protein, 5 aa, Homo sapiens
MSPIN

SEQ ID NO: 1136 - protein, 5 aa, Homo sapiens
SPINM

SEQ ID NO: 1137 - protein, 5 aa, Homo sapiens
PINML

SEQ ID NO: 1138 - protein, 5 aa, Homo sapiens
INMLY

SEQ ID NO: 1139 - protein, 5 aa, Homo sapiens
NMLYF

SEQ ID NO: 1140 - protein, 5 aa, Homo sapiens
MLYFN

SEQ ID NO: 1141 - protein, 5 aa, Homo sapiens
LYFND

SEQ ID NO: 1142 - protein, 5 aa, Homo sapiens
YFNDK

SEQ ID NO: 1143 - protein, 5 aa, Homo sapiens
FNDKQ

SEQ ID NO: 1144 - protein, 5 aa, Homo sapiens
NDKQQ

SEQ ID NO: 1145 - protein, 5 aa, Homo sapiens
DKQQI

SEQ ID NO: 1146 - protein, 5 aa, Homo sapiens
KQQII

SEQ ID NO: 1147 - protein, 5 aa, Homo sapiens
QQIIY

SEQ ID NO: 1148 - protein, 5 aa, Homo sapiens
QIIYG

SEQ ID NO: 1149 - protein, 5 aa, Homo sapiens
IIYGK

SEQ ID NO: 1150 - protein, 5 aa, Homo sapiens
IYGKI

SEQ ID NO: 1151 - protein, 5 aa, Homo sapiens
YGKIP

SEQ ID NO: 1152 - protein, 5 aa, Homo sapiens
GKIPG

SEQ ID NO: 1153 - protein, 5 aa, Homo sapiens
KIPGM

SEQ ID NO: 1154 - protein, 5 aa, Homo sapiens
IPGMV

SEQ ID NO: 1155 - protein, 5 aa, Homo sapiens
PGMVV

SEQ ID NO: 1156 - protein, 5 aa, Homo sapiens
GMVVD

SEQ ID NO: 1157 - protein, 5 aa, Homo sapiens
MVVDR

SEQ ID NO: 1158 - protein, 5 aa, Homo sapiens
VVDRC

SEQ ID NO: 1159 - protein, 5 aa, Homo sapiens
VDRCG

SEQ ID NO: 1160 - protein, 5 aa, Homo sapiens
DRCGC

SEQ ID NO: 1161 - protein, 5 aa, Homo sapiens
RCGCS

SEQ ID NO: 1162 - protein, 6 aa, Homo sapiens
MVLAAP

SEQ ID NO: 1163 - protein, 6 aa, Homo sapiens
VLAAPL

SEQ ID NO: 1164 - protein, 6 aa, Homo sapiens
LAAPLL

SEQ ID NO: 1165 - protein, 6 aa, Homo sapiens
AAPLLL

SEQ ID NO: 1166 - protein, 6 aa, Homo sapiens
APLLLG

SEQ ID NO: 1167 - protein, 6 aa, Homo sapiens
PLLLGF

SEQ ID NO: 1168 - protein, 6 aa, Homo sapiens
LLLGFL

SEQ ID NO: 1169 - protein, 6 aa, Homo sapiens
LLGFLL

SEQ ID NO: 1170 - protein, 6 aa, Homo sapiens
LGFLLL

SEQ ID NO: 1171 - protein, 6 aa, Homo sapiens
GFLLLA

SEQ ID NO: 1172 - protein, 6 aa, Homo sapiens
FLLLAL

SEQ ID NO: 1173 - protein, 6 aa, Homo sapiens
LLLALE

SEQ ID NO: 1174 - protein, 6 aa, Homo sapiens
LLALEL

SEQ ID NO: 1175 - protein, 6 aa, Homo sapiens
LALELR

SEQ ID NO: 1176 - protein, 6 aa, Homo sapiens
ALELRP

SEQ ID NO: 1177 - protein, 6 aa, Homo sapiens
LELRPR

SEQ ID NO: 1178 - protein, 6 aa, Homo sapiens
ELRPRG

SEQ ID NO: 1179 - protein, 6 aa, Homo sapiens
LRPRGE

SEQ ID NO: 1180 - protein, 6 aa, Homo sapiens
RPRGEA

SEQ ID NO: 1181 - protein, 6 aa, Homo sapiens
PRGEAA

SEQ ID NO: 1182 - protein, 6 aa, Homo sapiens
RGEAAE

SEQ ID NO: 1183 - protein, 6 aa, Homo sapiens
GEAAEG

SEQ ID NO: 1184 - protein, 6 aa, Homo sapiens
EAAEGP

SEQ ID NO: 1185 - protein, 6 aa, Homo sapiens
AAEGPA

SEQ ID NO: 1186 - protein, 6 aa, Homo sapiens
AEGPAA

SEQ ID NO: 1187 - protein, 6 aa, Homo sapiens
EGPAAA

SEQ ID NO: 1188 - protein, 6 aa, Homo sapiens
GPAAAA

SEQ ID NO: 1189 - protein, 6 aa, Homo sapiens
PAAAAA

SEQ ID NO: 1190 - protein, 6 aa, Homo sapiens
AAAAAA

SEQ ID NO: 1191 - protein, 6 aa, Homo sapiens
AAAAAG

SEQ ID NO: 1192 - protein, 6 aa, Homo sapiens
AAAAGV

SEQ ID NO: 1193 - protein, 6 aa, Homo sapiens
AAAGVG

SEQ ID NO: 1194 - protein, 6 aa, Homo sapiens
AAGVGG

SEQ ID NO: 1195 - protein, 6 aa, Homo sapiens
AGVGGE

SEQ ID NO: 1196 - protein, 6 aa, Homo sapiens
GVGGER

SEQ ID NO: 1197 - protein, 6 aa, Homo sapiens
VGGERS

SEQ ID NO: 1198 - protein, 6 aa, Homo sapiens
GGERSS

SEQ ID NO: 1199 - protein, 6 aa, Homo sapiens
GERSSR

SEQ ID NO: 1200 - protein, 6 aa, Homo sapiens
ERSSRP

SEQ ID NO: 1201 - protein, 6 aa, Homo sapiens
RSSRPA

SEQ ID NO: 1202 - protein, 6 aa, Homo sapiens
SSRPAP

SEQ ID NO: 1203 - protein, 6 aa, Homo sapiens
SRPAPS

SEQ ID NO: 1204 - protein, 6 aa, Homo sapiens
RPAPSV

SEQ ID NO: 1205 - protein, 6 aa, Homo sapiens
PAPSVA

SEQ ID NO: 1206 - protein, 6 aa, Homo sapiens
APSVAP

SEQ ID NO: 1207 - protein, 6 aa, Homo sapiens
PSVAPE

SEQ ID NO: 1208 - protein, 6 aa, Homo sapiens
SVAPEP

SEQ ID NO: 1209 - protein, 6 aa, Homo sapiens
VAPEPD

SEQ ID NO: 1210 - protein, 6 aa, Homo sapiens
APEPDG

SEQ ID NO: 1211 - protein, 6 aa, Homo sapiens
PEPDGC

SEQ ID NO: 1212 - protein, 6 aa, Homo sapiens
EPDGCP

SEQ ID NO: 1213 - protein, 6 aa, Homo sapiens
PDGCPV

SEQ ID NO: 1214 - protein, 6 aa, Homo sapiens
DGCPVC

SEQ ID NO: 1215 - protein, 6 aa, Homo sapiens
GCPVCV

SEQ ID NO: 1216 - protein, 6 aa, Homo sapiens
CPVCVW

SEQ ID NO: 1217 - protein, 6 aa, Homo sapiens
PVCVWR

SEQ ID NO: 1218 - protein, 6 aa, Homo sapiens
VCVWRQ

SEQ ID NO: 1219 - protein, 6 aa, Homo sapiens
CVWRQH

SEQ ID NO: 1220 - protein, 6 aa, Homo sapiens
VWRQHS

SEQ ID NO: 1221 - protein, 6 aa, Homo sapiens
WRQHSR

SEQ ID NO: 1222 - protein, 6 aa, Homo sapiens
RQHSRE

SEQ ID NO: 1223 - protein, 6 aa, Homo sapiens
QHSREL

SEQ ID NO: 1224 - protein, 6 aa, Homo sapiens
HSRELR

SEQ ID NO: 1225 - protein, 6 aa, Homo sapiens
SRELRL

SEQ ID NO: 1226 - protein, 6 aa, Homo sapiens
RELRLE

SEQ ID NO: 1227 - protein, 6 aa, Homo sapiens
ELRLES

SEQ ID NO: 1228 - protein, 6 aa, Homo sapiens
LRLESI

SEQ ID NO: 1229 - protein, 6 aa, Homo sapiens
RLESIK

SEQ ID NO: 1230 - protein, 6 aa, Homo sapiens
LESIKS

SEQ ID NO: 1231 - protein, 6 aa, Homo sapiens
ESIKSQ

SEQ ID NO: 1232 - protein, 6 aa, Homo sapiens
SIKSQI

SEQ ID NO: 1233 - protein, 6 aa, Homo sapiens
IKSQIL

SEQ ID NO: 1234 - protein, 6 aa, Homo sapiens
KSQILS

SEQ ID NO: 1235 - protein, 6 aa, Homo sapiens
SQILSK

SEQ ID NO: 1236 - protein, 6 aa, Homo sapiens
QILSKL

SEQ ID NO: 1237 - protein, 6 aa, Homo sapiens
ILSKLR

SEQ ID NO: 1238 - protein, 6 aa, Homo sapiens
LSKLRL

SEQ ID NO: 1239 - protein, 6 aa, Homo sapiens
SKLRLK

SEQ ID NO: 1240 - protein, 6 aa, Homo sapiens
KLRLKE

SEQ ID NO: 1241 - protein, 6 aa, Homo sapiens
LRLKEA

SEQ ID NO: 1242 - protein, 6 aa, Homo sapiens
RLKEAP

SEQ ID NO: 1243 - protein, 6 aa, Homo sapiens
LKEAPN

SEQ ID NO: 1244 - protein, 6 aa, Homo sapiens
KEAPNI

SEQ ID NO: 1245 - protein, 6 aa, Homo sapiens
EAPNIS

SEQ ID NO: 1246 - protein, 6 aa, Homo sapiens
APNISR

SEQ ID NO: 1247 - protein, 6 aa, Homo sapiens
PNISRE

SEQ ID NO: 1248 - protein, 6 aa, Homo sapiens
NISREV

SEQ ID NO: 1249 - protein, 6 aa, Homo sapiens
ISREVV

SEQ ID NO: 1250 - protein, 6 aa, Homo sapiens
SREVVK

SEQ ID NO: 1251 - protein, 6 aa, Homo sapiens
REVVKQ

SEQ ID NO: 1252 - protein, 6 aa, Homo sapiens
EVVKQL

SEQ ID NO: 1253 - protein, 6 aa, Homo sapiens
VVKQLL

SEQ ID NO: 1254 - protein, 6 aa, Homo sapiens
VKQLLP

SEQ ID NO: 1255 - protein, 6 aa, Homo sapiens
KQLLPK

SEQ ID NO: 1256 - protein, 6 aa, Homo sapiens
QLLPKA

SEQ ID NO: 1257 - protein, 6 aa, Homo sapiens
LLPKAP

SEQ ID NO: 1258 - protein, 6 aa, Homo sapiens
LPKAPP

SEQ ID NO: 1259 - protein, 6 aa, Homo sapiens
PKAPPL

SEQ ID NO: 1260 - protein, 6 aa, Homo sapiens
KAPPLQ

SEQ ID NO: 1261 - protein, 6 aa, Homo sapiens
APPLQQ

SEQ ID NO: 1262 - protein, 6 aa, Homo sapiens
PPLQQI

SEQ ID NO: 1263 - protein, 6 aa, Homo sapiens
PLQQIL

SEQ ID NO: 1264 - protein, 6 aa, Homo sapiens
LQQILD

SEQ ID NO: 1265 - protein, 6 aa, Homo sapiens
QQILDL

SEQ ID NO: 1266 - protein, 6 aa, Homo sapiens
QILDLH

SEQ ID NO: 1267 - protein, 6 aa, Homo sapiens
ILDLHD

SEQ ID NO: 1268 - protein, 6 aa, Homo sapiens
LDLHDF

SEQ ID NO: 1269 - protein, 6 aa, Homo sapiens
DLHDFQ

SEQ ID NO: 1270 - protein, 6 aa, Homo sapiens
LHDFQG

SEQ ID NO: 1271 - protein, 6 aa, Homo sapiens
HDFQGD

SEQ ID NO: 1272 - protein, 6 aa, Homo sapiens
DFQGDA

SEQ ID NO: 1273 - protein, 6 aa, Homo sapiens
FQGDAL

SEQ ID NO: 1274 - protein, 6 aa, Homo sapiens
QGDALQ

SEQ ID NO: 1275 - protein, 6 aa, Homo sapiens
GDALQP

SEQ ID NO: 1276 - protein, 6 aa, Homo sapiens
DALQPE

SEQ ID NO: 1277 - protein, 6 aa, Homo sapiens
ALQPED

SEQ ID NO: 1278 - protein, 6 aa, Homo sapiens
LQPEDF

SEQ ID NO: 1279 - protein, 6 aa, Homo sapiens
QPEDFL

SEQ ID NO: 1280 - protein, 6 aa, Homo sapiens
PEDFLE

SEQ ID NO: 1281 - protein, 6 aa, Homo sapiens
EDFLEE

SEQ ID NO: 1282 - protein, 6 aa, Homo sapiens
DFLEED

SEQ ID NO: 1283 - protein, 6 aa, Homo sapiens
FLEEDE

SEQ ID NO: 1284 - protein, 6 aa, Homo sapiens
LEEDEY

SEQ ID NO: 1285 - protein, 6 aa, Homo sapiens
EEDEYH

SEQ ID NO: 1286 - protein, 6 aa, Homo sapiens
EDEYHA

SEQ ID NO: 1287 - protein, 6 aa, Homo sapiens
DEYHAT

SEQ ID NO: 1288 - protein, 6 aa, Homo sapiens
EYHATT

SEQ ID NO: 1289 - protein, 6 aa, Homo sapiens
YHATTE

SEQ ID NO: 1290 - protein, 6 aa, Homo sapiens
HATTET

SEQ ID NO: 1291 - protein, 6 aa, Homo sapiens
ATTETV

SEQ ID NO: 1292 - protein, 6 aa, Homo sapiens
TTETVI

SEQ ID NO: 1293 - protein, 6 aa, Homo sapiens
TETVIS

SEQ ID NO: 1294 - protein, 6 aa, Homo sapiens
ETVISM

SEQ ID NO: 1295 - protein, 6 aa, Homo sapiens
TVISMA

SEQ ID NO: 1296 - protein, 6 aa, Homo sapiens
VISMAQ

SEQ ID NO: 1297 - protein, 6 aa, Homo sapiens
ISMAQE

SEQ ID NO: 1298 - protein, 6 aa, Homo sapiens
SMAQET

SEQ ID NO: 1299 - protein, 6 aa, Homo sapiens
MAQETD

SEQ ID NO: 1300 - protein, 6 aa, Homo sapiens
AQETDP

SEQ ID NO: 1301 - protein, 6 aa, Homo sapiens
QETDPA

SEQ ID NO: 1302 - protein, 6 aa, Homo sapiens
ETDPAV

SEQ ID NO: 1303 - protein, 6 aa, Homo sapiens
TDPAVQ

SEQ ID NO: 1304 - protein, 6 aa, Homo sapiens
DPAVQT

SEQ ID NO: 1305 - protein, 6 aa, Homo sapiens
PAVQTD

SEQ ID NO: 1306 - protein, 6 aa, Homo sapiens
AVQTDG

SEQ ID NO: 1307 - protein, 6 aa, Homo sapiens
VQTDGS

SEQ ID NO: 1308 - protein, 6 aa, Homo sapiens
QTDGSP

SEQ ID NO: 1309 - protein, 6 aa, Homo sapiens
TDGSPL

SEQ ID NO: 1310 - protein, 6 aa, Homo sapiens
DGSPLC

SEQ ID NO: 1311 - protein, 6 aa, Homo sapiens
GSPLCC

SEQ ID NO: 1312 - protein, 6 aa, Homo sapiens
SPLCCH

SEQ ID NO: 1313 - protein, 6 aa, Homo sapiens
PLCCHF

SEQ ID NO: 1314 - protein, 6 aa, Homo sapiens
LCCHFH

SEQ ID NO: 1315 - protein, 6 aa, Homo sapiens
CCHFHF

SEQ ID NO: 1316 - protein, 6 aa, Homo sapiens
CHFHFS

SEQ ID NO: 1317 - protein, 6 aa, Homo sapiens
HFHFSP

SEQ ID NO: 1318 - protein, 6 aa, Homo sapiens
FHFSPK

SEQ ID NO: 1319 - protein, 6 aa, Homo sapiens
HFSPKV

SEQ ID NO: 1320 - protein, 6 aa, Homo sapiens
FSPKVM

SEQ ID NO: 1321 - protein, 6 aa, Homo sapiens
SPKVMF

SEQ ID NO: 1322 - protein, 6 aa, Homo sapiens
PKVMFT

SEQ ID NO: 1323 - protein, 6 aa, Homo sapiens
KVMFTK

SEQ ID NO: 1324 - protein, 6 aa, Homo sapiens
VMFTKV

SEQ ID NO: 1325 - protein, 6 aa, Homo sapiens
MFTKVL

SEQ ID NO: 1326 - protein, 6 aa, Homo sapiens
FTKVLK

SEQ ID NO: 1327 - protein, 6 aa, Homo sapiens
TKVLKA

SEQ ID NO: 1328 - protein, 6 aa, Homo sapiens
KVLKAQ

SEQ ID NO: 1329 - protein, 6 aa, Homo sapiens
VLKAQL

SEQ ID NO: 1330 - protein, 6 aa, Homo sapiens
LKAQLW

SEQ ID NO: 1331 - protein, 6 aa, Homo sapiens
KAQLWV

SEQ ID NO: 1332 - protein, 6 aa, Homo sapiens
AQLWVY

SEQ ID NO: 1333 - protein, 6 aa, Homo sapiens
QLWVYL

SEQ ID NO: 1334 - protein, 6 aa, Homo sapiens
LWVYLR

SEQ ID NO: 1335 - protein, 6 aa, Homo sapiens
WVYLRP

SEQ ID NO: 1336 - protein, 6 aa, Homo sapiens
VYLRPV

SEQ ID NO: 1337 - protein, 6 aa, Homo sapiens
YLRPVP

SEQ ID NO: 1338 - protein, 6 aa, Homo sapiens
LRPVPR

SEQ ID NO: 1339 - protein, 6 aa, Homo sapiens
RPVPRP

SEQ ID NO: 1340 - protein, 6 aa, Homo sapiens
PVPRPA

SEQ ID NO: 1341 - protein, 6 aa, Homo sapiens
VPRPAT

SEQ ID NO: 1342 - protein, 6 aa, Homo sapiens
PRPATV

SEQ ID NO: 1343 - protein, 6 aa, Homo sapiens
RPATVY

SEQ ID NO: 1344 - protein, 6 aa, Homo sapiens
PATVYL

SEQ ID NO: 1345 - protein, 6 aa, Homo sapiens
ATVYLQ

SEQ ID NO: 1346 - protein, 6 aa, Homo sapiens
TVYLQI

SEQ ID NO: 1347 - protein, 6 aa, Homo sapiens
VYLQIL

SEQ ID NO: 1348 - protein, 6 aa, Homo sapiens
YLQILR

SEQ ID NO: 1349 - protein, 6 aa, Homo sapiens
LQILRL

SEQ ID NO: 1350 - protein, 6 aa, Homo sapiens
QILRLK

SEQ ID NO: 1351 - protein, 6 aa, Homo sapiens
ILRLKP

SEQ ID NO: 1352 - protein, 6 aa, Homo sapiens
LRLKPL

SEQ ID NO: 1353 - protein, 6 aa, Homo sapiens
RLKPLT

SEQ ID NO: 1354 - protein, 6 aa, Homo sapiens
LKPLTG

SEQ ID NO: 1355 - protein, 6 aa, Homo sapiens
KPLTGE

SEQ ID NO: 1356 - protein, 6 aa, Homo sapiens
PLTGEG

SEQ ID NO: 1357 - protein, 6 aa, Homo sapiens
LTGEGT

SEQ ID NO: 1358 - protein, 6 aa, Homo sapiens
TGEGTA

SEQ ID NO: 1359 - protein, 6 aa, Homo sapiens
GEGTAG

SEQ ID NO: 1360 - protein, 6 aa, Homo sapiens
EGTAGG

SEQ ID NO: 1361 - protein, 6 aa, Homo sapiens
GTAGGG

SEQ ID NO: 1362 - protein, 6 aa, Homo sapiens
TAGGGG

SEQ ID NO: 1363 - protein, 6 aa, Homo sapiens
AGGGGG

SEQ ID NO: 1364 - protein, 6 aa, Homo sapiens
GGGGGG

SEQ ID NO: 1365 - protein, 6 aa, Homo sapiens
GGGGGR

SEQ ID NO: 1366 - protein, 6 aa, Homo sapiens
GGGGRR

SEQ ID NO: 1367 - protein, 6 aa, Homo sapiens
GGGRRH

SEQ ID NO: 1368 - protein, 6 aa, Homo sapiens
GGRRHI

SEQ ID NO: 1369 - protein, 6 aa, Homo sapiens
GRRHIR

SEQ ID NO: 1370 - protein, 6 aa, Homo sapiens
RRHIRI

SEQ ID NO: 1371 - protein, 6 aa, Homo sapiens
RHIRIR

SEQ ID NO: 1372 - protein, 6 aa, Homo sapiens
HIRIRS

SEQ ID NO: 1373 - protein, 6 aa, Homo sapiens
IRIRSL

SEQ ID NO: 1374 - protein, 6 aa, Homo sapiens
RIRSLK

SEQ ID NO: 1375 - protein, 6 aa, Homo sapiens
IRSLKI

SEQ ID NO: 1376 - protein, 6 aa, Homo sapiens
RSLKIE

SEQ ID NO: 1377 - protein, 6 aa, Homo sapiens
SLKIEL

SEQ ID NO: 1378 - protein, 6 aa, Homo sapiens
LKIELH

SEQ ID NO: 1379 - protein, 6 aa, Homo sapiens
KIELHS

SEQ ID NO: 1380 - protein, 6 aa, Homo sapiens
IELHSR

SEQ ID NO: 1381 - protein, 6 aa, Homo sapiens
ELHSRS

SEQ ID NO: 1382 - protein, 6 aa, Homo sapiens
LHSRSG

SEQ ID NO: 1383 - protein, 6 aa, Homo sapiens
HSRSGH

SEQ ID NO: 1384 - protein, 6 aa, Homo sapiens
SRSGHW

SEQ ID NO: 1385 - protein, 6 aa, Homo sapiens
RSGHWQ

SEQ ID NO: 1386 - protein, 6 aa, Homo sapiens
SGHWQS

SEQ ID NO: 1387 - protein, 6 aa, Homo sapiens
GHWQSI

SEQ ID NO: 1388 - protein, 6 aa, Homo sapiens
HWQSID

SEQ ID NO: 1389 - protein, 6 aa, Homo sapiens
WQSIDF

SEQ ID NO: 1390 - protein, 6 aa, Homo sapiens
QSIDFK

SEQ ID NO: 1391 - protein, 6 aa, Homo sapiens
SIDFKQ

SEQ ID NO: 1392 - protein, 6 aa, Homo sapiens
IDFKQV

SEQ ID NO: 1393 - protein, 6 aa, Homo sapiens
DFKQVL

SEQ ID NO: 1394 - protein, 6 aa, Homo sapiens
FKQVLH

SEQ ID NO: 1395 - protein, 6 aa, Homo sapiens
KQVLHS

SEQ ID NO: 1396 - protein, 6 aa, Homo sapiens
QVLHSW

SEQ ID NO: 1397 - protein, 6 aa, Homo sapiens
VLHSWF

SEQ ID NO: 1398 - protein, 6 aa, Homo sapiens
LHSWFR

SEQ ID NO: 1399 - protein, 6 aa, Homo sapiens
HSWFRQ

SEQ ID NO: 1400 - protein, 6 aa, Homo sapiens
SWFRQP

SEQ ID NO: 1401 - protein, 6 aa, Homo sapiens
WFRQPQ

SEQ ID NO: 1402 - protein, 6 aa, Homo sapiens
FRQPQS

SEQ ID NO: 1403 - protein, 6 aa, Homo sapiens
RQPQSN

SEQ ID NO: 1404 - protein, 6 aa, Homo sapiens
QPQSNW

SEQ ID NO: 1405 - protein, 6 aa, Homo sapiens
PQSNWG

SEQ ID NO: 1406 - protein, 6 aa, Homo sapiens
QSNWGI

SEQ ID NO: 1407 - protein, 6 aa, Homo sapiens
SNWGIE

SEQ ID NO: 1408 - protein, 6 aa, Homo sapiens
NWGIEI

SEQ ID NO: 1409 - protein, 6 aa, Homo sapiens
WGIEIN

SEQ ID NO: 1410 - protein, 6 aa, Homo sapiens
GIEINA

SEQ ID NO: 1411 - protein, 6 aa, Homo sapiens
IEINAF

SEQ ID NO: 1412 - protein, 6 aa, Homo sapiens
EINAFD

SEQ ID NO: 1413 - protein, 6 aa, Homo sapiens
INAFDP

SEQ ID NO: 1414 - protein, 6 aa, Homo sapiens
NAFDPS

SEQ ID NO: 1415 - protein, 6 aa, Homo sapiens
AFDPSG

SEQ ID NO: 1416 - protein, 6 aa, Homo sapiens
FDPSGT

SEQ ID NO: 1417 - protein, 6 aa, Homo sapiens
DPSGTD

SEQ ID NO: 1418 - protein, 6 aa, Homo sapiens
PSGTDL

SEQ ID NO: 1419 - protein, 6 aa, Homo sapiens
SGTDLA

SEQ ID NO: 1420 - protein, 6 aa, Homo sapiens
GTDLAV

SEQ ID NO: 1421 - protein, 6 aa, Homo sapiens
TDLAVT

SEQ ID NO: 1422 - protein, 6 aa, Homo sapiens
DLAVTS

SEQ ID NO: 1423 - protein, 6 aa, Homo sapiens
LAVTSL

SEQ ID NO: 1424 - protein, 6 aa, Homo sapiens
AVTSLG

SEQ ID NO: 1425 - protein, 6 aa, Homo sapiens
VTSLGP

SEQ ID NO: 1426 - protein, 6 aa, Homo sapiens
TSLGPG

SEQ ID NO: 1427 - protein, 6 aa, Homo sapiens
SLGPGA

SEQ ID NO: 1428 - protein, 6 aa, Homo sapiens
LGPGAE

SEQ ID NO: 1429 - protein, 6 aa, Homo sapiens
GPGAEG

SEQ ID NO: 1430 - protein, 6 aa, Homo sapiens
PGAEGL

SEQ ID NO: 1431 - protein, 6 aa, Homo sapiens
GAEGLH

SEQ ID NO: 1432 - protein, 6 aa, Homo sapiens
AEGLHP

SEQ ID NO: 1433 - protein, 6 aa, Homo sapiens
EGLHPF

SEQ ID NO: 1434 - protein, 6 aa, Homo sapiens
GLHPFM

SEQ ID NO: 1435 - protein, 6 aa, Homo sapiens
LHPFME

SEQ ID NO: 1436 - protein, 6 aa, Homo sapiens
HPFMEL

SEQ ID NO: 1437 - protein, 6 aa, Homo sapiens
PFMELR

SEQ ID NO: 1438 - protein, 6 aa, Homo sapiens
FMELRV

SEQ ID NO: 1439 - protein, 6 aa, Homo sapiens
MELRVL

SEQ ID NO: 1440 - protein, 6 aa, Homo sapiens
ELRVLE

SEQ ID NO: 1441 - protein, 6 aa, Homo sapiens
LRVLEN

SEQ ID NO: 1442 - protein, 6 aa, Homo sapiens
RVLENT

SEQ ID NO: 1443 - protein, 6 aa, Homo sapiens
VLENTK

SEQ ID NO: 1444 - protein, 6 aa, Homo sapiens
LENTKR

SEQ ID NO: 1445 - protein, 6 aa, Homo sapiens
ENTKRS

SEQ ID NO: 1446 - protein, 6 aa, Homo sapiens
NTKRSR

SEQ ID NO: 1447 - protein, 6 aa, Homo sapiens
TKRSRR

SEQ ID NO: 1448 - protein, 6 aa, Homo sapiens
KRSRRN

SEQ ID NO: 1449 - protein, 6 aa, Homo sapiens
RSRRNL

SEQ ID NO: 1450 - protein, 6 aa, Homo sapiens
SRRNLG

SEQ ID NO: 1451 - protein, 6 aa, Homo sapiens
RRNLGL

SEQ ID NO: 1452 - protein, 6 aa, Homo sapiens
RNLGLD

SEQ ID NO: 1453 - protein, 6 aa, Homo sapiens
NLGLDC

SEQ ID NO: 1454 - protein, 6 aa, Homo sapiens
LGLDCD

SEQ ID NO: 1455 - protein, 6 aa, Homo sapiens
GLDCDE

SEQ ID NO: 1456 - protein, 6 aa, Homo sapiens
LDCDEH

SEQ ID NO: 1457 - protein, 6 aa, Homo sapiens
DCDEHS

SEQ ID NO: 1458 - protein, 6 aa, Homo sapiens
CDEHSS

SEQ ID NO: 1459 - protein, 6 aa, Homo sapiens
DEHSSE

SEQ ID NO: 1460 - protein, 6 aa, Homo sapiens
EHSSES

SEQ ID NO: 1461 - protein, 6 aa, Homo sapiens
HSSESR

SEQ ID NO: 1462 - protein, 6 aa, Homo sapiens
SSESRC

SEQ ID NO: 1463 - protein, 6 aa, Homo sapiens
SESRCC

SEQ ID NO: 1464 - protein, 6 aa, Homo sapiens
ESRCCR

SEQ ID NO: 1465 - protein, 6 aa, Homo sapiens
SRCCRY

SEQ ID NO: 1466 - protein, 6 aa, Homo sapiens
RCCRYP

SEQ ID NO: 1467 - protein, 6 aa, Homo sapiens
CCRYPL

SEQ ID NO: 1468 - protein, 6 aa, Homo sapiens
CRYPLT

SEQ ID NO: 1469 - protein, 6 aa, Homo sapiens
RYPLTV

SEQ ID NO: 1470 - protein, 6 aa, Homo sapiens
YPLTVD

SEQ ID NO: 1471 - protein, 6 aa, Homo sapiens
PLTVDF

SEQ ID NO: 1472 - protein, 6 aa, Homo sapiens
LTVDFE

SEQ ID NO: 1473 - protein, 6 aa, Homo sapiens
TVDFEA

SEQ ID NO: 1474 - protein, 6 aa, Homo sapiens
VDFEAF

SEQ ID NO: 1475 - protein, 6 aa, Homo sapiens
DFEAFG

SEQ ID NO: 1476 - protein, 6 aa, Homo sapiens
FEAFGW

SEQ ID NO: 1477 - protein, 6 aa, Homo sapiens
EAFGWD

SEQ ID NO: 1478 - protein, 6 aa, Homo sapiens
AFGWDW

SEQ ID NO: 1479 - protein, 6 aa, Homo sapiens
FGWDWI

SEQ ID NO: 1480 - protein, 6 aa, Homo sapiens
GWDWII

SEQ ID NO: 1481 - protein, 6 aa, Homo sapiens
WDWIIA

SEQ ID NO: 1482 - protein, 6 aa, Homo sapiens
DWIIAP

SEQ ID NO: 1483 - protein, 6 aa, Homo sapiens
WIIAPK

SEQ ID NO: 1484 - protein, 6 aa, Homo sapiens
IIAPKR

SEQ ID NO: 1485 - protein, 6 aa, Homo sapiens
IAPKRY

SEQ ID NO: 1486 - protein, 6 aa, Homo sapiens
APKRYK

SEQ ID NO: 1487 - protein, 6 aa, Homo sapiens
PKRYKA

SEQ ID NO: 1488 - protein, 6 aa, Homo sapiens
KRYKAN

SEQ ID NO: 1489 - protein, 6 aa, Homo sapiens
RYKANY

SEQ ID NO: 1490 - protein, 6 aa, Homo sapiens
YKANYC

SEQ ID NO: 1491 - protein, 6 aa, Homo sapiens
KANYCS

SEQ ID NO: 1492 - protein, 6 aa, Homo sapiens
ANYCSG

SEQ ID NO: 1493 - protein, 6 aa, Homo sapiens
NYCSGQ

SEQ ID NO: 1494 - protein, 6 aa, Homo sapiens
YCSGQC

SEQ ID NO: 1495 - protein, 6 aa, Homo sapiens
CSGQCE

SEQ ID NO: 1496 - protein, 6 aa, Homo sapiens
SGQCEY

SEQ ID NO: 1497 - protein, 6 aa, Homo sapiens
GQCEYM

SEQ ID NO: 1498 - protein, 6 aa, Homo sapiens
QCEYMF

SEQ ID NO: 1499 - protein, 6 aa, Homo sapiens
CEYMFM

SEQ ID NO: 1500 - protein, 6 aa, Homo sapiens
EYMFMQ

SEQ ID NO: 1501 - protein, 6 aa, Homo sapiens
YMFMQK

SEQ ID NO: 1502 - protein, 6 aa, Homo sapiens
MFMQKY

SEQ ID NO: 1503 - protein, 6 aa, Homo sapiens
FMQKYP

SEQ ID NO: 1504 - protein, 6 aa, Homo sapiens
MQKYPH

SEQ ID NO: 1505 - protein, 6 aa, Homo sapiens
QKYPHT

SEQ ID NO: 1506 - protein, 6 aa, Homo sapiens
KYPHTH

SEQ ID NO: 1507 - protein, 6 aa, Homo sapiens
YPHTHL

SEQ ID NO: 1508 - protein, 6 aa, Homo sapiens
PHTHLV

SEQ ID NO: 1509 - protein, 6 aa, Homo sapiens
HTHLVQ

SEQ ID NO: 1510 - protein, 6 aa, Homo sapiens
THLVQQ

SEQ ID NO: 1511 - protein, 6 aa, Homo sapiens
HLVQQA

SEQ ID NO: 1512 - protein, 6 aa, Homo sapiens
LVQQAN

SEQ ID NO: 1513 - protein, 6 aa, Homo sapiens
VQQANP

SEQ ID NO: 1514 - protein, 6 aa, Homo sapiens
QQANPR

SEQ ID NO: 1515 - protein, 6 aa, Homo sapiens
QANPRG

SEQ ID NO: 1516 - protein, 6 aa, Homo sapiens
ANPRGS

SEQ ID NO: 1517 - protein, 6 aa, Homo sapiens
NPRGSA

SEQ ID NO: 1518 - protein, 6 aa, Homo sapiens
PRGSAG

SEQ ID NO: 1519 - protein, 6 aa, Homo sapiens
RGSAGP

SEQ ID NO: 1520 - protein, 6 aa, Homo sapiens
GSAGPC

SEQ ID NO: 1521 - protein, 6 aa, Homo sapiens
SAGPCC

SEQ ID NO: 1522 - protein, 6 aa, Homo sapiens
AGPCCT

SEQ ID NO: 1523 - protein, 6 aa, Homo sapiens
GPCCTP

SEQ ID NO: 1524 - protein, 6 aa, Homo sapiens
PCCTPT

SEQ ID NO: 1525 - protein, 6 aa, Homo sapiens
CCTPTK

SEQ ID NO: 1526 - protein, 6 aa, Homo sapiens
CTPTKM

SEQ ID NO: 1527 - protein, 6 aa, Homo sapiens
TPTKMS

SEQ ID NO: 1528 - protein, 6 aa, Homo sapiens
PTKMSP

SEQ ID NO: 1529 - protein, 6 aa, Homo sapiens
TKMSPI

SEQ ID NO: 1530 - protein, 6 aa, Homo sapiens
KMSPIN

SEQ ID NO: 1531 - protein, 6 aa, Homo sapiens
MSPINM

SEQ ID NO: 1532 - protein, 6 aa, Homo sapiens
SPINML

SEQ ID NO: 1533 - protein, 6 aa, Homo sapiens
PINMLY

SEQ ID NO: 1534 - protein, 6 aa, Homo sapiens
INMLYF

SEQ ID NO: 1535 - protein, 6 aa, Homo sapiens
NMLYFN

SEQ ID NO: 1536 - protein, 6 aa, Homo sapiens
MLYFND

SEQ ID NO: 1537 - protein, 6 aa, Homo sapiens
LYFNDK

SEQ ID NO: 1538 - protein, 6 aa, Homo sapiens
YFNDKQ

SEQ ID NO: 1539 - protein, 6 aa, Homo sapiens
FNDKQQ

SEQ ID NO: 1540 - protein, 6 aa, Homo sapiens
NDKQQI

SEQ ID NO: 1541 - protein, 6 aa, Homo sapiens
DKQQII

SEQ ID NO: 1542 - protein, 6 aa, Homo sapiens
KQQIIY

SEQ ID NO: 1543 - protein, 6 aa, Homo sapiens
QQIIYG

SEQ ID NO: 1544 - protein, 6 aa, Homo sapiens
QIIYGK

SEQ ID NO: 1545 - protein, 6 aa, Homo sapiens
IIYGKI

SEQ ID NO: 1546 - protein, 6 aa, Homo sapiens
IYGKIP

SEQ ID NO: 1547 - protein, 6 aa, Homo sapiens
YGKIPG

SEQ ID NO: 1548 - protein, 6 aa, Homo sapiens
GKIPGM

SEQ ID NO: 1549 - protein, 6 aa, Homo sapiens
KIPGMV

SEQ ID NO: 1550 - protein, 6 aa, Homo sapiens
IPGMVV

SEQ ID NO: 1551 - protein, 6 aa, Homo sapiens
PGMVVD

SEQ ID NO: 1552 - protein, 6 aa, Homo sapiens
GMVVDR

SEQ ID NO: 1553 - protein, 6 aa, Homo sapiens
MVVDRC

SEQ ID NO: 1554 - protein, 6 aa, Homo sapiens
VVDRCG

SEQ ID NO: 1555 - protein, 6 aa, Homo sapiens
VDRCGC

SEQ ID NO: 1556 - protein, 6 aa, Homo sapiens
DRCGCS

SEQ ID NO: 1557 - protein, 7 aa, Homo sapiens
MVLAAPL

SEQ ID NO: 1558 - protein, 7 aa, Homo sapiens
VLAAPLL

SEQ ID NO: 1559 - protein, 7 aa, Homo sapiens
LAAPLLL

SEQ ID NO: 1560 - protein, 7 aa, Homo sapiens
AAPLLLG

SEQ ID NO: 1561 - protein, 7 aa, Homo sapiens
APLLLGF

SEQ ID NO: 1562 - protein, 7 aa, Homo sapiens
PLLLGFL

SEQ ID NO: 1563 - protein, 7 aa, Homo sapiens
LLLGFLL

SEQ ID NO: 1564 - protein, 7 aa, Homo sapiens
LLGFLLL

SEQ ID NO: 1565 - protein, 7 aa, Homo sapiens
LGFLLLA

SEQ ID NO: 1566 - protein, 7 aa, Homo sapiens
GFLLLAL

SEQ ID NO: 1567 - protein, 7 aa, Homo sapiens
FLLLALE

SEQ ID NO: 1568 - protein, 7 aa, Homo sapiens
LLLALEL

SEQ ID NO: 1569 - protein, 7 aa, Homo sapiens
LLALELR

SEQ ID NO: 1570 - protein, 7 aa, Homo sapiens
LALELRP

SEQ ID NO: 1571 - protein, 7 aa, Homo sapiens
ALELRPR

SEQ ID NO: 1572 - protein, 7 aa, Homo sapiens
LELRPRG

SEQ ID NO: 1573 - protein, 7 aa, Homo sapiens
ELRPRGE

SEQ ID NO: 1574 - protein, 7 aa, Homo sapiens
LRPRGEA

SEQ ID NO: 1575 - protein, 7 aa, Homo sapiens
RPRGEAA

SEQ ID NO: 1576 - protein, 7 aa, Homo sapiens
PRGEAAE

SEQ ID NO: 1577 - protein, 7 aa, Homo sapiens
RGEAAEG

SEQ ID NO: 1578 - protein, 7 aa, Homo sapiens
GEAAEGP

SEQ ID NO: 1579 - protein, 7 aa, Homo sapiens
EAAEGPA

SEQ ID NO: 1580 - protein, 7 aa, Homo sapiens
AAEGPAA

SEQ ID NO: 1581 - protein, 7 aa, Homo sapiens
AEGPAAA

SEQ ID NO: 1582 - protein, 7 aa, Homo sapiens
EGPAAAA

SEQ ID NO: 1583 - protein, 7 aa, Homo sapiens
GPAAAAA

SEQ ID NO: 1584 - protein, 7 aa, Homo sapiens
PAAAAAA

SEQ ID NO: 1585 - protein, 7 aa, Homo sapiens
AAAAAAA

SEQ ID NO: 1586 - protein, 7 aa, Homo sapiens
AAAAAAG

SEQ ID NO: 1587 - protein, 7 aa, Homo sapiens
AAAAAGV

SEQ ID NO: 1588 - protein, 7 aa, Homo sapiens
AAAAGVG

SEQ ID NO: 1589 - protein, 7 aa, Homo sapiens
AAAGVGG

SEQ ID NO: 1590 - protein, 7 aa, Homo sapiens
AAGVGGE

SEQ ID NO: 1591 - protein, 7 aa, Homo sapiens
AGVGGER

SEQ ID NO: 1592 - protein, 7 aa, Homo sapiens
GVGGERS

SEQ ID NO: 1593 - protein, 7 aa, Homo sapiens
VGGERSS

SEQ ID NO: 1594 - protein, 7 aa, Homo sapiens
GGERSSR

SEQ ID NO: 1595 - protein, 7 aa, Homo sapiens
GERSSRP

SEQ ID NO: 1596 - protein, 7 aa, Homo sapiens
ERSSRPA

SEQ ID NO: 1597 - protein, 7 aa, Homo sapiens
RSSRPAP

SEQ ID NO: 1598 - protein, 7 aa, Homo sapiens
SSRPAPS

SEQ ID NO: 1599 - protein, 7 aa, Homo sapiens
SRPAPSV

SEQ ID NO: 1600 - protein, 7 aa, Homo sapiens
RPAPSVA

SEQ ID NO: 1601 - protein, 7 aa, Homo sapiens
PAPSVAP

SEQ ID NO: 1602 - protein, 7 aa, Homo sapiens
APSVAPE

SEQ ID NO: 1603 - protein, 7 aa, Homo sapiens
PSVAPEP

SEQ ID NO: 1604 - protein, 7 aa, Homo sapiens
SVAPEPD

SEQ ID NO: 1605 - protein, 7 aa, Homo sapiens
VAPEPDG

SEQ ID NO: 1606 - protein, 7 aa, Homo sapiens
APEPDGC

SEQ ID NO: 1607 - protein, 7 aa, Homo sapiens
PEPDGCP

SEQ ID NO: 1608 - protein, 7 aa, Homo sapiens
EPDGCPV

SEQ ID NO: 1609 - protein, 7 aa, Homo sapiens
PDGCPVC

SEQ ID NO: 1610 - protein, 7 aa, Homo sapiens
DGCPVCV

SEQ ID NO: 1611 - protein, 7 aa, Homo sapiens
GCPVCVW

SEQ ID NO: 1612 - protein, 7 aa, Homo sapiens
CPVCVWR

SEQ ID NO: 1613 - protein, 7 aa, Homo sapiens
PVCVWRQ

SEQ ID NO: 1614 - protein, 7 aa, Homo sapiens
VCVWRQH

SEQ ID NO: 1615 - protein, 7 aa, Homo sapiens
CVWRQHS

SEQ ID NO: 1616 - protein, 7 aa, Homo sapiens
VWRQHSR

SEQ ID NO: 1617 - protein, 7 aa, Homo sapiens
WRQHSRE

SEQ ID NO: 1618 - protein, 7 aa, Homo sapiens
RQHSREL

SEQ ID NO: 1619 - protein, 7 aa, Homo sapiens
QHSRELR

SEQ ID NO: 1620 - protein, 7 aa, Homo sapiens
HSRELRL

SEQ ID NO: 1621 - protein, 7 aa, Homo sapiens
SRELRLE

SEQ ID NO: 1622 - protein, 7 aa, Homo sapiens
RELRLES

SEQ ID NO: 1623 - protein, 7 aa, Homo sapiens
ELRLESI

SEQ ID NO: 1624 - protein, 7 aa, Homo sapiens
LRLESIK

SEQ ID NO: 1625 - protein, 7 aa, Homo sapiens
RLESIKS

SEQ ID NO: 1626 - protein, 7 aa, Homo sapiens
LESIKSQ

SEQ ID NO: 1627 - protein, 7 aa, Homo sapiens
ESIKSQI

SEQ ID NO: 1628 - protein, 7 aa, Homo sapiens
SIKSQIL

SEQ ID NO: 1629 - protein, 7 aa, Homo sapiens
IKSQILS

SEQ ID NO: 1630 - protein, 7 aa, Homo sapiens
KSQILSK

SEQ ID NO: 1631 - protein, 7 aa, Homo sapiens
SQILSKL

SEQ ID NO: 1632 - protein, 7 aa, Homo sapiens
QILSKLR

SEQ ID NO: 1633 - protein, 7 aa, Homo sapiens
ILSKLRL

SEQ ID NO: 1634 - protein, 7 aa, Homo sapiens
LSKLRLK

SEQ ID NO: 1635 - protein, 7 aa, Homo sapiens
SKLRLKE

SEQ ID NO: 1636 - protein, 7 aa, Homo sapiens
KLRLKEA

SEQ ID NO: 1637 - protein, 7 aa, Homo sapiens
LRLKEAP

SEQ ID NO: 1638 - protein, 7 aa, Homo sapiens
RLKEAPN

SEQ ID NO: 1639 - protein, 7 aa, Homo sapiens
LKEAPNI

SEQ ID NO: 1640 - protein, 7 aa, Homo sapiens
KEAPNIS

SEQ ID NO: 1641 - protein, 7 aa, Homo sapiens
EAPNISR

SEQ ID NO: 1642 - protein, 7 aa, Homo sapiens
APNISRE

SEQ ID NO: 1643 - protein, 7 aa, Homo sapiens
PNISREV

SEQ ID NO: 1644 - protein, 7 aa, Homo sapiens
NISREVV

SEQ ID NO: 1645 - protein, 7 aa, Homo sapiens
ISREVVK

SEQ ID NO: 1646 - protein, 7 aa, Homo sapiens
SREVVKQ

SEQ ID NO: 1647 - protein, 7 aa, Homo sapiens
REVVKQL

SEQ ID NO: 1648 - protein, 7 aa, Homo sapiens
EVVKQLL

SEQ ID NO: 1649 - protein, 7 aa, Homo sapiens
VVKQLLP

SEQ ID NO: 1650 - protein, 7 aa, Homo sapiens
VKQLLPK

SEQ ID NO: 1651 - protein, 7 aa, Homo sapiens
KQLLPKA

SEQ ID NO: 1652 - protein, 7 aa, Homo sapiens
QLLPKAP

SEQ ID NO: 1653 - protein, 7 aa, Homo sapiens
LLPKAPP

SEQ ID NO: 1654 - protein, 7 aa, Homo sapiens
LPKAPPL

SEQ ID NO: 1655 - protein, 7 aa, Homo sapiens
PKAPPLQ

SEQ ID NO: 1656 - protein, 7 aa, Homo sapiens
KAPPLQQ

SEQ ID NO: 1657 - protein, 7 aa, Homo sapiens
APPLQQI

SEQ ID NO: 1658 - protein, 7 aa, Homo sapiens
PPLQQIL

SEQ ID NO: 1659 - protein, 7 aa, Homo sapiens
PLQQILD

SEQ ID NO: 1660 - protein, 7 aa, Homo sapiens
LQQILDL

SEQ ID NO: 1661 - protein, 7 aa, Homo sapiens
QQILDLH

SEQ ID NO: 1662 - protein, 7 aa, Homo sapiens
QILDLHD

SEQ ID NO: 1663 - protein, 7 aa, Homo sapiens
ILDLHDF

SEQ ID NO: 1664 - protein, 7 aa, Homo sapiens
LDLHDFQ

SEQ ID NO: 1665 - protein, 7 aa, Homo sapiens
DLHDFQG

SEQ ID NO: 1666 - protein, 7 aa, Homo sapiens
LHDFQGD

SEQ ID NO: 1667 - protein, 7 aa, Homo sapiens
HDFQGDA

SEQ ID NO: 1668 - protein, 7 aa, Homo sapiens
DFQGDAL

SEQ ID NO: 1669 - protein, 7 aa, Homo sapiens
FQGDALQ

SEQ ID NO: 1670 - protein, 7 aa, Homo sapiens
QGDALQP

SEQ ID NO: 1671 - protein, 7 aa, Homo sapiens
GDALQPE

SEQ ID NO: 1672 - protein, 7 aa, Homo sapiens
DALQPED

SEQ ID NO: 1673 - protein, 7 aa, Homo sapiens
ALQPEDF

SEQ ID NO: 1674 - protein, 7 aa, Homo sapiens
LQPEDFL

SEQ ID NO: 1675 - protein, 7 aa, Homo sapiens
QPEDFLE

SEQ ID NO: 1676 - protein, 7 aa, Homo sapiens
PEDFLEE

SEQ ID NO: 1677 - protein, 7 aa, Homo sapiens
EDFLEED

SEQ ID NO: 1678 - protein, 7 aa, Homo sapiens
DFLEEDE

SEQ ID NO: 1679 - protein, 7 aa, Homo sapiens
FLEEDEY

SEQ ID NO: 1680 - protein, 7 aa, Homo sapiens
LEEDEYH

SEQ ID NO: 1681 - protein, 7 aa, Homo sapiens
EEDEYHA

SEQ ID NO: 1682 - protein, 7 aa, Homo sapiens
EDEYHAT

SEQ ID NO: 1683 - protein, 7 aa, Homo sapiens
DEYHATT

SEQ ID NO: 1684 - protein, 7 aa, Homo sapiens
EYHATTE

SEQ ID NO: 1685 - protein, 7 aa, Homo sapiens
YHATTET

SEQ ID NO: 1686 - protein, 7 aa, Homo sapiens
HATTETV

SEQ ID NO: 1687 - protein, 7 aa, Homo sapiens
ATTETVI

SEQ ID NO: 1688 - protein, 7 aa, Homo sapiens
TTETVIS

SEQ ID NO: 1689 - protein, 7 aa, Homo sapiens
TETVISM

SEQ ID NO: 1690 - protein, 7 aa, Homo sapiens
ETVISMA

SEQ ID NO: 1691 - protein, 7 aa, Homo sapiens
TVISMAQ

SEQ ID NO: 1692 - protein, 7 aa, Homo sapiens
VISMAQE

SEQ ID NO: 1693 - protein, 7 aa, Homo sapiens
ISMAQET

SEQ ID NO: 1694 - protein, 7 aa, Homo sapiens
SMAQETD

SEQ ID NO: 1695 - protein, 7 aa, Homo sapiens
MAQETDP

SEQ ID NO: 1696 - protein, 7 aa, Homo sapiens
AQETDPA

SEQ ID NO: 1697 - protein, 7 aa, Homo sapiens
QETDPAV

SEQ ID NO: 1698 - protein, 7 aa, Homo sapiens
ETDPAVQ

SEQ ID NO: 1699 - protein, 7 aa, Homo sapiens
TDPAVQT

SEQ ID NO: 1700 - protein, 7 aa, Homo sapiens
DPAVQTD

SEQ ID NO: 1701 - protein, 7 aa, Homo sapiens
PAVQTDG

SEQ ID NO: 1702 - protein, 7 aa, Homo sapiens
AVQTDGS

SEQ ID NO: 1703 - protein, 7 aa, Homo sapiens
VQTDGSP

SEQ ID NO: 1704 - protein, 7 aa, Homo sapiens
QTDGSPL

SEQ ID NO: 1705 - protein, 7 aa, Homo sapiens
TDGSPLC

SEQ ID NO: 1706 - protein, 7 aa, Homo sapiens
DGSPLCC

SEQ ID NO: 1707 - protein, 7 aa, Homo sapiens
GSPLCCH

SEQ ID NO: 1708 - protein, 7 aa, Homo sapiens
SPLCCHF

SEQ ID NO: 1709 - protein, 7 aa, Homo sapiens
PLCCHFH

SEQ ID NO: 1710 - protein, 7 aa, Homo sapiens
LCCHFHF

SEQ ID NO: 1711 - protein, 7 aa, Homo sapiens
CCHFHFS

SEQ ID NO: 1712 - protein, 7 aa, Homo sapiens
CHFHFSP

SEQ ID NO: 1713 - protein, 7 aa, Homo sapiens
HFHFSPK

SEQ ID NO: 1714 - protein, 7 aa, Homo sapiens
FHFSPKV

SEQ ID NO: 1715 - protein, 7 aa, Homo sapiens
HFSPKVM

SEQ ID NO: 1716 - protein, 7 aa, Homo sapiens
FSPKVMF

SEQ ID NO: 1717 - protein, 7 aa, Homo sapiens
SPKVMFT

SEQ ID NO: 1718 - protein, 7 aa, Homo sapiens
PKVMFTK

SEQ ID NO: 1719 - protein, 7 aa, Homo sapiens
KVMFTKV

SEQ ID NO: 1720 - protein, 7 aa, Homo sapiens
VMFTKVL

SEQ ID NO: 1721 - protein, 7 aa, Homo sapiens
MFTKVLK

SEQ ID NO: 1722 - protein, 7 aa, Homo sapiens
FTKVLKA

SEQ ID NO: 1723 - protein, 7 aa, Homo sapiens
TKVLKAQ

SEQ ID NO: 1724 - protein, 7 aa, Homo sapiens
KVLKAQL

SEQ ID NO: 1725 - protein, 7 aa, Homo sapiens
VLKAQLW

SEQ ID NO: 1726 - protein, 7 aa, Homo sapiens
LKAQLWV

SEQ ID NO: 1727 - protein, 7 aa, Homo sapiens
KAQLWVY

SEQ ID NO: 1728 - protein, 7 aa, Homo sapiens
AQLWVYL

SEQ ID NO: 1729 - protein, 7 aa, Homo sapiens
QLWVYLR

SEQ ID NO: 1730 - protein, 7 aa, Homo sapiens
LWVYLRP

SEQ ID NO: 1731 - protein, 7 aa, Homo sapiens
WVYLRPV

SEQ ID NO: 1732 - protein, 7 aa, Homo sapiens
VYLRPVP

SEQ ID NO: 1733 - protein, 7 aa, Homo sapiens
YLRPVPR

SEQ ID NO: 1734 - protein, 7 aa, Homo sapiens
LRPVPRP

SEQ ID NO: 1735 - protein, 7 aa, Homo sapiens
RPVPRPA

SEQ ID NO: 1736 - protein, 7 aa, Homo sapiens
PVPRPAT

SEQ ID NO: 1737 - protein, 7 aa, Homo sapiens
VPRPATV

SEQ ID NO: 1738 - protein, 7 aa, Homo sapiens
PRPATVY

SEQ ID NO: 1739 - protein, 7 aa, Homo sapiens
RPATVYL

SEQ ID NO: 1740 - protein, 7 aa, Homo sapiens
PATVYLQ

SEQ ID NO: 1741 - protein, 7 aa, Homo sapiens
ATVYLQI

SEQ ID NO: 1742 - protein, 7 aa, Homo sapiens
TVYLQIL

SEQ ID NO: 1743 - protein, 7 aa, Homo sapiens
VYLQILR

SEQ ID NO: 1744 - protein, 7 aa, Homo sapiens
YLQILRL

SEQ ID NO: 1745 - protein, 7 aa, Homo sapiens
LQILRLK

SEQ ID NO: 1746 - protein, 7 aa, Homo sapiens
QILRLKP

SEQ ID NO: 1747 - protein, 7 aa, Homo sapiens
ILRLKPL

SEQ ID NO: 1748 - protein, 7 aa, Homo sapiens
LRLKPLT

SEQ ID NO: 1749 - protein, 7 aa, Homo sapiens
RLKPLTG

SEQ ID NO: 1750 - protein, 7 aa, Homo sapiens
LKPLTGE

SEQ ID NO: 1751 - protein, 7 aa, Homo sapiens
KPLTGEG

SEQ ID NO: 1752 - protein, 7 aa, Homo sapiens
PLTGEGT

SEQ ID NO: 1753 - protein, 7 aa, Homo sapiens
LTGEGTA

SEQ ID NO: 1754 - protein, 7 aa, Homo sapiens
TGEGTAG

SEQ ID NO: 1755 - protein, 7 aa, Homo sapiens
GEGTAGG

SEQ ID NO: 1756 - protein, 7 aa, Homo sapiens
EGTAGGG

SEQ ID NO: 1757 - protein, 7 aa, Homo sapiens
GTAGGGG

SEQ ID NO: 1758 - protein, 7 aa, Homo sapiens
TAGGGGG

SEQ ID NO: 1759 - protein, 7 aa, Homo sapiens
AGGGGGG

SEQ ID NO: 1760 - protein, 7 aa, Homo sapiens
GGGGGGR

SEQ ID NO: 1761 - protein, 7 aa, Homo sapiens
GGGGGRR

SEQ ID NO: 1762 - protein, 7 aa, Homo sapiens
GGGGRRH

SEQ ID NO: 1763 - protein, 7 aa, Homo sapiens
GGGRRHI

SEQ ID NO: 1764 - protein, 7 aa, Homo sapiens
GGRRHIR

SEQ ID NO: 1765 - protein, 7 aa, Homo sapiens
GRRHIRI

SEQ ID NO: 1766 - protein, 7 aa, Homo sapiens
RRHIRIR

SEQ ID NO: 1767 - protein, 7 aa, Homo sapiens
RHIRIRS

SEQ ID NO: 1768 - protein, 7 aa, Homo sapiens
HIRIRSL

SEQ ID NO: 1769 - protein, 7 aa, Homo sapiens
IRIRSLK

SEQ ID NO: 1770 - protein, 7 aa, Homo sapiens
RIRSLKI

SEQ ID NO: 1771 - protein, 7 aa, Homo sapiens
IRSLKIE

SEQ ID NO: 1772 - protein, 7 aa, Homo sapiens
RSLKIEL

SEQ ID NO: 1773 - protein, 7 aa, Homo sapiens
SLKIELH

SEQ ID NO: 1774 - protein, 7 aa, Homo sapiens
LKIELHS

SEQ ID NO: 1775 - protein, 7 aa, Homo sapiens
KIELHSR

SEQ ID NO: 1776 - protein, 7 aa, Homo sapiens
IELHSRS

SEQ ID NO: 1777 - protein, 7 aa, Homo sapiens
ELHSRSG

SEQ ID NO: 1778 - protein, 7 aa, Homo sapiens
LHSRSGH

SEQ ID NO: 1779 - protein, 7 aa, Homo sapiens
HSRSGHW

SEQ ID NO: 1780 - protein, 7 aa, Homo sapiens
SRSGHWQ

SEQ ID NO: 1781 - protein, 7 aa, Homo sapiens
RSGHWQS

SEQ ID NO: 1782 - protein, 7 aa, Homo sapiens
SGHWQSI

SEQ ID NO: 1783 - protein, 7 aa, Homo sapiens
GHWQSID

SEQ ID NO: 1784 - protein, 7 aa, Homo sapiens
HWQSIDF

SEQ ID NO: 1785 - protein, 7 aa, Homo sapiens
WQSIDFK

SEQ ID NO: 1786 - protein, 7 aa, Homo sapiens
QSIDFKQ

SEQ ID NO: 1787 - protein, 7 aa, Homo sapiens
SIDFKQV

SEQ ID NO: 1788 - protein, 7 aa, Homo sapiens
IDFKQVL

SEQ ID NO: 1789 - protein, 7 aa, Homo sapiens
DFKQVLH

SEQ ID NO: 1790 - protein, 7 aa, Homo sapiens
FKQVLHS

SEQ ID NO: 1791 - protein, 7 aa, Homo sapiens
KQVLHSW

SEQ ID NO: 1792 - protein, 7 aa, Homo sapiens
QVLHSWF

SEQ ID NO: 1793 - protein, 7 aa, Homo sapiens
VLHSWFR

SEQ ID NO: 1794 - protein, 7 aa, Homo sapiens
LHSWFRQ

SEQ ID NO: 1795 - protein, 7 aa, Homo sapiens
HSWFRQP

SEQ ID NO: 1796 - protein, 7 aa, Homo sapiens
SWFRQPQ

SEQ ID NO: 1797 - protein, 7 aa, Homo sapiens
WFRQPQS

SEQ ID NO: 1798 - protein, 7 aa, Homo sapiens
FRQPQSN

SEQ ID NO: 1799 - protein, 7 aa, Homo sapiens
RQPQSNW

SEQ ID NO: 1800 - protein, 7 aa, Homo sapiens
QPQSNWG

SEQ ID NO: 1801 - protein, 7 aa, Homo sapiens
PQSNWGI

SEQ ID NO: 1802 - protein, 7 aa, Homo sapiens
QSNWGIE

SEQ ID NO: 1803 - protein, 7 aa, Homo sapiens
SNWGIEI

SEQ ID NO: 1804 - protein, 7 aa, Homo sapiens
NWGIEIN

SEQ ID NO: 1805 - protein, 7 aa, Homo sapiens
WGIEINA

SEQ ID NO: 1806 - protein, 7 aa, Homo sapiens
GIEINAF

SEQ ID NO: 1807 - protein, 7 aa, Homo sapiens
IEINAFD

SEQ ID NO: 1808 - protein, 7 aa, Homo sapiens
EINAFDP

SEQ ID NO: 1809 - protein, 7 aa, Homo sapiens
INAFDPS

SEQ ID NO: 1810 - protein, 7 aa, Homo sapiens
NAFDPSG

SEQ ID NO: 1811 - protein, 7 aa, Homo sapiens
AFDPSGT

SEQ ID NO: 1812 - protein, 7 aa, Homo sapiens
FDPSGTD

SEQ ID NO: 1813 - protein, 7 aa, Homo sapiens
DPSGTDL

SEQ ID NO: 1814 - protein, 7 aa, Homo sapiens
PSGTDLA

SEQ ID NO: 1815 - protein, 7 aa, Homo sapiens
SGTDLAV

SEQ ID NO: 1816 - protein, 7 aa, Homo sapiens
GTDLAVT

SEQ ID NO: 1817 - protein, 7 aa, Homo sapiens
TDLAVTS

SEQ ID NO: 1818 - protein, 7 aa, Homo sapiens
DLAVTSL

SEQ ID NO: 1819 - protein, 7 aa, Homo sapiens
LAVTSLG

SEQ ID NO: 1820 - protein, 7 aa, Homo sapiens
AVTSLGP

SEQ ID NO: 1821 - protein, 7 aa, Homo sapiens
VTSLGPG

SEQ ID NO: 1822 - protein, 7 aa, Homo sapiens
TSLGPGA

SEQ ID NO: 1823 - protein, 7 aa, Homo sapiens
SLGPGAE

SEQ ID NO: 1824 - protein, 7 aa, Homo sapiens
LGPGAEG

SEQ ID NO: 1825 - protein, 7 aa, Homo sapiens
GPGAEGL

SEQ ID NO: 1826 - protein, 7 aa, Homo sapiens
PGAEGLH

SEQ ID NO: 1827 - protein, 7 aa, Homo sapiens
GAEGLHP

SEQ ID NO: 1828 - protein, 7 aa, Homo sapiens
AEGLHPF

SEQ ID NO: 1829 - protein, 7 aa, Homo sapiens
EGLHPFM

SEQ ID NO: 1830 - protein, 7 aa, Homo sapiens
GLHPFME

SEQ ID NO: 1831 - protein, 7 aa, Homo sapiens
LHPFMEL

SEQ ID NO: 1832 - protein, 7 aa, Homo sapiens
HPFMELR

SEQ ID NO: 1833 - protein, 7 aa, Homo sapiens
PFMELRV

SEQ ID NO: 1834 - protein, 7 aa, Homo sapiens
FMELRVL

SEQ ID NO: 1835 - protein, 7 aa, Homo sapiens
MELRVLE

SEQ ID NO: 1836 - protein, 7 aa, Homo sapiens
ELRVLEN

SEQ ID NO: 1837 - protein, 7 aa, Homo sapiens
LRVLENT

SEQ ID NO: 1838 - protein, 7 aa, Homo sapiens
RVLENTK

SEQ ID NO: 1839 - protein, 7 aa, Homo sapiens
VLENTKR

SEQ ID NO: 1840 - protein, 7 aa, Homo sapiens
LENTKRS

SEQ ID NO: 1841 - protein, 7 aa, Homo sapiens
ENTKRSR

SEQ ID NO: 1842 - protein, 7 aa, Homo sapiens
NTKRSRR

SEQ ID NO: 1843 - protein, 7 aa, Homo sapiens
TKRSRRN

SEQ ID NO: 1844 - protein, 7 aa, Homo sapiens
KRSRRNL

SEQ ID NO: 1845 - protein, 7 aa, Homo sapiens
RSRRNLG

SEQ ID NO: 1846 - protein, 7 aa, Homo sapiens
SRRNLGL

SEQ ID NO: 1847 - protein, 7 aa, Homo sapiens
RRNLGLD

SEQ ID NO: 1848 - protein, 7 aa, Homo sapiens
RNLGLDC

SEQ ID NO: 1849 - protein, 7 aa, Homo sapiens
NLGLDCD

SEQ ID NO: 1850 - protein, 7 aa, Homo sapiens
LGLDCDE

SEQ ID NO: 1851 - protein, 7 aa, Homo sapiens
GLDCDEH

SEQ ID NO: 1852 - protein, 7 aa, Homo sapiens
LDCDEHS

SEQ ID NO: 1853 - protein, 7 aa, Homo sapiens
DCDEHSS

SEQ ID NO: 1854 - protein, 7 aa, Homo sapiens
CDEHSSE

SEQ ID NO: 1855 - protein, 7 aa, Homo sapiens
DEHSSES

SEQ ID NO: 1856 - protein, 7 aa, Homo sapiens
EHSSESR

SEQ ID NO: 1857 - protein, 7 aa, Homo sapiens
HSSESRC

SEQ ID NO: 1858 - protein, 7 aa, Homo sapiens
SSESRCC

SEQ ID NO: 1859 - protein, 7 aa, Homo sapiens
SESRCCR

SEQ ID NO: 1860 - protein, 7 aa, Homo sapiens
ESRCCRY

SEQ ID NO: 1861 - protein, 7 aa, Homo sapiens
SRCCRYP

SEQ ID NO: 1862 - protein, 7 aa, Homo sapiens
RCCRYPL

SEQ ID NO: 1863 - protein, 7 aa, Homo sapiens
CCRYPLT

SEQ ID NO: 1864 - protein, 7 aa, Homo sapiens
CRYPLTV

SEQ ID NO: 1865 - protein, 7 aa, Homo sapiens
RYPLTVD

SEQ ID NO: 1866 - protein, 7 aa, Homo sapiens
YPLTVDF

SEQ ID NO: 1867 - protein, 7 aa, Homo sapiens
PLTVDFE

SEQ ID NO: 1868 - protein, 7 aa, Homo sapiens
LTVDFEA

SEQ ID NO: 1869 - protein, 7 aa, Homo sapiens
TVDFEAF

SEQ ID NO: 1870 - protein, 7 aa, Homo sapiens
VDFEAFG

SEQ ID NO: 1871 - protein, 7 aa, Homo sapiens
DFEAFGW

SEQ ID NO: 1872 - protein, 7 aa, Homo sapiens
FEAFGWD

SEQ ID NO: 1873 - protein, 7 aa, Homo sapiens
EAFGWDW

SEQ ID NO: 1874 - protein, 7 aa, Homo sapiens
AFGWDWI

SEQ ID NO: 1875 - protein, 7 aa, Homo sapiens
FGWDWII

SEQ ID NO: 1876 - protein, 7 aa, Homo sapiens
GWDWIIA

SEQ ID NO: 1877 - protein, 7 aa, Homo sapiens
WDWIIAP

SEQ ID NO: 1878 - protein, 7 aa, Homo sapiens
DWIIAPK

SEQ ID NO: 1879 - protein, 7 aa, Homo sapiens
WIIAPKR

SEQ ID NO: 1880 - protein, 7 aa, Homo sapiens
IIAPKRY

SEQ ID NO: 1881 - protein, 7 aa, Homo sapiens
IAPKRYK

SEQ ID NO: 1882 - protein, 7 aa, Homo sapiens
APKRYKA

SEQ ID NO: 1883 - protein, 7 aa, Homo sapiens
PKRYKAN

SEQ ID NO: 1884 - protein, 7 aa, Homo sapiens
KRYKANY

SEQ ID NO: 1885 - protein, 7 aa, Homo sapiens
RYKANYC

SEQ ID NO: 1886 - protein, 7 aa, Homo sapiens
YKANYCS

SEQ ID NO: 1887 - protein, 7 aa, Homo sapiens
KANYCSG

SEQ ID NO: 1888 - protein, 7 aa, Homo sapiens
ANYCSGQ

SEQ ID NO: 1889 - protein, 7 aa, Homo sapiens
NYCSGQC

SEQ ID NO: 1890 - protein, 7 aa, Homo sapiens
YCSGQCE

SEQ ID NO: 1891 - protein, 7 aa, Homo sapiens
CSGQCEY

SEQ ID NO: 1892 - protein, 7 aa, Homo sapiens
SGQCEYM

SEQ ID NO: 1893 - protein, 7 aa, Homo sapiens
GQCEYMF

SEQ ID NO: 1894 - protein, 7 aa, Homo sapiens
QCEYMFM

SEQ ID NO: 1895 - protein, 7 aa, Homo sapiens
CEYMFMQ

SEQ ID NO: 1896 - protein, 7 aa, Homo sapiens
EYMFMQK

SEQ ID NO: 1897 - protein, 7 aa, Homo sapiens
YMFMQKY

SEQ ID NO: 1898 - protein, 7 aa, Homo sapiens
MFMQKYP

SEQ ID NO: 1899 - protein, 7 aa, Homo sapiens
FMQKYPH

SEQ ID NO: 1900 - protein, 7 aa, Homo sapiens
MQKYPHT

SEQ ID NO: 1901 - protein, 7 aa, Homo sapiens
QKYPHTH

SEQ ID NO: 1902 - protein, 7 aa, Homo sapiens
KYPHTHL

SEQ ID NO: 1903 - protein, 7 aa, Homo sapiens
YPHTHLV

SEQ ID NO: 1904 - protein, 7 aa, Homo sapiens
PHTHLVQ

SEQ ID NO: 1905 - protein, 7 aa, Homo sapiens
HTHLVQQ

SEQ ID NO: 1906 - protein, 7 aa, Homo sapiens
THLVQQA

SEQ ID NO: 1907 - protein, 7 aa, Homo sapiens
HLVQQAN

SEQ ID NO: 1908 - protein, 7 aa, Homo sapiens
LVQQANP

SEQ ID NO: 1909 - protein, 7 aa, Homo sapiens
VQQANPR

SEQ ID NO: 1910 - protein, 7 aa, Homo sapiens
QQANPRG

SEQ ID NO: 1911 - protein, 7 aa, Homo sapiens
QANPRGS

SEQ ID NO: 1912 - protein, 7 aa, Homo sapiens
ANPRGSA

SEQ ID NO: 1913 - protein, 7 aa, Homo sapiens
NPRGSAG

SEQ ID NO: 1914 - protein, 7 aa, Homo sapiens
PRGSAGP

SEQ ID NO: 1915 - protein, 7 aa, Homo sapiens
RGSAGPC

SEQ ID NO: 1916 - protein, 7 aa, Homo sapiens
GSAGPCC

SEQ ID NO: 1917 - protein, 7 aa, Homo sapiens
SAGPCCT

SEQ ID NO: 1918 - protein, 7 aa, Homo sapiens
AGPCCTP

SEQ ID NO: 1919 - protein, 7 aa, Homo sapiens
GPCCTPT

SEQ ID NO: 1920 - protein, 7 aa, Homo sapiens
PCCTPTK

SEQ ID NO: 1921 - protein, 7 aa, Homo sapiens
CCTPTKM

SEQ ID NO: 1922 - protein, 7 aa, Homo sapiens
CTPTKMS

SEQ ID NO: 1923 - protein, 7 aa, Homo sapiens
TPTKMSP

SEQ ID NO: 1924 - protein, 7 aa, Homo sapiens
PTKMSPI

SEQ ID NO: 1925 - protein, 7 aa, Homo sapiens
TKMSPIN

SEQ ID NO: 1926 - protein, 7 aa, Homo sapiens
KMSPINM

SEQ ID NO: 1927 - protein, 7 aa, Homo sapiens
MSPINML

SEQ ID NO: 1928 - protein, 7 aa, Homo sapiens
SPINMLY

SEQ ID NO: 1929 - protein, 7 aa, Homo sapiens
PINMLYF

SEQ ID NO: 1930 - protein, 7 aa, Homo sapiens
INMLYFN

SEQ ID NO: 1931 - protein, 7 aa, Homo sapiens
NMLYFND

SEQ ID NO: 1932 - protein, 7 aa, Homo sapiens
MLYFNDK

SEQ ID NO: 1933 - protein, 7 aa, Homo sapiens
LYFNDKQ

SEQ ID NO: 1934 - protein, 7 aa, Homo sapiens
YFNDKQQ

SEQ ID NO: 1935 - protein, 7 aa, Homo sapiens
FNDKQQI

SEQ ID NO: 1936 - protein, 7 aa, Homo sapiens
NDKQQII

SEQ ID NO: 1937 - protein, 7 aa, Homo sapiens
DKQQIIY

SEQ ID NO: 1938 - protein, 7 aa, Homo sapiens
KQQIIYG

SEQ ID NO: 1939 - protein, 7 aa, Homo sapiens
QQIIYGK

SEQ ID NO: 1940 - protein, 7 aa, Homo sapiens
QIIYGKI

SEQ ID NO: 1941 - protein, 7 aa, Homo sapiens
IIYGKIP

SEQ ID NO: 1942 - protein, 7 aa, Homo sapiens
IYGKIPG

SEQ ID NO: 1943 - protein, 7 aa, Homo sapiens
YGKIPGM

SEQ ID NO: 1944 - protein, 7 aa, Homo sapiens
GKIPGMV

SEQ ID NO: 1945 - protein, 7 aa, Homo sapiens
KIPGMVV

SEQ ID NO: 1946 - protein, 7 aa, Homo sapiens
IPGMVVD

SEQ ID NO: 1947 - protein, 7 aa, Homo sapiens
PGMVVDR

SEQ ID NO: 1948 - protein, 7 aa, Homo sapiens
GMVVDRC

SEQ ID NO: 1949 - protein, 7 aa, Homo sapiens
MVVDRCG

SEQ ID NO: 1950 - protein, 7 aa, Homo sapiens
VVDRCGC

SEQ ID NO: 1951 - protein, 7 aa, Homo sapiens
VDRCGCS

SEQ ID NO: 1952 - protein, 8 aa, Homo sapiens
MVLAAPLL

SEQ ID NO: 1953 - protein, 8 aa, Homo sapiens
VLAAPLLL

SEQ ID NO: 1954 - protein, 8 aa, Homo sapiens
LAAPLLLG

SEQ ID NO: 1955 - protein, 8 aa, Homo sapiens
AAPLLLGF

SEQ ID NO: 1956 - protein, 8 aa, Homo sapiens
APLLLGFL

SEQ ID NO: 1957 - protein, 8 aa, Homo sapiens
PLLLGFLL

SEQ ID NO: 1958 - protein, 8 aa, Homo sapiens
LLLGFLLL

SEQ ID NO: 1959 - protein, 8 aa, Homo sapiens
LLGFLLLA

SEQ ID NO: 1960 - protein, 8 aa, Homo sapiens
LGFLLLAL

SEQ ID NO: 1961 - protein, 8 aa, Homo sapiens
GFLLLALE

SEQ ID NO: 1962 - protein, 8 aa, Homo sapiens
FLLLALEL

SEQ ID NO: 1963 - protein, 8 aa, Homo sapiens
LLLALELR

SEQ ID NO: 1964 - protein, 8 aa, Homo sapiens
LLALELRP

SEQ ID NO: 1965 - protein, 8 aa, Homo sapiens
LALELRPR

SEQ ID NO: 1966 - protein, 8 aa, Homo sapiens
ALELRPRG

SEQ ID NO: 1967 - protein, 8 aa, Homo sapiens
LELRPRGE

SEQ ID NO: 1968 - protein, 8 aa, Homo sapiens
ELRPRGEA

SEQ ID NO: 1969 - protein, 8 aa, Homo sapiens
LRPRGEAA

SEQ ID NO: 1970 - protein, 8 aa, Homo sapiens
RPRGEAAE

SEQ ID NO: 1971 - protein, 8 aa, Homo sapiens
PRGEAAEG

SEQ ID NO: 1972 - protein, 8 aa, Homo sapiens
RGEAAEGP

SEQ ID NO: 1973 - protein, 8 aa, Homo sapiens
GEAAEGPA

SEQ ID NO: 1974 - protein, 8 aa, Homo sapiens
EAAEGPAA

SEQ ID NO: 1975 - protein, 8 aa, Homo sapiens
AAEGPAAA

SEQ ID NO: 1976 - protein, 8 aa, Homo sapiens
AEGPAAAA

SEQ ID NO: 1977 - protein, 8 aa, Homo sapiens
EGPAAAAA

SEQ ID NO: 1978 - protein, 8 aa, Homo sapiens
GPAAAAAA

SEQ ID NO: 1979 - protein, 8 aa, Homo sapiens
PAAAAAAA

SEQ ID NO: 1980 - protein, 8 aa, Homo sapiens
AAAAAAAA

SEQ ID NO: 1981 - protein, 8 aa, Homo sapiens
AAAAAAAG

SEQ ID NO: 1982 - protein, 8 aa, Homo sapiens
AAAAAAGV

SEQ ID NO: 1983 - protein, 8 aa, Homo sapiens
AAAAAGVG

SEQ ID NO: 1984 - protein, 8 aa, Homo sapiens
AAAAGVGG

SEQ ID NO: 1985 - protein, 8 aa, Homo sapiens
AAAGVGGE

SEQ ID NO: 1986 - protein, 8 aa, Homo sapiens
AAGVGGER

SEQ ID NO: 1987 - protein, 8 aa, Homo sapiens
AGVGGERS

SEQ ID NO: 1988 - protein, 8 aa, Homo sapiens
GVGGERSS

SEQ ID NO: 1989 - protein, 8 aa, Homo sapiens
VGGERSSR

SEQ ID NO: 1990 - protein, 8 aa, Homo sapiens
GGERSSRP

SEQ ID NO: 1991 - protein, 8 aa, Homo sapiens
GERSSRPA

SEQ ID NO: 1992 - protein, 8 aa, Homo sapiens
ERSSRPAP

SEQ ID NO: 1993 - protein, 8 aa, Homo sapiens
RSSRPAPS

SEQ ID NO: 1994 - protein, 8 aa, Homo sapiens
SSRPAPSV

SEQ ID NO: 1995 - protein, 8 aa, Homo sapiens
SRPAPSVA

SEQ ID NO: 1996 - protein, 8 aa, Homo sapiens
RPAPSVAP

SEQ ID NO: 1997 - protein, 8 aa, Homo sapiens
PAPSVAPE

SEQ ID NO: 1998 - protein, 8 aa, Homo sapiens
APSVAPEP

SEQ ID NO: 1999 - protein, 8 aa, Homo sapiens
PSVAPEPD

SEQ ID NO: 2000 - protein, 8 aa, Homo sapiens
SVAPEPDG

SEQ ID NO: 2001 - protein, 8 aa, Homo sapiens
VAPEPDGC

SEQ ID NO: 2002 - protein, 8 aa, Homo sapiens
APEPDGCP

SEQ ID NO: 2003 - protein, 8 aa, Homo sapiens
PEPDGCPV

SEQ ID NO: 2004 - protein, 8 aa, Homo sapiens
EPDGCPVC

SEQ ID NO: 2005 - protein, 8 aa, Homo sapiens
PDGCPVCV

SEQ ID NO: 2006 - protein, 8 aa, Homo sapiens
DGCPVCVW

SEQ ID NO: 2007 - protein, 8 aa, Homo sapiens
GCPVCVWR

SEQ ID NO: 2008 - protein, 8 aa, Homo sapiens
CPVCVWRQ

SEQ ID NO: 2009 - protein, 8 aa, Homo sapiens
PVCVWRQH

SEQ ID NO: 2010 - protein, 8 aa, Homo sapiens
VCVWRQHS

SEQ ID NO: 2011 - protein, 8 aa, Homo sapiens
CVWRQHSR

SEQ ID NO: 2012 - protein, 8 aa, Homo sapiens
VWRQHSRE

SEQ ID NO: 2013 - protein, 8 aa, Homo sapiens
WRQHSREL

SEQ ID NO: 2014 - protein, 8 aa, Homo sapiens
RQHSRELR

SEQ ID NO: 2015 - protein, 8 aa, Homo sapiens
QHSRELRL

SEQ ID NO: 2016 - protein, 8 aa, Homo sapiens
HSRELRLE

SEQ ID NO: 2017 - protein, 8 aa, Homo sapiens
SRELRLES

SEQ ID NO: 2018 - protein, 8 aa, Homo sapiens
RELRLESI

SEQ ID NO: 2019 - protein, 8 aa, Homo sapiens
ELRLESIK

SEQ ID NO: 2020 - protein, 8 aa, Homo sapiens
LRLESIKS

SEQ ID NO: 2021 - protein, 8 aa, Homo sapiens
RLESIKSQ

SEQ ID NO: 2022 - protein, 8 aa, Homo sapiens
LESIKSQI

SEQ ID NO: 2023 - protein, 8 aa, Homo sapiens
ESIKSQIL

SEQ ID NO: 2024 - protein, 8 aa, Homo sapiens
SIKSQILS

SEQ ID NO: 2025 - protein, 8 aa, Homo sapiens
IKSQILSK

SEQ ID NO: 2026 - protein, 8 aa, Homo sapiens
KSQILSKL

SEQ ID NO: 2027 - protein, 8 aa, Homo sapiens
SQILSKLR

SEQ ID NO: 2028 - protein, 8 aa, Homo sapiens
QILSKLRL

SEQ ID NO: 2029 - protein, 8 aa, Homo sapiens
ILSKLRLK

SEQ ID NO: 2030 - protein, 8 aa, Homo sapiens
LSKLRLKE

SEQ ID NO: 2031 - protein, 8 aa, Homo sapiens
SKLRLKEA

SEQ ID NO: 2032 - protein, 8 aa, Homo sapiens
KLRLKEAP

SEQ ID NO: 2033 - protein, 8 aa, Homo sapiens
LRLKEAPN

SEQ ID NO: 2034 - protein, 8 aa, Homo sapiens
RLKEAPNI

SEQ ID NO: 2035 - protein, 8 aa, Homo sapiens
LKEAPNIS

SEQ ID NO: 2036 - protein, 8 aa, Homo sapiens
KEAPNISR

SEQ ID NO: 2037 - protein, 8 aa, Homo sapiens
EAPNISRE

SEQ ID NO: 2038 - protein, 8 aa, Homo sapiens
APNISREV

SEQ ID NO: 2039 - protein, 8 aa, Homo sapiens
PNISREVV

SEQ ID NO: 2040 - protein, 8 aa, Homo sapiens
NISREVVK

SEQ ID NO: 2041 - protein, 8 aa, Homo sapiens
ISREVVKQ

SEQ ID NO: 2042 - protein, 8 aa, Homo sapiens
SREVVKQL

SEQ ID NO: 2043 - protein, 8 aa, Homo sapiens
REVVKQLL

SEQ ID NO: 2044 - protein, 8 aa, Homo sapiens
EVVKQLLP

SEQ ID NO: 2045 - protein, 8 aa, Homo sapiens
VVKQLLPK

SEQ ID NO: 2046 - protein, 8 aa, Homo sapiens
VKQLLPKA

SEQ ID NO: 2047 - protein, 8 aa, Homo sapiens
KQLLPKAP

SEQ ID NO: 2048 - protein, 8 aa, Homo sapiens
QLLPKAPP

SEQ ID NO: 2049 - protein, 8 aa, Homo sapiens
LLPKAPPL

SEQ ID NO: 2050 - protein, 8 aa, Homo sapiens
LPKAPPLQ

SEQ ID NO: 2051 - protein, 8 aa, Homo sapiens
PKAPPLQQ

SEQ ID NO: 2052 - protein, 8 aa, Homo sapiens
KAPPLQQI

SEQ ID NO: 2053 - protein, 8 aa, Homo sapiens
APPLQQIL

SEQ ID NO: 2054 - protein, 8 aa, Homo sapiens
PPLQQILD

SEQ ID NO: 2055 - protein, 8 aa, Homo sapiens
PLQQILDL

SEQ ID NO: 2056 - protein, 8 aa, Homo sapiens
LQQILDLH

SEQ ID NO: 2057 - protein, 8 aa, Homo sapiens
QQILDLHD

SEQ ID NO: 2058 - protein, 8 aa, Homo sapiens
QILDLHDF

SEQ ID NO: 2059 - protein, 8 aa, Homo sapiens
ILDLHDFQ

SEQ ID NO: 2060 - protein, 8 aa, Homo sapiens
LDLHDFQG

SEQ ID NO: 2061 - protein, 8 aa, Homo sapiens
DLHDFQGD

SEQ ID NO: 2062 - protein, 8 aa, Homo sapiens
LHDFQGDA

SEQ ID NO: 2063 - protein, 8 aa, Homo sapiens
HDFQGDAL

SEQ ID NO: 2064 - protein, 8 aa, Homo sapiens
DFQGDALQ

SEQ ID NO: 2065 - protein, 8 aa, Homo sapiens
FQGDALQP

SEQ ID NO: 2066 - protein, 8 aa, Homo sapiens
QGDALQPE

SEQ ID NO: 2067 - protein, 8 aa, Homo sapiens
GDALQPED

SEQ ID NO: 2068 - protein, 8 aa, Homo sapiens
DALQPEDF

SEQ ID NO: 2069 - protein, 8 aa, Homo sapiens
ALQPEDFL

SEQ ID NO: 2070 - protein, 8 aa, Homo sapiens
LQPEDFLE

SEQ ID NO: 2071 - protein, 8 aa, Homo sapiens
QPEDFLEE

SEQ ID NO: 2072 - protein, 8 aa, Homo sapiens
PEDFLEED

SEQ ID NO: 2073 - protein, 8 aa, Homo sapiens
EDFLEEDE

SEQ ID NO: 2074 - protein, 8 aa, Homo sapiens
DFLEEDEY

SEQ ID NO: 2075 - protein, 8 aa, Homo sapiens
FLEEDEYH

SEQ ID NO: 2076 - protein, 8 aa, Homo sapiens
LEEDEYHA

SEQ ID NO: 2077 - protein, 8 aa, Homo sapiens
EEDEYHAT

SEQ ID NO: 2078 - protein, 8 aa, Homo sapiens
EDEYHATT

SEQ ID NO: 2079 - protein, 8 aa, Homo sapiens
DEYHATTE

SEQ ID NO: 2080 - protein, 8 aa, Homo sapiens
EYHATTET

SEQ ID NO: 2081 - protein, 8 aa, Homo sapiens
YHATTETV

SEQ ID NO: 2082 - protein, 8 aa, Homo sapiens
HATTETVI

SEQ ID NO: 2083 - protein, 8 aa, Homo sapiens
ATTETVIS

SEQ ID NO: 2084 - protein, 8 aa, Homo sapiens
TTETVISM

SEQ ID NO: 2085 - protein, 8 aa, Homo sapiens
TETVISMA

SEQ ID NO: 2086 - protein, 8 aa, Homo sapiens
ETVISMAQ

SEQ ID NO: 2087 - protein, 8 aa, Homo sapiens
TVISMAQE

SEQ ID NO: 2088 - protein, 8 aa, Homo sapiens
VISMAQET

SEQ ID NO: 2089 - protein, 8 aa, Homo sapiens
ISMAQETD

SEQ ID NO: 2090 - protein, 8 aa, Homo sapiens
SMAQETDP

SEQ ID NO: 2091 - protein, 8 aa, Homo sapiens
MAQETDPA

SEQ ID NO: 2092 - protein, 8 aa, Homo sapiens
AQETDPAV

SEQ ID NO: 2093 - protein, 8 aa, Homo sapiens
QETDPAVQ

SEQ ID NO: 2094 - protein, 8 aa, Homo sapiens
ETDPAVQT

SEQ ID NO: 2095 - protein, 8 aa, Homo sapiens
TDPAVQTD

SEQ ID NO: 2096 - protein, 8 aa, Homo sapiens
DPAVQTDG

SEQ ID NO: 2097 - protein, 8 aa, Homo sapiens
PAVQTDGS

SEQ ID NO: 2098 - protein, 8 aa, Homo sapiens
AVQTDGSP

SEQ ID NO: 2099 - protein, 8 aa, Homo sapiens
VQTDGSPL

SEQ ID NO: 2100 - protein, 8 aa, Homo sapiens
QTDGSPLC

SEQ ID NO: 2101 - protein, 8 aa, Homo sapiens
TDGSPLCC

SEQ ID NO: 2102 - protein, 8 aa, Homo sapiens
DGSPLCCH

SEQ ID NO: 2103 - protein, 8 aa, Homo sapiens
GSPLCCHF

SEQ ID NO: 2104 - protein, 8 aa, Homo sapiens
SPLCCHFH

SEQ ID NO: 2105 - protein, 8 aa, Homo sapiens
PLCCHFHF

SEQ ID NO: 2106 - protein, 8 aa, Homo sapiens
LCCHFHFS

SEQ ID NO: 2107 - protein, 8 aa, Homo sapiens
CCHFHFSP

SEQ ID NO: 2108 - protein, 8 aa, Homo sapiens
CHFHFSPK

SEQ ID NO: 2109 - protein, 8 aa, Homo sapiens
HFHFSPKV

SEQ ID NO: 2110 - protein, 8 aa, Homo sapiens
FHFSPKVM

SEQ ID NO: 2111 - protein, 8 aa, Homo sapiens
HFSPKVMF

SEQ ID NO: 2112 - protein, 8 aa, Homo sapiens
FSPKVMFT

SEQ ID NO: 2113 - protein, 8 aa, Homo sapiens
SPKVMFTK

SEQ ID NO: 2114 - protein, 8 aa, Homo sapiens
PKVMFTKV

SEQ ID NO: 2115 - protein, 8 aa, Homo sapiens
KVMFTKVL

SEQ ID NO: 2116 - protein, 8 aa, Homo sapiens
VMFTKVLK

SEQ ID NO: 2117 - protein, 8 aa, Homo sapiens
MFTKVLKA

SEQ ID NO: 2118 - protein, 8 aa, Homo sapiens
FTKVLKAQ

SEQ ID NO: 2119 - protein, 8 aa, Homo sapiens
TKVLKAQL

SEQ ID NO: 2120 - protein, 8 aa, Homo sapiens
KVLKAQLW

SEQ ID NO: 2121 - protein, 8 aa, Homo sapiens
VLKAQLWV

SEQ ID NO: 2122 - protein, 8 aa, Homo sapiens
LKAQLWVY

SEQ ID NO: 2123 - protein, 8 aa, Homo sapiens
KAQLWVYL

SEQ ID NO: 2124 - protein, 8 aa, Homo sapiens
AQLWVYLR

SEQ ID NO: 2125 - protein, 8 aa, Homo sapiens
QLWVYLRP

SEQ ID NO: 2126 - protein, 8 aa, Homo sapiens
LWVYLRPV

SEQ ID NO: 2127 - protein, 8 aa, Homo sapiens
WVYLRPVP

SEQ ID NO: 2128 - protein, 8 aa, Homo sapiens
VYLRPVPR

SEQ ID NO: 2129 - protein, 8 aa, Homo sapiens
YLRPVPRP

SEQ ID NO: 2130 - protein, 8 aa, Homo sapiens
LRPVPRPA

SEQ ID NO: 2131 - protein, 8 aa, Homo sapiens
RPVPRPAT

SEQ ID NO: 2132 - protein, 8 aa, Homo sapiens
PVPRPATV

SEQ ID NO: 2133 - protein, 8 aa, Homo sapiens
VPRPATVY

SEQ ID NO: 2134 - protein, 8 aa, Homo sapiens
PRPATVYL

SEQ ID NO: 2135 - protein, 8 aa, Homo sapiens
RPATVYLQ

SEQ ID NO: 2136 - protein, 8 aa, Homo sapiens
PATVYLQI

SEQ ID NO: 2137 - protein, 8 aa, Homo sapiens
ATVYLQIL

SEQ ID NO: 2138 - protein, 8 aa, Homo sapiens
TVYLQILR

SEQ ID NO: 2139 - protein, 8 aa, Homo sapiens
VYLQILRL

SEQ ID NO: 2140 - protein, 8 aa, Homo sapiens
YLQILRLK

SEQ ID NO: 2141 - protein, 8 aa, Homo sapiens
LQILRLKP

SEQ ID NO: 2142 - protein, 8 aa, Homo sapiens
QILRLKPL

SEQ ID NO: 2143 - protein, 8 aa, Homo sapiens
ILRLKPLT

SEQ ID NO: 2144 - protein, 8 aa, Homo sapiens
LRLKPLTG

SEQ ID NO: 2145 - protein, 8 aa, Homo sapiens
RLKPLTGE

SEQ ID NO: 2146 - protein, 8 aa, Homo sapiens
LKPLTGEG

SEQ ID NO: 2147 - protein, 8 aa, Homo sapiens
KPLTGEGT

SEQ ID NO: 2148 - protein, 8 aa, Homo sapiens
PLTGEGTA

SEQ ID NO: 2149 - protein, 8 aa, Homo sapiens
LTGEGTAG

SEQ ID NO: 2150 - protein, 8 aa, Homo sapiens
TGEGTAGG

SEQ ID NO: 2151 - protein, 8 aa, Homo sapiens
GEGTAGGG

SEQ ID NO: 2152 - protein, 8 aa, Homo sapiens
EGTAGGGG

SEQ ID NO: 2153 - protein, 8 aa, Homo sapiens
GTAGGGGG

SEQ ID NO: 2154 - protein, 8 aa, Homo sapiens
TAGGGGGG

SEQ ID NO: 2155 - protein, 8 aa, Homo sapiens
AGGGGGGR

SEQ ID NO: 2156 - protein, 8 aa, Homo sapiens
GGGGGGRR

SEQ ID NO: 2157 - protein, 8 aa, Homo sapiens
GGGGGRRH

SEQ ID NO: 2158 - protein, 8 aa, Homo sapiens
GGGGRRHI

SEQ ID NO: 2159 - protein, 8 aa, Homo sapiens
GGGRRHIR

SEQ ID NO: 2160 - protein, 8 aa, Homo sapiens
GGRRHIRI

SEQ ID NO: 2161 - protein, 8 aa, Homo sapiens
GRRHIRIR

SEQ ID NO: 2162 - protein, 8 aa, Homo sapiens
RRHIRIRS

SEQ ID NO: 2163 - protein, 8 aa, Homo sapiens
RHIRIRSL

SEQ ID NO: 2164 - protein, 8 aa, Homo sapiens
HIRIRSLK

SEQ ID NO: 2165 - protein, 8 aa, Homo sapiens
IRIRSLKI

SEQ ID NO: 2166 - protein, 8 aa, Homo sapiens
RIRSLKIE

SEQ ID NO: 2167 - protein, 8 aa, Homo sapiens
IRSLKIEL

SEQ ID NO: 2168 - protein, 8 aa, Homo sapiens
RSLKIELH

SEQ ID NO: 2169 - protein, 8 aa, Homo sapiens
SLKIELHS

SEQ ID NO: 2170 - protein, 8 aa, Homo sapiens
LKIELHSR

SEQ ID NO: 2171 - protein, 8 aa, Homo sapiens
KIELHSRS

SEQ ID NO: 2172 - protein, 8 aa, Homo sapiens
IELHSRSG

SEQ ID NO: 2173 - protein, 8 aa, Homo sapiens
ELHSRSGH

SEQ ID NO: 2174 - protein, 8 aa, Homo sapiens
LHSRSGHW

SEQ ID NO: 2175 - protein, 8 aa, Homo sapiens
HSRSGHWQ

SEQ ID NO: 2176 - protein, 8 aa, Homo sapiens
SRSGHWQS

SEQ ID NO: 2177 - protein, 8 aa, Homo sapiens
RSGHWQSI

SEQ ID NO: 2178 - protein, 8 aa, Homo sapiens
SGHWQSID

SEQ ID NO: 2179 - protein, 8 aa, Homo sapiens
GHWQSIDF

SEQ ID NO: 2180 - protein, 8 aa, Homo sapiens
HWQSIDFK

SEQ ID NO: 2181 - protein, 8 aa, Homo sapiens
WQSIDFKQ

SEQ ID NO: 2182 - protein, 8 aa, Homo sapiens
QSIDFKQV

SEQ ID NO: 2183 - protein, 8 aa, Homo sapiens
SIDFKQVL

SEQ ID NO: 2184 - protein, 8 aa, Homo sapiens
IDFKQVLH

SEQ ID NO: 2185 - protein, 8 aa, Homo sapiens
DFKQVLHS

SEQ ID NO: 2186 - protein, 8 aa, Homo sapiens
FKQVLHSW

SEQ ID NO: 2187 - protein, 8 aa, Homo sapiens
KQVLHSWF

SEQ ID NO: 2188 - protein, 8 aa, Homo sapiens
QVLHSWFR

SEQ ID NO: 2189 - protein, 8 aa, Homo sapiens
VLHSWFRQ

SEQ ID NO: 2190 - protein, 8 aa, Homo sapiens
LHSWFRQP

SEQ ID NO: 2191 - protein, 8 aa, Homo sapiens
HSWFRQPQ

SEQ ID NO: 2192 - protein, 8 aa, Homo sapiens
SWFRQPQS

SEQ ID NO: 2193 - protein, 8 aa, Homo sapiens
WFRQPQSN

SEQ ID NO: 2194 - protein, 8 aa, Homo sapiens
FRQPQSNW

SEQ ID NO: 2195 - protein, 8 aa, Homo sapiens
RQPQSNWG

SEQ ID NO: 2196 - protein, 8 aa, Homo sapiens
QPQSNWGI

SEQ ID NO: 2197 - protein, 8 aa, Homo sapiens
PQSNWGIE

SEQ ID NO: 2198 - protein, 8 aa, Homo sapiens
QSNWGIEI

SEQ ID NO: 2199 - protein, 8 aa, Homo sapiens
SNWGIEIN

SEQ ID NO: 2200 - protein, 8 aa, Homo sapiens
NWGIEINA

SEQ ID NO: 2201 - protein, 8 aa, Homo sapiens
WGIEINAF

SEQ ID NO: 2202 - protein, 8 aa, Homo sapiens
GIEINAFD

SEQ ID NO: 2203 - protein, 8 aa, Homo sapiens
IEINAFDP

SEQ ID NO: 2204 - protein, 8 aa, Homo sapiens
EINAFDPS

SEQ ID NO: 2205 - protein, 8 aa, Homo sapiens
INAFDPSG

SEQ ID NO: 2206 - protein, 8 aa, Homo sapiens
NAFDPSGT

SEQ ID NO: 2207 - protein, 8 aa, Homo sapiens
AFDPSGTD

SEQ ID NO: 2208 - protein, 8 aa, Homo sapiens
FDPSGTDL

SEQ ID NO: 2209 - protein, 8 aa, Homo sapiens
DPSGTDLA

SEQ ID NO: 2210 - protein, 8 aa, Homo sapiens
PSGTDLAV

SEQ ID NO: 2211 - protein, 8 aa, Homo sapiens
SGTDLAVT

SEQ ID NO: 2212 - protein, 8 aa, Homo sapiens
GTDLAVTS

SEQ ID NO: 2213 - protein, 8 aa, Homo sapiens
TDLAVTSL

SEQ ID NO: 2214 - protein, 8 aa, Homo sapiens
DLAVTSLG

SEQ ID NO: 2215 - protein, 8 aa, Homo sapiens
LAVTSLGP

SEQ ID NO: 2216 - protein, 8 aa, Homo sapiens
AVTSLGPG

SEQ ID NO: 2217 - protein, 8 aa, Homo sapiens
VTSLGPGA

SEQ ID NO: 2218 - protein, 8 aa, Homo sapiens
TSLGPGAE

SEQ ID NO: 2219 - protein, 8 aa, Homo sapiens
SLGPGAEG

SEQ ID NO: 2220 - protein, 8 aa, Homo sapiens
LGPGAEGL

SEQ ID NO: 2221 - protein, 8 aa, Homo sapiens
GPGAEGLH

SEQ ID NO: 2222 - protein, 8 aa, Homo sapiens
PGAEGLHP

SEQ ID NO: 2223 - protein, 8 aa, Homo sapiens
GAEGLHPF

SEQ ID NO: 2224 - protein, 8 aa, Homo sapiens
AEGLHPFM

SEQ ID NO: 2225 - protein, 8 aa, Homo sapiens
EGLHPFME

SEQ ID NO: 2226 - protein, 8 aa, Homo sapiens
GLHPFMEL

SEQ ID NO: 2227 - protein, 8 aa, Homo sapiens
LHPFMELR

SEQ ID NO: 2228 - protein, 8 aa, Homo sapiens
HPFMELRV

SEQ ID NO: 2229 - protein, 8 aa, Homo sapiens
PFMELRVL

SEQ ID NO: 2230 - protein, 8 aa, Homo sapiens
FMELRVLE

SEQ ID NO: 2231 - protein, 8 aa, Homo sapiens
MELRVLEN

SEQ ID NO: 2232 - protein, 8 aa, Homo sapiens
ELRVLENT

SEQ ID NO: 2233 - protein, 8 aa, Homo sapiens
LRVLENTK

SEQ ID NO: 2234 - protein, 8 aa, Homo sapiens
RVLENTKR

SEQ ID NO: 2235 - protein, 8 aa, Homo sapiens
VLENTKRS

SEQ ID NO: 2236 - protein, 8 aa, Homo sapiens
LENTKRSR

SEQ ID NO: 2237 - protein, 8 aa, Homo sapiens
ENTKRSRR

SEQ ID NO: 2238 - protein, 8 aa, Homo sapiens
NTKRSRRN

SEQ ID NO: 2239 - protein, 8 aa, Homo sapiens
TKRSRRNL

SEQ ID NO: 2240 - protein, 8 aa, Homo sapiens
KRSRRNLG

SEQ ID NO: 2241 - protein, 8 aa, Homo sapiens
RSRRNLGL

SEQ ID NO: 2242 - protein, 8 aa, Homo sapiens
SRRNLGLD

SEQ ID NO: 2243 - protein, 8 aa, Homo sapiens
RRNLGLDC

SEQ ID NO: 2244 - protein, 8 aa, Homo sapiens
RNLGLDCD

SEQ ID NO: 2245 - protein, 8 aa, Homo sapiens
NLGLDCDE

SEQ ID NO: 2246 - protein, 8 aa, Homo sapiens
LGLDCDEH

SEQ ID NO: 2247 - protein, 8 aa, Homo sapiens
GLDCDEHS

SEQ ID NO: 2248 - protein, 8 aa, Homo sapiens
LDCDEHSS

SEQ ID NO: 2249 - protein, 8 aa, Homo sapiens
DCDEHSSE

SEQ ID NO: 2250 - protein, 8 aa, Homo sapiens
CDEHSSES

SEQ ID NO: 2251 - protein, 8 aa, Homo sapiens
DEHSSESR

SEQ ID NO: 2252 - protein, 8 aa, Homo sapiens
EHSSESRC

SEQ ID NO: 2253 - protein, 8 aa, Homo sapiens
HSSESRCC

SEQ ID NO: 2254 - protein, 8 aa, Homo sapiens
SSESRCCR

SEQ ID NO: 2255 - protein, 8 aa, Homo sapiens
SESRCCRY

SEQ ID NO: 2256 - protein, 8 aa, Homo sapiens
ESRCCRYP

SEQ ID NO: 2257 - protein, 8 aa, Homo sapiens
SRCCRYPL

SEQ ID NO: 2258 - protein, 8 aa, Homo sapiens
RCCRYPLT

SEQ ID NO: 2259 - protein, 8 aa, Homo sapiens
CCRYPLTV

SEQ ID NO: 2260 - protein, 8 aa, Homo sapiens
CRYPLTVD

SEQ ID NO: 2261 - protein, 8 aa, Homo sapiens
RYPLTVDF

SEQ ID NO: 2262 - protein, 8 aa, Homo sapiens
YPLTVDFE

SEQ ID NO: 2263 - protein, 8 aa, Homo sapiens
PLTVDFEA

SEQ ID NO: 2264 - protein, 8 aa, Homo sapiens
LTVDFEAF

SEQ ID NO: 2265 - protein, 8 aa, Homo sapiens
TVDFEAFG

SEQ ID NO: 2266 - protein, 8 aa, Homo sapiens
VDFEAFGW

SEQ ID NO: 2267 - protein, 8 aa, Homo sapiens
DFEAFGWD

SEQ ID NO: 2268 - protein, 8 aa, Homo sapiens
FEAFGWDW

SEQ ID NO: 2269 - protein, 8 aa, Homo sapiens
EAFGWDWI

SEQ ID NO: 2270 - protein, 8 aa, Homo sapiens
AFGWDWII

SEQ ID NO: 2271 - protein, 8 aa, Homo sapiens
FGWDWIIA

SEQ ID NO: 2272 - protein, 8 aa, Homo sapiens
GWDWIIAP

SEQ ID NO: 2273 - protein, 8 aa, Homo sapiens
WDWIIAPK

SEQ ID NO: 2274 - protein, 8 aa, Homo sapiens
DWIIAPKR

SEQ ID NO: 2275 - protein, 8 aa, Homo sapiens
WIIAPKRY

SEQ ID NO: 2276 - protein, 8 aa, Homo sapiens
IIAPKRYK

SEQ ID NO: 2277 - protein, 8 aa, Homo sapiens
IAPKRYKA

SEQ ID NO: 2278 - protein, 8 aa, Homo sapiens
APKRYKAN

SEQ ID NO: 2279 - protein, 8 aa, Homo sapiens
PKRYKANY

SEQ ID NO: 2280 - protein, 8 aa, Homo sapiens
KRYKANYC

SEQ ID NO: 2281 - protein, 8 aa, Homo sapiens
RYKANYCS

SEQ ID NO: 2282 - protein, 8 aa, Homo sapiens
YKANYCSG

SEQ ID NO: 2283 - protein, 8 aa, Homo sapiens
KANYCSGQ

SEQ ID NO: 2284 - protein, 8 aa, Homo sapiens
ANYCSGQC

SEQ ID NO: 2285 - protein, 8 aa, Homo sapiens
NYCSGQCE

SEQ ID NO: 2286 - protein, 8 aa, Homo sapiens
YCSGQCEY

SEQ ID NO: 2287 - protein, 8 aa, Homo sapiens
CSGQCEYM

SEQ ID NO: 2288 - protein, 8 aa, Homo sapiens
SGQCEYMF

SEQ ID NO: 2289 - protein, 8 aa, Homo sapiens
GQCEYMFM

SEQ ID NO: 2290 - protein, 8 aa, Homo sapiens
QCEYMFMQ

SEQ ID NO: 2291 - protein, 8 aa, Homo sapiens
CEYMFMQK

SEQ ID NO: 2292 - protein, 8 aa, Homo sapiens
EYMFMQKY

SEQ ID NO: 2293 - protein, 8 aa, Homo sapiens
YMFMQKYP

SEQ ID NO: 2294 - protein, 8 aa, Homo sapiens
MFMQKYPH

SEQ ID NO: 2295 - protein, 8 aa, Homo sapiens
FMQKYPHT

SEQ ID NO: 2296 - protein, 8 aa, Homo sapiens
MQKYPHTH

SEQ ID NO: 2297 - protein, 8 aa, Homo sapiens
QKYPHTHL

SEQ ID NO: 2298 - protein, 8 aa, Homo sapiens
KYPHTHLV

SEQ ID NO: 2299 - protein, 8 aa, Homo sapiens
YPHTHLVQ

SEQ ID NO: 2300 - protein, 8 aa, Homo sapiens
PHTHLVQQ

SEQ ID NO: 2301 - protein, 8 aa, Homo sapiens
HTHLVQQA

SEQ ID NO: 2302 - protein, 8 aa, Homo sapiens
THLVQQAN

SEQ ID NO: 2303 - protein, 8 aa, Homo sapiens
HLVQQANP

SEQ ID NO: 2304 - protein, 8 aa, Homo sapiens
LVQQANPR

SEQ ID NO: 2305 - protein, 8 aa, Homo sapiens
VQQANPRG

SEQ ID NO: 2306 - protein, 8 aa, Homo sapiens
QQANPRGS

SEQ ID NO: 2307 - protein, 8 aa, Homo sapiens
QANPRGSA

SEQ ID NO: 2308 - protein, 8 aa, Homo sapiens
ANPRGSAG

SEQ ID NO: 2309 - protein, 8 aa, Homo sapiens
NPRGSAGP

SEQ ID NO: 2310 - protein, 8 aa, Homo sapiens
PRGSAGPC

SEQ ID NO: 2311 - protein, 8 aa, Homo sapiens
RGSAGPCC

SEQ ID NO: 2312 - protein, 8 aa, Homo sapiens
GSAGPCCT

SEQ ID NO: 2313 - protein, 8 aa, Homo sapiens
SAGPCCTP

SEQ ID NO: 2314 - protein, 8 aa, Homo sapiens
AGPCCTPT

SEQ ID NO: 2315 - protein, 8 aa, Homo sapiens
GPCCTPTK

SEQ ID NO: 2316 - protein, 8 aa, Homo sapiens
PCCTPTKM

SEQ ID NO: 2317 - protein, 8 aa, Homo sapiens
CCTPTKMS

SEQ ID NO: 2318 - protein, 8 aa, Homo sapiens
CTPTKMSP

SEQ ID NO: 2319 - protein, 8 aa, Homo sapiens
TPTKMSPI

SEQ ID NO: 2320 - protein, 8 aa, Homo sapiens
PTKMSPIN

SEQ ID NO: 2321 - protein, 8 aa, Homo sapiens
TKMSPINM

SEQ ID NO: 2322 - protein, 8 aa, Homo sapiens
KMSPINML

SEQ ID NO: 2323 - protein, 8 aa, Homo sapiens
MSPINMLY

SEQ ID NO: 2324 - protein, 8 aa, Homo sapiens
SPINMLYF

SEQ ID NO: 2325 - protein, 8 aa, Homo sapiens
PINMLYFN

SEQ ID NO: 2326 - protein, 8 aa, Homo sapiens
INMLYFND

SEQ ID NO: 2327 - protein, 8 aa, Homo sapiens
NMLYFNDK

SEQ ID NO: 2328 - protein, 8 aa, Homo sapiens
MLYFNDKQ

SEQ ID NO: 2329 - protein, 8 aa, Homo sapiens
LYFNDKQQ

SEQ ID NO: 2330 - protein, 8 aa, Homo sapiens
YFNDKQQI

SEQ ID NO: 2331 - protein, 8 aa, Homo sapiens
FNDKQQII

SEQ ID NO: 2332 - protein, 8 aa, Homo sapiens
NDKQQIIY

SEQ ID NO: 2333 - protein, 8 aa, Homo sapiens
DKQQIIYG

SEQ ID NO: 2334 - protein, 8 aa, Homo sapiens
KQQIIYGK

SEQ ID NO: 2335 - protein, 8 aa, Homo sapiens
QQIIYGKI

SEQ ID NO: 2336 - protein, 8 aa, Homo sapiens
QIIYGKIP

SEQ ID NO: 2337 - protein, 8 aa, Homo sapiens
IIYGKIPG

SEQ ID NO: 2338 - protein, 8 aa, Homo sapiens
IYGKIPGM

SEQ ID NO: 2339 - protein, 8 aa, Homo sapiens
YGKIPGMV

SEQ ID NO: 2340 - protein, 8 aa, Homo sapiens
GKIPGMVV

SEQ ID NO: 2341 - protein, 8 aa, Homo sapiens
KIPGMVVD

SEQ ID NO: 2342 - protein, 8 aa, Homo sapiens
IPGMVVDR

SEQ ID NO: 2343 - protein, 8 aa, Homo sapiens
PGMVVDRC

SEQ ID NO: 2344 - protein, 8 aa, Homo sapiens
GMVVDRCG

SEQ ID NO: 2345 - protein, 8 aa, Homo sapiens
MVVDRCGC

SEQ ID NO: 2346 - protein, 8 aa, Homo sapiens
VVDRCGCS

SEQ ID NO: 2347 - protein, 9 aa, Homo sapiens
MVLAAPLLL

SEQ ID NO: 2348 - protein, 9 aa, Homo sapiens
VLAAPLLLG

SEQ ID NO: 2349 - protein, 9 aa, Homo sapiens
LAAPLLLGF

SEQ ID NO: 2350 - protein, 9 aa, Homo sapiens
AAPLLLGFL

SEQ ID NO: 2351 - protein, 9 aa, Homo sapiens
APLLLGFLL

SEQ ID NO: 2352 - protein, 9 aa, Homo sapiens
PLLLGFLLL

SEQ ID NO: 2353 - protein, 9 aa, Homo sapiens
LLLGFLLLA

SEQ ID NO: 2354 - protein, 9 aa, Homo sapiens
LLGFLLLAL

SEQ ID NO: 2355 - protein, 9 aa, Homo sapiens
LGFLLLALE

SEQ ID NO: 2356 - protein, 9 aa, Homo sapiens
GFLLLALEL

SEQ ID NO: 2357 - protein, 9 aa, Homo sapiens
FLLLALELR

SEQ ID NO: 2358 - protein, 9 aa, Homo sapiens
LLLALELRP

SEQ ID NO: 2359 - protein, 9 aa, Homo sapiens
LLALELRPR

SEQ ID NO: 2360 - protein, 9 aa, Homo sapiens
LALELRPRG

SEQ ID NO: 2361 - protein, 9 aa, Homo sapiens
ALELRPRGE

SEQ ID NO: 2362 - protein, 9 aa, Homo sapiens
LELRPRGEA

SEQ ID NO: 2363 - protein, 9 aa, Homo sapiens
ELRPRGEAA

SEQ ID NO: 2364 - protein, 9 aa, Homo sapiens
LRPRGEAAE

SEQ ID NO: 2365 - protein, 9 aa, Homo sapiens
RPRGEAAEG

SEQ ID NO: 2366 - protein, 9 aa, Homo sapiens
PRGEAAEGP

SEQ ID NO: 2367 - protein, 9 aa, Homo sapiens
RGEAAEGPA

SEQ ID NO: 2368 - protein, 9 aa, Homo sapiens
GEAAEGPAA

SEQ ID NO: 2369 - protein, 9 aa, Homo sapiens
EAAEGPAAA

SEQ ID NO: 2370 - protein, 9 aa, Homo sapiens
AAEGPAAAA

SEQ ID NO: 2371 - protein, 9 aa, Homo sapiens
AEGPAAAAA

SEQ ID NO: 2372 - protein, 9 aa, Homo sapiens
EGPAAAAAA

SEQ ID NO: 2373 - protein, 9 aa, Homo sapiens
GPAAAAAAA

SEQ ID NO: 2374 - protein, 9 aa, Homo sapiens
PAAAAAAAA

SEQ ID NO: 2375 - protein, 9 aa, Homo sapiens
AAAAAAAAA

SEQ ID NO: 2376 - protein, 9 aa, Homo sapiens
AAAAAAAAG

SEQ ID NO: 2377 - protein, 9 aa, Homo sapiens
AAAAAAAGV

SEQ ID NO: 2378 - protein, 9 aa, Homo sapiens
AAAAAAGVG

SEQ ID NO: 2379 - protein, 9 aa, Homo sapiens
AAAAAGVGG

SEQ ID NO: 2380 - protein, 9 aa, Homo sapiens
AAAAGVGGE

SEQ ID NO: 2381 - protein, 9 aa, Homo sapiens
AAAGVGGER

SEQ ID NO: 2382 - protein, 9 aa, Homo sapiens
AAGVGGERS

SEQ ID NO: 2383 - protein, 9 aa, Homo sapiens
AGVGGERSS

SEQ ID NO: 2384 - protein, 9 aa, Homo sapiens
GVGGERSSR

SEQ ID NO: 2385 - protein, 9 aa, Homo sapiens
VGGERSSRP

SEQ ID NO: 2386 - protein, 9 aa, Homo sapiens
GGERSSRPA

SEQ ID NO: 2387 - protein, 9 aa, Homo sapiens
GERSSRPAP

SEQ ID NO: 2388 - protein, 9 aa, Homo sapiens
ERSSRPAPS

SEQ ID NO: 2389 - protein, 9 aa, Homo sapiens
RSSRPAPSV

SEQ ID NO: 2390 - protein, 9 aa, Homo sapiens
SSRPAPSVA

SEQ ID NO: 2391 - protein, 9 aa, Homo sapiens
SRPAPSVAP

SEQ ID NO: 2392 - protein, 9 aa, Homo sapiens
RPAPSVAPE

SEQ ID NO: 2393 - protein, 9 aa, Homo sapiens
PAPSVAPEP

SEQ ID NO: 2394 - protein, 9 aa, Homo sapiens
APSVAPEPD

SEQ ID NO: 2395 - protein, 9 aa, Homo sapiens
PSVAPEPDG

SEQ ID NO: 2396 - protein, 9 aa, Homo sapiens
SVAPEPDGC

SEQ ID NO: 2397 - protein, 9 aa, Homo sapiens
VAPEPDGCP

SEQ ID NO: 2398 - protein, 9 aa, Homo sapiens
APEPDGCPV

SEQ ID NO: 2399 - protein, 9 aa, Homo sapiens
PEPDGCPVC

SEQ ID NO: 2400 - protein, 9 aa, Homo sapiens
EPDGCPVCV

SEQ ID NO: 2401 - protein, 9 aa, Homo sapiens
PDGCPVCVW

SEQ ID NO: 2402 - protein, 9 aa, Homo sapiens
DGCPVCVWR

SEQ ID NO: 2403 - protein, 9 aa, Homo sapiens
GCPVCVWRQ

SEQ ID NO: 2404 - protein, 9 aa, Homo sapiens
CPVCVWRQH

SEQ ID NO: 2405 - protein, 9 aa, Homo sapiens
PVCVWRQHS

SEQ ID NO: 2406 - protein, 9 aa, Homo sapiens
VCVWRQHSR

SEQ ID NO: 2407 - protein, 9 aa, Homo sapiens
CVWRQHSRE

SEQ ID NO: 2408 - protein, 9 aa, Homo sapiens
VWRQHSREL

SEQ ID NO: 2409 - protein, 9 aa, Homo sapiens
WRQHSRELR

SEQ ID NO: 2410 - protein, 9 aa, Homo sapiens
RQHSRELRL

SEQ ID NO: 2411 - protein, 9 aa, Homo sapiens
QHSRELRLE

SEQ ID NO: 2412 - protein, 9 aa, Homo sapiens
HSRELRLES

SEQ ID NO: 2413 - protein, 9 aa, Homo sapiens
SRELRLESI

SEQ ID NO: 2414 - protein, 9 aa, Homo sapiens
RELRLESIK

SEQ ID NO: 2415 - protein, 9 aa, Homo sapiens
ELRLESIKS

SEQ ID NO: 2416 - protein, 9 aa, Homo sapiens
LRLESIKSQ

SEQ ID NO: 2417 - protein, 9 aa, Homo sapiens
RLESIKSQI

SEQ ID NO: 2418 - protein, 9 aa, Homo sapiens
LESIKSQIL

SEQ ID NO: 2419 - protein, 9 aa, Homo sapiens
ESIKSQILS

SEQ ID NO: 2420 - protein, 9 aa, Homo sapiens
SIKSQILSK

SEQ ID NO: 2421 - protein, 9 aa, Homo sapiens
IKSQILSKL

SEQ ID NO: 2422 - protein, 9 aa, Homo sapiens
KSQILSKLR

SEQ ID NO: 2423 - protein, 9 aa, Homo sapiens
SQILSKLRL

SEQ ID NO: 2424 - protein, 9 aa, Homo sapiens
QILSKLRLK

SEQ ID NO: 2425 - protein, 9 aa, Homo sapiens
ILSKLRLKE

SEQ ID NO: 2426 - protein, 9 aa, Homo sapiens
LSKLRLKEA

SEQ ID NO: 2427 - protein, 9 aa, Homo sapiens
SKLRLKEAP

SEQ ID NO: 2428 - protein, 9 aa, Homo sapiens
KLRLKEAPN

SEQ ID NO: 2429 - protein, 9 aa, Homo sapiens
LRLKEAPNI

SEQ ID NO: 2430 - protein, 9 aa, Homo sapiens
RLKEAPNIS

SEQ ID NO: 2431 - protein, 9 aa, Homo sapiens
LKEAPNISR

SEQ ID NO: 2432 - protein, 9 aa, Homo sapiens
KEAPNISRE

SEQ ID NO: 2433 - protein, 9 aa, Homo sapiens
EAPNISREV

SEQ ID NO: 2434 - protein, 9 aa, Homo sapiens
APNISREVV

SEQ ID NO: 2435 - protein, 9 aa, Homo sapiens
PNISREVVK

SEQ ID NO: 2436 - protein, 9 aa, Homo sapiens
NISREVVKQ

SEQ ID NO: 2437 - protein, 9 aa, Homo sapiens
ISREVVKQL

SEQ ID NO: 2438 - protein, 9 aa, Homo sapiens
SREVVKQLL

SEQ ID NO: 2439 - protein, 9 aa, Homo sapiens
REVVKQLLP

SEQ ID NO: 2440 - protein, 9 aa, Homo sapiens
EVVKQLLPK

SEQ ID NO: 2441 - protein, 9 aa, Homo sapiens
VVKQLLPKA

SEQ ID NO: 2442 - protein, 9 aa, Homo sapiens
VKQLLPKAP

SEQ ID NO: 2443 - protein, 9 aa, Homo sapiens
KQLLPKAPP

SEQ ID NO: 2444 - protein, 9 aa, Homo sapiens
QLLPKAPPL

SEQ ID NO: 2445 - protein, 9 aa, Homo sapiens
LLPKAPPLQ

SEQ ID NO: 2446 - protein, 9 aa, Homo sapiens
LPKAPPLQQ

SEQ ID NO: 2447 - protein, 9 aa, Homo sapiens
PKAPPLQQI

SEQ ID NO: 2448 - protein, 9 aa, Homo sapiens
KAPPLQQIL

SEQ ID NO: 2449 - protein, 9 aa, Homo sapiens
APPLQQILD

SEQ ID NO: 2450 - protein, 9 aa, Homo sapiens
PPLQQILDL

SEQ ID NO: 2451 - protein, 9 aa, Homo sapiens
PLQQILDLH

SEQ ID NO: 2452 - protein, 9 aa, Homo sapiens
LQQILDLHD

SEQ ID NO: 2453 - protein, 9 aa, Homo sapiens
QQILDLHDF

SEQ ID NO: 2454 - protein, 9 aa, Homo sapiens
QILDLHDFQ

SEQ ID NO: 2455 - protein, 9 aa, Homo sapiens
ILDLHDFQG

SEQ ID NO: 2456 - protein, 9 aa, Homo sapiens
LDLHDFQGD

SEQ ID NO: 2457 - protein, 9 aa, Homo sapiens
DLHDFQGDA

SEQ ID NO: 2458 - protein, 9 aa, Homo sapiens
LHDFQGDAL

SEQ ID NO: 2459 - protein, 9 aa, Homo sapiens
HDFQGDALQ

SEQ ID NO: 2460 - protein, 9 aa, Homo sapiens
DFQGDALQP

SEQ ID NO: 2461 - protein, 9 aa, Homo sapiens
FQGDALQPE

SEQ ID NO: 2462 - protein, 9 aa, Homo sapiens
QGDALQPED

SEQ ID NO: 2463 - protein, 9 aa, Homo sapiens
GDALQPEDF

SEQ ID NO: 2464 - protein, 9 aa, Homo sapiens
DALQPEDFL

SEQ ID NO: 2465 - protein, 9 aa, Homo sapiens
ALQPEDFLE

SEQ ID NO: 2466 - protein, 9 aa, Homo sapiens
LQPEDFLEE

SEQ ID NO: 2467 - protein, 9 aa, Homo sapiens
QPEDFLEED

SEQ ID NO: 2468 - protein, 9 aa, Homo sapiens
PEDFLEEDE

SEQ ID NO: 2469 - protein, 9 aa, Homo sapiens
EDFLEEDEY

SEQ ID NO: 2470 - protein, 9 aa, Homo sapiens
DFLEEDEYH

SEQ ID NO: 2471 - protein, 9 aa, Homo sapiens
FLEEDEYHA

SEQ ID NO: 2472 - protein, 9 aa, Homo sapiens
LEEDEYHAT

SEQ ID NO: 2473 - protein, 9 aa, Homo sapiens
EEDEYHATT

SEQ ID NO: 2474 - protein, 9 aa, Homo sapiens
EDEYHATTE

SEQ ID NO: 2475 - protein, 9 aa, Homo sapiens
DEYHATTET

SEQ ID NO: 2476 - protein, 9 aa, Homo sapiens
EYHATTETV

SEQ ID NO: 2477 - protein, 9 aa, Homo sapiens
YHATTETVI

SEQ ID NO: 2478 - protein, 9 aa, Homo sapiens
HATTETVIS

SEQ ID NO: 2479 - protein, 9 aa, Homo sapiens
ATTETVISM

SEQ ID NO: 2480 - protein, 9 aa, Homo sapiens
TTETVISMA

SEQ ID NO: 2481 - protein, 9 aa, Homo sapiens
TETVISMAQ

SEQ ID NO: 2482 - protein, 9 aa, Homo sapiens
ETVISMAQE

SEQ ID NO: 2483 - protein, 9 aa, Homo sapiens
TVISMAQET

SEQ ID NO: 2484 - protein, 9 aa, Homo sapiens
VISMAQETD

SEQ ID NO: 2485 - protein, 9 aa, Homo sapiens
ISMAQETDP

SEQ ID NO: 2486 - protein, 9 aa, Homo sapiens
SMAQETDPA

SEQ ID NO: 2487 - protein, 9 aa, Homo sapiens
MAQETDPAV

SEQ ID NO: 2488 - protein, 9 aa, Homo sapiens
AQETDPAVQ

SEQ ID NO: 2489 - protein, 9 aa, Homo sapiens
QETDPAVQT

SEQ ID NO: 2490 - protein, 9 aa, Homo sapiens
ETDPAVQTD

SEQ ID NO: 2491 - protein, 9 aa, Homo sapiens
TDPAVQTDG

SEQ ID NO: 2492 - protein, 9 aa, Homo sapiens
DPAVQTDGS

SEQ ID NO: 2493 - protein, 9 aa, Homo sapiens
PAVQTDGSP

SEQ ID NO: 2494 - protein, 9 aa, Homo sapiens
AVQTDGSPL

SEQ ID NO: 2495 - protein, 9 aa, Homo sapiens
VQTDGSPLC

SEQ ID NO: 2496 - protein, 9 aa, Homo sapiens
QTDGSPLCC

SEQ ID NO: 2497 - protein, 9 aa, Homo sapiens
TDGSPLCCH

SEQ ID NO: 2498 - protein, 9 aa, Homo sapiens
DGSPLCCHF

SEQ ID NO: 2499 - protein, 9 aa, Homo sapiens
GSPLCCHFH

SEQ ID NO: 2500 - protein, 9 aa, Homo sapiens
SPLCCHFHF

SEQ ID NO: 2501 - protein, 9 aa, Homo sapiens
PLCCHFHFS

SEQ ID NO: 2502 - protein, 9 aa, Homo sapiens
LCCHFHFSP

SEQ ID NO: 2503 - protein, 9 aa, Homo sapiens
CCHFHFSPK

SEQ ID NO: 2504 - protein, 9 aa, Homo sapiens
CHFHFSPKV

SEQ ID NO: 2505 - protein, 9 aa, Homo sapiens
HFHFSPKVM

SEQ ID NO: 2506 - protein, 9 aa, Homo sapiens
FHFSPKVMF

SEQ ID NO: 2507 - protein, 9 aa, Homo sapiens
HFSPKVMFT

SEQ ID NO: 2508 - protein, 9 aa, Homo sapiens
FSPKVMFTK

SEQ ID NO: 2509 - protein, 9 aa, Homo sapiens
SPKVMFTKV

SEQ ID NO: 2510 - protein, 9 aa, Homo sapiens
PKVMFTKVL

SEQ ID NO: 2511 - protein, 9 aa, Homo sapiens
KVMFTKVLK

SEQ ID NO: 2512 - protein, 9 aa, Homo sapiens
VMFTKVLKA

SEQ ID NO: 2513 - protein, 9 aa, Homo sapiens
MFTKVLKAQ

SEQ ID NO: 2514 - protein, 9 aa, Homo sapiens
FTKVLKAQL

SEQ ID NO: 2515 - protein, 9 aa, Homo sapiens
TKVLKAQLW

SEQ ID NO: 2516 - protein, 9 aa, Homo sapiens
KVLKAQLWV

SEQ ID NO: 2517 - protein, 9 aa, Homo sapiens
VLKAQLWVY

SEQ ID NO: 2518 - protein, 9 aa, Homo sapiens
LKAQLWVYL

SEQ ID NO: 2519 - protein, 9 aa, Homo sapiens
KAQLWVYLR

SEQ ID NO: 2520 - protein, 9 aa, Homo sapiens
AQLWVYLRP

SEQ ID NO: 2521 - protein, 9 aa, Homo sapiens
QLWVYLRPV

SEQ ID NO: 2522 - protein, 9 aa, Homo sapiens
LWVYLRPVP

SEQ ID NO: 2523 - protein, 9 aa, Homo sapiens
WVYLRPVPR

SEQ ID NO: 2524 - protein, 9 aa, Homo sapiens
VYLRPVPRP

SEQ ID NO: 2525 - protein, 9 aa, Homo sapiens
YLRPVPRPA

SEQ ID NO: 2526 - protein, 9 aa, Homo sapiens
LRPVPRPAT

SEQ ID NO: 2527 - protein, 9 aa, Homo sapiens
RPVPRPATV

SEQ ID NO: 2528 - protein, 9 aa, Homo sapiens
PVPRPATVY

SEQ ID NO: 2529 - protein, 9 aa, Homo sapiens
VPRPATVYL

SEQ ID NO: 2530 - protein, 9 aa, Homo sapiens
PRPATVYLQ

SEQ ID NO: 2531 - protein, 9 aa, Homo sapiens
RPATVYLQI

SEQ ID NO: 2532 - protein, 9 aa, Homo sapiens
PATVYLQIL

SEQ ID NO: 2533 - protein, 9 aa, Homo sapiens
ATVYLQILR

SEQ ID NO: 2534 - protein, 9 aa, Homo sapiens
TVYLQILRL

SEQ ID NO: 2535 - protein, 9 aa, Homo sapiens
VYLQILRLK

SEQ ID NO: 2536 - protein, 9 aa, Homo sapiens
YLQILRLKP

SEQ ID NO: 2537 - protein, 9 aa, Homo sapiens
LQILRLKPL

SEQ ID NO: 2538 - protein, 9 aa, Homo sapiens
QILRLKPLT

SEQ ID NO: 2539 - protein, 9 aa, Homo sapiens
ILRLKPLTG

SEQ ID NO: 2540 - protein, 9 aa, Homo sapiens
LRLKPLTGE

SEQ ID NO: 2541 - protein, 9 aa, Homo sapiens
RLKPLTGEG

SEQ ID NO: 2542 - protein, 9 aa, Homo sapiens
LKPLTGEGT

SEQ ID NO: 2543 - protein, 9 aa, Homo sapiens
KPLTGEGTA

SEQ ID NO: 2544 - protein, 9 aa, Homo sapiens
PLTGEGTAG

SEQ ID NO: 2545 - protein, 9 aa, Homo sapiens
LTGEGTAGG

SEQ ID NO: 2546 - protein, 9 aa, Homo sapiens
TGEGTAGGG

SEQ ID NO: 2547 - protein, 9 aa, Homo sapiens
GEGTAGGGG

SEQ ID NO: 2548 - protein, 9 aa, Homo sapiens
EGTAGGGGG

SEQ ID NO: 2549 - protein, 9 aa, Homo sapiens
GTAGGGGGG

SEQ ID NO: 2550 - protein, 9 aa, Homo sapiens
TAGGGGGGR

SEQ ID NO: 2551 - protein, 9 aa, Homo sapiens
AGGGGGGRR

SEQ ID NO: 2552 - protein, 9 aa, Homo sapiens
GGGGGGRRH

SEQ ID NO: 2553 - protein, 9 aa, Homo sapiens
GGGGGRRHI

SEQ ID NO: 2554 - protein, 9 aa, Homo sapiens
GGGGRRHIR

SEQ ID NO: 2555 - protein, 9 aa, Homo sapiens
GGGRRHIRI

SEQ ID NO: 2556 - protein, 9 aa, Homo sapiens
GGRRHIRIR

SEQ ID NO: 2557 - protein, 9 aa, Homo sapiens
GRRHIRIRS

SEQ ID NO: 2558 - protein, 9 aa, Homo sapiens
RRHIRIRSL

SEQ ID NO: 2559 - protein, 9 aa, Homo sapiens
RHIRIRSLK

SEQ ID NO: 2560 - protein, 9 aa, Homo sapiens
HIRIRSLKI

SEQ ID NO: 2561 - protein, 9 aa, Homo sapiens
IRIRSLKIE

SEQ ID NO: 2562 - protein, 9 aa, Homo sapiens
RIRSLKIEL

SEQ ID NO: 2563 - protein, 9 aa, Homo sapiens
IRSLKIELH

SEQ ID NO: 2564 - protein, 9 aa, Homo sapiens
RSLKIELHS

SEQ ID NO: 2565 - protein, 9 aa, Homo sapiens
SLKIELHSR

SEQ ID NO: 2566 - protein, 9 aa, Homo sapiens
LKIELHSRS

SEQ ID NO: 2567 - protein, 9 aa, Homo sapiens
KIELHSRSG

SEQ ID NO: 2568 - protein, 9 aa, Homo sapiens
IELHSRSGH

SEQ ID NO: 2569 - protein, 9 aa, Homo sapiens
ELHSRSGHW

SEQ ID NO: 2570 - protein, 9 aa, Homo sapiens
LHSRSGHWQ

SEQ ID NO: 2571 - protein, 9 aa, Homo sapiens
HSRSGHWQS

SEQ ID NO: 2572 - protein, 9 aa, Homo sapiens
SRSGHWQSI

SEQ ID NO: 2573 - protein, 9 aa, Homo sapiens
RSGHWQSID

SEQ ID NO: 2574 - protein, 9 aa, Homo sapiens
SGHWQSIDF

SEQ ID NO: 2575 - protein, 9 aa, Homo sapiens
GHWQSIDFK

SEQ ID NO: 2576 - protein, 9 aa, Homo sapiens
HWQSIDFKQ

SEQ ID NO: 2577 - protein, 9 aa, Homo sapiens
WQSIDFKQV

SEQ ID NO: 2578 - protein, 9 aa, Homo sapiens
QSIDFKQVL

SEQ ID NO: 2579 - protein, 9 aa, Homo sapiens
SIDFKQVLH

SEQ ID NO: 2580 - protein, 9 aa, Homo sapiens
IDFKQVLHS

SEQ ID NO: 2581 - protein, 9 aa, Homo sapiens
DFKQVLHSW

SEQ ID NO: 2582 - protein, 9 aa, Homo sapiens
FKQVLHSWF

SEQ ID NO: 2583 - protein, 9 aa, Homo sapiens
KQVLHSWFR

SEQ ID NO: 2584 - protein, 9 aa, Homo sapiens
QVLHSWFRQ

SEQ ID NO: 2585 - protein, 9 aa, Homo sapiens
VLHSWFRQP

SEQ ID NO: 2586 - protein, 9 aa, Homo sapiens
LHSWFRQPQ

SEQ ID NO: 2587 - protein, 9 aa, Homo sapiens
HSWFRQPQS

SEQ ID NO: 2588 - protein, 9 aa, Homo sapiens
SWFRQPQSN

SEQ ID NO: 2589 - protein, 9 aa, Homo sapiens
WFRQPQSNW

SEQ ID NO: 2590 - protein, 9 aa, Homo sapiens
FRQPQSNWG

SEQ ID NO: 2591 - protein, 9 aa, Homo sapiens
RQPQSNWGI

SEQ ID NO: 2592 - protein, 9 aa, Homo sapiens
QPQSNWGIE

SEQ ID NO: 2593 - protein, 9 aa, Homo sapiens
PQSNWGIEI

SEQ ID NO: 2594 - protein, 9 aa, Homo sapiens
QSNWGIEIN

SEQ ID NO: 2595 - protein, 9 aa, Homo sapiens
SNWGIEINA

SEQ ID NO: 2596 - protein, 9 aa, Homo sapiens
NWGIEINAF

SEQ ID NO: 2597 - protein, 9 aa, Homo sapiens
WGIEINAFD

SEQ ID NO: 2598 - protein, 9 aa, Homo sapiens
GIEINAFDP

SEQ ID NO: 2599 - protein, 9 aa, Homo sapiens
IEINAFDPS

SEQ ID NO: 2600 - protein, 9 aa, Homo sapiens
EINAFDPSG

SEQ ID NO: 2601 - protein, 9 aa, Homo sapiens
INAFDPSGT

SEQ ID NO: 2602 - protein, 9 aa, Homo sapiens
NAFDPSGTD

SEQ ID NO: 2603 - protein, 9 aa, Homo sapiens
AFDPSGTDL

SEQ ID NO: 2604 - protein, 9 aa, Homo sapiens
FDPSGTDLA

SEQ ID NO: 2605 - protein, 9 aa, Homo sapiens
DPSGTDLAV

SEQ ID NO: 2606 - protein, 9 aa, Homo sapiens
PSGTDLAVT

SEQ ID NO: 2607 - protein, 9 aa, Homo sapiens
SGTDLAVTS

SEQ ID NO: 2608 - protein, 9 aa, Homo sapiens
GTDLAVTSL

SEQ ID NO: 2609 - protein, 9 aa, Homo sapiens
TDLAVTSLG

SEQ ID NO: 2610 - protein, 9 aa, Homo sapiens
DLAVTSLGP

SEQ ID NO: 2611 - protein, 9 aa, Homo sapiens
LAVTSLGPG

SEQ ID NO: 2612 - protein, 9 aa, Homo sapiens
AVTSLGPGA

SEQ ID NO: 2613 - protein, 9 aa, Homo sapiens
VTSLGPGAE

SEQ ID NO: 2614 - protein, 9 aa, Homo sapiens
TSLGPGAEG

SEQ ID NO: 2615 - protein, 9 aa, Homo sapiens
SLGPGAEGL

SEQ ID NO: 2616 - protein, 9 aa, Homo sapiens
LGPGAEGLH

SEQ ID NO: 2617 - protein, 9 aa, Homo sapiens
GPGAEGLHP

SEQ ID NO: 2618 - protein, 9 aa, Homo sapiens
PGAEGLHPF

SEQ ID NO: 2619 - protein, 9 aa, Homo sapiens
GAEGLHPFM

SEQ ID NO: 2620 - protein, 9 aa, Homo sapiens
AEGLHPFME

SEQ ID NO: 2621 - protein, 9 aa, Homo sapiens
EGLHPFMEL

SEQ ID NO: 2622 - protein, 9 aa, Homo sapiens
GLHPFMELR

SEQ ID NO: 2623 - protein, 9 aa, Homo sapiens
LHPFMELRV

SEQ ID NO: 2624 - protein, 9 aa, Homo sapiens
HPFMELRVL

SEQ ID NO: 2625 - protein, 9 aa, Homo sapiens
PFMELRVLE

SEQ ID NO: 2626 - protein, 9 aa, Homo sapiens
FMELRVLEN

SEQ ID NO: 2627 - protein, 9 aa, Homo sapiens
MELRVLENT

SEQ ID NO: 2628 - protein, 9 aa, Homo sapiens
ELRVLENTK

SEQ ID NO: 2629 - protein, 9 aa, Homo sapiens
LRVLENTKR

SEQ ID NO: 2630 - protein, 9 aa, Homo sapiens
RVLENTKRS

SEQ ID NO: 2631 - protein, 9 aa, Homo sapiens
VLENTKRSR

SEQ ID NO: 2632 - protein, 9 aa, Homo sapiens
LENTKRSRR

SEQ ID NO: 2633 - protein, 9 aa, Homo sapiens
ENTKRSRRN

SEQ ID NO: 2634 - protein, 9 aa, Homo sapiens
NTKRSRRNL

SEQ ID NO: 2635 - protein, 9 aa, Homo sapiens
TKRSRRNLG

SEQ ID NO: 2636 - protein, 9 aa, Homo sapiens
KRSRRNLGL

SEQ ID NO: 2637 - protein, 9 aa, Homo sapiens
RSRRNLGLD

SEQ ID NO: 2638 - protein, 9 aa, Homo sapiens
SRRNLGLDC

SEQ ID NO: 2639 - protein, 9 aa, Homo sapiens
RRNLGLDCD

SEQ ID NO: 2640 - protein, 9 aa, Homo sapiens
RNLGLDCDE

SEQ ID NO: 2641 - protein, 9 aa, Homo sapiens
NLGLDCDEH

SEQ ID NO: 2642 - protein, 9 aa, Homo sapiens
LGLDCDEHS

SEQ ID NO: 2643 - protein, 9 aa, Homo sapiens
GLDCDEHSS

SEQ ID NO: 2644 - protein, 9 aa, Homo sapiens
LDCDEHSSE

SEQ ID NO: 2645 - protein, 9 aa, Homo sapiens
DCDEHSSES

SEQ ID NO: 2646 - protein, 9 aa, Homo sapiens
CDEHSSESR

SEQ ID NO: 2647 - protein, 9 aa, Homo sapiens
DEHSSESRC

SEQ ID NO: 2648 - protein, 9 aa, Homo sapiens
EHSSESRCC

SEQ ID NO: 2649 - protein, 9 aa, Homo sapiens
HSSESRCCR

SEQ ID NO: 2650 - protein, 9 aa, Homo sapiens
SSESRCCRY

SEQ ID NO: 2651 - protein, 9 aa, Homo sapiens
SESRCCRYP

SEQ ID NO: 2652 - protein, 9 aa, Homo sapiens
ESRCCRYPL

SEQ ID NO: 2653 - protein, 9 aa, Homo sapiens
SRCCRYPLT

SEQ ID NO: 2654 - protein, 9 aa, Homo sapiens
RCCRYPLTV

SEQ ID NO: 2655 - protein, 9 aa, Homo sapiens
CCRYPLTVD

SEQ ID NO: 2656 - protein, 9 aa, Homo sapiens
CRYPLTVDF

SEQ ID NO: 2657 - protein, 9 aa, Homo sapiens
RYPLTVDFE

SEQ ID NO: 2658 - protein, 9 aa, Homo sapiens
YPLTVDFEA

SEQ ID NO: 2659 - protein, 9 aa, Homo sapiens
PLTVDFEAF

SEQ ID NO: 2660 - protein, 9 aa, Homo sapiens
LTVDFEAFG

SEQ ID NO: 2661 - protein, 9 aa, Homo sapiens
TVDFEAFGW

SEQ ID NO: 2662 - protein, 9 aa, Homo sapiens
VDFEAFGWD

SEQ ID NO: 2663 - protein, 9 aa, Homo sapiens
DFEAFGWDW

SEQ ID NO: 2664 - protein, 9 aa, Homo sapiens
FEAFGWDWI

SEQ ID NO: 2665 - protein, 9 aa, Homo sapiens
EAFGWDWII

SEQ ID NO: 2666 - protein, 9 aa, Homo sapiens
AFGWDWIIA

SEQ ID NO: 2667 - protein, 9 aa, Homo sapiens
FGWDWIIAP

SEQ ID NO: 2668 - protein, 9 aa, Homo sapiens
GWDWIIAPK

SEQ ID NO: 2669 - protein, 9 aa, Homo sapiens
WDWIIAPKR

SEQ ID NO: 2670 - protein, 9 aa, Homo sapiens
DWIIAPKRY

SEQ ID NO: 2671 - protein, 9 aa, Homo sapiens
WIIAPKRYK

SEQ ID NO: 2672 - protein, 9 aa, Homo sapiens
IIAPKRYKA

SEQ ID NO: 2673 - protein, 9 aa, Homo sapiens
IAPKRYKAN

SEQ ID NO: 2674 - protein, 9 aa, Homo sapiens
APKRYKANY

SEQ ID NO: 2675 - protein, 9 aa, Homo sapiens
PKRYKANYC

SEQ ID NO: 2676 - protein, 9 aa, Homo sapiens
KRYKANYCS

SEQ ID NO: 2677 - protein, 9 aa, Homo sapiens
RYKANYCSG

SEQ ID NO: 2678 - protein, 9 aa, Homo sapiens
YKANYCSGQ

SEQ ID NO: 2679 - protein, 9 aa, Homo sapiens
KANYCSGQC

SEQ ID NO: 2680 - protein, 9 aa, Homo sapiens
ANYCSGQCE

SEQ ID NO: 2681 - protein, 9 aa, Homo sapiens
NYCSGQCEY

SEQ ID NO: 2682 - protein, 9 aa, Homo sapiens
YCSGQCEYM

SEQ ID NO: 2683 - protein, 9 aa, Homo sapiens
CSGQCEYMF

SEQ ID NO: 2684 - protein, 9 aa, Homo sapiens
SGQCEYMFM

SEQ ID NO: 2685 - protein, 9 aa, Homo sapiens
GQCEYMFMQ

SEQ ID NO: 2686 - protein, 9 aa, Homo sapiens
QCEYMFMQK

SEQ ID NO: 2687 - protein, 9 aa, Homo sapiens
CEYMFMQKY

SEQ ID NO: 2688 - protein, 9 aa, Homo sapiens
EYMFMQKYP

SEQ ID NO: 2689 - protein, 9 aa, Homo sapiens
YMFMQKYPH

SEQ ID NO: 2690 - protein, 9 aa, Homo sapiens
MFMQKYPHT

SEQ ID NO: 2691 - protein, 9 aa, Homo sapiens
FMQKYPHTH

SEQ ID NO: 2692 - protein, 9 aa, Homo sapiens
MQKYPHTHL

SEQ ID NO: 2693 - protein, 9 aa, Homo sapiens
QKYPHTHLV

SEQ ID NO: 2694 - protein, 9 aa, Homo sapiens
KYPHTHLVQ

SEQ ID NO: 2695 - protein, 9 aa, Homo sapiens
YPHTHLVQQ

SEQ ID NO: 2696 - protein, 9 aa, Homo sapiens
PHTHLVQQA

SEQ ID NO: 2697 - protein, 9 aa, Homo sapiens
HTHLVQQAN

SEQ ID NO: 2698 - protein, 9 aa, Homo sapiens
THLVQQANP

SEQ ID NO: 2699 - protein, 9 aa, Homo sapiens
HLVQQANPR

SEQ ID NO: 2700 - protein, 9 aa, Homo sapiens
LVQQANPRG

SEQ ID NO: 2701 - protein, 9 aa, Homo sapiens
VQQANPRGS

SEQ ID NO: 2702 - protein, 9 aa, Homo sapiens
QQANPRGSA

SEQ ID NO: 2703 - protein, 9 aa, Homo sapiens
QANPRGSAG

SEQ ID NO: 2704 - protein, 9 aa, Homo sapiens
ANPRGSAGP

SEQ ID NO: 2705 - protein, 9 aa, Homo sapiens
NPRGSAGPC

SEQ ID NO: 2706 - protein, 9 aa, Homo sapiens
PRGSAGPCC

SEQ ID NO: 2707 - protein, 9 aa, Homo sapiens
RGSAGPCCT

SEQ ID NO: 2708 - protein, 9 aa, Homo sapiens
GSAGPCCTP

SEQ ID NO: 2709 - protein, 9 aa, Homo sapiens
SAGPCCTPT

SEQ ID NO: 2710 - protein, 9 aa, Homo sapiens
AGPCCTPTK

SEQ ID NO: 2711 - protein, 9 aa, Homo sapiens
GPCCTPTKM

SEQ ID NO: 2712 - protein, 9 aa, Homo sapiens
PCCTPTKMS

SEQ ID NO: 2713 - protein, 9 aa, Homo sapiens
CCTPTKMSP

SEQ ID NO: 2714 - protein, 9 aa, Homo sapiens
CTPTKMSPI

SEQ ID NO: 2715 - protein, 9 aa, Homo sapiens
TPTKMSPIN

SEQ ID NO: 2716 - protein, 9 aa, Homo sapiens
PTKMSPINM

SEQ ID NO: 2717 - protein, 9 aa, Homo sapiens
TKMSPINML

SEQ ID NO: 2718 - protein, 9 aa, Homo sapiens
KMSPINMLY

SEQ ID NO: 2719 - protein, 9 aa, Homo sapiens
MSPINMLYF

SEQ ID NO: 2720 - protein, 9 aa, Homo sapiens
SPINMLYFN

SEQ ID NO: 2721 - protein, 9 aa, Homo sapiens
PINMLYFND

SEQ ID NO: 2722 - protein, 9 aa, Homo sapiens
INMLYFNDK

SEQ ID NO: 2723 - protein, 9 aa, Homo sapiens
NMLYFNDKQ

SEQ ID NO: 2724 - protein, 9 aa, Homo sapiens
MLYFNDKQQ

SEQ ID NO: 2725 - protein, 9 aa, Homo sapiens
LYFNDKQQI

SEQ ID NO: 2726 - protein, 9 aa, Homo sapiens
YFNDKQQII

SEQ ID NO: 2727 - protein, 9 aa, Homo sapiens
FNDKQQIIY

SEQ ID NO: 2728 - protein, 9 aa, Homo sapiens
NDKQQIIYG

SEQ ID NO: 2729 - protein, 9 aa, Homo sapiens
DKQQIIYGK

SEQ ID NO: 2730 - protein, 9 aa, Homo sapiens
KQQIIYGKI

SEQ ID NO: 2731 - protein, 9 aa, Homo sapiens
QQIIYGKIP

SEQ ID NO: 2732 - protein, 9 aa, Homo sapiens
QIIYGKIPG

SEQ ID NO: 2733 - protein, 9 aa, Homo sapiens
IIYGKIPGM

SEQ ID NO: 2734 - protein, 9 aa, Homo sapiens
IYGKIPGMV

SEQ ID NO: 2735 - protein, 9 aa, Homo sapiens
YGKIPGMVV

SEQ ID NO: 2736 - protein, 9 aa, Homo sapiens
GKIPGMVVD

SEQ ID NO: 2737 - protein, 9 aa, Homo sapiens
KIPGMVVDR

SEQ ID NO: 2738 - protein, 9 aa, Homo sapiens
IPGMVVDRC

SEQ ID NO: 2739 - protein, 9 aa, Homo sapiens
PGMVVDRCG

SEQ ID NO: 2740 - protein, 9 aa, Homo sapiens
GMVVDRCGC

SEQ ID NO: 2741 - protein, 9 aa, Homo sapiens
MVVDRCGCS

SEQ ID NO: 2742 - protein, 10 aa, Homo sapiens
MVLAAPLLLG

SEQ ID NO: 2743 - protein, 10 aa, Homo sapiens
VLAAPLLLGF

SEQ ID NO: 2744 - protein, 10 aa, Homo sapiens
LAAPLLLGFL

SEQ ID NO: 2745 - protein, 10 aa, Homo sapiens
AAPLLLGFLL

SEQ ID NO: 2746 - protein, 10 aa, Homo sapiens
APLLLGFLLL

SEQ ID NO: 2747 - protein, 10 aa, Homo sapiens
PLLLGFLLLA

SEQ ID NO: 2748 - protein, 10 aa, Homo sapiens
LLLGFLLLAL

SEQ ID NO: 2749 - protein, 10 aa, Homo sapiens
LLGFLLLALE

SEQ ID NO: 2750 - protein, 10 aa, Homo sapiens
LGFLLLALEL

SEQ ID NO: 2751 - protein, 10 aa, Homo sapiens
GFLLLALELR

SEQ ID NO: 2752 - protein, 10 aa, Homo sapiens
FLLLALELRP

SEQ ID NO: 2753 - protein, 10 aa, Homo sapiens
LLLALELRPR

SEQ ID NO: 2754 - protein, 10 aa, Homo sapiens
LLALELRPRG

SEQ ID NO: 2755 - protein, 10 aa, Homo sapiens
LALELRPRGE

SEQ ID NO: 2756 - protein, 10 aa, Homo sapiens
ALELRPRGEA

SEQ ID NO: 2757 - protein, 10 aa, Homo sapiens
LELRPRGEAA

SEQ ID NO: 2758 - protein, 10 aa, Homo sapiens
ELRPRGEAAE

SEQ ID NO: 2759 - protein, 10 aa, Homo sapiens
LRPRGEAAEG

SEQ ID NO: 2760 - protein, 10 aa, Homo sapiens
RPRGEAAEGP

SEQ ID NO: 2761 - protein, 10 aa, Homo sapiens
PRGEAAEGPA

SEQ ID NO: 2762 - protein, 10 aa, Homo sapiens
RGEAAEGPAA

SEQ ID NO: 2763 - protein, 10 aa, Homo sapiens
GEAAEGPAAA

SEQ ID NO: 2764 - protein, 10 aa, Homo sapiens
EAAEGPAAAA

SEQ ID NO: 2765 - protein, 10 aa, Homo sapiens
AAEGPAAAAA

SEQ ID NO: 2766 - protein, 10 aa, Homo sapiens
AEGPAAAAAA

SEQ ID NO: 2767 - protein, 10 aa, Homo sapiens
EGPAAAAAAA

SEQ ID NO: 2768 - protein, 10 aa, Homo sapiens
GPAAAAAAAA

SEQ ID NO: 2769 - protein, 10 aa, Homo sapiens
PAAAAAAAAA

SEQ ID NO: 2770 - protein, 10 aa, Homo sapiens
AAAAAAAAAA

SEQ ID NO: 2771 - protein, 10 aa, Homo sapiens
AAAAAAAAAG

SEQ ID NO: 2772 - protein, 10 aa, Homo sapiens
AAAAAAAAGV

SEQ ID NO: 2773 - protein, 10 aa, Homo sapiens
AAAAAAAGVG

SEQ ID NO: 2774 - protein, 10 aa, Homo sapiens
AAAAAAGVGG

SEQ ID NO: 2775 - protein, 10 aa, Homo sapiens
AAAAAGVGGE

SEQ ID NO: 2776 - protein, 10 aa, Homo sapiens
AAAAGVGGER

SEQ ID NO: 2777 - protein, 10 aa, Homo sapiens
AAAGVGGERS

SEQ ID NO: 2778 - protein, 10 aa, Homo sapiens
AAGVGGERSS

SEQ ID NO: 2779 - protein, 10 aa, Homo sapiens
AGVGGERSSR

SEQ ID NO: 2780 - protein, 10 aa, Homo sapiens
GVGGERSSRP

SEQ ID NO: 2781 - protein, 10 aa, Homo sapiens
VGGERSSRPA

SEQ ID NO: 2782 - protein, 10 aa, Homo sapiens
GGERSSRPAP

SEQ ID NO: 2783 - protein, 10 aa, Homo sapiens
GERSSRPAPS

SEQ ID NO: 2784 - protein, 10 aa, Homo sapiens
ERSSRPAPSV

SEQ ID NO: 2785 - protein, 10 aa, Homo sapiens
RSSRPAPSVA

SEQ ID NO: 2786 - protein, 10 aa, Homo sapiens
SSRPAPSVAP

SEQ ID NO: 2787 - protein, 10 aa, Homo sapiens
SRPAPSVAPE

SEQ ID NO: 2788 - protein, 10 aa, Homo sapiens
RPAPSVAPEP

SEQ ID NO: 2789 - protein, 10 aa, Homo sapiens
PAPSVAPEPD

SEQ ID NO: 2790 - protein, 10 aa, Homo sapiens
APSVAPEPDG

SEQ ID NO: 2791 - protein, 10 aa, Homo sapiens
PSVAPEPDGC

SEQ ID NO: 2792 - protein, 10 aa, Homo sapiens
SVAPEPDGCP

SEQ ID NO: 2793 - protein, 10 aa, Homo sapiens
VAPEPDGCPV

SEQ ID NO: 2794 - protein, 10 aa, Homo sapiens
APEPDGCPVC

SEQ ID NO: 2795 - protein, 10 aa, Homo sapiens
PEPDGCPVCV

SEQ ID NO: 2796 - protein, 10 aa, Homo sapiens
EPDGCPVCVW

SEQ ID NO: 2797 - protein, 10 aa, Homo sapiens
PDGCPVCVWR

SEQ ID NO: 2798 - protein, 10 aa, Homo sapiens
DGCPVCVWRQ

SEQ ID NO: 2799 - protein, 10 aa, Homo sapiens
GCPVCVWRQH

SEQ ID NO: 2800 - protein, 10 aa, Homo sapiens
CPVCVWRQHS

SEQ ID NO: 2801 - protein, 10 aa, Homo sapiens
PVCVWRQHSR

SEQ ID NO: 2802 - protein, 10 aa, Homo sapiens
VCVWRQHSRE

SEQ ID NO: 2803 - protein, 10 aa, Homo sapiens
CVWRQHSREL

SEQ ID NO: 2804 - protein, 10 aa, Homo sapiens
VWRQHSRELR

SEQ ID NO: 2805 - protein, 10 aa, Homo sapiens
WRQHSRELRL

SEQ ID NO: 2806 - protein, 10 aa, Homo sapiens
RQHSRELRLE

SEQ ID NO: 2807 - protein, 10 aa, Homo sapiens
QHSRELRLES

SEQ ID NO: 2808 - protein, 10 aa, Homo sapiens
HSRELRLESI

SEQ ID NO: 2809 - protein, 10 aa, Homo sapiens
SRELRLESIK

SEQ ID NO: 2810 - protein, 10 aa, Homo sapiens
RELRLESIKS

SEQ ID NO: 2811 - protein, 10 aa, Homo sapiens
ELRLESIKSQ

SEQ ID NO: 2812 - protein, 10 aa, Homo sapiens
LRLESIKSQI

SEQ ID NO: 2813 - protein, 10 aa, Homo sapiens
RLESIKSQIL

SEQ ID NO: 2814 - protein, 10 aa, Homo sapiens
LESIKSQILS

SEQ ID NO: 2815 - protein, 10 aa, Homo sapiens
ESIKSQILSK

SEQ ID NO: 2816 - protein, 10 aa, Homo sapiens
SIKSQILSKL

SEQ ID NO: 2817 - protein, 10 aa, Homo sapiens
IKSQILSKLR

SEQ ID NO: 2818 - protein, 10 aa, Homo sapiens
KSQILSKLRL

SEQ ID NO: 2819 - protein, 10 aa, Homo sapiens
SQILSKLRLK

SEQ ID NO: 2820 - protein, 10 aa, Homo sapiens
QILSKLRLKE

SEQ ID NO: 2821 - protein, 10 aa, Homo sapiens
ILSKLRLKEA

SEQ ID NO: 2822 - protein, 10 aa, Homo sapiens
LSKLRLKEAP

SEQ ID NO: 2823 - protein, 10 aa, Homo sapiens
SKLRLKEAPN

SEQ ID NO: 2824 - protein, 10 aa, Homo sapiens
KLRLKEAPNI

SEQ ID NO: 2825 - protein, 10 aa, Homo sapiens
LRLKEAPNIS

SEQ ID NO: 2826 - protein, 10 aa, Homo sapiens
RLKEAPNISR

SEQ ID NO: 2827 - protein, 10 aa, Homo sapiens
LKEAPNISRE

SEQ ID NO: 2828 - protein, 10 aa, Homo sapiens
KEAPNISREV

SEQ ID NO: 2829 - protein, 10 aa, Homo sapiens
EAPNISREVV

SEQ ID NO: 2830 - protein, 10 aa, Homo sapiens
APNISREVVK

SEQ ID NO: 2831 - protein, 10 aa, Homo sapiens
PNISREVVKQ

SEQ ID NO: 2832 - protein, 10 aa, Homo sapiens
NISREVVKQL

SEQ ID NO: 2833 - protein, 10 aa, Homo sapiens
ISREVVKQLL

SEQ ID NO: 2834 - protein, 10 aa, Homo sapiens
SREVVKQLLP

SEQ ID NO: 2835 - protein, 10 aa, Homo sapiens
REVVKQLLPK

SEQ ID NO: 2836 - protein, 10 aa, Homo sapiens
EVVKQLLPKA

SEQ ID NO: 2837 - protein, 10 aa, Homo sapiens
VVKQLLPKAP

SEQ ID NO: 2838 - protein, 10 aa, Homo sapiens
VKQLLPKAPP

SEQ ID NO: 2839 - protein, 10 aa, Homo sapiens
KQLLPKAPPL

SEQ ID NO: 2840 - protein, 10 aa, Homo sapiens
QLLPKAPPLQ

SEQ ID NO: 2841 - protein, 10 aa, Homo sapiens
LLPKAPPLQQ

SEQ ID NO: 2842 - protein, 10 aa, Homo sapiens
LPKAPPLQQI

SEQ ID NO: 2843 - protein, 10 aa, Homo sapiens
PKAPPLQQIL

SEQ ID NO: 2844 - protein, 10 aa, Homo sapiens
KAPPLQQILD

SEQ ID NO: 2845 - protein, 10 aa, Homo sapiens
APPLQQILDL

SEQ ID NO: 2846 - protein, 10 aa, Homo sapiens
PPLQQILDLH

SEQ ID NO: 2847 - protein, 10 aa, Homo sapiens
PLQQILDLHD

SEQ ID NO: 2848 - protein, 10 aa, Homo sapiens
LQQILDLHDF

SEQ ID NO: 2849 - protein, 10 aa, Homo sapiens
QQILDLHDFQ

SEQ ID NO: 2850 - protein, 10 aa, Homo sapiens
QILDLHDFQG

SEQ ID NO: 2851 - protein, 10 aa, Homo sapiens
ILDLHDFQGD

SEQ ID NO: 2852 - protein, 10 aa, Homo sapiens
LDLHDFQGDA

SEQ ID NO: 2853 - protein, 10 aa, Homo sapiens
DLHDFQGDAL

SEQ ID NO: 2854 - protein, 10 aa, Homo sapiens
LHDFQGDALQ

SEQ ID NO: 2855 - protein, 10 aa, Homo sapiens
HDFQGDALQP

SEQ ID NO: 2856 - protein, 10 aa, Homo sapiens
DFQGDALQPE

SEQ ID NO: 2857 - protein, 10 aa, Homo sapiens
FQGDALQPED

SEQ ID NO: 2858 - protein, 10 aa, Homo sapiens
QGDALQPEDF

SEQ ID NO: 2859 - protein, 10 aa, Homo sapiens
GDALQPEDFL

SEQ ID NO: 2860 - protein, 10 aa, Homo sapiens
DALQPEDFLE

SEQ ID NO: 2861 - protein, 10 aa, Homo sapiens
ALQPEDFLEE

SEQ ID NO: 2862 - protein, 10 aa, Homo sapiens
LQPEDFLEED

SEQ ID NO: 2863 - protein, 10 aa, Homo sapiens
QPEDFLEEDE

SEQ ID NO: 2864 - protein, 10 aa, Homo sapiens
PEDFLEEDEY

SEQ ID NO: 2865 - protein, 10 aa, Homo sapiens
EDFLEEDEYH

SEQ ID NO: 2866 - protein, 10 aa, Homo sapiens
DFLEEDEYHA

SEQ ID NO: 2867 - protein, 10 aa, Homo sapiens
FLEEDEYHAT

SEQ ID NO: 2868 - protein, 10 aa, Homo sapiens
LEEDEYHATT

SEQ ID NO: 2869 - protein, 10 aa, Homo sapiens
EEDEYHATTE

SEQ ID NO: 2870 - protein, 10 aa, Homo sapiens
EDEYHATTET

SEQ ID NO: 2871 - protein, 10 aa, Homo sapiens
DEYHATTETV

SEQ ID NO: 2872 - protein, 10 aa, Homo sapiens
EYHATTETVI

SEQ ID NO: 2873 - protein, 10 aa, Homo sapiens
YHATTETVIS

SEQ ID NO: 2874 - protein, 10 aa, Homo sapiens
HATTETVISM

SEQ ID NO: 2875 - protein, 10 aa, Homo sapiens
ATTETVISMA

SEQ ID NO: 2876 - protein, 10 aa, Homo sapiens
TTETVISMAQ

SEQ ID NO: 2877 - protein, 10 aa, Homo sapiens
TETVISMAQE

SEQ ID NO: 2878 - protein, 10 aa, Homo sapiens
ETVISMAQET

SEQ ID NO: 2879 - protein, 10 aa, Homo sapiens
TVISMAQETD

SEQ ID NO: 2880 - protein, 10 aa, Homo sapiens
VISMAQETDP

SEQ ID NO: 2881 - protein, 10 aa, Homo sapiens
ISMAQETDPA

SEQ ID NO: 2882 - protein, 10 aa, Homo sapiens
SMAQETDPAV

SEQ ID NO: 2883 - protein, 10 aa, Homo sapiens
MAQETDPAVQ

SEQ ID NO: 2884 - protein, 10 aa, Homo sapiens
AQETDPAVQT

SEQ ID NO: 2885 - protein, 10 aa, Homo sapiens
QETDPAVQTD

SEQ ID NO: 2886 - protein, 10 aa, Homo sapiens
ETDPAVQTDG

SEQ ID NO: 2887 - protein, 10 aa, Homo sapiens
TDPAVQTDGS

SEQ ID NO: 2888 - protein, 10 aa, Homo sapiens
DPAVQTDGSP

SEQ ID NO: 2889 - protein, 10 aa, Homo sapiens
PAVQTDGSPL

SEQ ID NO: 2890 - protein, 10 aa, Homo sapiens
AVQTDGSPLC

SEQ ID NO: 2891 - protein, 10 aa, Homo sapiens
VQTDGSPLCC

SEQ ID NO: 2892 - protein, 10 aa, Homo sapiens
QTDGSPLCCH

SEQ ID NO: 2893 - protein, 10 aa, Homo sapiens
TDGSPLCCHF

SEQ ID NO: 2894 - protein, 10 aa, Homo sapiens
DGSPLCCHFH

SEQ ID NO: 2895 - protein, 10 aa, Homo sapiens
GSPLCCHFHF

SEQ ID NO: 2896 - protein, 10 aa, Homo sapiens
SPLCCHFHFS

SEQ ID NO: 2897 - protein, 10 aa, Homo sapiens
PLCCHFHFSP

SEQ ID NO: 2898 - protein, 10 aa, Homo sapiens
LCCHFHFSPK

SEQ ID NO: 2899 - protein, 10 aa, Homo sapiens
CCHFHFSPKV

SEQ ID NO: 2900 - protein, 10 aa, Homo sapiens
CHFHFSPKVM

SEQ ID NO: 2901 - protein, 10 aa, Homo sapiens
HFHFSPKVMF

SEQ ID NO: 2902 - protein, 10 aa, Homo sapiens
FHFSPKVMFT

SEQ ID NO: 2903 - protein, 10 aa, Homo sapiens
HFSPKVMFTK

SEQ ID NO: 2904 - protein, 10 aa, Homo sapiens
FSPKVMFTKV

SEQ ID NO: 2905 - protein, 10 aa, Homo sapiens
SPKVMFTKVL

SEQ ID NO: 2906 - protein, 10 aa, Homo sapiens
PKVMFTKVLK

SEQ ID NO: 2907 - protein, 10 aa, Homo sapiens
KVMFTKVLKA

SEQ ID NO: 2908 - protein, 10 aa, Homo sapiens
VMFTKVLKAQ

SEQ ID NO: 2909 - protein, 10 aa, Homo sapiens
MFTKVLKAQL

SEQ ID NO: 2910 - protein, 10 aa, Homo sapiens
FTKVLKAQLW

SEQ ID NO: 2911 - protein, 10 aa, Homo sapiens
TKVLKAQLWV

SEQ ID NO: 2912 - protein, 10 aa, Homo sapiens
KVLKAQLWVY

SEQ ID NO: 2913 - protein, 10 aa, Homo sapiens
VLKAQLWVYL

SEQ ID NO: 2914 - protein, 10 aa, Homo sapiens
LKAQLWVYLR

SEQ ID NO: 2915 - protein, 10 aa, Homo sapiens
KAQLWVYLRP

SEQ ID NO: 2916 - protein, 10 aa, Homo sapiens
AQLWVYLRPV

SEQ ID NO: 2917 - protein, 10 aa, Homo sapiens
QLWVYLRPVP

SEQ ID NO: 2918 - protein, 10 aa, Homo sapiens
LWVYLRPVPR

SEQ ID NO: 2919 - protein, 10 aa, Homo sapiens
WVYLRPVPRP

SEQ ID NO: 2920 - protein, 10 aa, Homo sapiens
VYLRPVPRPA

SEQ ID NO: 2921 - protein, 10 aa, Homo sapiens
YLRPVPRPAT

SEQ ID NO: 2922 - protein, 10 aa, Homo sapiens
LRPVPRPATV

SEQ ID NO: 2923 - protein, 10 aa, Homo sapiens
RPVPRPATVY

SEQ ID NO: 2924 - protein, 10 aa, Homo sapiens
PVPRPATVYL

SEQ ID NO: 2925 - protein, 10 aa, Homo sapiens
VPRPATVYLQ

SEQ ID NO: 2926 - protein, 10 aa, Homo sapiens
PRPATVYLQI

SEQ ID NO: 2927 - protein, 10 aa, Homo sapiens
RPATVYLQIL

SEQ ID NO: 2928 - protein, 10 aa, Homo sapiens
PATVYLQILR

SEQ ID NO: 2929 - protein, 10 aa, Homo sapiens
ATVYLQILRL

SEQ ID NO: 2930 - protein, 10 aa, Homo sapiens
TVYLQILRLK

SEQ ID NO: 2931 - protein, 10 aa, Homo sapiens
VYLQILRLKP

SEQ ID NO: 2932 - protein, 10 aa, Homo sapiens
YLQILRLKPL

SEQ ID NO: 2933 - protein, 10 aa, Homo sapiens
LQILRLKPLT

SEQ ID NO: 2934 - protein, 10 aa, Homo sapiens
QILRLKPLTG

SEQ ID NO: 2935 - protein, 10 aa, Homo sapiens
ILRLKPLTGE

SEQ ID NO: 2936 - protein, 10 aa, Homo sapiens
LRLKPLTGEG

SEQ ID NO: 2937 - protein, 10 aa, Homo sapiens
RLKPLTGEGT

SEQ ID NO: 2938 - protein, 10 aa, Homo sapiens
LKPLTGEGTA

SEQ ID NO: 2939 - protein, 10 aa, Homo sapiens
KPLTGEGTAG

SEQ ID NO: 2940 - protein, 10 aa, Homo sapiens
PLTGEGTAGG

SEQ ID NO: 2941 - protein, 10 aa, Homo sapiens
LTGEGTAGGG

SEQ ID NO: 2942 - protein, 10 aa, Homo sapiens
TGEGTAGGGG

SEQ ID NO: 2943 - protein, 10 aa, Homo sapiens
GEGTAGGGGG

SEQ ID NO: 2944 - protein, 10 aa, Homo sapiens
EGTAGGGGGG

SEQ ID NO: 2945 - protein, 10 aa, Homo sapiens
GTAGGGGGGR

SEQ ID NO: 2946 - protein, 10 aa, Homo sapiens
TAGGGGGGRR

SEQ ID NO: 2947 - protein, 10 aa, Homo sapiens
AGGGGGGRRH

SEQ ID NO: 2948 - protein, 10 aa, Homo sapiens
GGGGGGRRHI

SEQ ID NO: 2949 - protein, 10 aa, Homo sapiens
GGGGGRRHIR

SEQ ID NO: 2950 - protein, 10 aa, Homo sapiens
GGGGRRHIRI

SEQ ID NO: 2951 - protein, 10 aa, Homo sapiens
GGGRRHIRIR

SEQ ID NO: 2952 - protein, 10 aa, Homo sapiens
GGRRHIRIRS

SEQ ID NO: 2953 - protein, 10 aa, Homo sapiens
GRRHIRIRSL

SEQ ID NO: 2954 - protein, 10 aa, Homo sapiens
RRHIRIRSLK

SEQ ID NO: 2955 - protein, 10 aa, Homo sapiens
RHIRIRSLKI

SEQ ID NO: 2956 - protein, 10 aa, Homo sapiens
HIRIRSLKIE

SEQ ID NO: 2957 - protein, 10 aa, Homo sapiens
IRIRSLKIEL

SEQ ID NO: 2958 - protein, 10 aa, Homo sapiens
RIRSLKIELH

SEQ ID NO: 2959 - protein, 10 aa, Homo sapiens
IRSLKIELHS

SEQ ID NO: 2960 - protein, 10 aa, Homo sapiens
RSLKIELHSR

SEQ ID NO: 2961 - protein, 10 aa, Homo sapiens
SLKIELHSRS

SEQ ID NO: 2962 - protein, 10 aa, Homo sapiens
LKIELHSRSG

SEQ ID NO: 2963 - protein, 10 aa, Homo sapiens
KIELHSRSGH

SEQ ID NO: 2964 - protein, 10 aa, Homo sapiens
IELHSRSGHW

SEQ ID NO: 2965 - protein, 10 aa, Homo sapiens
ELHSRSGHWQ

SEQ ID NO: 2966 - protein, 10 aa, Homo sapiens
LHSRSGHWQS

SEQ ID NO: 2967 - protein, 10 aa, Homo sapiens
HSRSGHWQSI

SEQ ID NO: 2968 - protein, 10 aa, Homo sapiens
SRSGHWQSID

SEQ ID NO: 2969 - protein, 10 aa, Homo sapiens
RSGHWQSIDF

SEQ ID NO: 2970 - protein, 10 aa, Homo sapiens
SGHWQSIDFK

SEQ ID NO: 2971 - protein, 10 aa, Homo sapiens
GHWQSIDFKQ

SEQ ID NO: 2972 - protein, 10 aa, Homo sapiens
HWQSIDFKQV

SEQ ID NO: 2973 - protein, 10 aa, Homo sapiens
WQSIDFKQVL

SEQ ID NO: 2974 - protein, 10 aa, Homo sapiens
QSIDFKQVLH

SEQ ID NO: 2975 - protein, 10 aa, Homo sapiens
SIDFKQVLHS

SEQ ID NO: 2976 - protein, 10 aa, Homo sapiens
IDFKQVLHSW

SEQ ID NO: 2977 - protein, 10 aa, Homo sapiens
DFKQVLHSWF

SEQ ID NO: 2978 - protein, 10 aa, Homo sapiens
FKQVLHSWFR

SEQ ID NO: 2979 - protein, 10 aa, Homo sapiens
KQVLHSWFRQ

SEQ ID NO: 2980 - protein, 10 aa, Homo sapiens
QVLHSWFRQP

SEQ ID NO: 2981 - protein, 10 aa, Homo sapiens
VLHSWFRQPQ

SEQ ID NO: 2982 - protein, 10 aa, Homo sapiens
LHSWFRQPQS

SEQ ID NO: 2983 - protein, 10 aa, Homo sapiens
HSWFRQPQSN

SEQ ID NO: 2984 - protein, 10 aa, Homo sapiens
SWFRQPQSNW

SEQ ID NO: 2985 - protein, 10 aa, Homo sapiens
WFRQPQSNWG

SEQ ID NO: 2986 - protein, 10 aa, Homo sapiens
FRQPQSNWGI

SEQ ID NO: 2987 - protein, 10 aa, Homo sapiens
RQPQSNWGIE

SEQ ID NO: 2988 - protein, 10 aa, Homo sapiens
QPQSNWGIEI

SEQ ID NO: 2989 - protein, 10 aa, Homo sapiens
PQSNWGIEIN

SEQ ID NO: 2990 - protein, 10 aa, Homo sapiens
QSNWGIEINA

SEQ ID NO: 2991 - protein, 10 aa, Homo sapiens
SNWGIEINAF

SEQ ID NO: 2992 - protein, 10 aa, Homo sapiens
NWGIEINAFD

SEQ ID NO: 2993 - protein, 10 aa, Homo sapiens
WGIEINAFDP

SEQ ID NO: 2994 - protein, 10 aa, Homo sapiens
GIEINAFDPS

SEQ ID NO: 2995 - protein, 10 aa, Homo sapiens
IEINAFDPSG

SEQ ID NO: 2996 - protein, 10 aa, Homo sapiens
EINAFDPSGT

SEQ ID NO: 2997 - protein, 10 aa, Homo sapiens
INAFDPSGTD

SEQ ID NO: 2998 - protein, 10 aa, Homo sapiens
NAFDPSGTDL

SEQ ID NO: 2999 - protein, 10 aa, Homo sapiens
AFDPSGTDLA

SEQ ID NO: 3000 - protein, 10 aa, Homo sapiens
FDPSGTDLAV

SEQ ID NO: 3001 - protein, 10 aa, Homo sapiens
DPSGTDLAVT

SEQ ID NO: 3002 - protein, 10 aa, Homo sapiens
PSGTDLAVTS

SEQ ID NO: 3003 - protein, 10 aa, Homo sapiens
SGTDLAVTSL

SEQ ID NO: 3004 - protein, 10 aa, Homo sapiens
GTDLAVTSLG

SEQ ID NO: 3005 - protein, 10 aa, Homo sapiens
TDLAVTSLGP

SEQ ID NO: 3006 - protein, 10 aa, Homo sapiens
DLAVTSLGPG

SEQ ID NO: 3007 - protein, 10 aa, Homo sapiens
LAVTSLGPGA

SEQ ID NO: 3008 - protein, 10 aa, Homo sapiens
AVTSLGPGAE

SEQ ID NO: 3009 - protein, 10 aa, Homo sapiens
VTSLGPGAEG

SEQ ID NO: 3010 - protein, 10 aa, Homo sapiens
TSLGPGAEGL

SEQ ID NO: 3011 - protein, 10 aa, Homo sapiens
SLGPGAEGLH

SEQ ID NO: 3012 - protein, 10 aa, Homo sapiens
LGPGAEGLHP

SEQ ID NO: 3013 - protein, 10 aa, Homo sapiens
GPGAEGLHPF

SEQ ID NO: 3014 - protein, 10 aa, Homo sapiens
PGAEGLHPFM

SEQ ID NO: 3015 - protein, 10 aa, Homo sapiens
GAEGLHPFME

SEQ ID NO: 3016 - protein, 10 aa, Homo sapiens
AEGLHPFMEL

SEQ ID NO: 3017 - protein, 10 aa, Homo sapiens
EGLHPFMELR

SEQ ID NO: 3018 - protein, 10 aa, Homo sapiens
GLHPFMELRV

SEQ ID NO: 3019 - protein, 10 aa, Homo sapiens
LHPFMELRVL

SEQ ID NO: 3020 - protein, 10 aa, Homo sapiens
HPFMELRVLE

SEQ ID NO: 3021 - protein, 10 aa, Homo sapiens
PFMELRVLEN

SEQ ID NO: 3022 - protein, 10 aa, Homo sapiens
FMELRVLENT

SEQ ID NO: 3023 - protein, 10 aa, Homo sapiens
MELRVLENTK

SEQ ID NO: 3024 - protein, 10 aa, Homo sapiens
ELRVLENTKR

SEQ ID NO: 3025 - protein, 10 aa, Homo sapiens
LRVLENTKRS

SEQ ID NO: 3026 - protein, 10 aa, Homo sapiens
RVLENTKRSR

SEQ ID NO: 3027 - protein, 10 aa, Homo sapiens
VLENTKRSRR

SEQ ID NO: 3028 - protein, 10 aa, Homo sapiens
LENTKRSRRN

SEQ ID NO: 3029 - protein, 10 aa, Homo sapiens
ENTKRSRRNL

SEQ ID NO: 3030 - protein, 10 aa, Homo sapiens
NTKRSRRNLG

SEQ ID NO: 3031 - protein, 10 aa, Homo sapiens
TKRSRRNLGL

SEQ ID NO: 3032 - protein, 10 aa, Homo sapiens
KRSRRNLGLD

SEQ ID NO: 3033 - protein, 10 aa, Homo sapiens
RSRRNLGLDC

SEQ ID NO: 3034 - protein, 10 aa, Homo sapiens
SRRNLGLDCD

SEQ ID NO: 3035 - protein, 10 aa, Homo sapiens
RRNLGLDCDE

SEQ ID NO: 3036 - protein, 10 aa, Homo sapiens
RNLGLDCDEH

SEQ ID NO: 3037 - protein, 10 aa, Homo sapiens
NLGLDCDEHS

SEQ ID NO: 3038 - protein, 10 aa, Homo sapiens
LGLDCDEHSS

SEQ ID NO: 3039 - protein, 10 aa, Homo sapiens
GLDCDEHSSE

SEQ ID NO: 3040 - protein, 10 aa, Homo sapiens
LDCDEHSSES

SEQ ID NO: 3041 - protein, 10 aa, Homo sapiens
DCDEHSSESR

SEQ ID NO: 3042 - protein, 10 aa, Homo sapiens
CDEHSSESRC

SEQ ID NO: 3043 - protein, 10 aa, Homo sapiens
DEHSSESRCC

SEQ ID NO: 3044 - protein, 10 aa, Homo sapiens
EHSSESRCCR

SEQ ID NO: 3045 - protein, 10 aa, Homo sapiens
HSSESRCCRY

SEQ ID NO: 3046 - protein, 10 aa, Homo sapiens
SSESRCCRYP

SEQ ID NO: 3047 - protein, 10 aa, Homo sapiens
SESRCCRYPL

SEQ ID NO: 3048 - protein, 10 aa, Homo sapiens
ESRCCRYPLT

SEQ ID NO: 3049 - protein, 10 aa, Homo sapiens
SRCCRYPLTV

SEQ ID NO: 3050 - protein, 10 aa, Homo sapiens
RCCRYPLTVD

SEQ ID NO: 3051 - protein, 10 aa, Homo sapiens
CCRYPLTVDF

SEQ ID NO: 3052 - protein, 10 aa, Homo sapiens
CRYPLTVDFE

SEQ ID NO: 3053 - protein, 10 aa, Homo sapiens
RYPLTVDFEA

SEQ ID NO: 3054 - protein, 10 aa, Homo sapiens
YPLTVDFEAF

SEQ ID NO: 3055 - protein, 10 aa, Homo sapiens
PLTVDFEAFG

SEQ ID NO: 3056 - protein, 10 aa, Homo sapiens
LTVDFEAFGW

SEQ ID NO: 3057 - protein, 10 aa, Homo sapiens
TVDFEAFGWD

SEQ ID NO: 3058 - protein, 10 aa, Homo sapiens
VDFEAFGWDW

SEQ ID NO: 3059 - protein, 10 aa, Homo sapiens
DFEAFGWDWI

SEQ ID NO: 3060 - protein, 10 aa, Homo sapiens
FEAFGWDWII

SEQ ID NO: 3061 - protein, 10 aa, Homo sapiens
EAFGWDWIIA

SEQ ID NO: 3062 - protein, 10 aa, Homo sapiens
AFGWDWIIAP

SEQ ID NO: 3063 - protein, 10 aa, Homo sapiens
FGWDWIIAPK

SEQ ID NO: 3064 - protein, 10 aa, Homo sapiens
GWDWIIAPKR

SEQ ID NO: 3065 - protein, 10 aa, Homo sapiens
WDWIIAPKRY

SEQ ID NO: 3066 - protein, 10 aa, Homo sapiens
DWIIAPKRYK

SEQ ID NO: 3067 - protein, 10 aa, Homo sapiens
WIIAPKRYKA

SEQ ID NO: 3068 - protein, 10 aa, Homo sapiens
IIAPKRYKAN

SEQ ID NO: 3069 - protein, 10 aa, Homo sapiens
IAPKRYKANY

SEQ ID NO: 3070 - protein, 10 aa, Homo sapiens
APKRYKANYC

SEQ ID NO: 3071 - protein, 10 aa, Homo sapiens
PKRYKANYCS

SEQ ID NO: 3072 - protein, 10 aa, Homo sapiens
KRYKANYCSG

SEQ ID NO: 3073 - protein, 10 aa, Homo sapiens
RYKANYCSGQ

SEQ ID NO: 3074 - protein, 10 aa, Homo sapiens
YKANYCSGQC

SEQ ID NO: 3075 - protein, 10 aa, Homo sapiens
KANYCSGQCE

SEQ ID NO: 3076 - protein, 10 aa, Homo sapiens
ANYCSGQCEY

SEQ ID NO: 3077 - protein, 10 aa, Homo sapiens
NYCSGQCEYM

SEQ ID NO: 3078 - protein, 10 aa, Homo sapiens
YCSGQCEYMF

SEQ ID NO: 3079 - protein, 10 aa, Homo sapiens
CSGQCEYMFM

SEQ ID NO: 3080 - protein, 10 aa, Homo sapiens
SGQCEYMFMQ

SEQ ID NO: 3081 - protein, 10 aa, Homo sapiens
GQCEYMFMQK

SEQ ID NO: 3082 - protein, 10 aa, Homo sapiens
QCEYMFMQKY

SEQ ID NO: 3083 - protein, 10 aa, Homo sapiens
CEYMFMQKYP

SEQ ID NO: 3084 - protein, 10 aa, Homo sapiens
EYMFMQKYPH

SEQ ID NO: 3085 - protein, 10 aa, Homo sapiens
YMFMQKYPHT

SEQ ID NO: 3086 - protein, 10 aa, Homo sapiens
MFMQKYPHTH

SEQ ID NO: 3087 - protein, 10 aa, Homo sapiens
FMQKYPHTHL

SEQ ID NO: 3088 - protein, 10 aa, Homo sapiens
MQKYPHTHLV

SEQ ID NO: 3089 - protein, 10 aa, Homo sapiens
QKYPHTHLVQ

SEQ ID NO: 3090 - protein, 10 aa, Homo sapiens
KYPHTHLVQQ

SEQ ID NO: 3091 - protein, 10 aa, Homo sapiens
YPHTHLVQQA

SEQ ID NO: 3092 - protein, 10 aa, Homo sapiens
PHTHLVQQAN

SEQ ID NO: 3093 - protein, 10 aa, Homo sapiens
HTHLVQQANP

SEQ ID NO: 3094 - protein, 10 aa, Homo sapiens
THLVQQANPR

SEQ ID NO: 3095 - protein, 10 aa, Homo sapiens
HLVQQANPRG

SEQ ID NO: 3096 - protein, 10 aa, Homo sapiens
LVQQANPRGS

SEQ ID NO: 3097 - protein, 10 aa, Homo sapiens
VQQANPRGSA

SEQ ID NO: 3098 - protein, 10 aa, Homo sapiens
QQANPRGSAG

SEQ ID NO: 3099 - protein, 10 aa, Homo sapiens
QANPRGSAGP

SEQ ID NO: 3100 - protein, 10 aa, Homo sapiens
ANPRGSAGPC

SEQ ID NO: 3101 - protein, 10 aa, Homo sapiens
NPRGSAGPCC

SEQ ID NO: 3102 - protein, 10 aa, Homo sapiens
PRGSAGPCCT

SEQ ID NO: 3103 - protein, 10 aa, Homo sapiens
RGSAGPCCTP

SEQ ID NO: 3104 - protein, 10 aa, Homo sapiens
GSAGPCCTPT

SEQ ID NO: 3105 - protein, 10 aa, Homo sapiens
SAGPCCTPTK

SEQ ID NO: 3106 - protein, 10 aa, Homo sapiens
AGPCCTPTKM

SEQ ID NO: 3107 - protein, 10 aa, Homo sapiens
GPCCTPTKMS

SEQ ID NO: 3108 - protein, 10 aa, Homo sapiens
PCCTPTKMSP

SEQ ID NO: 3109 - protein, 10 aa, Homo sapiens
CCTPTKMSPI

SEQ ID NO: 3110 - protein, 10 aa, Homo sapiens
CTPTKMSPIN

SEQ ID NO: 3111 - protein, 10 aa, Homo sapiens
TPTKMSPINM

SEQ ID NO: 3112 - protein, 10 aa, Homo sapiens
PTKMSPINML

SEQ ID NO: 3113 - protein, 10 aa, Homo sapiens
TKMSPINMLY

SEQ ID NO: 3114 - protein, 10 aa, Homo sapiens
KMSPINMLYF

SEQ ID NO: 3115 - protein, 10 aa, Homo sapiens
MSPINMLYFN

SEQ ID NO: 3116 - protein, 10 aa, Homo sapiens
SPINMLYFND

SEQ ID NO: 3117 - protein, 10 aa, Homo sapiens
PINMLYFNDK

SEQ ID NO: 3118 - protein, 10 aa, Homo sapiens
INMLYFNDKQ

SEQ ID NO: 3119 - protein, 10 aa, Homo sapiens
NMLYFNDKQQ

SEQ ID NO: 3120 - protein, 10 aa, Homo sapiens
MLYFNDKQQI

SEQ ID NO: 3121 - protein, 10 aa, Homo sapiens
LYFNDKQQII

SEQ ID NO: 3122 - protein, 10 aa, Homo sapiens
YFNDKQQIIY

SEQ ID NO: 3123 - protein, 10 aa, Homo sapiens
FNDKQQIIYG

SEQ ID NO: 3124 - protein, 10 aa, Homo sapiens
NDKQQIIYGK

SEQ ID NO: 3125 - protein, 10 aa, Homo sapiens
DKQQIIYGKI

SEQ ID NO: 3126 - protein, 10 aa, Homo sapiens
KQQIIYGKIP

SEQ ID NO: 3127 - protein, 10 aa, Homo sapiens
QQIIYGKIPG

SEQ ID NO: 3128 - protein, 10 aa, Homo sapiens
QIIYGKIPGM

SEQ ID NO: 3129 - protein, 10 aa, Homo sapiens
IIYGKIPGMV

SEQ ID NO: 3130 - protein, 10 aa, Homo sapiens
IYGKIPGMVV

SEQ ID NO: 3131 - protein, 10 aa, Homo sapiens
YGKIPGMVVD

SEQ ID NO: 3132 - protein, 10 aa, Homo sapiens
GKIPGMVVDR

SEQ ID NO: 3133 - protein, 10 aa, Homo sapiens
KIPGMVVDRC

SEQ ID NO: 3134 - protein, 10 aa, Homo sapiens
IPGMVVDRCG

SEQ ID NO: 3135 - protein, 10 aa, Homo sapiens
PGMVVDRCGC

SEQ ID NO: 3136 - protein, 10 aa, Homo sapiens
GMVVDRCGCS

SEQ ID NO: 3137 - protein, 11 aa, Homo sapiens
MVLAAPLLLGF

SEQ ID NO: 3138 - protein, 11 aa, Homo sapiens
VLAAPLLLGFL

SEQ ID NO: 3139 - protein, 11 aa, Homo sapiens
LAAPLLLGFLL

SEQ ID NO: 3140 - protein, 11 aa, Homo sapiens
AAPLLLGFLLL

SEQ ID NO: 3141 - protein, 11 aa, Homo sapiens
APLLLGFLLLA

SEQ ID NO: 3142 - protein, 11 aa, Homo sapiens
PLLLGFLLLAL

SEQ ID NO: 3143 - protein, 11 aa, Homo sapiens
LLLGFLLLALE

SEQ ID NO: 3144 - protein, 11 aa, Homo sapiens
LLGFLLLALEL

SEQ ID NO: 3145 - protein, 11 aa, Homo sapiens
LGFLLLALELR

SEQ ID NO: 3146 - protein, 11 aa, Homo sapiens
GFLLLALELRP

SEQ ID NO: 3147 - protein, 11 aa, Homo sapiens
FLLLALELRPR

SEQ ID NO: 3148 - protein, 11 aa, Homo sapiens
LLLALELRPRG

SEQ ID NO: 3149 - protein, 11 aa, Homo sapiens
LLALELRPRGE

SEQ ID NO: 3150 - protein, 11 aa, Homo sapiens
LALELRPRGEA

SEQ ID NO: 3151 - protein, 11 aa, Homo sapiens
ALELRPRGEAA

SEQ ID NO: 3152 - protein, 11 aa, Homo sapiens
LELRPRGEAAE

SEQ ID NO: 3153 - protein, 11 aa, Homo sapiens
ELRPRGEAAEG

SEQ ID NO: 3154 - protein, 11 aa, Homo sapiens
LRPRGEAAEGP

SEQ ID NO: 3155 - protein, 11 aa, Homo sapiens
RPRGEAAEGPA

SEQ ID NO: 3156 - protein, 11 aa, Homo sapiens
PRGEAAEGPAA

SEQ ID NO: 3157 - protein, 11 aa, Homo sapiens
RGEAAEGPAAA

SEQ ID NO: 3158 - protein, 11 aa, Homo sapiens
GEAAEGPAAAA

SEQ ID NO: 3159 - protein, 11 aa, Homo sapiens
EAAEGPAAAAA

SEQ ID NO: 3160 - protein, 11 aa, Homo sapiens
AAEGPAAAAAA

SEQ ID NO: 3161 - protein, 11 aa, Homo sapiens
AEGPAAAAAAA

SEQ ID NO: 3162 - protein, 11 aa, Homo sapiens
EGPAAAAAAAA

SEQ ID NO: 3163 - protein, 11 aa, Homo sapiens
GPAAAAAAAAA

SEQ ID NO: 3164 - protein, 11 aa, Homo sapiens
PAAAAAAAAAA

SEQ ID NO: 3165 - protein, 11 aa, Homo sapiens
AAAAAAAAAAA

SEQ ID NO: 3166 - protein, 11 aa, Homo sapiens
AAAAAAAAAAG

SEQ ID NO: 3167 - protein, 11 aa, Homo sapiens
AAAAAAAAAGV

SEQ ID NO: 3168 - protein, 11 aa, Homo sapiens
AAAAAAAAGVG

SEQ ID NO: 3169 - protein, 11 aa, Homo sapiens
AAAAAAAGVGG

SEQ ID NO: 3170 - protein, 11 aa, Homo sapiens
AAAAAAGVGGE

SEQ ID NO: 3171 - protein, 11 aa, Homo sapiens
AAAAAGVGGER

SEQ ID NO: 3172 - protein, 11 aa, Homo sapiens
AAAAGVGGERS

SEQ ID NO: 3173 - protein, 11 aa, Homo sapiens
AAAGVGGERSS

SEQ ID NO: 3174 - protein, 11 aa, Homo sapiens
AAGVGGERSSR

SEQ ID NO: 3175 - protein, 11 aa, Homo sapiens
AGVGGERSSRP

SEQ ID NO: 3176 - protein, 11 aa, Homo sapiens
GVGGERSSRPA

SEQ ID NO: 3177 - protein, 11 aa, Homo sapiens
VGGERSSRPAP

SEQ ID NO: 3178 - protein, 11 aa, Homo sapiens
GGERSSRPAPS

SEQ ID NO: 3179 - protein, 11 aa, Homo sapiens
GERSSRPAPSV

SEQ ID NO: 3180 - protein, 11 aa, Homo sapiens
ERSSRPAPSVA

SEQ ID NO: 3181 - protein, 11 aa, Homo sapiens
RSSRPAPSVAP

SEQ ID NO: 3182 - protein, 11 aa, Homo sapiens
SSRPAPSVAPE

SEQ ID NO: 3183 - protein, 11 aa, Homo sapiens
SRPAPSVAPEP

SEQ ID NO: 3184 - protein, 11 aa, Homo sapiens
RPAPSVAPEPD

SEQ ID NO: 3185 - protein, 11 aa, Homo sapiens
PAPSVAPEPDG

SEQ ID NO: 3186 - protein, 11 aa, Homo sapiens
APSVAPEPDGC

SEQ ID NO: 3187 - protein, 11 aa, Homo sapiens
PSVAPEPDGCP

SEQ ID NO: 3188 - protein, 11 aa, Homo sapiens
SVAPEPDGCPV

SEQ ID NO: 3189 - protein, 11 aa, Homo sapiens
VAPEPDGCPVC

SEQ ID NO: 3190 - protein, 11 aa, Homo sapiens
APEPDGCPVCV

SEQ ID NO: 3191 - protein, 11 aa, Homo sapiens
PEPDGCPVCVW

SEQ ID NO: 3192 - protein, 11 aa, Homo sapiens
EPDGCPVCVWR

SEQ ID NO: 3193 - protein, 11 aa, Homo sapiens
PDGCPVCVWRQ

SEQ ID NO: 3194 - protein, 11 aa, Homo sapiens
DGCPVCVWRQH

SEQ ID NO: 3195 - protein, 11 aa, Homo sapiens
GCPVCVWRQHS

SEQ ID NO: 3196 - protein, 11 aa, Homo sapiens
CPVCVWRQHSR

SEQ ID NO: 3197 - protein, 11 aa, Homo sapiens
PVCVWRQHSRE

SEQ ID NO: 3198 - protein, 11 aa, Homo sapiens
VCVWRQHSREL

SEQ ID NO: 3199 - protein, 11 aa, Homo sapiens
CVWRQHSRELR

SEQ ID NO: 3200 - protein, 11 aa, Homo sapiens
VWRQHSRELRL

SEQ ID NO: 3201 - protein, 11 aa, Homo sapiens
WRQHSRELRLE

SEQ ID NO: 3202 - protein, 11 aa, Homo sapiens
RQHSRELRLES

SEQ ID NO: 3203 - protein, 11 aa, Homo sapiens
QHSRELRLESI

SEQ ID NO: 3204 - protein, 11 aa, Homo sapiens
HSRELRLESIK

SEQ ID NO: 3205 - protein, 11 aa, Homo sapiens
SRELRLESIKS

SEQ ID NO: 3206 - protein, 11 aa, Homo sapiens
RELRLESIKSQ

SEQ ID NO: 3207 - protein, 11 aa, Homo sapiens
ELRLESIKSQI

SEQ ID NO: 3208 - protein, 11 aa, Homo sapiens
LRLESIKSQIL

SEQ ID NO: 3209 - protein, 11 aa, Homo sapiens
RLESIKSQILS

SEQ ID NO: 3210 - protein, 11 aa, Homo sapiens
LESIKSQILSK

SEQ ID NO: 3211 - protein, 11 aa, Homo sapiens
ESIKSQILSKL

SEQ ID NO: 3212 - protein, 11 aa, Homo sapiens
SIKSQILSKLR

SEQ ID NO: 3213 - protein, 11 aa, Homo sapiens
IKSQILSKLRL

SEQ ID NO: 3214 - protein, 11 aa, Homo sapiens
KSQILSKLRLK

SEQ ID NO: 3215 - protein, 11 aa, Homo sapiens
SQILSKLRLKE

SEQ ID NO: 3216 - protein, 11 aa, Homo sapiens
QILSKLRLKEA

SEQ ID NO: 3217 - protein, 11 aa, Homo sapiens
ILSKLRLKEAP

SEQ ID NO: 3218 - protein, 11 aa, Homo sapiens
LSKLRLKEAPN

SEQ ID NO: 3219 - protein, 11 aa, Homo sapiens
SKLRLKEAPNI

SEQ ID NO: 3220 - protein, 11 aa, Homo sapiens
KLRLKEAPNIS

SEQ ID NO: 3221 - protein, 11 aa, Homo sapiens
LRLKEAPNISR

SEQ ID NO: 3222 - protein, 11 aa, Homo sapiens
RLKEAPNISRE

SEQ ID NO: 3223 - protein, 11 aa, Homo sapiens
LKEAPNISREV

SEQ ID NO: 3224 - protein, 11 aa, Homo sapiens
KEAPNISREVV

SEQ ID NO: 3225 - protein, 11 aa, Homo sapiens
EAPNISREVVK

SEQ ID NO: 3226 - protein, 11 aa, Homo sapiens
APNISREVVKQ

SEQ ID NO: 3227 - protein, 11 aa, Homo sapiens
PNISREVVKQL

SEQ ID NO: 3228 - protein, 11 aa, Homo sapiens
NISREVVKQLL

SEQ ID NO: 3229 - protein, 11 aa, Homo sapiens
ISREVVKQLLP

SEQ ID NO: 3230 - protein, 11 aa, Homo sapiens
SREVVKQLLPK

SEQ ID NO: 3231 - protein, 11 aa, Homo sapiens
REVVKQLLPKA

SEQ ID NO: 3232 - protein, 11 aa, Homo sapiens
EVVKQLLPKAP

SEQ ID NO: 3233 - protein, 11 aa, Homo sapiens
VVKQLLPKAPP

SEQ ID NO: 3234 - protein, 11 aa, Homo sapiens
VKQLLPKAPPL

SEQ ID NO: 3235 - protein, 11 aa, Homo sapiens
KQLLPKAPPLQ

SEQ ID NO: 3236 - protein, 11 aa, Homo sapiens
QLLPKAPPLQQ

SEQ ID NO: 3237 - protein, 11 aa, Homo sapiens
LLPKAPPLQQI

SEQ ID NO: 3238 - protein, 11 aa, Homo sapiens
LPKAPPLQQIL

SEQ ID NO: 3239 - protein, 11 aa, Homo sapiens
PKAPPLQQILD

SEQ ID NO: 3240 - protein, 11 aa, Homo sapiens
KAPPLQQILDL

SEQ ID NO: 3241 - protein, 11 aa, Homo sapiens
APPLQQILDLH

SEQ ID NO: 3242 - protein, 11 aa, Homo sapiens
PPLQQILDLHD

SEQ ID NO: 3243 - protein, 11 aa, Homo sapiens
PLQQILDLHDF

SEQ ID NO: 3244 - protein, 11 aa, Homo sapiens
LQQILDLHDFQ

SEQ ID NO: 3245 - protein, 11 aa, Homo sapiens
QQILDLHDFQG

SEQ ID NO: 3246 - protein, 11 aa, Homo sapiens
QILDLHDFQGD

SEQ ID NO: 3247 - protein, 11 aa, Homo sapiens
ILDLHDFQGDA

SEQ ID NO: 3248 - protein, 11 aa, Homo sapiens
LDLHDFQGDAL

SEQ ID NO: 3249 - protein, 11 aa, Homo sapiens
DLHDFQGDALQ

SEQ ID NO: 3250 - protein, 11 aa, Homo sapiens
LHDFQGDALQP

SEQ ID NO: 3251 - protein, 11 aa, Homo sapiens
HDFQGDALQPE

SEQ ID NO: 3252 - protein, 11 aa, Homo sapiens
DFQGDALQPED

SEQ ID NO: 3253 - protein, 11 aa, Homo sapiens
FQGDALQPEDF

SEQ ID NO: 3254 - protein, 11 aa, Homo sapiens
QGDALQPEDFL

SEQ ID NO: 3255 - protein, 11 aa, Homo sapiens
GDALQPEDFLE

SEQ ID NO: 3256 - protein, 11 aa, Homo sapiens
DALQPEDFLEE

SEQ ID NO: 3257 - protein, 11 aa, Homo sapiens
ALQPEDFLEED

SEQ ID NO: 3258 - protein, 11 aa, Homo sapiens
LQPEDFLEEDE

SEQ ID NO: 3259 - protein, 11 aa, Homo sapiens
QPEDFLEEDEY

SEQ ID NO: 3260 - protein, 11 aa, Homo sapiens
PEDFLEEDEYH

SEQ ID NO: 3261 - protein, 11 aa, Homo sapiens
EDFLEEDEYHA

SEQ ID NO: 3262 - protein, 11 aa, Homo sapiens
DFLEEDEYHAT

SEQ ID NO: 3263 - protein, 11 aa, Homo sapiens
FLEEDEYHATT

SEQ ID NO: 3264 - protein, 11 aa, Homo sapiens
LEEDEYHATTE

SEQ ID NO: 3265 - protein, 11 aa, Homo sapiens
EEDEYHATTET

SEQ ID NO: 3266 - protein, 11 aa, Homo sapiens
EDEYHATTETV

SEQ ID NO: 3267 - protein, 11 aa, Homo sapiens
DEYHATTETVI

SEQ ID NO: 3268 - protein, 11 aa, Homo sapiens
EYHATTETVIS

SEQ ID NO: 3269 - protein, 11 aa, Homo sapiens
YHATTETVISM

SEQ ID NO: 3270 - protein, 11 aa, Homo sapiens
HATTETVISMA

SEQ ID NO: 3271 - protein, 11 aa, Homo sapiens
ATTETVISMAQ

SEQ ID NO: 3272 - protein, 11 aa, Homo sapiens
TTETVISMAQE

SEQ ID NO: 3273 - protein, 11 aa, Homo sapiens
TETVISMAQET

SEQ ID NO: 3274 - protein, 11 aa, Homo sapiens
ETVISMAQETD

SEQ ID NO: 3275 - protein, 11 aa, Homo sapiens
TVISMAQETDP

SEQ ID NO: 3276 - protein, 11 aa, Homo sapiens
VISMAQETDPA

SEQ ID NO: 3277 - protein, 11 aa, Homo sapiens
ISMAQETDPAV

SEQ ID NO: 3278 - protein, 11 aa, Homo sapiens
SMAQETDPAVQ

SEQ ID NO: 3279 - protein, 11 aa, Homo sapiens
MAQETDPAVQT

SEQ ID NO: 3280 - protein, 11 aa, Homo sapiens
AQETDPAVQTD

SEQ ID NO: 3281 - protein, 11 aa, Homo sapiens
QETDPAVQTDG

SEQ ID NO: 3282 - protein, 11 aa, Homo sapiens
ETDPAVQTDGS

SEQ ID NO: 3283 - protein, 11 aa, Homo sapiens
TDPAVQTDGSP

SEQ ID NO: 3284 - protein, 11 aa, Homo sapiens
DPAVQTDGSPL

SEQ ID NO: 3285 - protein, 11 aa, Homo sapiens
PAVQTDGSPLC

SEQ ID NO: 3286 - protein, 11 aa, Homo sapiens
AVQTDGSPLCC

SEQ ID NO: 3287 - protein, 11 aa, Homo sapiens
VQTDGSPLCCH

SEQ ID NO: 3288 - protein, 11 aa, Homo sapiens
QTDGSPLCCHF

SEQ ID NO: 3289 - protein, 11 aa, Homo sapiens
TDGSPLCCHFH

SEQ ID NO: 3290 - protein, 11 aa, Homo sapiens
DGSPLCCHFHF

SEQ ID NO: 3291 - protein, 11 aa, Homo sapiens
GSPLCCHFHFS

SEQ ID NO: 3292 - protein, 11 aa, Homo sapiens
SPLCCHFHFSP

SEQ ID NO: 3293 - protein, 11 aa, Homo sapiens
PLCCHFHFSPK

SEQ ID NO: 3294 - protein, 11 aa, Homo sapiens
LCCHFHFSPKV

SEQ ID NO: 3295 - protein, 11 aa, Homo sapiens
CCHFHFSPKVM

SEQ ID NO: 3296 - protein, 11 aa, Homo sapiens
CHFHFSPKVMF

SEQ ID NO: 3297 - protein, 11 aa, Homo sapiens
HFHFSPKVMFT

SEQ ID NO: 3298 - protein, 11 aa, Homo sapiens
FHFSPKVMFTK

SEQ ID NO: 3299 - protein, 11 aa, Homo sapiens
HFSPKVMFTKV

SEQ ID NO: 3300 - protein, 11 aa, Homo sapiens
FSPKVMFTKVL

SEQ ID NO: 3301 - protein, 11 aa, Homo sapiens
SPKVMFTKVLK

SEQ ID NO: 3302 - protein, 11 aa, Homo sapiens
PKVMFTKVLKA

SEQ ID NO: 3303 - protein, 11 aa, Homo sapiens
KVMFTKVLKAQ

SEQ ID NO: 3304 - protein, 11 aa, Homo sapiens
VMFTKVLKAQL

SEQ ID NO: 3305 - protein, 11 aa, Homo sapiens
MFTKVLKAQLW

SEQ ID NO: 3306 - protein, 11 aa, Homo sapiens
FTKVLKAQLWV

SEQ ID NO: 3307 - protein, 11 aa, Homo sapiens
TKVLKAQLWVY

SEQ ID NO: 3308 - protein, 11 aa, Homo sapiens
KVLKAQLWVYL

SEQ ID NO: 3309 - protein, 11 aa, Homo sapiens
VLKAQLWVYLR

SEQ ID NO: 3310 - protein, 11 aa, Homo sapiens
LKAQLWVYLRP

SEQ ID NO: 3311 - protein, 11 aa, Homo sapiens
KAQLWVYLRPV

SEQ ID NO: 3312 - protein, 11 aa, Homo sapiens
AQLWVYLRPVP

SEQ ID NO: 3313 - protein, 11 aa, Homo sapiens
QLWVYLRPVPR

SEQ ID NO: 3314 - protein, 11 aa, Homo sapiens
LWVYLRPVPRP

SEQ ID NO: 3315 - protein, 11 aa, Homo sapiens
WVYLRPVPRPA

SEQ ID NO: 3316 - protein, 11 aa, Homo sapiens
VYLRPVPRPAT

SEQ ID NO: 3317 - protein, 11 aa, Homo sapiens
YLRPVPRPATV

SEQ ID NO: 3318 - protein, 11 aa, Homo sapiens
LRPVPRPATVY

SEQ ID NO: 3319 - protein, 11 aa, Homo sapiens
RPVPRPATVYL

SEQ ID NO: 3320 - protein, 11 aa, Homo sapiens
PVPRPATVYLQ

SEQ ID NO: 3321 - protein, 11 aa, Homo sapiens
VPRPATVYLQI

SEQ ID NO: 3322 - protein, 11 aa, Homo sapiens
PRPATVYLQIL

SEQ ID NO: 3323 - protein, 11 aa, Homo sapiens
RPATVYLQILR

SEQ ID NO: 3324 - protein, 11 aa, Homo sapiens
PATVYLQILRL

SEQ ID NO: 3325 - protein, 11 aa, Homo sapiens
ATVYLQILRLK

SEQ ID NO: 3326 - protein, 11 aa, Homo sapiens
TVYLQILRLKP

SEQ ID NO: 3327 - protein, 11 aa, Homo sapiens
VYLQILRLKPL

SEQ ID NO: 3328 - protein, 11 aa, Homo sapiens
YLQILRLKPLT

SEQ ID NO: 3329 - protein, 11 aa, Homo sapiens
LQILRLKPLTG

SEQ ID NO: 3330 - protein, 11 aa, Homo sapiens
QILRLKPLTGE

SEQ ID NO: 3331 - protein, 11 aa, Homo sapiens
ILRLKPLTGEG

SEQ ID NO: 3332 - protein, 11 aa, Homo sapiens
LRLKPLTGEGT

SEQ ID NO: 3333 - protein, 11 aa, Homo sapiens
RLKPLTGEGTA

SEQ ID NO: 3334 - protein, 11 aa, Homo sapiens
LKPLTGEGTAG

SEQ ID NO: 3335 - protein, 11 aa, Homo sapiens
KPLTGEGTAGG

SEQ ID NO: 3336 - protein, 11 aa, Homo sapiens
PLTGEGTAGGG

SEQ ID NO: 3337 - protein, 11 aa, Homo sapiens
LTGEGTAGGGG

SEQ ID NO: 3338 - protein, 11 aa, Homo sapiens
TGEGTAGGGGG

SEQ ID NO: 3339 - protein, 11 aa, Homo sapiens
GEGTAGGGGGG

SEQ ID NO: 3340 - protein, 11 aa, Homo sapiens
EGTAGGGGGGR

SEQ ID NO: 3341 - protein, 11 aa, Homo sapiens
GTAGGGGGGRR

SEQ ID NO: 3342 - protein, 11 aa, Homo sapiens
TAGGGGGGRRH

SEQ ID NO: 3343 - protein, 11 aa, Homo sapiens
AGGGGGGRRHI

SEQ ID NO: 3344 - protein, 11 aa, Homo sapiens
GGGGGGRRHIR

SEQ ID NO: 3345 - protein, 11 aa, Homo sapiens
GGGGGRRHIRI

SEQ ID NO: 3346 - protein, 11 aa, Homo sapiens
GGGGRRHIRIR

SEQ ID NO: 3347 - protein, 11 aa, Homo sapiens
GGGRRHIRIRS

SEQ ID NO: 3348 - protein, 11 aa, Homo sapiens
GGRRHIRIRSL

SEQ ID NO: 3349 - protein, 11 aa, Homo sapiens
GRRHIRIRSLK

SEQ ID NO: 3350 - protein, 11 aa, Homo sapiens
RRHIRIRSLKI

SEQ ID NO: 3351 - protein, 11 aa, Homo sapiens
RHIRIRSLKIE

SEQ ID NO: 3352 - protein, 11 aa, Homo sapiens
HIRIRSLKIEL

SEQ ID NO: 3353 - protein, 11 aa, Homo sapiens
IRIRSLKIELH

SEQ ID NO: 3354 - protein, 11 aa, Homo sapiens
RIRSLKIELHS

SEQ ID NO: 3355 - protein, 11 aa, Homo sapiens
IRSLKIELHSR

SEQ ID NO: 3356 - protein, 11 aa, Homo sapiens
RSLKIELHSRS

SEQ ID NO: 3357 - protein, 11 aa, Homo sapiens
SLKIELHSRSG

SEQ ID NO: 3358 - protein, 11 aa, Homo sapiens
LKIELHSRSGH

SEQ ID NO: 3359 - protein, 11 aa, Homo sapiens
KIELHSRSGHW

SEQ ID NO: 3360 - protein, 11 aa, Homo sapiens
IELHSRSGHWQ

SEQ ID NO: 3361 - protein, 11 aa, Homo sapiens
ELHSRSGHWQS

SEQ ID NO: 3362 - protein, 11 aa, Homo sapiens
LHSRSGHWQSI

SEQ ID NO: 3363 - protein, 11 aa, Homo sapiens
HSRSGHWQSID

SEQ ID NO: 3364 - protein, 11 aa, Homo sapiens
SRSGHWQSIDF

SEQ ID NO: 3365 - protein, 11 aa, Homo sapiens
RSGHWQSIDFK

SEQ ID NO: 3366 - protein, 11 aa, Homo sapiens
SGHWQSIDFKQ

SEQ ID NO: 3367 - protein, 11 aa, Homo sapiens
GHWQSIDFKQV

SEQ ID NO: 3368 - protein, 11 aa, Homo sapiens
HWQSIDFKQVL

SEQ ID NO: 3369 - protein, 11 aa, Homo sapiens
WQSIDFKQVLH

SEQ ID NO: 3370 - protein, 11 aa, Homo sapiens
QSIDFKQVLHS

SEQ ID NO: 3371 - protein, 11 aa, Homo sapiens
SIDFKQVLHSW

SEQ ID NO: 3372 - protein, 11 aa, Homo sapiens
IDFKQVLHSWF

SEQ ID NO: 3373 - protein, 11 aa, Homo sapiens
DFKQVLHSWFR

SEQ ID NO: 3374 - protein, 11 aa, Homo sapiens
FKQVLHSWFRQ

SEQ ID NO: 3375 - protein, 11 aa, Homo sapiens
KQVLHSWFRQP

SEQ ID NO: 3376 - protein, 11 aa, Homo sapiens
QVLHSWFRQPQ

SEQ ID NO: 3377 - protein, 11 aa, Homo sapiens
VLHSWFRQPQS

SEQ ID NO: 3378 - protein, 11 aa, Homo sapiens
LHSWFRQPQSN

SEQ ID NO: 3379 - protein, 11 aa, Homo sapiens
HSWFRQPQSNW

SEQ ID NO: 3380 - protein, 11 aa, Homo sapiens
SWFRQPQSNWG

SEQ ID NO: 3381 - protein, 11 aa, Homo sapiens
WFRQPQSNWGI

SEQ ID NO: 3382 - protein, 11 aa, Homo sapiens
FRQPQSNWGIE

SEQ ID NO: 3383 - protein, 11 aa, Homo sapiens
RQPQSNWGIEI

SEQ ID NO: 3384 - protein, 11 aa, Homo sapiens
QPQSNWGIEIN

SEQ ID NO: 3385 - protein, 11 aa, Homo sapiens
PQSNWGIEINA

SEQ ID NO: 3386 - protein, 11 aa, Homo sapiens
QSNWGIEINAF

SEQ ID NO: 3387 - protein, 11 aa, Homo sapiens
SNWGIEINAFD

SEQ ID NO: 3388 - protein, 11 aa, Homo sapiens
NWGIEINAFDP

SEQ ID NO: 3389 - protein, 11 aa, Homo sapiens
WGIEINAFDPS

SEQ ID NO: 3390 - protein, 11 aa, Homo sapiens
GIEINAFDPSG

SEQ ID NO: 3391 - protein, 11 aa, Homo sapiens
IEINAFDPSGT

SEQ ID NO: 3392 - protein, 11 aa, Homo sapiens
EINAFDPSGTD

SEQ ID NO: 3393 - protein, 11 aa, Homo sapiens
INAFDPSGTDL

SEQ ID NO: 3394 - protein, 11 aa, Homo sapiens
NAFDPSGTDLA

SEQ ID NO: 3395 - protein, 11 aa, Homo sapiens
AFDPSGTDLAV

SEQ ID NO: 3396 - protein, 11 aa, Homo sapiens
FDPSGTDLAVT

SEQ ID NO: 3397 - protein, 11 aa, Homo sapiens
DPSGTDLAVTS

SEQ ID NO: 3398 - protein, 11 aa, Homo sapiens
PSGTDLAVTSL

SEQ ID NO: 3399 - protein, 11 aa, Homo sapiens
SGTDLAVTSLG

SEQ ID NO: 3400 - protein, 11 aa, Homo sapiens
GTDLAVTSLGP

SEQ ID NO: 3401 - protein, 11 aa, Homo sapiens
TDLAVTSLGPG

SEQ ID NO: 3402 - protein, 11 aa, Homo sapiens
DLAVTSLGPGA

SEQ ID NO: 3403 - protein, 11 aa, Homo sapiens
LAVTSLGPGAE

SEQ ID NO: 3404 - protein, 11 aa, Homo sapiens
AVTSLGPGAEG

SEQ ID NO: 3405 - protein, 11 aa, Homo sapiens
VTSLGPGAEGL

SEQ ID NO: 3406 - protein, 11 aa, Homo sapiens
TSLGPGAEGLH

SEQ ID NO: 3407 - protein, 11 aa, Homo sapiens
SLGPGAEGLHP

SEQ ID NO: 3408 - protein, 11 aa, Homo sapiens
LGPGAEGLHPF

SEQ ID NO: 3409 - protein, 11 aa, Homo sapiens
GPGAEGLHPFM

SEQ ID NO: 3410 - protein, 11 aa, Homo sapiens
PGAEGLHPFME

SEQ ID NO: 3411 - protein, 11 aa, Homo sapiens
GAEGLHPFMEL

SEQ ID NO: 3412 - protein, 11 aa, Homo sapiens
AEGLHPFMELR

SEQ ID NO: 3413 - protein, 11 aa, Homo sapiens
EGLHPFMELRV

SEQ ID NO: 3414 - protein, 11 aa, Homo sapiens
GLHPFMELRVL

SEQ ID NO: 3415 - protein, 11 aa, Homo sapiens
LHPFMELRVLE

SEQ ID NO: 3416 - protein, 11 aa, Homo sapiens
HPFMELRVLEN

SEQ ID NO: 3417 - protein, 11 aa, Homo sapiens
PFMELRVLENT

SEQ ID NO: 3418 - protein, 11 aa, Homo sapiens
FMELRVLENTK

SEQ ID NO: 3419 - protein, 11 aa, Homo sapiens
MELRVLENTKR

SEQ ID NO: 3420 - protein, 11 aa, Homo sapiens
ELRVLENTKRS

SEQ ID NO: 3421 - protein, 11 aa, Homo sapiens
LRVLENTKRSR

SEQ ID NO: 3422 - protein, 11 aa, Homo sapiens
RVLENTKRSRR

SEQ ID NO: 3423 - protein, 11 aa, Homo sapiens
VLENTKRSRRN

SEQ ID NO: 3424 - protein, 11 aa, Homo sapiens
LENTKRSRRNL

SEQ ID NO: 3425 - protein, 11 aa, Homo sapiens
ENTKRSRRNLG

SEQ ID NO: 3426 - protein, 11 aa, Homo sapiens
NTKRSRRNLGL

SEQ ID NO: 3427 - protein, 11 aa, Homo sapiens
TKRSRRNLGLD

SEQ ID NO: 3428 - protein, 11 aa, Homo sapiens
KRSRRNLGLDC

SEQ ID NO: 3429 - protein, 11 aa, Homo sapiens
RSRRNLGLDCD

SEQ ID NO: 3430 - protein, 11 aa, Homo sapiens
SRRNLGLDCDE

SEQ ID NO: 3431 - protein, 11 aa, Homo sapiens
RRNLGLDCDEH

SEQ ID NO: 3432 - protein, 11 aa, Homo sapiens
RNLGLDCDEHS

SEQ ID NO: 3433 - protein, 11 aa, Homo sapiens
NLGLDCDEHSS

SEQ ID NO: 3434 - protein, 11 aa, Homo sapiens
LGLDCDEHSSE

SEQ ID NO: 3435 - protein, 11 aa, Homo sapiens
GLDCDEHSSES

SEQ ID NO: 3436 - protein, 11 aa, Homo sapiens
LDCDEHSSESR

SEQ ID NO: 3437 - protein, 11 aa, Homo sapiens
DCDEHSSESRC

SEQ ID NO: 3438 - protein, 11 aa, Homo sapiens
CDEHSSESRCC

SEQ ID NO: 3439 - protein, 11 aa, Homo sapiens
DEHSSESRCCR

SEQ ID NO: 3440 - protein, 11 aa, Homo sapiens
EHSSESRCCRY

SEQ ID NO: 3441 - protein, 11 aa, Homo sapiens
HSSESRCCRYP

SEQ ID NO: 3442 - protein, 11 aa, Homo sapiens
SSESRCCRYPL

SEQ ID NO: 3443 - protein, 11 aa, Homo sapiens
SESRCCRYPLT

SEQ ID NO: 3444 - protein, 11 aa, Homo sapiens
ESRCCRYPLTV

SEQ ID NO: 3445 - protein, 11 aa, Homo sapiens
SRCCRYPLTVD

SEQ ID NO: 3446 - protein, 11 aa, Homo sapiens
RCCRYPLTVDF

SEQ ID NO: 3447 - protein, 11 aa, Homo sapiens
CCRYPLTVDFE

SEQ ID NO: 3448 - protein, 11 aa, Homo sapiens
CRYPLTVDFEA

SEQ ID NO: 3449 - protein, 11 aa, Homo sapiens
RYPLTVDFEAF

SEQ ID NO: 3450 - protein, 11 aa, Homo sapiens
YPLTVDFEAFG

SEQ ID NO: 3451 - protein, 11 aa, Homo sapiens
PLTVDFEAFGW

SEQ ID NO: 3452 - protein, 11 aa, Homo sapiens
LTVDFEAFGWD

SEQ ID NO: 3453 - protein, 11 aa, Homo sapiens
TVDFEAFGWDW

SEQ ID NO: 3454 - protein, 11 aa, Homo sapiens
VDFEAFGWDWI

SEQ ID NO: 3455 - protein, 11 aa, Homo sapiens
DFEAFGWDWII

SEQ ID NO: 3456 - protein, 11 aa, Homo sapiens
FEAFGWDWIIA

SEQ ID NO: 3457 - protein, 11 aa, Homo sapiens
EAFGWDWIIAP

SEQ ID NO: 3458 - protein, 11 aa, Homo sapiens
AFGWDWIIAPK

SEQ ID NO: 3459 - protein, 11 aa, Homo sapiens
FGWDWIIAPKR

SEQ ID NO: 3460 - protein, 11 aa, Homo sapiens
GWDWIIAPKRY

SEQ ID NO: 3461 - protein, 11 aa, Homo sapiens
WDWIIAPKRYK

SEQ ID NO: 3462 - protein, 11 aa, Homo sapiens
DWIIAPKRYKA

SEQ ID NO: 3463 - protein, 11 aa, Homo sapiens
WIIAPKRYKAN

SEQ ID NO: 3464 - protein, 11 aa, Homo sapiens
IIAPKRYKANY

SEQ ID NO: 3465 - protein, 11 aa, Homo sapiens
IAPKRYKANYC

SEQ ID NO: 3466 - protein, 11 aa, Homo sapiens
APKRYKANYCS

SEQ ID NO: 3467 - protein, 11 aa, Homo sapiens
PKRYKANYCSG

SEQ ID NO: 3468 - protein, 11 aa, Homo sapiens
KRYKANYCSGQ

SEQ ID NO: 3469 - protein, 11 aa, Homo sapiens
RYKANYCSGQC

SEQ ID NO: 3470 - protein, 11 aa, Homo sapiens
YKANYCSGQCE

SEQ ID NO: 3471 - protein, 11 aa, Homo sapiens
KANYCSGQCEY

SEQ ID NO: 3472 - protein, 11 aa, Homo sapiens
ANYCSGQCEYM

SEQ ID NO: 3473 - protein, 11 aa, Homo sapiens
NYCSGQCEYMF

SEQ ID NO: 3474 - protein, 11 aa, Homo sapiens
YCSGQCEYMFM

SEQ ID NO: 3475 - protein, 11 aa, Homo sapiens
CSGQCEYMFMQ

SEQ ID NO: 3476 - protein, 11 aa, Homo sapiens
SGQCEYMFMQK

SEQ ID NO: 3477 - protein, 11 aa, Homo sapiens
GQCEYMFMQKY

SEQ ID NO: 3478 - protein, 11 aa, Homo sapiens
QCEYMFMQKYP

SEQ ID NO: 3479 - protein, 11 aa, Homo sapiens
CEYMFMQKYPH

SEQ ID NO: 3480 - protein, 11 aa, Homo sapiens
EYMFMQKYPHT

SEQ ID NO: 3481 - protein, 11 aa, Homo sapiens
YMFMQKYPHTH

SEQ ID NO: 3482 - protein, 11 aa, Homo sapiens
MFMQKYPHTHL

SEQ ID NO: 3483 - protein, 11 aa, Homo sapiens
FMQKYPHTHLV

SEQ ID NO: 3484 - protein, 11 aa, Homo sapiens
MQKYPHTHLVQ

SEQ ID NO: 3485 - protein, 11 aa, Homo sapiens
QKYPHTHLVQQ

SEQ ID NO: 3486 - protein, 11 aa, Homo sapiens
KYPHTHLVQQA

SEQ ID NO: 3487 - protein, 11 aa, Homo sapiens
YPHTHLVQQAN

SEQ ID NO: 3488 - protein, 11 aa, Homo sapiens
PHTHLVQQANP

SEQ ID NO: 3489 - protein, 11 aa, Homo sapiens
HTHLVQQANPR

SEQ ID NO: 3490 - protein, 11 aa, Homo sapiens
THLVQQANPRG

SEQ ID NO: 3491 - protein, 11 aa, Homo sapiens
HLVQQANPRGS

SEQ ID NO: 3492 - protein, 11 aa, Homo sapiens
LVQQANPRGSA

SEQ ID NO: 3493 - protein, 11 aa, Homo sapiens
VQQANPRGSAG

SEQ ID NO: 3494 - protein, 11 aa, Homo sapiens
QQANPRGSAGP

SEQ ID NO: 3495 - protein, 11 aa, Homo sapiens
QANPRGSAGPC

SEQ ID NO: 3496 - protein, 11 aa, Homo sapiens
ANPRGSAGPCC

SEQ ID NO: 3497 - protein, 11 aa, Homo sapiens
NPRGSAGPCCT

SEQ ID NO: 3498 - protein, 11 aa, Homo sapiens
PRGSAGPCCTP

SEQ ID NO: 3499 - protein, 11 aa, Homo sapiens
RGSAGPCCTPT

SEQ ID NO: 3500 - protein, 11 aa, Homo sapiens
GSAGPCCTPTK

SEQ ID NO: 3501 - protein, 11 aa, Homo sapiens
SAGPCCTPTKM

SEQ ID NO: 3502 - protein, 11 aa, Homo sapiens
AGPCCTPTKMS

SEQ ID NO: 3503 - protein, 11 aa, Homo sapiens
GPCCTPTKMSP

SEQ ID NO: 3504 - protein, 11 aa, Homo sapiens
PCCTPTKMSPI

SEQ ID NO: 3505 - protein, 11 aa, Homo sapiens
CCTPTKMSPIN

SEQ ID NO: 3506 - protein, 11 aa, Homo sapiens
CTPTKMSPINM

SEQ ID NO: 3507 - protein, 11 aa, Homo sapiens
TPTKMSPINML

SEQ ID NO: 3508 - protein, 11 aa, Homo sapiens
PTKMSPINMLY

SEQ ID NO: 3509 - protein, 11 aa, Homo sapiens
TKMSPINMLYF

SEQ ID NO: 3510 - protein, 11 aa, Homo sapiens
KMSPINMLYFN

SEQ ID NO: 3511 - protein, 11 aa, Homo sapiens
MSPINMLYFND

SEQ ID NO: 3512 - protein, 11 aa, Homo sapiens
SPINMLYFNDK

SEQ ID NO: 3513 - protein, 11 aa, Homo sapiens
PINMLYFNDKQ

SEQ ID NO: 3514 - protein, 11 aa, Homo sapiens
INMLYFNDKQQ

SEQ ID NO: 3515 - protein, 11 aa, Homo sapiens
NMLYFNDKQQI

SEQ ID NO: 3516 - protein, 11 aa, Homo sapiens
MLYFNDKQQII

SEQ ID NO: 3517 - protein, 11 aa, Homo sapiens
LYFNDKQQIIY

SEQ ID NO: 3518 - protein, 11 aa, Homo sapiens
YFNDKQQIIYG

SEQ ID NO: 3519 - protein, 11 aa, Homo sapiens
FNDKQQIIYGK

SEQ ID NO: 3520 - protein, 11 aa, Homo sapiens
NDKQQIIYGKI

SEQ ID NO: 3521 - protein, 11 aa, Homo sapiens
DKQQIIYGKIP

SEQ ID NO: 3522 - protein, 11 aa, Homo sapiens
KQQIIYGKIPG

SEQ ID NO: 3523 - protein, 11 aa, Homo sapiens
QQIIYGKIPGM

SEQ ID NO: 3524 - protein, 11 aa, Homo sapiens
QIIYGKIPGMV

SEQ ID NO: 3525 - protein, 11 aa, Homo sapiens
IIYGKIPGMVV

SEQ ID NO: 3526 - protein, 11 aa, Homo sapiens
IYGKIPGMVVD

SEQ ID NO: 3527 - protein, 11 aa, Homo sapiens
YGKIPGMVVDR

SEQ ID NO: 3528 - protein, 11 aa, Homo sapiens
GKIPGMVVDRC

SEQ ID NO: 3529 - protein, 11 aa, Homo sapiens
KIPGMVVDRCG

SEQ ID NO: 3530 - protein, 11 aa, Homo sapiens
IPGMVVDRCGC

SEQ ID NO: 3531 - protein, 11 aa, Homo sapiens
PGMVVDRCGCS

(19) United States
(12) Patent Application Publication
(10) Pub. No.: US 2021/0338557 A1
(43) Pub. Date: Nov. 4, 2021

(54) Oxidized Derivatives of GDF-11 Fragments

(71) Applicant: Avon Products, Inc., Suffern, NY (US)

(72) Inventors: Jolanta IDKOWIAK-BALDYS, Allendale, NJ (US); John W. LYGA, Basking Ridge, NJ (US); Robert Zhiyong LUO, New City, NY (US)

(21) Appl. No.: 17/294,532

(22) PCT Filed: Jan. 4, 2019
(86) PCT No.: PCT/US2019/012325

§ 371 Date: May 17, 2021

(51) Int. Cl. A61K 8/64; A61K 8/365; A61K 8/67; A61K 8/34; A61K 8/46; A61Q 19/08

(52) U.S. Cl. CPC A61K 8/64; A61K 8/365; A61K 8/675; A61Q 19/08; A61K 8/342; A61K 8/46; A61K 8/347

(57) ABSTRACT

Peptides (and derivatives thereof), topical compositions, and methods of diminishing signs of aging and/or improving health of human integuments are provided. The peptides are derived from human Growth Differentiation Factor 11 (GDF-11) and typically have one or more chemical modifications of the amino acid residues. Preferred modifications of GDF-11 fragments include methionine oxidation.

### CROSS-REFERENCE TO RELATED APPLICATION

This patent application claims priority to PCT Application No. PCT/US19/12325, filed on Jan. 4, 2019. The entirety of the aforementioned application is incorporated herein in its entirety by reference.

### SEQUENCE LISTING

The instant application contains a Sequence Listing which has been submitted electronically in ASCII format and is hereby incorporated by reference in its entirety. Said ASCII copy, created on Oct. 2, 2018, is named SC195U-WO_SL.txt and is 462,000 bytes in size.

### FIELD OF INVENTION

The present invention relates generally to peptides derivatives, in particular peptides derived from the Growth Differentiation Factor 11 (GDF-11) protein and topical formulations containing them, as well as associated methods for improving the health of skin and/or diminishing the dermatological signs of aging in human skin. The peptide derivatives derived from GDF-11 protein exhibit increased bioactivity than the nonderivatized GDF-11 fragments. For example, oxidation of one or more amino acid residues of GDF-11 fragments leads to increases in hyaluronic acid production and/or collagen production in fibroblasts.

### BACKGROUND

Growth factors are naturally occurring substances, usually proteins, that act as signaling molecules between cells. Their primary function is promoting cell differentiation and maturation. Growth factors play an important role in many functions, such as stimulating cell growth, proliferation, and wound healing. Many large classes, or superfamilies, of related growth factors are known.

Growth Differentiation Factor 11 (GDF-11) is a protein belonging to the transforming growth factor (TGF) superfamily (e.g., TGF-β), which encompasses a group of structurally-related proteins. Blood-derived GDF-11 was recently shown to be involved in reverting the aging phenotype in mice, including cardiac hypertrophy (see Loffredo et al., Cell, 2013, 153, 828-839), age-related sarcopenia (see Sinha et al., Science, 2014, 344:649-52), and decreased cognitive functions (see Villeda et al., Nat. Med. 2014, 20:659-63). Due to the many important roles growth factors play in maintaining healthy tissues, there has been some interest in using them in dermatological formulations. There are, however, drawbacks associated with the use of growth factors in topical formulations. Additionally, GDF-11 fragments have been shown to result in increases in hyaluronic acid and/or collagen production in fibroblasts. However, these fragments are also beholden to various drawbacks including stability issues.

Additionally, derivatizing peptides has known drawbacks as well. For example, oxidation of peptide residues such as methionine may reduce efficacy (see Unnikrishnan et al., Agents andActions, 1990, 31:110-112) or may destabilize the peptides.

It is therefore an object of the invention to provide new derivatives of peptides thereof derived from GDF-11 and topical compositions containing them unhibitited by these drawbacks. It is also an object of the invention to provide methods for improving the health and/or appearance of skin, combatting signs of intrinsic and photoaging, and/or treating skin disorders. It is a further object of the invention to provide compositions and methods for treating, reversing, forestalling and/or ameliorating skin wrinkles and fine lines, tightening sagging skin, firming skin, and for treating hyperpigmentation and unwanted pigmentation with cosmetic compositions comprising effective amounts of a peptide derivative of the invention.

The foregoing discussion is presented solely to provide a better understanding of the nature of the problems confronting the art and should not be construed in any way as an admission as to prior art.

### SUMMARY OF THE INVENTION

In accordance with the foregoing objectives and others, the present invention provides active agents comprising peptide derivatives and topical formulations. The active agents are believed to be useful for improving one or more signs of dermatological aging when topically applied to human integuments (e.g., skin, lips, nails, hair, etc.), particularly skin. They are also contemplated to be useful in treating a variety of dermatological disorders and improving the overall health of skin. The peptides of the invention are derived from human growth factor GDF-11. In some embodiments, the peptide derivatives are oxidized GDF-11 fragments (e.g., small peptide sequences of GDF-11 wherein one or more amino acid residues have bene oxidized, etc.). In some embodiments, the active agents of the invention are capable of increasing collagen and/or hyaluronic acid (HA) production of skin cells and therefore will have a beneficial effect on reducing the appearance of aging on skin (e.g., diminishing the appearance of wrinkles and/or fine lines, tightening sagging skin, thickening thinning skin, evening skin tone, treating hyperpigmentation and unwanted pigmentation, etc.).

The present invention relates to smaller peptide sequence derivatives (e.g., 3-11 or 3 or 4 or 5 or 6 or 7 or 8 or 9 or 10 or 11 amino acids) of the full-length GDF-11 protein which increase hyaluronic acid (HA) and/or collagen in fibroblasts as compared to the nonderivatized versions of the peptide. Nonderivatized versions of the peptide may be disclosed in U.S. Pat. No. 10,034,826, hereby incorporated by reference in its entirety. These peptides, especially peptides similar or homologous to putative functional regions of the protein, are contemplated to have biological activity, including antiaging benefits in skin. It has been found that derivatives of these smaller peptides have increased biological activity than the nonderivatized peptide. For example, smaller sequences of the GDF-11 protein may be derivatized by modification of one or more amino acid residue. In certain embodiments, the smaller sequences of the GDF-11 protein may be derivatized by oxidation of one or more amino acid residues. In some embodiments, the smaller peptide sequence of GDF-11 comprises methionine and/or cysteine. In some embodiments, the peptide is MVV. In certain embodiments, the peptide derivative of the invention comprises a thiol or thioether functional group which has been oxidized. For example, the peptide derivative may comprise methionine sulfone, methionine sulfoxide, and/or cysteine disulfide. In some embodiments, the peptide derivative is methionine sulfone valine valine (M(O)₂VV) which has the structure:

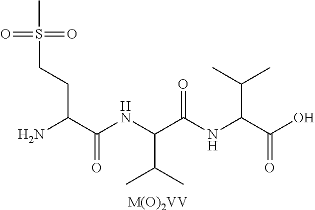

or a derivative thereof (including without limitation C₁₋₁₈ acyl derivatives at the amino terminus and/or C₁₋₁₈ ester derivatives at the acid terminus) or a salt thereof. In some embodiments, the peptide derivative is methionine sulfoxide valine valine (M(O)VV) which has the structure:

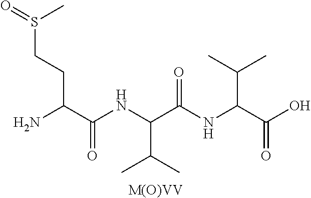

or a derivative thereof (including without limitation C₁₋₁₈ acyl derivatives at the amino terminus and/or C₁₋₁₈ ester derivatives at the acid terminus) or a salt thereof.

Each amino acid residue or derivatized residue may be in the natural enantiomeric form. In some embodiments, the amino acid residues or derivatized residues may independently be in the L optical form or the D enantiomeric form. In some embodiments, each amino acid residue and derivatized residue may have the L form. In some embodiments, the stereochemical assignment of the amino acid or derivatized residue may be independently selected from R or S. For example, peptide derivative may have the structure:

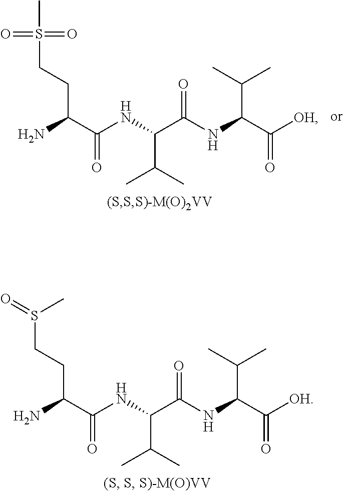

The peptide derivative may also be independently functionalized at the amino and/or carboxylic acid terminus with one or more hydrocarbons. In some embodiments, the peptide derivative (including M(O)VV and M(O)₂VV) may acetylated at the amino terminus. For example, the peptide derivative may be Ac-M(O)₂VV which has the structure:

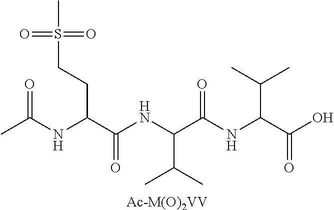

or a derivative thereof (including without limitation C₁₋₁₈ acyl derivatives at the amino terminus and/or C₁₋₁₈ ester derivatives at the acid terminus) or a salt thereof. In some embodiments, the peptide derivative may be Ac-M(O)VV which has the structure:

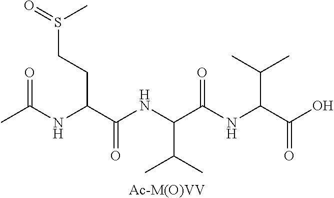

or a derivative thereof (including without limitation C₁₋₁₈ acyl derivatives at the amino terminus and/or C₁₋₁₈ ester derivatives at the acid terminus) or a salt thereof.

One aspect of the invention provides compositions for topical use comprising an active agent comprising one or more GDF-11-derived peptides (e.g., having from 3-11 consecutive amino acids from the GDF-11 sequence) including cyclic peptide fragments of the invention in a physiologically acceptable carrier, wherein the GDF-11 derived peptide fragment has been oxidized. In certain embodiments, one or more (e.g., one, two, three, four, five, six, seven, etc.) amino acid residues of the peptide sequence have been derivatized (e.g., oxidized, etc.). In some embodiments, the peptide sequence of GDF-11 comprises cysteine or methionine. In some embodiments, the peptide derivative comprises oxidized methionine and/or oxidized cysteine. The one or more GDF-11 derived peptide derivatives may be present in the composition in an amount between about 0.000001% to about 10% (e.g., 0.0001-1%) by weight of the composition. Peptides useful in the practice of the invention include, for example, those comprising 3 amino acids (SEQ ID NO: 2-375); 4 amino acids (SEQ ID NO: 376-767); 5 amino acids (SEQ ID NO:768-1161); 6 amino acids (SEQ ID NO: 1162-1556); 7 amino acids (SEQ ID NO: 1557-1951); 8 amino acids (SEQ ID NO: 1952-2346); 9 amino acids (SEQ ID NO: 2347-2741); 10 amino acids (SEQ ID NO: 2742-3136); 11 amino acids (SEQ ID NO: 3137-3531) or even larger fragments of GDF-11. The peptide derivative may be derived from GDF-11 fragments wherein one or more amino acid residues have been oxidized. In another aspect, methods are provided for ameliorating and/or preventing signs of human skin photoaging and intrinsic aging (e.g., diminishing the appearance of wrinkles and/or fine lines, tightening sagging skin, thickening thinning skin, evening skin tone, treating hyperpigmentation, etc.) comprising topically applying to the skin (e.g., skin of the face) a composition comprising, in a topically acceptable vehicle, one or more GDF-11-derived peptide derivatives of the invention.

These and other aspects of the present invention will become apparent to those skilled in the art after a reading of the following detailed description of the invention, including the illustrative embodiments and examples.

### BRIEF DESCRIPTION OF THE FIGURES

FIG. 1A shows the results of administration of various peptides or peptide derivatives in an in vitro assay at 0.001% by weight (10 ppm) in control (DMSO). FIG. 1B shows the results of administration of various peptides or peptide derivatives in an in vitro assay at 0.0005% by weight (5 ppm) in control (DMSO). FIG. 1C shows the results of administration of various peptides or peptide derivatives in an in vitro assay at 0.0001% by weight (1 ppm) in control (DMSO). FIG. 1D shows the results of administration of various peptides or peptide derivatives in an in vitro assay at 0.00001% by weight (0.1 ppm) in control (DMSO).

FIG. 2 shows the measured HA score of the ex vivo assay following administration of either M(O)₂VV or Ac-M(O)₂VV at 0.0005%, 0.001%, or 0.01% by weight of the composition. Columns marked with “**” were highly significant (p<0.05) with respect to untreated samples and with respect to samples administered vehicle alone (DMSO).

FIG. 3 shows the measured collagen score of the ex vivo assay following administration of either M(O)₂VV or Ac-M(O)₂VV at 0.0005%, 0.001%, or 0.01% by weight of the composition. Columns marked with “**” were highly significant (p<0.05) with respect to untreated samples and with respect to samples administered vehicle alone.

### DETAILED DESCRIPTION

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive. Therefore, specific structural and functional details disclosed herein are not to be interpreted as limiting, but merely as a representative basis for teaching one skilled in the art to variously employ the present invention.

All percentages given herein refer to the weight percentages of a particular component relative to the entire composition, including the vehicle, unless otherwise indicated. It will be understood that the sum of all weight % of individual components within a composition will not exceed 100%. If the amount of an ingredient is not otherwise specified, it may be present in an amount from 0.00001-90% by weight.

All terms used herein are intended to have their ordinary meaning unless otherwise provided. The phrases “physiologically acceptable,” “topically acceptable,” and “dermatologically acceptable” are used interchangeably and are intended to mean that a particular component is generally regarded as safe and non-toxic for application to a human integument (e.g., skin) at the levels employed. The term “prevent,” as used herein, includes delaying, slowing or forestalling the onset of or progression of a particular sign of skin aging. The phrase “individual in need thereof” refers to a human that could benefit from improved dermal appearance or health, including males or females. In some embodiments, the individual in need thereof is a female. The term “skin” includes, without limitation, the lips, skin of the face, hands, arms, neck, scalp, and chest. The term “thin” skin includes, but is not limited to, skin that is prematurely thinned, and may be diagnosed as such by a dermatologist. In some embodiments, the thin skin is skin of a female under the age of 60; 50; 40; and/or skin of a pre-menopausal, peri-menopausal or post-menopausal female.

As used herein, the term “consisting essentially of” is intended to limit the invention to the specified materials or steps and those that do not materially affect the basic and novel characteristics of the claimed invention, as understood from a reading of this specification.

As used herein, a hydrocarbon, alkyl, alkenyl, alkynyl, aryl, aryl-alkyl, alkyl-aryl, heteroaryl, or combination of any of those will have from 1-30 carbon atoms, optionally substituted with O, N, S, unless otherwise specified. Any of the alkyl, alkenyl, and alkynyl groups disclosed herein, unless otherwise specified, may be straight-chained, branched, and/or cyclic.

The term “amino acid” is intended to include naturally occurring amino acids and non-proteinogenic amino acids as well as non-naturally occurring amino acids and includes any small molecule (MW<1,000 Daltons) having at least one carboxyl group and at least one primary or secondary amine group capable of forming peptide bonds. The term “peptide” is intended to include any molecule comprising at least two amino acids joined by a peptide bond and therefore includes di-peptides, tri-peptides, oligopeptides, and polypeptides having up to about 20 consecutive amino acid residues linked by peptide bonds. The term “peptide” also embraces structures having one or more linkers, spacers, or terminal groups which are not amino acids. It also includes cyclic peptides.

The term “derivative” of a peptide refers to a chemically modified peptide (e.g., polypeptide) that has been chemically modified, for example, by natural processes such as processing and other post-translational modifications and/or by chemical modification techniques such as addition of one or more polyethylene glycol molecules, sugars, and phosphates. The modification may occur anywhere in the peptide to produce the peptide derivative including the peptide backbone, the amino acid side-chains, and the amino and/or carboxyl termini. In most embodiments, the peptide derivatives described herein comprise one or more amino acid residues that have been chemically modified. The peptide derivative may have the same modification in two or more locations or different modifications in two or more locations. In some embodiments, the peptide derivative comprises one or more amino acid residues that have been chemically modified in addition to one or more modifications on the amino or carboxyl termini. Modifications include, for example, acetylation, acylation, amidation, covalent attachment of a lipid, covalent attachment of a fatty acid acyl residue, cross-linking, cyclization, disulfide bond formation, demthylation, formation of covalent cross-links, hydroxylation, iodination, methylation, myritoylation, oxidation, phosphorylation, prenylation, racemization, glycosylation, selenoylation, or sulfation. Suitable modifications may be found, for example, in Proteins—Structure and Molecular Properties 2^{nd} Ed., T. E. Crieghton, W.H. Freeman and Company, New York (1993), hereby incorporated by reference in its entirety. In certain embodiments, the peptide derivative comprises one or more (e.g., two, three, four, five, etc.) amino acid residues (e.g., methionine, cysteine) modified by oxidation. In some implementations, the peptide derivative comprises one or more (e.g., two, three, four, five, etc.) amino acid residues modified by oxidation and acetylation at the amino and/or carboxylic acid terminus.

Peptides

The compositions comprise one or more peptide fragments of GDF-11 which have been derivatized. In most embodiments, the peptide derivatives comprise one or more amino acid residues modified by oxidation. The peptides comprise, consist essentially of, or consist of amino acid sequences derived from the Growth Differentiation Factor 11 (GDF-11) protein. Consisting essentially of, as used herein, is intended to mean that additional amino acids may be present at either terminus provided they do not substantially impair the activity of the peptide. For example, in embodiments where a peptide “consists essentially of” SEQ ID NOs 2-3531, any additional amino acids may be excluded from the peptide if their inclusion produces a measurable improvement (e.g., greater than 50% reduction) of the beneficial activity, including, without limitation, upregulation of pro-collagen, collagen, elastin, fibronectin, and/or hyaluronic acid.

In one embodiment, the peptide comprises from 3-11 (e.g., 3, 4, 5, 6, 7, 8, 9, 10, or 11) consecutive amino acids derived from the sequence of Growth Differentiation Factor 11 (GDF-11) precursor [Homo sapiens], NCBI Reference Sequence Accession No.: NP_005802.1, shown in Table 1 SEQ ID NO: 1).

TABLE 1
Sequence of GDF-11 Precursor [Homo Sapiens]

| | |
|---|---|
| 1 | mvlaaplllg flllalelrp rgeaaegpaa aaaaaaaaaa agvggerssr papsvapepd |
| 61 | gcpvcvwrqh srelrlesik sqilsklrlk eapnisrevv kqllpkappl qqildlhdfq |
| 121 | gdalqpedfl eedeyhatte tvismaqetd pavqtdgspl cchfhfspkv mftkvlkaql |
| 181 | wvylrpvprp atvylqilrl kpltgegtag gggggrrhir irslkielhs rsghwqsidf |
| 241 | kqvlhswfrq pqsnwgiein afdpsgtdla vtslgpgaeg lhpfmelrvl entkrsrrnl |
| 301 | gldcdehsse srccrypltv dfeafgwdwi iapkrykany csgqceymfm qkyphthlvq |
| 361 | qanprgsagp cctptkmspi nmlyfndkqq iiygkipgmv vdrcgcs |

In some embodiments, the peptide comprises, consists essentially of, or consists of any one or more of the 3-mer amino acid sequences (SEQ ID NO: 2-375) listed in Table 2.

TABLE 2

| | | |
|---|---|---|
| SEQ ID | 2 | MVL |
| SEQ ID | 3 | VLA |
| SEQ ID | 4 | LAA |
| SEQ ID | 5 | AAP |
| SEQ ID | 6 | APL |
| SEQ ID | 7 | PLL |
| SEQ ID | 8 | LLL |
| SEQ ID | 9 | LLG |
| SEQ ID | 10 | LGF |
| SEQ ID | 11 | GFL |
| SEQ ID | 12 | FLL |
| SEQ ID | 13 | LLA |
| SEQ ID | 14 | LAL |
| SEQ ID | 15 | ALE |
| SEQ ID | 16 | LEL |
| SEQ ID | 17 | ELR |
| SEQ ID | 18 | LRP |
| SEQ ID | 19 | RPR |
| SEQ ID | 20 | PRG |
| SEQ ID | 21 | RGE |
| SEQ ID | 22 | GEA |
| SEQ ID | 23 | EAA |
| SEQ ID | 24 | AAE |
| SEQ ID | 25 | AEG |
| SEQ ID | 26 | EGP |
| SEQ ID | 27 | GPA |
| SEQ ID | 28 | PAA |
| SEQ ID | 29 | AAA |
| SEQ ID | 30 | AAG |
| SEQ ID | 31 | AGV |
| SEQ ID | 32 | GVG |
| SEQ ID | 33 | VGG |
| SEQ ID | 34 | GGE |
| SEQ ID | 35 | GER |
| SEQ ID | 36 | ERS |
| SEQ ID | 37 | RSS |
| SEQ ID | 38 | SSR |
| SEQ ID | 39 | SRP |
| SEQ ID | 40 | RPA |
| SEQ ID | 41 | PAP |
| SEQ ID | 42 | APS |
| SEQ ID | 43 | PSV |
| SEQ ID | 44 | SVA |
| SEQ ID | 45 | VAP |
| SEQ ID | 46 | APE |
| SEQ ID | 47 | PEP |
| SEQ ID | 48 | EPD |
| SEQ ID | 49 | PDG |
| SEQ ID | 50 | DGC |
| SEQ ID | 51 | GCP |
| SEQ ID | 52 | CPV |
| SEQ ID | 53 | PVC |
| SEQ ID | 54 | VCV |
| SEQ ID | 55 | CVW |
| SEQ ID | 56 | VWR |
| SEQ ID | 57 | WRQ |
| SEQ ID | 58 | RQH |
| SEQ ID | 59 | QHS |
| SEQ ID | 60 | HSR |
| SEQ ID | 61 | SRE |
| SEQ ID | 62 | REL |
| SEQ ID | 63 | LRL |
| SEQ ID | 64 | RLE |
| SEQ ID | 65 | LES |
| SEQ ID | 66 | ESI |
| SEQ ID | 67 | SIK |
| SEQ ID | 68 | IKS |
| SEQ ID | 69 | KSQ |
| SEQ ID | 70 | SQI |
| SEQ ID | 71 | QIL |
| SEQ ID | 72 | ILS |
| SEQ ID | 73 | LSK |
| SEQ ID | 74 | SKL |
| SEQ ID | 75 | KLR |
| SEQ ID | 76 | RLK |
| SEQ ID | 77 | LKE |
| SEQ ID | 78 | KEA |
| SEQ ID | 79 | EAP |
| SEQ ID | 80 | APN |
| SEQ ID | 81 | PNI |
| SEQ ID | 82 | NIS |
| SEQ ID | 83 | ISR |
| SEQ ID | 84 | REV |
| SEQ ID | 85 | EVV |
| SEQ ID | 86 | VVK |
| SEQ ID | 87 | VKQ |
| SEQ ID | 88 | KQL |
| SEQ ID | 89 | QLL |
| SEQ ID | 90 | LLP |
| SEQ ID | 91 | LPK |
| SEQ ID | 92 | PKA |
| SEQ ID | 93 | KAP |
| SEQ ID | 94 | APP |
| SEQ ID | 95 | PPL |
| SEQ ID | 96 | PLQ |
| SEQ ID | 97 | LQQ |
| SEQ ID | 98 | QQI |
| SEQ ID | 99 | ILD |
| SEQ ID | 100 | LDL |
| SEQ ID | 101 | DLH |
| SEQ ID | 102 | LHD |
| SEQ ID | 103 | HDF |
| SEQ ID | 104 | DFQ |
| SEQ ID | 105 | FQG |
| SEQ ID | 106 | QGD |
| SEQ ID | 107 | GDA |
| SEQ ID | 108 | DAL |
| SEQ ID | 109 | ALQ |
| SEQ ID | 110 | LQP |
| SEQ ID | 111 | QPE |
| SEQ ID | 112 | PED |
| SEQ ID | 113 | EDF |
| SEQ ID | 114 | DFL |
| SEQ ID | 115 | FLE |
| SEQ ID | 116 | LEE |
| SEQ ID | 117 | EED |
| SEQ ID | 118 | EDE |
| SEQ ID | 119 | DEY |
| SEQ ID | 120 | EYH |
| SEQ ID | 121 | YHA |
| SEQ ID | 122 | HAT |
| SEQ ID | 123 | ATT |
| SEQ ID | 124 | TTE |
| SEQ ID | 125 | TET |
| SEQ ID | 126 | ETV |
| SEQ ID | 127 | TVI |
| SEQ ID | 128 | VIS |
| SEQ ID | 129 | ISM |
| SEQ ID | 130 | SMA |
| SEQ ID | 131 | MAQ |
| SEQ ID | 132 | AQE |
| SEQ ID | 133 | QET |
| SEQ ID | 134 | ETD |
| SEQ ID | 135 | TDP |
| SEQ ID | 136 | DPA |
| SEQ ID | 137 | PAV |
| SEQ ID | 138 | AVQ |
| SEQ ID | 139 | VQT |
| SEQ ID | 140 | QTD |
| SEQ ID | 141 | TDG |
| SEQ ID | 142 | DGS |
| SEQ ID | 143 | GSP |
| SEQ ID | 144 | SPL |
| SEQ ID | 145 | PLC |
| SEQ ID | 146 | LCC |
| SEQ ID | 147 | CCH |
| SEQ ID | 148 | CHF |
| SEQ ID | 149 | HFH |
| SEQ ID | 150 | FHF |
| SEQ ID | 151 | HFS |
| SEQ ID | 152 | FSP |
| SEQ ID | 153 | SPK |
| SEQ ID | 154 | PKV |
| SEQ ID | 155 | KVM |
| SEQ ID | 156 | VMF |
| SEQ ID | 157 | MFT |
| SEQ ID | 158 | FTK |
| SEQ ID | 159 | TKV |
| SEQ ID | 160 | KVL |
| SEQ ID | 161 | VLK |
| SEQ ID | 162 | LKA |
| SEQ ID | 163 | KAQ |
| SEQ ID | 164 | AQL |
| SEQ ID | 165 | QLW |
| SEQ ID | 166 | LWV |
| SEQ ID | 167 | WVY |
| SEQ ID | 168 | VYL |
| SEQ ID | 169 | YLR |
| SEQ ID | 170 | RPV |
| SEQ ID | 171 | PVP |
| SEQ ID | 172 | VPR |
| SEQ ID | 173 | PRP |
| SEQ ID | 174 | PAT |
| SEQ ID | 175 | ATV |
| SEQ ID | 176 | TVY |
| SEQ ID | 177 | YLQ |
| SEQ ID | 178 | LQI |
| SEQ ID | 179 | ILR |
| SEQ ID | 180 | LKP |
| SEQ ID | 181 | KPL |
| SEQ ID | 182 | PLT |
| SEQ ID | 183 | LTG |
| SEQ ID | 184 | TGE |
| SEQ ID | 185 | GEG |
| SEQ ID | 186 | EGT |
| SEQ ID | 187 | GTA |
| SEQ ID | 188 | TAG |
| SEQ ID | 189 | AGG |
| SEQ ID | 190 | GGG |
| SEQ ID | 191 | GGR |
| SEQ ID | 192 | GRR |
| SEQ ID | 193 | RRH |
| SEQ ID | 194 | RHI |
| SEQ ID | 195 | HIR |
| SEQ ID | 196 | IRI |
| SEQ ID | 197 | RIR |
| SEQ ID | 198 | IRS |
| SEQ ID | 199 | RSL |
| SEQ ID | 200 | SLK |
| SEQ ID | 201 | LKI |
| SEQ ID | 202 | KIE |
| SEQ ID | 203 | IEL |
| SEQ ID | 204 | ELH |
| SEQ ID | 205 | LHS |
| SEQ ID | 206 | SRS |
| SEQ ID | 207 | RSG |
| SEQ ID | 208 | SGH |
| SEQ ID | 209 | GHW |
| SEQ ID | 210 | HWQ |
| SEQ ID | 211 | WQS |
| SEQ ID | 212 | QSI |
| SEQ ID | 213 | SID |
| SEQ ID | 214 | IDF |
| SEQ ID | 215 | DFK |
| SEQ ID | 216 | FKQ |
| SEQ ID | 217 | KQV |
| SEQ ID | 218 | QVL |
| SEQ ID | 219 | VLH |
| SEQ ID | 220 | HSW |
| SEQ ID | 221 | SWF |
| SEQ ID | 222 | WFR |
| SEQ ID | 223 | FRQ |
| SEQ ID | 224 | RQP |
| SEQ ID | 225 | QPQ |
| SEQ ID | 226 | PQS |
| SEQ ID | 227 | QSN |
| SEQ ID | 228 | SNW |
| SEQ ID | 229 | NWG |
| SEQ ID | 230 | WGI |
| SEQ ID | 231 | GIE |
| SEQ ID | 232 | IEI |
| SEQ ID | 233 | EIN |
| SEQ ID | 234 | INA |
| SEQ ID | 235 | NAF |
| SEQ ID | 236 | AFD |
| SEQ ID | 237 | FDP |
| SEQ ID | 238 | DPS |
| SEQ ID | 239 | PSG |
| SEQ ID | 240 | SGT |
| SEQ ID | 241 | GTD |
| SEQ ID | 242 | TDL |
| SEQ ID | 243 | DLA |
| SEQ ID | 244 | LAV |
| SEQ ID | 245 | AVT |
| SEQ ID | 246 | VTS |
| SEQ ID | 247 | TSL |
| SEQ ID | 248 | SLG |
| SEQ ID | 249 | LGP |
| SEQ ID | 250 | GPG |
| SEQ ID | 251 | PGA |
| SEQ ID | 252 | GAE |
| SEQ ID | 253 | EGL |
| SEQ ID | 254 | GLH |
| SEQ ID | 255 | LHP |
| SEQ ID | 256 | HPF |
| SEQ ID | 257 | PFM |
| SEQ ID | 258 | FME |
| SEQ ID | 259 | MEL |
| SEQ ID | 260 | LRV |
| SEQ ID | 261 | RVL |
| SEQ ID | 262 | VLE |
| SEQ ID | 263 | LEN |
| SEQ ID | 264 | ENT |
| SEQ ID | 265 | NTK |
| SEQ ID | 266 | TKR |
| SEQ ID | 267 | KRS |
| SEQ ID | 268 | RSR |
| SEQ ID | 269 | SRR |
| SEQ ID | 270 | RRN |
| SEQ ID | 271 | RNL |
| SEQ ID | 272 | NLG |
| SEQ ID | 273 | LGL |
| SEQ ID | 274 | GLD |
| SEQ ID | 275 | LDC |
| SEQ ID | 276 | DCD |
| SEQ ID | 277 | CDE |
| SEQ ID | 278 | DEH |
| SEQ ID | 279 | EHS |
| SEQ ID | 280 | HSS |
| SEQ ID | 281 | SSE |
| SEQ ID | 282 | SES |
| SEQ ID | 283 | ESR |
| SEQ ID | 284 | SRC |
| SEQ ID | 285 | RCC |
| SEQ ID | 286 | CCR |
| SEQ ID | 287 | CRY |
| SEQ ID | 288 | RYP |
| SEQ ID | 289 | YPL |
| SEQ ID | 290 | LTV |
| SEQ ID | 291 | TVD |
| SEQ ID | 292 | VDF |
| SEQ ID | 293 | DFE |
| SEQ ID | 294 | FEA |
| SEQ ID | 295 | EAF |
| SEQ ID | 296 | AFG |
| SEQ ID | 297 | FGW |
| SEQ ID | 298 | GWD |
| SEQ ID | 299 | WDW |
| SEQ ID | 300 | DWI |
| SEQ ID | 301 | WII |
| SEQ ID | 302 | IIA |
| SEQ ID | 303 | IAP |
| SEQ ID | 304 | APK |
| SEQ ID | 305 | PKR |
| SEQ ID | 306 | KRY |
| SEQ ID | 307 | RYK |
| SEQ ID | 308 | YKA |
| SEQ ID | 309 | KAN |
| SEQ ID | 310 | ANY |
| SEQ ID | 311 | NYC |
| SEQ ID | 312 | YCS |
| SEQ ID | 313 | CSG |
| SEQ ID | 314 | SGQ |
| SEQ ID | 315 | GQC |
| SEQ ID | 316 | QCE |
| SEQ ID | 317 | CEY |
| SEQ ID | 318 | EYM |
| SEQ ID | 319 | YMF |
| SEQ ID | 320 | MFM |
| SEQ ID | 321 | FMQ |
| SEQ ID | 322 | MQK |
| SEQ ID | 323 | QKY |
| SEQ ID | 324 | KYP |
| SEQ ID | 325 | YPH |
| SEQ ID | 326 | PHT |
| SEQ ID | 327 | HTH |
| SEQ ID | 328 | THL |
| SEQ ID | 329 | HLV |
| SEQ ID | 330 | LVQ |
| SEQ ID | 331 | VQQ |
| SEQ ID | 332 | QQA |
| SEQ ID | 333 | QAN |
| SEQ ID | 334 | ANP |
| SEQ ID | 335 | NPR |
| SEQ ID | 336 | RGS |
| SEQ ID | 337 | GSA |
| SEQ ID | 338 | SAG |
| SEQ ID | 339 | AGP |
| SEQ ID | 340 | GPC |
| SEQ ID | 341 | PCC |
| SEQ ID | 342 | CCT |
| SEQ ID | 343 | CTP |
| SEQ ID | 344 | TPT |
| SEQ ID | 345 | PTK |
| SEQ ID | 346 | TKM |
| SEQ ID | 347 | KMS |
| SEQ ID | 348 | MSP |
| SEQ ID | 349 | SPI |
| SEQ ID | 350 | PIN |
| SEQ ID | 351 | INM |
| SEQ ID | 352 | NML |
| SEQ ID | 353 | MLY |
| SEQ ID | 354 | LYF |
| SEQ ID | 355 | YFN |
| SEQ ID | 356 | FND |
| SEQ ID | 357 | NDK |
| SEQ ID | 358 | DKQ |
| SEQ ID | 359 | KQQ |
| SEQ ID | 360 | QII |
| SEQ ID | 361 | IIY |
| SEQ ID | 362 | IYG |
| SEQ ID | 363 | YGK |
| SEQ ID | 364 | GKI |
| SEQ ID | 365 | KIP |
| SEQ ID | 366 | IPG |
| SEQ ID | 367 | PGM |
| SEQ ID | 368 | GMV |
| SEQ ID | 369 | MVV |
| SEQ ID | 370 | VVD |
| SEQ ID | 371 | VDR |
| SEQ ID | 372 | DRC |
| SEQ ID | 373 | RCG |
| SEQ ID | 374 | CGC |
| SEQ ID | 375 | GCS |

In some embodiments, the peptide comprises, consists essentially of, or consists of any one or more of the 4-mer amino acid sequences (SEQ ID NO: 376-767) listed in Table 3.

TABLE 3

| | | |
|---|---|---|
| SEQ ID | 376 | MVLA |
| SEQ ID | 377 | VLAA |
| SEQ ID | 378 | LAAP |
| SEQ ID | 379 | AAPL |
| SEQ ID | 380 | APLL |
| SEQ ID | 381 | PLLL |
| SEQ ID | 382 | LLLG |
| SEQ ID | 383 | LLGF |
| SEQ ID | 384 | LGFL |
| SEQ ID | 385 | GFLL |
| SEQ ID | 386 | FLLL |
| SEQ ID | 387 | LLLA |
| SEQ ID | 388 | LLAL |
| SEQ ID | 389 | LALE |
| SEQ ID | 390 | ALEL |
| SEQ ID | 391 | LELR |
| SEQ ID | 392 | ELRP |
| SEQ ID | 393 | LRPR |
| SEQ ID | 394 | RPRG |
| SEQ ID | 395 | PRGE |
| SEQ ID | 396 | RGEA |
| SEQ ID | 397 | GEAA |
| SEQ ID | 398 | EAAE |
| SEQ ID | 399 | AAEG |
| SEQ ID | 400 | AEGP |
| SEQ ID | 401 | EGPA |
| SEQ ID | 402 | GPAA |
| SEQ ID | 403 | PAAA |
| SEQ ID | 404 | AAAA |
| SEQ ID | 405 | AAAG |
| SEQ ID | 406 | AAGV |
| SEQ ID | 407 | AGVG |
| SEQ ID | 408 | GVGG |
| SEQ ID | 409 | VGGE |
| SEQ ID | 410 | GGER |
| SEQ ID | 411 | GERS |
| SEQ ID | 412 | ERSS |
| SEQ ID | 413 | RSSR |
| SEQ ID | 414 | SSRP |
| SEQ ID | 415 | SRPA |
| SEQ ID | 416 | RPAP |
| SEQ ID | 417 | PAPS |
| SEQ ID | 418 | APSV |
| SEQ ID | 419 | PSVA |
| SEQ ID | 420 | SVAP |
| SEQ ID | 421 | VAPE |
| SEQ ID | 422 | APEP |
| SEQ ID | 423 | PEPD |
| SEQ ID | 424 | EPDG |
| SEQ ID | 425 | PDGC |
| SEQ ID | 426 | DGCP |
| SEQ ID | 427 | GCPV |
| SEQ ID | 428 | CPVC |
| SEQ ID | 429 | PVCV |
| SEQ ID | 430 | VCVW |
| SEQ ID | 431 | CVWR |
| SEQ ID | 432 | VWRQ |
| SEQ ID | 433 | WRQH |
| SEQ ID | 434 | RQHS |
| SEQ ID | 435 | QHSR |
| SEQ ID | 436 | HSRE |
| SEQ ID | 437 | SREL |
| SEQ ID | 438 | RELR |
| SEQ ID | 439 | ELRL |
| SEQ ID | 440 | LRLE |
| SEQ ID | 441 | RLES |
| SEQ ID | 442 | LESI |
| SEQ ID | 443 | ESIK |
| SEQ ID | 444 | SIKS |
| SEQ ID | 445 | IKSQ |
| SEQ ID | 446 | KSQI |
| SEQ ID | 447 | SQIL |
| SEQ ID | 448 | QILS |
| SEQ ID | 449 | ILSK |
| SEQ ID | 450 | LSKL |
| SEQ ID | 451 | SKLR |
| SEQ ID | 452 | KLRL |
| SEQ ID | 453 | LRLK |
| SEQ ID | 454 | RLKE |
| SEQ ID | 455 | LKEA |
| SEQ ID | 456 | KEAP |
| SEQ ID | 457 | EAPN |
| SEQ ID | 458 | APNI |
| SEQ ID | 459 | PNIS |
| SEQ ID | 460 | NISR |
| SEQ ID | 461 | ISRE |
| SEQ ID | 462 | SREV |
| SEQ ID | 463 | REVV |
| SEQ ID | 464 | EVVK |
| SEQ ID | 465 | VVKQ |
| SEQ ID | 466 | VKQL |
| SEQ ID | 467 | KQLL |
| SEQ ID | 468 | QLLP |
| SEQ ID | 469 | LLPK |
| SEQ ID | 470 | LPKA |
| SEQ ID | 471 | PKAP |
| SEQ ID | 472 | KAPP |
| SEQ ID | 473 | APPL |
| SEQ ID | 474 | PPLQ |
| SEQ ID | 475 | PLQQ |
| SEQ ID | 476 | LQQI |
| SEQ ID | 477 | QQIL |
| SEQ ID | 478 | QILD |
| SEQ ID | 479 | ILDL |
| SEQ ID | 480 | LDLH |
| SEQ ID | 481 | DLHD |
| SEQ ID | 482 | LHDF |
| SEQ ID | 483 | HDFQ |
| SEQ ID | 484 | DFQG |
| SEQ ID | 485 | FQGD |
| SEQ ID | 486 | QGDA |
| SEQ ID | 487 | GDAL |
| SEQ ID | 488 | DALQ |
| SEQ ID | 489 | ALQP |
| SEQ ID | 490 | LQPE |
| SEQ ID | 491 | QPED |
| SEQ ID | 492 | PEDF |
| SEQ ID | 493 | EDFL |
| SEQ ID | 494 | DFLE |
| SEQ ID | 495 | FLEE |
| SEQ ID | 496 | LEED |
| SEQ ID | 497 | EEDE |
| SEQ ID | 498 | EDEY |
| SEQ ID | 499 | DEYH |
| SEQ ID | 500 | EYHA |
| SEQ ID | 501 | YHAT |
| SEQ ID | 502 | HATT |
| SEQ ID | 503 | ATTE |
| SEQ ID | 504 | TTET |
| SEQ ID | 505 | TETV |
| SEQ ID | 506 | ETVI |
| SEQ ID | 507 | TVIS |
| SEQ ID | 508 | VISM |
| SEQ ID | 509 | ISMA |
| SEQ ID | 510 | SMAQ |
| SEQ ID | 511 | MAQE |
| SEQ ID | 512 | AQET |
| SEQ ID | 513 | QETD |
| SEQ ID | 514 | ETDP |
| SEQ ID | 515 | TDPA |
| SEQ ID | 516 | DPAV |
| SEQ ID | 517 | PAVQ |
| SEQ ID | 518 | AVQT |
| SEQ ID | 519 | VQTD |
| SEQ ID | 520 | QTDG |
| SEQ ID | 521 | TDGS |
| SEQ ID | 522 | DGSP |
| SEQ ID | 523 | GSPL |
| SEQ ID | 524 | SPLC |
| SEQ ID | 525 | PLCC |
| SEQ ID | 526 | LCCH |
| SEQ ID | 527 | CCHF |
| SEQ ID | 528 | CHFH |
| SEQ ID | 529 | HFHF |
| SEQ ID | 530 | FHFS |
| SEQ ID | 531 | HFSP |
| SEQ ID | 532 | FSPK |
| SEQ ID | 533 | SPKV |
| SEQ ID | 534 | PKVM |
| SEQ ID | 535 | KVMF |
| SEQ ID | 536 | VMFT |
| SEQ ID | 537 | MFTK |
| SEQ ID | 538 | FTKV |
| SEQ ID | 539 | TKVL |
| SEQ ID | 540 | KVLK |
| SEQ ID | 541 | VLKA |
| SEQ ID | 542 | LKAQ |
| SEQ ID | 543 | KAQL |
| SEQ ID | 544 | AQLW |
| SEQ ID | 545 | QLWV |
| SEQ ID | 546 | LWVY |
| SEQ ID | 547 | WVYL |
| SEQ ID | 548 | VYLR |
| SEQ ID | 549 | YLRP |
| SEQ ID | 550 | LRPV |
| SEQ ID | 551 | RPVP |
| SEQ ID | 552 | PVPR |
| SEQ ID | 553 | VPRP |
| SEQ ID | 554 | PRPA |
| SEQ ID | 555 | RPAT |
| SEQ ID | 556 | PATV |
| SEQ ID | 557 | ATVY |
| SEQ ID | 558 | TVYL |
| SEQ ID | 559 | VYLQ |
| SEQ ID | 560 | YLQI |
| SEQ ID | 561 | LQIL |
| SEQ ID | 562 | QILR |
| SEQ ID | 563 | ILRL |
| SEQ ID | 564 | RLKP |
| SEQ ID | 565 | LKPL |
| SEQ ID | 566 | KPLT |
| SEQ ID | 567 | PLTG |
| SEQ ID | 568 | LTGE |
| SEQ ID | 569 | TGEG |
| SEQ ID | 570 | GEGT |
| SEQ ID | 571 | EGTA |
| SEQ ID | 572 | GTAG |
| SEQ ID | 573 | TAGG |
| SEQ ID | 574 | AGGG |
| SEQ ID | 575 | GGGG |
| SEQ ID | 576 | GGGR |
| SEQ ID | 577 | GGRR |
| SEQ ID | 578 | GRRH |
| SEQ ID | 579 | RRHI |
| SEQ ID | 580 | RHIR |
| SEQ ID | 581 | HIRT |
| SEQ ID | 582 | IRIR |
| SEQ ID | 583 | RIRS |
| SEQ ID | 584 | IRSL |
| SEQ ID | 585 | RSLK |
| SEQ ID | 586 | SLKI |
| SEQ ID | 587 | LKIE |
| SEQ ID | 588 | KIEL |
| SEQ ID | 589 | IELH |
| SEQ ID | 590 | ELHS |
| SEQ ID | 591 | LHSR |
| SEQ ID | 592 | HSRS |
| SEQ ID | 593 | SRSG |
| SEQ ID | 594 | RSGH |
| SEQ ID | 595 | SGHW |
| SEQ ID | 596 | GHWQ |
| SEQ ID | 597 | HWQS |
| SEQ ID | 598 | WQSI |
| SEQ ID | 599 | QSID |
| SEQ ID | 600 | SIDF |
| SEQ ID | 601 | IDFK |
| SEQ ID | 602 | DFKQ |
| SEQ ID | 603 | FKQV |
| SEQ ID | 604 | KQVL |
| SEQ ID | 605 | QVLH |
| SEQ ID | 606 | VLHS |
| SEQ ID | 607 | LHSW |
| SEQ ID | 608 | HSWF |
| SEQ ID | 609 | SWFR |
| SEQ ID | 610 | WFRQ |
| SEQ ID | 611 | FRQP |
| SEQ ID | 612 | RQPQ |
| SEQ ID | 613 | QPQS |
| SEQ ID | 614 | PQSN |
| SEQ ID | 615 | QSNW |
| SEQ ID | 616 | SNWG |
| SEQ ID | 617 | NWGI |
| SEQ ID | 618 | WGIE |
| SEQ ID | 619 | GIEI |
| SEQ ID | 620 | IEIN |
| SEQ ID | 621 | EINA |
| SEQ ID | 622 | INAF |
| SEQ ID | 623 | NAFD |
| SEQ ID | 624 | AFDP |
| SEQ ID | 625 | FDPS |
| SEQ ID | 626 | DPSG |
| SEQ ID | 627 | PSGT |
| SEQ ID | 628 | SGTD |
| SEQ ID | 629 | GTDL |
| SEQ ID | 630 | TDLA |
| SEQ ID | 631 | DLAV |
| SEQ ID | 632 | LAVT |
| SEQ ID | 633 | AVTS |
| SEQ ID | 634 | VTSL |
| SEQ ID | 635 | TSLG |
| SEQ ID | 636 | SLGP |
| SEQ ID | 637 | LGPG |
| SEQ ID | 638 | GPGA |
| SEQ ID | 639 | PGAE |
| SEQ ID | 640 | GAEG |
| SEQ ID | 641 | AEGL |
| SEQ ID | 642 | EGLH |
| SEQ ID | 643 | GLHP |
| SEQ ID | 644 | LHPF |
| SEQ ID | 645 | HPFM |
| SEQ ID | 646 | PFME |
| SEQ ID | 647 | FMEL |
| SEQ ID | 648 | MELR |
| SEQ ID | 649 | ELRV |
| SEQ ID | 650 | LRVL |
| SEQ ID | 651 | RVLE |
| SEQ ID | 652 | VLEN |
| SEQ ID | 653 | LENT |
| SEQ ID | 654 | ENTK |
| SEQ ID | 655 | NTKR |
| SEQ ID | 656 | TKRS |
| SEQ ID | 657 | KRSR |
| SEQ ID | 658 | RSRR |
| SEQ ID | 659 | SRRN |
| SEQ ID | 660 | RRNL |
| SEQ ID | 661 | RNLG |
| SEQ ID | 662 | NLGL |
| SEQ ID | 663 | LGLD |
| SEQ ID | 664 | GLDC |
| SEQ ID | 665 | LDCD |
| SEQ ID | 666 | DCDE |
| SEQ ID | 667 | CDEH |
| SEQ ID | 668 | DEHS |
| SEQ ID | 669 | EHSS |
| SEQ ID | 670 | HSSE |
| SEQ ID | 671 | SSES |
| SEQ ID | 672 | SESR |
| SEQ ID | 673 | ESRC |
| SEQ ID | 674 | SRCC |
| SEQ ID | 675 | RCCR |
| SEQ ID | 676 | CCRY |
| SEQ ID | 677 | CRYP |
| SEQ ID | 678 | RYPL |
| SEQ ID | 679 | YPLT |
| SEQ ID | 680 | PLTV |
| SEQ ID | 681 | LTVD |
| SEQ ID | 682 | TVDF |
| SEQ ID | 683 | VDFE |
| SEQ ID | 684 | DFEA |
| SEQ ID | 685 | FEAF |
| SEQ ID | 686 | EAFG |
| SEQ ID | 687 | AFGW |
| SEQ ID | 688 | FGWD |
| SEQ ID | 689 | GWDW |
| SEQ ID | 690 | WDWI |
| SEQ ID | 691 | DWII |
| SEQ ID | 692 | WIIA |
| SEQ ID | 693 | IIAP |
| SEQ ID | 694 | IAPK |
| SEQ ID | 695 | APKR |
| SEQ ID | 696 | PKRY |
| SEQ ID | 697 | KRYK |
| SEQ ID | 698 | RYKA |
| SEQ ID | 699 | YKAN |
| SEQ ID | 700 | KANY |
| SEQ ID | 701 | ANYC |
| SEQ ID | 702 | NYCS |
| SEQ ID | 703 | YCSG |
| SEQ ID | 704 | CSGQ |
| SEQ ID | 705 | SGQC |
| SEQ ID | 706 | GQCE |
| SEQ ID | 707 | QCEY |
| SEQ ID | 708 | CEYM |
| SEQ ID | 709 | EYMF |
| SEQ ID | 710 | YMFM |
| SEQ ID | 711 | MFMQ |
| SEQ ID | 712 | FMQK |
| SEQ ID | 713 | MQKY |
| SEQ ID | 714 | QKYP |
| SEQ ID | 715 | KYPH |
| SEQ ID | 716 | YPHT |
| SEQ ID | 717 | PHTH |
| SEQ ID | 718 | HTHL |
| SEQ ID | 719 | THLV |
| SEQ ID | 720 | HLVQ |
| SEQ ID | 721 | LVQQ |
| SEQ ID | 722 | VQQA |
| SEQ ID | 723 | QQAN |
| SEQ ID | 724 | QANP |
| SEQ ID | 725 | ANPR |
| SEQ ID | 726 | NPRG |
| SEQ ID | 727 | PRGS |
| SEQ ID | 728 | RGSA |
| SEQ ID | 729 | GSAG |
| SEQ ID | 730 | SAGP |
| SEQ ID | 731 | AGPC |
| SEQ ID | 732 | GPCC |
| SEQ ID | 733 | PCCT |
| SEQ ID | 734 | CCTP |
| SEQ ID | 735 | CTPT |
| SEQ ID | 736 | TPTK |
| SEQ ID | 737 | PTKM |
| SEQ ID | 738 | TKMS |
| SEQ ID | 739 | KMSP |
| SEQ ID | 740 | MSPI |
| SEQ ID | 741 | SPIN |
| SEQ ID | 742 | PINM |
| SEQ ID | 743 | INML |
| SEQ ID | 744 | NMLY |
| SEQ ID | 745 | MLYF |
| SEQ ID | 746 | LYFN |
| SEQ ID | 747 | YFND |
| SEQ ID | 748 | FNDK |
| SEQ ID | 749 | NDKQ |
| SEQ ID | 750 | DKQQ |
| SEQ ID | 751 | KQQI |
| SEQ ID | 752 | QQII |
| SEQ ID | 753 | QIIY |
| SEQ ID | 754 | IIYG |
| SEQ ID | 755 | IYGK |
| SEQ ID | 756 | YGKI |
| SEQ ID | 757 | GKIP |
| SEQ ID | 758 | KIPG |
| SEQ ID | 759 | IPGM |
| SEQ ID | 760 | PGMV |
| SEQ ID | 761 | GMVV |
| SEQ ID | 762 | MVVD |
| SEQ ID | 763 | VVDR |
| SEQ ID | 764 | VDRC |
| SEQ ID | 765 | DRCG |
| SEQ ID | 766 | RCGC |
| SEQ ID | 767 | CGCS |

In some embodiments, the active agent comprises a peptide which comprises, consists essentially of, or consists of any one or more of the 5-mer amino acid sequences (SEQ ID NO: 768-1161) listed in Table 4.

TABLE 4

| | | |
|---|---|---|
| SEQ ID | 768 | MVLAA |
| SEQ ID | 769 | VLAAP |
| SEQ ID | 770 | LAAPL |
| SEQ ID | 771 | AAPLL |
| SEQ ID | 772 | APLLL |
| SEQ ID | 773 | PLLLG |
| SEQ ID | 774 | LLLGF |
| SEQ ID | 775 | LLGFL |
| SEQ ID | 776 | LGFLL |
| SEQ ID | 777 | GFLLL |
| SEQ ID | 778 | FLLLA |
| SEQ ID | 779 | LLLAL |
| SEQ ID | 780 | LLALE |
| SEQ ID | 781 | LALEL |
| SEQ ID | 782 | ALELR |
| SEQ ID | 783 | LELRP |
| SEQ ID | 784 | ELRPR |
| SEQ ID | 785 | LRPRG |
| SEQ ID | 786 | RPRGE |
| SEQ ID | 787 | PRGEA |
| SEQ ID | 788 | RGEAA |
| SEQ ID | 789 | GEAAE |
| SEQ ID | 790 | EAAEG |
| SEQ ID | 791 | AAEGP |
| SEQ ID | 792 | AEGPA |
| SEQ ID | 793 | EGPAA |
| SEQ ID | 794 | GPAAA |
| SEQ ID | 795 | PAAAA |
| SEQ ID | 796 | AAAAA |
| SEQ ID | 797 | AAAAG |
| SEQ ID | 798 | AAAGV |
| SEQ ID | 799 | AAGVG |
| SEQ ID | 800 | AGVGG |
| SEQ ID | 801 | GVGGE |
| SEQ ID | 802 | VGGER |
| SEQ ID | 803 | GGERS |
| SEQ ID | 804 | GERSS |
| SEQ ID | 805 | ERSSR |
| SEQ ID | 806 | RSSRP |
| SEQ ID | 807 | SSRPA |
| SEQ ID | 808 | SRPAP |
| SEQ ID | 809 | RPAPS |
| SEQ ID | 810 | PAPSV |
| SEQ ID | 811 | APSVA |
| SEQ ID | 812 | PSVAP |
| SEQ ID | 813 | SVAPE |
| SEQ ID | 814 | VAPEP |
| SEQ ID | 815 | APEPD |
| SEQ ID | 816 | PEPDG |
| SEQ ID | 817 | EPDGC |
| SEQ ID | 818 | PDGCP |
| SEQ ID | 819 | DGCPV |
| SEQ ID | 820 | GCPVC |
| SEQ ID | 821 | CPVCV |
| SEQ ID | 822 | PVCVW |
| SEQ ID | 823 | VCVWR |
| SEQ ID | 824 | CVWRQ |
| SEQ ID | 825 | VWRQH |
| SEQ ID | 826 | WRQHS |
| SEQ ID | 827 | RQHSR |
| SEQ ID | 828 | QHSRE |
| SEQ ID | 829 | HSREL |
| SEQ ID | 830 | SRELR |
| SEQ ID | 831 | RELRL |
| SEQ ID | 832 | ELRLE |
| SEQ ID | 833 | LRLES |
| SEQ ID | 834 | RLESI |
| SEQ ID | 835 | LESIK |
| SEQ ID | 836 | ESIKS |
| SEQ ID | 837 | SIKSQ |
| SEQ ID | 838 | IKSQI |
| SEQ ID | 839 | KSQIL |
| SEQ ID | 840 | SQILS |
| SEQ ID | 841 | QILSK |
| SEQ ID | 842 | ILSKL |
| SEQ ID | 843 | LSKLR |
| SEQ ID | 844 | SKLRL |
| SEQ ID | 845 | KLRLK |
| SEQ ID | 846 | LRLKE |
| SEQ ID | 847 | RLKEA |
| SEQ ID | 848 | LKEAP |
| SEQ ID | 849 | KEAPN |
| SEQ ID | 850 | EAPNI |
| SEQ ID | 851 | APNIS |
| SEQ ID | 852 | PNISR |
| SEQ ID | 853 | NISRE |
| SEQ ID | 854 | ISREV |
| SEQ ID | 855 | SREVV |
| SEQ ID | 856 | REVVK |
| SEQ ID | 857 | EVVKQ |
| SEQ ID | 858 | VVKQL |
| SEQ ID | 859 | VKQLL |
| SEQ ID | 860 | KQLLP |
| SEQ ID | 861 | QLLPK |
| SEQ ID | 862 | LLPKA |
| SEQ ID | 863 | LPKAP |
| SEQ ID | 864 | PKAPP |
| SEQ ID | 865 | KAPPL |
| SEQ ID | 866 | APPLQ |
| SEQ ID | 867 | PPLQQ |
| SEQ ID | 868 | PLQQI |
| SEQ ID | 869 | LQQIL |
| SEQ ID | 870 | QQILD |
| SEQ ID | 871 | QILDL |
| SEQ ID | 872 | ILDLH |
| SEQ ID | 873 | LDLHD |
| SEQ ID | 874 | DLHDF |
| SEQ ID | 875 | LHDFQ |
| SEQ ID | 876 | HDFQG |
| SEQ ID | 877 | DFQGD |
| SEQ ID | 878 | FQGDA |
| SEQ ID | 879 | QGDAL |
| SEQ ID | 880 | GDALQ |
| SEQ ID | 881 | DALQP |
| SEQ ID | 882 | ALQPE |
| SEQ ID | 883 | LQPED |
| SEQ ID | 884 | QPEDF |
| SEQ ID | 885 | PEDFL |
| SEQ ID | 886 | EDFLE |
| SEQ ID | 887 | DFLEE |
| SEQ ID | 888 | FLEED |
| SEQ ID | 889 | LEEDE |
| SEQ ID | 890 | EEDEY |
| SEQ ID | 891 | EDEYH |
| SEQ ID | 892 | DEYHA |
| SEQ ID | 893 | EYHAT |
| SEQ ID | 894 | YHATT |
| SEQ ID | 895 | HATTE |
| SEQ ID | 896 | ATTET |
| SEQ ID | 897 | TTETV |
| SEQ ID | 898 | TETVI |
| SEQ ID | 899 | ETVIS |
| SEQ ID | 900 | TVISM |
| SEQ ID | 901 | VISMA |
| SEQ ID | 902 | ISMAQ |
| SEQ ID | 903 | SMAQE |
| SEQ ID | 904 | MAQET |
| SEQ ID | 905 | AQETD |
| SEQ ID | 906 | QETDP |
| SEQ ID | 907 | ETDPA |
| SEQ ID | 908 | TDPAV |
| SEQ ID | 909 | DPAVQ |
| SEQ ID | 910 | PAVQT |
| SEQ ID | 911 | AVQTD |
| SEQ ID | 912 | VQTDG |
| SEQ ID | 913 | QTDGS |
| SEQ ID | 914 | TDGSP |
| SEQ ID | 915 | DGSPL |
| SEQ ID | 916 | GSPLC |
| SEQ ID | 917 | SPLCC |
| SEQ ID | 918 | PLCCH |
| SEQ ID | 919 | LCCHF |
| SEQ ID | 920 | CCHFH |
| SEQ ID | 921 | CHFHF |
| SEQ ID | 922 | HFHFS |
| SEQ ID | 923 | FHFSP |
| SEQ ID | 924 | HFSPK |
| SEQ ID | 925 | FSPKV |
| SEQ ID | 926 | SPKVM |
| SEQ ID | 927 | PKVMF |
| SEQ ID | 928 | KVMFT |
| SEQ ID | 929 | VMFTK |
| SEQ ID | 930 | MFTKV |
| SEQ ID | 931 | FTKVL |
| SEQ ID | 932 | TKVLK |
| SEQ ID | 933 | KVLKA |
| SEQ ID | 934 | VLKAQ |
| SEQ ID | 935 | LKAQL |
| SEQ ID | 936 | KAQLW |
| SEQ ID | 937 | AQLWV |
| SEQ ID | 938 | QLWVY |
| SEQ ID | 939 | LWVYL |
| SEQ ID | 940 | WVYLR |
| SEQ ID | 941 | VYLRP |
| SEQ ID | 942 | YLRPV |
| SEQ ID | 943 | LRPVP |
| SEQ ID | 944 | RPVPR |
| SEQ ID | 945 | PVPRP |
| SEQ ID | 946 | VPRPA |
| SEQ ID | 947 | PRPAT |
| SEQ ID | 948 | RPATV |
| SEQ ID | 949 | PATVY |
| SEQ ID | 950 | ATVYL |
| SEQ ID | 951 | TVYLQ |
| SEQ ID | 952 | VYLQI |
| SEQ ID | 953 | YLQIL |
| SEQ ID | 954 | LQILR |
| SEQ ID | 955 | QILRL |
| SEQ ID | 956 | ILRLK |
| SEQ ID | 957 | LRLKP |
| SEQ ID | 958 | RLKPL |
| SEQ ID | 959 | LKPLT |
| SEQ ID | 960 | KPLTG |
| SEQ ID | 961 | PLTGE |
| SEQ ID | 962 | LTGEG |
| SEQ ID | 963 | TGEGT |
| SEQ ID | 964 | GEGTA |
| SEQ ID | 965 | EGTAG |
| SEQ ID | 966 | GTAGG |
| SEQ ID | 967 | TAGGG |
| SEQ ID | 968 | AGGGG |
| SEQ ID | 969 | GGGGG |
| SEQ ID | 970 | GGGGR |
| SEQ ID | 971 | GGGRR |
| SEQ ID | 972 | GGRRH |
| SEQ ID | 973 | GRRHI |
| SEQ ID | 974 | RRHIR |
| SEQ ID | 975 | RHIRI |
| SEQ ID | 976 | HIRIR |
| SEQ ID | 977 | IRIRS |
| SEQ ID | 978 | RIRSL |
| SEQ ID | 979 | IRSLK |
| SEQ ID | 980 | RSLKI |
| SEQ ID | 981 | SLKIE |
| SEQ ID | 982 | LKIEL |
| SEQ ID | 983 | KIELH |
| SEQ ID | 984 | IELHS |
| SEQ ID | 985 | ELHSR |
| SEQ ID | 986 | LHSRS |
| SEQ ID | 987 | HSRSG |
| SEQ ID | 988 | SRSGH |
| SEQ ID | 989 | RSGHW |
| SEQ ID | 990 | SGHWQ |
| SEQ ID | 991 | GHWQS |
| SEQ ID | 992 | HWQSI |
| SEQ ID | 993 | WQSID |
| SEQ ID | 994 | QSIDF |
| SEQ ID | 995 | SIDFK |
| SEQ ID | 996 | IDFKQ |
| SEQ ID | 997 | DFKQV |
| SEQ ID | 998 | FKQVL |
| SEQ ID | 999 | KQVLH |
| SEQ ID | 1000 | QVLHS |
| SEQ ID | 1001 | VLHSW |
| SEQ ID | 1002 | LHSWF |
| SEQ ID | 1003 | HSWFR |
| SEQ ID | 1004 | SWFRQ |
| SEQ ID | 1005 | WFRQP |
| SEQ ID | 1006 | FRQPQ |
| SEQ ID | 1007 | RQPQS |
| SEQ ID | 1008 | QPQSN |
| SEQ ID | 1009 | PQSNW |
| SEQ ID | 1010 | QSNWG |
| SEQ ID | 1011 | SNWGI |
| SEQ ID | 1012 | NWGIE |
| SEQ ID | 1013 | WGIEI |
| SEQ ID | 1014 | GIEIN |
| SEQ ID | 1015 | IEINA |
| SEQ ID | 1016 | EINAF |
| SEQ ID | 1017 | INAFD |
| SEQ ID | 1018 | NAFDP |
| SEQ ID | 1019 | AFDPS |
| SEQ ID | 1020 | FDPSG |
| SEQ ID | 1021 | DPSGT |
| SEQ ID | 1022 | PSGTD |
| SEQ ID | 1023 | SGTDL |
| SEQ ID | 1024 | GTDLA |
| SEQ ID | 1025 | TDLAV |
| SEQ ID | 1026 | DLAVT |
| SEQ ID | 1027 | LAVTS |
| SEQ ID | 1028 | AVTSL |
| SEQ ID | 1029 | VTSLG |
| SEQ ID | 1030 | TSLGP |
| SEQ ID | 1031 | SLGPG |
| SEQ ID | 1032 | LGPGA |
| SEQ ID | 1033 | GPGAE |
| SEQ ID | 1034 | PGAEG |
| SEQ ID | 1035 | GAEGL |
| SEQ ID | 1036 | AEGLH |
| SEQ ID | 1037 | EGLHP |
| SEQ ID | 1038 | GLHPF |
| SEQ ID | 1039 | LHPFM |
| SEQ ID | 1040 | HPFME |
| SEQ ID | 1041 | PFMEL |
| SEQ ID | 1042 | FMELR |
| SEQ ID | 1043 | MELRV |
| SEQ ID | 1044 | ELRVL |
| SEQ ID | 1045 | LRVLE |
| SEQ ID | 1046 | RVLEN |
| SEQ ID | 1047 | VLENT |
| SEQ ID | 1048 | LENTK |
| SEQ ID | 1049 | ENTKR |
| SEQ ID | 1050 | NTKRS |
| SEQ ID | 1051 | TKRSR |
| SEQ ID | 1052 | KRSRR |
| SEQ ID | 1053 | RSRRN |
| SEQ ID | 1054 | SRRNL |
| SEQ ID | 1055 | RRNLG |
| SEQ ID | 1056 | RNLGL |
| SEQ ID | 1057 | NLGLD |
| SEQ ID | 1058 | LGLDC |
| SEQ ID | 1059 | GLDCD |
| SEQ ID | 1060 | LDCDE |
| SEQ ID | 1061 | DCDEH |
| SEQ ID | 1062 | CDEHS |
| SEQ ID | 1063 | DEHSS |
| SEQ ID | 1064 | EHSSE |
| SEQ ID | 1065 | HSSES |
| SEQ ID | 1066 | SSESR |
| SEQ ID | 1067 | SESRC |
| SEQ ID | 1068 | ESRCC |
| SEQ ID | 1069 | SRCCR |
| SEQ ID | 1070 | RCCRY |
| SEQ ID | 1071 | CCRYP |
| SEQ ID | 1072 | CRYPL |
| SEQ ID | 1073 | RYPLT |
| SEQ ID | 1074 | YPLTV |
| SEQ ID | 1075 | PLTVD |
| SEQ ID | 1076 | LTVDF |
| SEQ ID | 1077 | TVDFE |
| SEQ ID | 1078 | VDFEA |
| SEQ ID | 1079 | DFEAF |
| SEQ ID | 1080 | FEAFG |
| SEQ ID | 1081 | EAFGW |
| SEQ ID | 1082 | AFGWD |
| SEQ ID | 1083 | FGWDW |
| SEQ ID | 1084 | GWDWI |
| SEQ ID | 1085 | WDWII |
| SEQ ID | 1086 | DWIIA |
| SEQ ID | 1087 | WIIAP |
| SEQ ID | 1088 | IIAPK |
| SEQ ID | 1089 | IAPKR |
| SEQ ID | 1090 | APKRY |
| SEQ ID | 1091 | PKRYK |
| SEQ ID | 1092 | KRYKA |
| SEQ ID | 1093 | RYKAN |
| SEQ ID | 1094 | YKANY |
| SEQ ID | 1095 | KANYC |
| SEQ ID | 1096 | ANYCS |
| SEQ ID | 1097 | NYCSG |
| SEQ ID | 1098 | YCSGQ |
| SEQ ID | 1099 | CSGQC |
| SEQ ID | 1100 | SGQCE |
| SEQ ID | 1101 | GQCEY |
| SEQ ID | 1102 | QCEYM |
| SEQ ID | 1103 | CEYMF |
| SEQ ID | 1104 | EYMFM |
| SEQ ID | 1105 | YMFMQ |
| SEQ ID | 1106 | MFMQK |
| SEQ ID | 1107 | FMQKY |
| SEQ ID | 1108 | MQKYP |
| SEQ ID | 1109 | QKYPH |
| SEQ ID | 1110 | KYPHT |
| SEQ ID | 1111 | YPHTH |
| SEQ ID | 1112 | PHTHL |
| SEQ ID | 1113 | HTHLV |
| SEQ ID | 1114 | THLVQ |
| SEQ ID | 1115 | HLVQQ |
| SEQ ID | 1116 | LVQQA |
| SEQ ID | 1117 | VQQAN |
| SEQ ID | 1118 | QQANP |
| SEQ ID | 1119 | QANPR |
| SEQ ID | 1120 | ANPRG |
| SEQ ID | 1121 | NPRGS |
| SEQ ID | 1122 | PRGSA |
| SEQ ID | 1123 | RGSAG |
| SEQ ID | 1124 | GSAGP |
| SEQ ID | 1125 | SAGPC |
| SEQ ID | 1126 | AGPCC |
| SEQ ID | 1127 | GPCCT |
| SEQ ID | 1128 | PCCTP |
| SEQ ID | 1129 | CCTPT |
| SEQ ID | 1130 | CTPTK |
| SEQ ID | 1131 | TPTKM |
| SEQ ID | 1132 | PTKMS |
| SEQ ID | 1133 | TKMSP |
| SEQ ID | 1134 | KMSPI |
| SEQ ID | 1135 | MSPIN |
| SEQ ID | 1136 | SPINM |
| SEQ ID | 1137 | PINML |
| SEQ ID | 1138 | INMLY |
| SEQ ID | 1139 | NMLYF |
| SEQ ID | 1140 | MLYFN |
| SEQ ID | 1141 | LYFND |
| SEQ ID | 1142 | YFNDK |
| SEQ ID | 1143 | FNDKQ |
| SEQ ID | 1144 | NDKQQ |
| SEQ ID | 1145 | DKQQI |
| SEQ ID | 1146 | KQQII |
| SEQ ID | 1147 | QQIIY |
| SEQ ID | 1148 | QIIYG |
| SEQ ID | 1149 | IIYGK |
| SEQ ID | 1150 | IYGKI |
| SEQ ID | 1151 | YGKIP |
| SEQ ID | 1152 | GKIPG |
| SEQ ID | 1153 | KIPGM |
| SEQ ID | 1154 | IPGMV |
| SEQ ID | 1155 | PGMVV |
| SEQ ID | 1156 | GMVVD |
| SEQ ID | 1157 | MVVDR |
| SEQ ID | 1158 | VVDRC |
| SEQ ID | 1159 | VDRCG |
| SEQ ID | 1160 | DRCGC |
| SEQ ID | 1161 | RCGCS |

In some embodiments, the active agent comprises a peptide which comprises, consists essentially of, or consists of any one or more of the 6-mer amino acid sequences (SEQ ID NO: 1162-1556) listed in Table 5.

TABLE 5

| | | |
|---|---|---|
| SEQ ID | 1162 | MVLAAP |
| SEQ ID | 1163 | VLAAPL |
| SEQ ID | 1164 | LAAPLL |
| SEQ ID | 1165 | AAPLLL |
| SEQ ID | 1166 | APLLLG |
| SEQ ID | 1167 | PLLLGF |
| SEQ ID | 1168 | LLLGFL |
| SEQ ID | 1169 | LLGFLL |
| SEQ ID | 1170 | LGFLLL |
| SEQ ID | 1171 | GFLLLA |
| SEQ ID | 1172 | FLLLAL |
| SEQ ID | 1173 | LLLALE |
| SEQ ID | 1174 | LLALEL |
| SEQ ID | 1175 | LALELR |
| SEQ ID | 1176 | ALELRP |
| SEQ ID | 1177 | LELRPR |
| SEQ ID | 1178 | ELRPRG |
| SEQ ID | 1179 | LRPRGE |
| SEQ ID | 1180 | RPRGEA |
| SEQ ID | 1181 | PRGEAA |
| SEQ ID | 1182 | RGEAAE |
| SEQ ID | 1183 | GEAAEG |
| SEQ ID | 1184 | EAAEGP |
| SEQ ID | 1185 | AAEGPA |
| SEQ ID | 1186 | AEGPAA |
| SEQ ID | 1187 | EGPAAA |
| SEQ ID | 1188 | GPAAAA |
| SEQ ID | 1189 | PAAAAA |
| SEQ ID | 1190 | AAAAAA |
| SEQ ID | 1191 | AAAAAG |
| SEQ ID | 1192 | AAAAGV |
| SEQ ID | 1193 | AAAGVG |
| SEQ ID | 1194 | AAGVGG |
| SEQ ID | 1195 | AGVGGE |
| SEQ ID | 1196 | GVGGER |
| SEQ ID | 1197 | VGGERS |
| SEQ ID | 1198 | GGERSS |
| SEQ ID | 1199 | GERSSR |
| SEQ ID | 1200 | ERSSRP |
| SEQ ID | 1201 | RSSRPA |
| SEQ ID | 1202 | SSRPAP |
| SEQ ID | 1203 | SRPAPS |
| SEQ ID | 1204 | RPAPSV |
| SEQ ID | 1205 | PAPSVA |
| SEQ ID | 1206 | APSVAP |
| SEQ ID | 1207 | PSVAPE |
| SEQ ID | 1208 | SVAPEP |
| SEQ ID | 1209 | VAPEPD |
| SEQ ID | 1210 | APEPDG |
| SEQ ID | 1211 | PEPDGC |
| SEQ ID | 1212 | EPDGCP |
| SEQ ID | 1213 | PDGCPV |
| SEQ ID | 1214 | DGCPVC |
| SEQ ID | 1215 | GCPVCV |
| SEQ ID | 1216 | CPVCVW |
| SEQ ID | 1217 | PVCVWR |
| SEQ ID | 1218 | VCVWRQ |
| SEQ ID | 1219 | CVWRQH |
| SEQ ID | 1220 | VWRQHS |
| SEQ ID | 1221 | WRQHSR |
| SEQ ID | 1222 | RQHSRE |
| SEQ ID | 1223 | QHSREL |
| SEQ ID | 1224 | HSRELR |
| SEQ ID | 1225 | SRELRL |
| SEQ ID | 1226 | RELRLE |
| SEQ ID | 1227 | ELRLES |
| SEQ ID | 1228 | LRLESI |
| SEQ ID | 1229 | RLESIK |
| SEQ ID | 1230 | LESIKS |
| SEQ ID | 1231 | ESIKSQ |
| SEQ ID | 1232 | SIKSQI |
| SEQ ID | 1233 | IKSQIL |
| SEQ ID | 1234 | KSQILS |
| SEQ ID | 1235 | SQILSK |
| SEQ ID | 1236 | QILSKL |
| SEQ ID | 1237 | ILSKLR |
| SEQ ID | 1238 | LSKLRL |
| SEQ ID | 1239 | SKLRLK |
| SEQ ID | 1240 | KLRLKE |
| SEQ ID | 1241 | LRLKEA |
| SEQ ID | 1242 | RLKEAP |
| SEQ ID | 1243 | LKEAPN |
| SEQ ID | 1244 | KEAPNI |
| SEQ ID | 1245 | EAPNIS |
| SEQ ID | 1246 | APNISR |
| SEQ ID | 1247 | PNISRE |
| SEQ ID | 1248 | NISREV |
| SEQ ID | 1249 | ISREVV |
| SEQ ID | 1250 | SREVVK |
| SEQ ID | 1251 | REVVKQ |
| SEQ ID | 1252 | EVVKQL |
| SEQ ID | 1253 | VVKQLL |
| SEQ ID | 1254 | VKQLLP |
| SEQ ID | 1255 | KQLLPK |
| SEQ ID | 1256 | QLLPKA |
| SEQ ID | 1257 | LLPKAP |
| SEQ ID | 1258 | LPKAPP |
| SEQ ID | 1259 | PKAPPL |
| SEQ ID | 1260 | KAPPLQ |
| SEQ ID | 1261 | APPLQQ |
| SEQ ID | 1262 | PPLQQI |
| SEQ ID | 1263 | PLQQIL |
| SEQ ID | 1264 | LQQILD |
| SEQ ID | 1265 | QQILDL |
| SEQ ID | 1266 | QILDLH |
| SEQ ID | 1267 | ILDLHD |
| SEQ ID | 1268 | LDLHDF |
| SEQ ID | 1269 | DLHDFQ |
| SEQ ID | 1270 | LHDFQG |
| SEQ ID | 1271 | HDFQGD |
| SEQ ID | 1272 | DFQGDA |
| SEQ ID | 1273 | FQGDAL |
| SEQ ID | 1274 | QGDALQ |
| SEQ ID | 1275 | GDALQP |
| SEQ ID | 1276 | DALQPE |
| SEQ ID | 1277 | ALQPED |
| SEQ ID | 1278 | LQPEDF |
| SEQ ID | 1279 | QPEDFL |
| SEQ ID | 1280 | PEDFLE |
| SEQ ID | 1281 | EDFLEE |
| SEQ ID | 1282 | DFLEED |
| SEQ ID | 1283 | FLEEDE |
| SEQ ID | 1284 | LEEDEY |
| SEQ ID | 1285 | EEDEYH |
| SEQ ID | 1286 | EDEYHA |
| SEQ ID | 1287 | DEYHAT |
| SEQ ID | 1288 | EYHATT |
| SEQ ID | 1289 | YHATTE |
| SEQ ID | 1290 | HATTET |
| SEQ ID | 1291 | ATTETV |
| SEQ ID | 1292 | TTETVI |
| SEQ ID | 1293 | TETVIS |
| SEQ ID | 1294 | ETVISM |
| SEQ ID | 1295 | TVISMA |
| SEQ ID | 1296 | VISMAQ |
| SEQ ID | 1297 | ISMAQE |
| SEQ ID | 1298 | SMAQET |
| SEQ ID | 1299 | MAQETD |
| SEQ ID | 1300 | AQETDP |
| SEQ ID | 1301 | QETDPA |
| SEQ ID | 1302 | ETDPAV |
| SEQ ID | 1303 | TDPAVQ |
| SEQ ID | 1304 | DPAVQT |
| SEQ ID | 1305 | PAVQTD |
| SEQ ID | 1306 | AVQTDG |
| SEQ ID | 1307 | VQTDGS |
| SEQ ID | 1308 | QTDGSP |
| SEQ ID | 1309 | TDGSPL |
| SEQ ID | 1310 | DGSPLC |
| SEQ ID | 1311 | GSPLCC |
| SEQ ID | 1312 | SPLCCH |
| SEQ ID | 1313 | PLCCHF |
| SEQ ID | 1314 | LCCHFH |
| SEQ ID | 1315 | CCHFHF |
| SEQ ID | 1316 | CHFHFS |
| SEQ ID | 1317 | HFHFSP |
| SEQ ID | 1318 | FHFSPK |
| SEQ ID | 1319 | HFSPKV |
| SEQ ID | 1320 | FSPKVM |
| SEQ ID | 1321 | SPKVMF |
| SEQ ID | 1322 | PKVMFT |
| SEQ ID | 1323 | KVMFTK |
| SEQ ID | 1324 | VMFTKV |
| SEQ ID | 1325 | MFTKVL |
| SEQ ID | 1326 | FTKVLK |
| SEQ ID | 1327 | TKVLKA |
| SEQ ID | 1328 | KVLKAQ |
| SEQ ID | 1329 | VLKAQL |
| SEQ ID | 1330 | LKAQLW |
| SEQ ID | 1331 | KAQLWV |
| SEQ ID | 1332 | AQLWVY |
| SEQ ID | 1333 | QLWVYL |
| SEQ ID | 1334 | LWVYLR |
| SEQ ID | 1335 | WVYLRP |
| SEQ ID | 1336 | VYLRPV |
| SEQ ID | 1337 | YLRPVP |
| SEQ ID | 1338 | LRPVPR |
| SEQ ID | 1339 | RPVPRP |
| SEQ ID | 1340 | PVPRPA |
| SEQ ID | 1341 | VPRPAT |
| SEQ ID | 1342 | PRPATV |
| SEQ ID | 1343 | RPATVY |
| SEQ ID | 1344 | PATVYL |
| SEQ ID | 1345 | ATVYLQ |
| SEQ ID | 1346 | TVYLQI |
| SEQ ID | 1347 | VYLQIL |
| SEQ ID | 1348 | YLQILR |
| SEQ ID | 1349 | LQILRL |
| SEQ ID | 1350 | QILRLK |
| SEQ ID | 1351 | ILRLKP |
| SEQ ID | 1352 | LRLKPL |
| SEQ ID | 1353 | RLKPLT |
| SEQ ID | 1354 | LKPLTG |
| SEQ ID | 1355 | KPLTGE |
| SEQ ID | 1356 | PLTGEG |
| SEQ ID | 1357 | LTGEGT |
| SEQ ID | 1358 | TGEGTA |
| SEQ ID | 1359 | GEGTAG |
| SEQ ID | 1360 | EGTAGG |
| SEQ ID | 1361 | GTAGGG |
| SEQ ID | 1362 | TAGGGG |
| SEQ ID | 1363 | AGGGGG |
| SEQ ID | 1364 | GGGGGG |
| SEQ ID | 1365 | GGGGGR |
| SEQ ID | 1366 | GGGGRR |
| SEQ ID | 1367 | GGGRRH |
| SEQ ID | 1368 | GGRRHI |
| SEQ ID | 1369 | GRRHIR |
| SEQ ID | 1370 | RRHIRI |
| SEQ ID | 1371 | RHIRIR |
| SEQ ID | 1372 | HIRIRS |
| SEQ ID | 1373 | IRIRSL |
| SEQ ID | 1374 | RIRSLK |
| SEQ ID | 1375 | IRSLKI |
| SEQ ID | 1376 | RSLKIE |
| SEQ ID | 1377 | SLKIEL |
| SEQ ID | 1378 | LKIELH |
| SEQ ID | 1379 | KIELHS |
| SEQ ID | 1380 | IELHSR |
| SEQ ID | 1381 | ELHSRS |
| SEQ ID | 1382 | LHSRSG |
| SEQ ID | 1383 | HSRSGH |
| SEQ ID | 1384 | SRSGHW |
| SEQ ID | 1385 | RSGHWQ |
| SEQ ID | 1386 | SGHWQS |
| SEQ ID | 1387 | GHWQSI |
| SEQ ID | 1388 | HWQSID |
| SEQ ID | 1389 | WQSIDF |
| SEQ ID | 1390 | QSIDFK |
| SEQ ID | 1391 | SIDFKQ |
| SEQ ID | 1392 | IDFKQV |
| SEQ ID | 1393 | DFKQVL |
| SEQ ID | 1394 | FKQVLH |
| SEQ ID | 1395 | KQVLHS |
| SEQ ID | 1396 | QVLHSW |
| SEQ ID | 1397 | VLHSWF |
| SEQ ID | 1398 | LHSWFR |
| SEQ ID | 1399 | HSWFRQ |
| SEQ ID | 1400 | SWFRQP |
| SEQ ID | 1401 | WFRQPQ |
| SEQ ID | 1402 | FRQPQS |
| SEQ ID | 1403 | RQPQSN |
| SEQ ID | 1404 | QPQSNW |
| SEQ ID | 1405 | PQSNWG |
| SEQ ID | 1406 | QSNWGI |
| SEQ ID | 1407 | SNWGIE |
| SEQ ID | 1408 | NWGIEI |
| SEQ ID | 1409 | WGIEIN |
| SEQ ID | 1410 | GIEINA |
| SEQ ID | 1411 | IEINAF |
| SEQ ID | 1412 | EINAFD |
| SEQ ID | 1413 | INAFDP |
| SEQ ID | 1414 | NAFDPS |
| SEQ ID | 1415 | AFDPSG |
| SEQ ID | 1416 | FDPSGT |
| SEQ ID | 1417 | DPSGTD |
| SEQ ID | 1418 | PSGTDL |
| SEQ ID | 1419 | SGTDLA |
| SEQ ID | 1420 | GTDLAV |
| SEQ ID | 1421 | TDLAVT |
| SEQ ID | 1422 | DLAVTS |
| SEQ ID | 1423 | LAVTSL |
| SEQ ID | 1424 | AVTSLG |
| SEQ ID | 1425 | VTSLGP |
| SEQ ID | 1426 | TSLGPG |
| SEQ ID | 1427 | SLGPGA |
| SEQ ID | 1428 | LGPGAE |
| SEQ ID | 1429 | GPGAEG |
| SEQ ID | 1430 | PGAEGL |
| SEQ ID | 1431 | GAEGLH |
| SEQ ID | 1432 | AEGLHP |
| SEQ ID | 1433 | EGLHPF |
| SEQ ID | 1434 | GLHPFM |
| SEQ ID | 1435 | LHPFME |
| SEQ ID | 1436 | HPFMEL |
| SEQ ID | 1437 | PFMELR |
| SEQ ID | 1438 | FMELRV |
| SEQ ID | 1439 | MELRVL |
| SEQ ID | 1440 | ELRVLE |
| SEQ ID | 1441 | LRVLEN |
| SEQ ID | 1442 | RVLENT |
| SEQ ID | 1443 | VLENTK |
| SEQ ID | 1444 | LENTKR |
| SEQ ID | 1445 | ENTKRS |
| SEQ ID | 1446 | NTKRSR |
| SEQ ID | 1447 | TKRSRR |
| SEQ ID | 1448 | KRSRRN |
| SEQ ID | 1449 | RSRRNL |
| SEQ ID | 1450 | SRRNLG |
| SEQ ID | 1451 | RRNLGL |
| SEQ ID | 1452 | RNLGLD |
| SEQ ID | 1453 | NLGLDC |
| SEQ ID | 1454 | LGLDCD |
| SEQ ID | 1455 | GLDCDE |
| SEQ ID | 1456 | LDCDEH |
| SEQ ID | 1457 | DCDEHS |
| SEQ ID | 1458 | CDEHSS |
| SEQ ID | 1459 | DEHSSE |
| SEQ ID | 1460 | EHSSES |
| SEQ ID | 1461 | HSSESR |
| SEQ ID | 1462 | SSESRC |
| SEQ ID | 1463 | SESRCC |
| SEQ ID | 1464 | ESRCCR |
| SEQ ID | 1465 | SRCCRY |
| SEQ ID | 1466 | RCCRYP |
| SEQ ID | 1467 | CCRYPL |
| SEQ ID | 1468 | CRYPLT |
| SEQ ID | 1469 | RYPLTV |
| SEQ ID | 1470 | YPLTVD |
| SEQ ID | 1471 | PLTVDF |
| SEQ ID | 1472 | LTVDFE |
| SEQ ID | 1473 | TVDFEA |
| SEQ ID | 1474 | VDFEAF |
| SEQ ID | 1475 | DFEAFG |
| SEQ ID | 1476 | FEAFGW |
| SEQ ID | 1477 | EAFGWD |
| SEQ ID | 1478 | AFGWDW |
| SEQ ID | 1479 | FGWDWI |
| SEQ ID | 1480 | GWDWII |
| SEQ ID | 1481 | WDWIIA |
| SEQ ID | 1482 | DWIIAP |
| SEQ ID | 1483 | WIIAPK |
| SEQ ID | 1484 | IIAPKR |
| SEQ ID | 1485 | IAPKRY |
| SEQ ID | 1486 | APKRYK |
| SEQ ID | 1487 | PKRYKA |
| SEQ ID | 1488 | KRYKAN |
| SEQ ID | 1489 | RYKANY |
| SEQ ID | 1490 | YKANYC |
| SEQ ID | 1491 | KANYCS |
| SEQ ID | 1492 | ANYCSG |
| SEQ ID | 1493 | NYCSGQ |
| SEQ ID | 1494 | YCSGQC |
| SEQ ID | 1495 | CSGQCE |
| SEQ ID | 1496 | SGQCEY |
| SEQ ID | 1497 | GQCEYM |
| SEQ ID | 1498 | QCEYMF |
| SEQ ID | 1499 | CEYMFM |
| SEQ ID | 1500 | EYMFMQ |
| SEQ ID | 1501 | YMFMQK |
| SEQ ID | 1502 | MFMQKY |
| SEQ ID | 1503 | FMQKYP |
| SEQ ID | 1504 | MQKYPH |
| SEQ ID | 1505 | QKYPHT |
| SEQ ID | 1506 | KYPHTH |
| SEQ ID | 1507 | YPHTHL |
| SEQ ID | 1508 | PHTHLV |
| SEQ ID | 1509 | HTHLVQ |
| SEQ ID | 1510 | THLVQQ |
| SEQ ID | 1511 | HLVQQA |
| SEQ ID | 1512 | LVQQAN |
| SEQ ID | 1513 | VQQANP |
| SEQ ID | 1514 | QQANPR |
| SEQ ID | 1515 | QANPRG |
| SEQ ID | 1516 | ANPRGS |
| SEQ ID | 1517 | NPRGSA |
| SEQ ID | 1518 | PRGSAG |
| SEQ ID | 1519 | RGSAGP |
| SEQ ID | 1520 | GSAGPC |
| SEQ ID | 1521 | SAGPCC |
| SEQ ID | 1522 | AGPCCT |
| SEQ ID | 1523 | GPCCTP |
| SEQ ID | 1524 | PCCTPT |
| SEQ ID | 1525 | CCTPTK |
| SEQ ID | 1526 | CTPTKM |
| SEQ ID | 1527 | TPTKMS |
| SEQ ID | 1528 | PTKMSP |
| SEQ ID | 1529 | TKMSPI |
| SEQ ID | 1530 | KMSPIN |
| SEQ ID | 1531 | MSPINM |
| SEQ ID | 1532 | SPINML |
| SEQ ID | 1533 | PINMLY |
| SEQ ID | 1534 | INMLYF |
| SEQ ID | 1535 | NMLYFN |
| SEQ ID | 1536 | MLYFND |
| SEQ ID | 1537 | LYFNDK |
| SEQ ID | 1538 | YFNDKQ |
| SEQ ID | 1539 | FNDKQQ |
| SEQ ID | 1540 | NDKQQI |
| SEQ ID | 1541 | DKQQII |
| SEQ ID | 1542 | KQQIIY |
| SEQ ID | 1543 | QQIIYG |
| SEQ ID | 1544 | QIIYGK |
| SEQ ID | 1545 | IIYGKI |
| SEQ ID | 1546 | IYGKIP |
| SEQ ID | 1547 | YGKIPG |
| SEQ ID | 1548 | GKIPGM |
| SEQ ID | 1549 | KIPGMV |
| SEQ ID | 1550 | IPGMVV |
| SEQ ID | 1551 | PGMVVD |
| SEQ ID | 1552 | GMVVDR |
| SEQ ID | 1553 | MVVDRC |
| SEQ ID | 1554 | VVDRCG |
| SEQ ID | 1555 | VDRCGC |
| SEQ ID | 1556 | DRCGCS |

In some embodiments, the active agent comprises a peptide which comprises, consists essentially of, or consists of any one or more of the 7-mer amino acid sequences (SEQ ID NO: 1557-1951) listed in Table 6.

TABLE 6

| | | |
|---|---|---|
| SEQ ID | 1557 | MVLAAPL |
| SEQ ID | 1558 | VLAAPLL |
| SEQ ID | 1559 | LAAPLLL |
| SEQ ID | 1560 | AAPLLLG |
| SEQ ID | 1561 | APLLLGF |
| SEQ ID | 1562 | PLLLGFL |
| SEQ ID | 1563 | LLLGFLL |
| SEQ ID | 1564 | LLGFLLL |
| SEQ ID | 1565 | LGFLLLA |
| SEQ ID | 1566 | GFLLLAL |
| SEQ ID | 1567 | FLLLALE |
| SEQ ID | 1568 | LLLALEL |
| SEQ ID | 1569 | LLALELR |
| SEQ ID | 1570 | LALELRP |
| SEQ ID | 1571 | ALELRPR |
| SEQ ID | 1572 | LELRPRG |
| SEQ ID | 1573 | ELRPRGE |
| SEQ ID | 1574 | LRPRGEA |
| SEQ ID | 1575 | RPRGEAA |
| SEQ ID | 1576 | PRGEAAE |
| SEQ ID | 1577 | RGEAAEG |
| SEQ ID | 1578 | GEAAEGP |
| SEQ ID | 1579 | EAAEGPA |
| SEQ ID | 1580 | AAEGPAA |
| SEQ ID | 1581 | AEGPAAA |
| SEQ ID | 1582 | EGPAAAA |
| SEQ ID | 1583 | GPAAAAA |
| SEQ ID | 1584 | PAAAAAA |
| SEQ ID | 1585 | AAAAAAA |
| SEQ ID | 1586 | AAAAAAG |
| SEQ ID | 1587 | AAAAAGV |
| SEQ ID | 1588 | AAAAGVG |
| SEQ ID | 1589 | AAAGVGG |
| SEQ ID | 1590 | AAGVGGE |
| SEQ ID | 1591 | AGVGGER |
| SEQ ID | 1592 | GVGGERS |
| SEQ ID | 1593 | VGGERSS |
| SEQ ID | 1594 | GGERSSR |
| SEQ ID | 1595 | GERSSRP |
| SEQ ID | 1596 | ERSSRPA |
| SEQ ID | 1597 | RSSRPAP |
| SEQ ID | 1598 | SSRPAPS |
| SEQ ID | 1599 | SRPAPSV |
| SEQ ID | 1600 | RPAPSVA |
| SEQ ID | 1601 | PAPSVAP |
| SEQ ID | 1602 | APSVAPE |
| SEQ ID | 1603 | PSVAPEP |
| SEQ ID | 1604 | SVAPEPD |
| SEQ ID | 1605 | VAPEPDG |
| SEQ ID | 1606 | APEPDGC |
| SEQ ID | 1607 | PEPDGCP |
| SEQ ID | 1608 | EPDGCPV |
| SEQ ID | 1609 | PDGCPVC |
| SEQ ID | 1610 | DGCPVCV |
| SEQ ID | 1611 | GCPVCVW |
| SEQ ID | 1612 | CPVCVWR |
| SEQ ID | 1613 | PVCVWRQ |
| SEQ ID | 1614 | VCVWRQH |
| SEQ ID | 1615 | CVWRQHS |
| SEQ ID | 1616 | VWRQHSR |
| SEQ ID | 1617 | WRQHSRE |
| SEQ ID | 1618 | RQHSREL |
| SEQ ID | 1619 | QHSRELR |
| SEQ ID | 1620 | HSRELRL |
| SEQ ID | 1621 | SRELRLE |
| SEQ ID | 1622 | RELRLES |
| SEQ ID | 1623 | ELRLESI |
| SEQ ID | 1624 | LRLESIK |
| SEQ ID | 1625 | RLESIKS |
| SEQ ID | 1626 | LESIKSQ |
| SEQ ID | 1627 | ESIKSQI |
| SEQ ID | 1628 | SIKSQIL |
| SEQ ID | 1629 | IKSQILS |
| SEQ ID | 1630 | KSQILSK |
| SEQ ID | 1631 | SQILSKL |
| SEQ ID | 1632 | QILSKLR |
| SEQ ID | 1633 | ILSKLRL |
| SEQ ID | 1634 | LSKLRLK |
| SEQ ID | 1635 | SKLRLKE |
| SEQ ID | 1636 | KLRLKEA |
| SEQ ID | 1637 | LRLKEAP |
| SEQ ID | 1638 | RLKEAPN |
| SEQ ID | 1639 | LKEAPNI |
| SEQ ID | 1640 | KEAPNIS |
| SEQ ID | 1641 | EAPNISR |
| SEQ ID | 1642 | APNISRE |
| SEQ ID | 1643 | PNISREV |
| SEQ ID | 1644 | NISREVV |
| SEQ ID | 1645 | ISREVVK |
| SEQ ID | 1646 | SREVVKQ |
| SEQ ID | 1647 | REVVKQL |
| SEQ ID | 1648 | EVVKQLL |
| SEQ ID | 1649 | VVKQLLP |
| SEQ ID | 1650 | VKQLLPK |
| SEQ ID | 1651 | KQLLPKA |
| SEQ ID | 1652 | QLLPKAP |
| SEQ ID | 1653 | LLPKAPP |
| SEQ ID | 1654 | LPKAPPL |
| SEQ ID | 1655 | PKAPPLQ |
| SEQ ID | 1656 | KAPPLQQ |
| SEQ ID | 1657 | APPLQQI |
| SEQ ID | 1658 | PPLQQIL |
| SEQ ID | 1659 | PLQQILD |
| SEQ ID | 1660 | LQQILDL |
| SEQ ID | 1661 | QQILDLH |
| SEQ ID | 1662 | QILDLHD |
| SEQ ID | 1663 | ILDLHDF |
| SEQ ID | 1664 | LDLHDFQ |
| SEQ ID | 1665 | DLHDFQG |
| SEQ ID | 1666 | LHDFQGD |
| SEQ ID | 1667 | HDFQGDA |
| SEQ ID | 1668 | DFQGDAL |
| SEQ ID | 1669 | FQGDALQ |
| SEQ ID | 1670 | QGDALQP |
| SEQ ID | 1671 | GDALQPE |
| SEQ ID | 1672 | DALQPED |
| SEQ ID | 1673 | ALQPEDF |
| SEQ ID | 1674 | LQPEDFL |
| SEQ ID | 1675 | QPEDFLE |
| SEQ ID | 1676 | PEDFLEE |
| SEQ ID | 1677 | EDFLEED |
| SEQ ID | 1678 | DFLEEDE |
| SEQ ID | 1679 | FLEEDEY |
| SEQ ID | 1680 | LEEDEYH |
| SEQ ID | 1681 | EEDEYHA |
| SEQ ID | 1682 | EDEYHAT |
| SEQ ID | 1683 | DEYHATT |
| SEQ ID | 1684 | EYHATTE |
| SEQ ID | 1685 | YHATTET |
| SEQ ID | 1686 | HATTETV |
| SEQ ID | 1687 | ATTETVI |
| SEQ ID | 1688 | TTETVIS |
| SEQ ID | 1689 | TETVISM |
| SEQ ID | 1690 | ETVISMA |
| SEQ ID | 1691 | TVISMAQ |
| SEQ ID | 1692 | VISMAQE |
| SEQ ID | 1693 | ISMAQET |
| SEQ ID | 1694 | SMAQETD |
| SEQ ID | 1695 | MAQETDP |
| SEQ ID | 1696 | AQETDPA |
| SEQ ID | 1697 | QETDPAV |
| SEQ ID | 1698 | ETDPAVQ |
| SEQ ID | 1699 | TDPAVQT |
| SEQ ID | 1700 | DPAVQTD |
| SEQ ID | 1701 | PAVQTDG |
| SEQ ID | 1702 | AVQTDGS |
| SEQ ID | 1703 | VQTDGSP |
| SEQ ID | 1704 | QTDGSPL |
| SEQ ID | 1705 | TDGSPLC |
| SEQ ID | 1706 | DGSPLCC |
| SEQ ID | 1707 | GSPLCCH |
| SEQ ID | 1708 | SPLCCHF |
| SEQ ID | 1709 | PLCCHFH |
| SEQ ID | 1710 | LCCHFHF |
| SEQ ID | 1711 | CCHFHFS |
| SEQ ID | 1712 | CHFHFSP |
| SEQ ID | 1713 | HFHFSPK |
| SEQ ID | 1714 | FHFSPKV |
| SEQ ID | 1715 | HFSPKVM |
| SEQ ID | 1716 | FSPKVMF |
| SEQ ID | 1717 | SPKVMFT |
| SEQ ID | 1718 | PKVMFTK |
| SEQ ID | 1719 | KVMFTKV |
| SEQ ID | 1720 | VMFTKVL |
| SEQ ID | 1721 | MFTKVLK |
| SEQ ID | 1722 | FTKVLKA |
| SEQ ID | 1723 | TKVLKAQ |
| SEQ ID | 1724 | KVLKAQL |
| SEQ ID | 1725 | VLKAQLW |
| SEQ ID | 1726 | LKAQLWV |
| SEQ ID | 1727 | KAQLWVY |
| SEQ ID | 1728 | AQLWVYL |
| SEQ ID | 1729 | QLWVYLR |
| SEQ ID | 1730 | LWVYLRP |
| SEQ ID | 1731 | WVYLRPV |
| SEQ ID | 1732 | VYLRPVP |
| SEQ ID | 1733 | YLRPVPR |
| SEQ ID | 1734 | LRPVPRP |
| SEQ ID | 1735 | RPVPRPA |
| SEQ ID | 1736 | PVPRPAT |
| SEQ ID | 1737 | VPRPATV |
| SEQ ID | 1738 | PRPATVY |
| SEQ ID | 1739 | RPATVYL |
| SEQ ID | 1740 | PATVYLQ |
| SEQ ID | 1741 | ATVYLQI |
| SEQ ID | 1742 | TVYLQIL |
| SEQ ID | 1743 | VYLQILR |
| SEQ ID | 1744 | YLQILRL |
| SEQ ID | 1745 | LQILRLK |
| SEQ ID | 1746 | QILRLKP |
| SEQ ID | 1747 | ILRLKPL |
| SEQ ID | 1748 | LRLKPLT |
| SEQ ID | 1749 | RLKPLTG |
| SEQ ID | 1750 | LKPLTGE |
| SEQ ID | 1751 | KPLTGEG |
| SEQ ID | 1752 | PLTGEGT |
| SEQ ID | 1753 | LTGEGTA |
| SEQ ID | 1754 | TGEGTAG |
| SEQ ID | 1755 | GEGTAGG |
| SEQ ID | 1756 | EGTAGGG |
| SEQ ID | 1757 | GTAGGGG |
| SEQ ID | 1758 | TAGGGGG |
| SEQ ID | 1759 | AGGGGGG |
| SEQ ID | 1760 | GGGGGGR |
| SEQ ID | 1761 | GGGGGRR |
| SEQ ID | 1762 | GGGGRRH |
| SEQ ID | 1763 | GGGRRHI |
| SEQ ID | 1764 | GGRRHIR |
| SEQ ID | 1765 | GRRHIRI |
| SEQ ID | 1766 | RRHIRIR |
| SEQ ID | 1767 | RHIRIRS |
| SEQ ID | 1768 | HIRIRSL |
| SEQ ID | 1769 | IRIRSLK |
| SEQ ID | 1770 | RIRSLKI |
| SEQ ID | 1771 | IRSLKIE |
| SEQ ID | 1772 | RSLKIEL |
| SEQ ID | 1773 | SLKIELH |
| SEQ ID | 1774 | LKIELHS |
| SEQ ID | 1775 | KIELHSR |
| SEQ ID | 1776 | IELHSRS |
| SEQ ID | 1777 | ELHSRSG |
| SEQ ID | 1778 | LHSRSGH |
| SEQ ID | 1779 | HSRSGHW |
| SEQ ID | 1780 | SRSGHWQ |
| SEQ ID | 1781 | RSGHWQS |
| SEQ ID | 1782 | SGHWQSI |
| SEQ ID | 1783 | GHWQSID |
| SEQ ID | 1784 | HWQSIDF |
| SEQ ID | 1785 | WQSIDFK |
| SEQ ID | 1786 | QSIDFKQ |
| SEQ ID | 1787 | SIDFKQV |
| SEQ ID | 1788 | IDFKQVL |
| SEQ ID | 1789 | DFKQVLH |
| SEQ ID | 1790 | FKQVLHS |
| SEQ ID | 1791 | KQVLHSW |
| SEQ ID | 1792 | QVLHSWF |
| SEQ ID | 1793 | VLHSWFR |
| SEQ ID | 1794 | LHSWFRQ |
| SEQ ID | 1795 | HSWFRQP |
| SEQ ID | 1796 | SWFRQPQ |
| SEQ ID | 1797 | WFRQPQS |
| SEQ ID | 1798 | FRQPQSN |
| SEQ ID | 1799 | RQPQSNW |
| SEQ ID | 1800 | QPQSNWG |
| SEQ ID | 1801 | PQSNWGI |
| SEQ ID | 1802 | QSNWGIE |
| SEQ ID | 1803 | SNWGIEI |
| SEQ ID | 1804 | NWGIEIN |
| SEQ ID | 1805 | WGIEINA |
| SEQ ID | 1806 | GIEINAF |
| SEQ ID | 1807 | IEINAFD |
| SEQ ID | 1808 | EINAFDP |
| SEQ ID | 1809 | INAFDPS |
| SEQ ID | 1810 | NAFDPSG |
| SEQ ID | 1811 | AFDPSGT |
| SEQ ID | 1812 | FDPSGTD |
| SEQ ID | 1813 | DPSGTDL |
| SEQ ID | 1814 | PSGTDLA |
| SEQ ID | 1815 | SGTDLAV |
| SEQ ID | 1816 | GTDLAVT |
| SEQ ID | 1817 | TDLAVTS |
| SEQ ID | 1818 | DLAVTSL |
| SEQ ID | 1819 | LAVTSLG |
| SEQ ID | 1820 | AVTSLGP |
| SEQ ID | 1821 | VTSLGPG |
| SEQ ID | 1822 | TSLGPGA |
| SEQ ID | 1823 | SLGPGAE |
| SEQ ID | 1824 | LGPGAEG |
| SEQ ID | 1825 | GPGAEGL |
| SEQ ID | 1826 | PGAEGLH |
| SEQ ID | 1827 | GAEGLHP |
| SEQ ID | 1828 | AEGLHPF |
| SEQ ID | 1829 | EGLHPFM |
| SEQ ID | 1830 | GLHPFME |
| SEQ ID | 1831 | LHPFMEL |
| SEQ ID | 1832 | HPFMELR |
| SEQ ID | 1833 | PFMELRV |
| SEQ ID | 1834 | FMELRVL |
| SEQ ID | 1835 | MELRVLE |
| SEQ ID | 1836 | ELRVLEN |
| SEQ ID | 1837 | LRVLENT |
| SEQ ID | 1838 | RVLENTK |
| SEQ ID | 1839 | VLENTKR |
| SEQ ID | 1840 | LENTKRS |
| SEQ ID | 1841 | ENTKRSR |
| SEQ ID | 1842 | NTKRSRR |
| SEQ ID | 1843 | TKRSRRN |
| SEQ ID | 1844 | KRSRRNL |
| SEQ ID | 1845 | RSRRNLG |
| SEQ ID | 1846 | SRRNLGL |
| SEQ ID | 1847 | RRNLGLD |
| SEQ ID | 1848 | RNLGLDC |
| SEQ ID | 1849 | NLGLDCD |
| SEQ ID | 1850 | LGLDCDE |
| SEQ ID | 1851 | GLDCDEH |
| SEQ ID | 1852 | LDCDEHS |
| SEQ ID | 1853 | DCDEHSS |
| SEQ ID | 1854 | CDEHSSE |
| SEQ ID | 1855 | DEHSSES |
| SEQ ID | 1856 | EHSSESR |
| SEQ ID | 1857 | HSSESRC |
| SEQ ID | 1858 | SSESRCC |
| SEQ ID | 1859 | SESRCCR |
| SEQ ID | 1860 | ESRCCRY |
| SEQ ID | 1861 | SRCCRYP |
| SEQ ID | 1862 | RCCRYPL |
| SEQ ID | 1863 | CCRYPLT |
| SEQ ID | 1864 | CRYPLTV |
| SEQ ID | 1865 | RYPLTVD |
| SEQ ID | 1866 | YPLTVDF |
| SEQ ID | 1867 | PLTVDFE |
| SEQ ID | 1868 | LTVDFEA |
| SEQ ID | 1869 | TVDFEAF |
| SEQ ID | 1870 | VDFEAFG |
| SEQ ID | 1871 | DFEAFGW |
| SEQ ID | 1872 | FEAFGWD |
| SEQ ID | 1873 | EAFGWDW |
| SEQ ID | 1874 | AFGWDWI |
| SEQ ID | 1875 | FGWDWII |
| SEQ ID | 1876 | GWDWIIA |
| SEQ ID | 1877 | WDWIIAP |
| SEQ ID | 1878 | DWIIAPK |
| SEQ ID | 1879 | WIIAPKR |
| SEQ ID | 1880 | IIAPKRY |
| SEQ ID | 1881 | IAPKRYK |
| SEQ ID | 1882 | APKRYKA |
| SEQ ID | 1883 | PKRYKAN |
| SEQ ID | 1884 | KRYKANY |
| SEQ ID | 1885 | RYKANYC |
| SEQ ID | 1886 | YKANYCS |
| SEQ ID | 1887 | KANYCSG |
| SEQ ID | 1888 | ANYCSGQ |
| SEQ ID | 1889 | NYCSGQC |
| SEQ ID | 1890 | YCSGQCE |
| SEQ ID | 1891 | CSGQCEY |
| SEQ ID | 1892 | SGQCEYM |
| SEQ ID | 1893 | GQCEYMF |
| SEQ ID | 1894 | QCEYMFM |
| SEQ ID | 1895 | CEYMFMQ |
| SEQ ID | 1896 | EYMFMQK |
| SEQ ID | 1897 | YMFMQKY |
| SEQ ID | 1898 | MFMQKYP |
| SEQ ID | 1899 | FMQKYPH |
| SEQ ID | 1900 | MQKYPHT |
| SEQ ID | 1901 | QKYPHTH |
| SEQ ID | 1902 | KYPHTHL |
| SEQ ID | 1903 | YPHTHLV |
| SEQ ID | 1904 | PHTHLVQ |
| SEQ ID | 1905 | HTHLVQQ |
| SEQ ID | 1906 | THLVQQA |
| SEQ ID | 1907 | HLVQQAN |
| SEQ ID | 1908 | LVQQANP |
| SEQ ID | 1909 | VQQANPR |
| SEQ ID | 1910 | QQANPRG |
| SEQ ID | 1911 | QANPRGS |
| SEQ ID | 1912 | ANPRGSA |
| SEQ ID | 1913 | NPRGSAG |
| SEQ ID | 1914 | PRGSAGP |
| SEQ ID | 1915 | RGSAGPC |
| SEQ ID | 1916 | GSAGPCC |
| SEQ ID | 1917 | SAGPCCT |
| SEQ ID | 1918 | AGPCCTP |
| SEQ ID | 1919 | GPCCTPT |
| SEQ ID | 1920 | PCCTPTK |
| SEQ ID | 1921 | CCTPTKM |
| SEQ ID | 1922 | CTPTKMS |
| SEQ ID | 1923 | TPTKMSP |
| SEQ ID | 1924 | PTKMSPI |
| SEQ ID | 1925 | TKMSPIN |
| SEQ ID | 1926 | KMSPINM |
| SEQ ID | 1927 | MSPINML |
| SEQ ID | 1928 | SPINMLY |
| SEQ ID | 1929 | PINMLYF |
| SEQ ID | 1930 | INMLYFN |
| SEQ ID | 1931 | NMLYFND |
| SEQ ID | 1932 | MLYFNDK |
| SEQ ID | 1933 | LYFNDKQ |
| SEQ ID | 1934 | YFNDKQQ |
| SEQ ID | 1935 | FNDKQQI |
| SEQ ID | 1936 | NDKQQII |
| SEQ ID | 1937 | DKQQIIY |
| SEQ ID | 1938 | KQQIIYG |
| SEQ ID | 1939 | QQIIYGK |
| SEQ ID | 1940 | QIIYGKI |
| SEQ ID | 1941 | IIYGKIP |
| SEQ ID | 1942 | IYGKIPG |
| SEQ ID | 1943 | YGKIPGM |
| SEQ ID | 1944 | GKIPGMV |
| SEQ ID | 1945 | KIPGMVV |
| SEQ ID | 1946 | IPGMVVD |
| SEQ ID | 1947 | PGMVVDR |
| SEQ ID | 1948 | GMVVDRC |
| SEQ ID | 1949 | MVVDRCG |
| SEQ ID | 1950 | VVDRCGC |
| SEQ ID | 1951 | VDRCGCS |

In some embodiments, the active agent comprises a peptide which comprises, consists essentially of, or consists of any one or more of the 8-mer amino acid sequences (SEQ ID NO: 1952-2346) listed in Table 7.

TABLE 7

| | |
|---|---|
| SEQ ID 1952 | MVLAAPLL |
| SEQ ID 1953 | VLAAPLLL |
| SEQ ID 1954 | LAAPLLLG |
| SEQ ID 1955 | AAPLLLGF |
| SEQ ID 1956 | APLLLGFL |
| SEQ ID 1957 | PLLLGFLL |
| SEQ ID 1958 | LLLGFLLL |
| SEQ ID 1959 | LLGFLLLA |
| SEQ ID 1960 | LGFLLLAL |
| SEQ ID 1961 | GFLLLALE |
| SEQ ID 1962 | FLLLALEL |
| SEQ ID 1963 | LLLALELR |
| SEQ ID 1964 | LLALELRP |
| SEQ ID 1965 | LALELRPR |
| SEQ ID 1966 | ALELRPRG |
| SEQ ID 1967 | LELRPRGE |
| SEQ ID 1968 | ELRPRGEA |
| SEQ ID 1969 | LRPRGEAA |
| SEQ ID 1970 | RPRGEAAE |
| SEQ ID 1971 | PRGEAAEG |
| SEQ ID 1972 | RGEAAEGP |
| SEQ ID 1973 | GEAAEGPA |
| SEQ ID 1974 | EAAEGPAA |
| SEQ ID 1975 | AAEGPAAA |
| SEQ ID 1976 | AEGPAAAA |
| SEQ ID 1977 | EGPAAAAA |
| SEQ ID 1978 | GPAAAAAA |
| SEQ ID 1979 | PAAAAAAA |
| SEQ ID 1980 | AAAAAAAA |
| SEQ ID 1981 | AAAAAAAG |
| SEQ ID 1982 | AAAAAAGV |
| SEQ ID 1983 | AAAAAGVG |
| SEQ ID 1984 | AAAAGVGG |
| SEQ ID 1985 | AAAGVGGE |
| SEQ ID 1986 | AAGVGGER |
| SEQ ID 1987 | AGVGGERS |
| SEQ ID 1988 | GVGGERSS |
| SEQ ID 1989 | VGGERSSR |
| SEQ ID 1990 | GGERSSRP |
| SEQ ID 1991 | GERSSRPA |
| SEQ ID 1992 | ERSSRPAP |
| SEQ ID 1993 | RSSRPAPS |
| SEQ ID 1994 | SSRPAPSV |
| SEQ ID 1995 | SRPAPSVA |
| SEQ ID 1996 | RPAPSVAP |
| SEQ ID 1997 | PAPSVAPE |
| SEQ ID 1998 | APSVAPEP |
| SEQ ID 1999 | PSVAPEPD |
| SEQ ID 2000 | SVAPEPDG |
| SEQ ID 2001 | VAPEPDGC |
| SEQ ID 2002 | APEPDGCP |
| SEQ ID 2003 | PEPDGCPV |
| SEQ ID 2004 | EPDGCPVC |
| SEQ ID 2005 | PDGCPVCV |
| SEQ ID 2006 | DGCPVCVW |
| SEQ ID 2007 | GCPVCVWR |
| SEQ ID 2008 | CPVCVWRQ |
| SEQ ID 2009 | PVCVWRQH |
| SEQ ID 2010 | VCVWRQHS |
| SEQ ID 2011 | CVWRQHSR |
| SEQ ID 2012 | VWRQHSRE |
| SEQ ID 2013 | WRQHSREL |
| SEQ ID 2014 | RQHSRELR |
| SEQ ID 2015 | QHSRELRL |
| SEQ ID 2016 | HSRELRLE |
| SEQ ID 2017 | SRELRLES |
| SEQ ID 2018 | RELRLESI |
| SEQ ID 2019 | ELRLESIK |
| SEQ ID 2020 | LRLESIKS |
| SEQ ID 2021 | RLESIKSQ |
| SEQ ID 2022 | LESIKSQI |
| SEQ ID 2023 | ESIKSQIL |
| SEQ ID 2024 | SIKSQILS |
| SEQ ID 2025 | IKSQILSK |
| SEQ ID 2026 | KSQILSKL |
| SEQ ID 2027 | SQILSKLR |
| SEQ ID 2028 | QILSKLRL |
| SEQ ID 2029 | ILSKLRLK |
| SEQ ID 2030 | LSKLRLKE |
| SEQ ID 2031 | SKLRLKEA |
| SEQ ID 2032 | KLRLKEAP |
| SEQ ID 2033 | LRLKEAPN |
| SEQ ID 2034 | RLKEAPNI |
| SEQ ID 2035 | LKEAPNIS |
| SEQ ID 2036 | KEAPNISR |
| SEQ ID 2037 | EAPNISRE |
| SEQ ID 2038 | APNISREV |
| SEQ ID 2039 | PNISREVV |
| SEQ ID 2040 | NISREVVK |
| SEQ ID 2041 | ISREVVKQ |
| SEQ ID 2042 | SREVVKQL |
| SEQ ID 2043 | REVVKQLL |
| SEQ ID 2044 | EVVKQLLP |
| SEQ ID 2045 | VVKQLLPK |
| SEQ ID 2046 | VKQLLPKA |
| SEQ ID 2047 | KQLLPKAP |
| SEQ ID 2048 | QLLPKAPP |
| SEQ ID 2049 | LLPKAPPL |
| SEQ ID 2050 | LPKAPPLQ |
| SEQ ID 2051 | PKAPPLQQ |
| SEQ ID 2052 | KAPPLQQI |
| SEQ ID 2053 | APPLQQIL |
| SEQ ID 2054 | PPLQQILD |
| SEQ ID 2055 | PLQQILDL |
| SEQ ID 2056 | LQQILDLH |
| SEQ ID 2057 | QQILDLHD |
| SEQ ID 2058 | QILDLHDF |
| SEQ ID 2059 | ILDLHDFQ |
| SEQ ID 2060 | LDLHDFQG |
| SEQ ID 2061 | DLHDFQGD |
| SEQ ID 2062 | LHDFQGDA |
| SEQ ID 2063 | HDFQGDAL |
| SEQ ID 2064 | DFQGDALQ |
| SEQ ID 2065 | FQGDALQP |
| SEQ ID 2066 | QGDALQPE |
| SEQ ID 2067 | GDALQPED |
| SEQ ID 2068 | DALQPEDF |
| SEQ ID 2069 | ALQPEDFL |
| SEQ ID 2070 | LQPEDFLE |
| SEQ ID 2071 | QPEDFLEE |
| SEQ ID 2072 | PEDFLEED |
| SEQ ID 2073 | EDFLEEDE |
| SEQ ID 2074 | DFLEEDEY |
| SEQ ID 2075 | FLEEDEYH |
| SEQ ID 2076 | LEEDEYHA |
| SEQ ID 2077 | EEDEYHAT |
| SEQ ID 2078 | EDEYHATT |
| SEQ ID 2079 | DEYHATTE |
| SEQ ID 2080 | EYHATTET |
| SEQ ID 2081 | YHATTETV |
| SEQ ID 2082 | HATTETVI |
| SEQ ID 2083 | ATTETVIS |
| SEQ ID 2084 | TTETVISM |
| SEQ ID 2085 | TETVISMA |
| SEQ ID 2086 | ETVISMAQ |
| SEQ ID 2087 | TVISMAQE |
| SEQ ID 2088 | VISMAQET |
| SEQ ID 2089 | ISMAQETD |
| SEQ ID 2090 | SMAQETDP |
| SEQ ID 2091 | MAQETDPA |
| SEQ ID 2092 | AQETDPAV |
| SEQ ID 2093 | QETDPAVQ |
| SEQ ID 2094 | ETDPAVQT |
| SEQ ID 2095 | TDPAVQTD |
| SEQ ID 2096 | DPAVQTDG |
| SEQ ID 2097 | PAVQTDGS |
| SEQ ID 2098 | AVQTDGSP |
| SEQ ID 2099 | VQTDGSPL |
| SEQ ID 2100 | QTDGSPLC |
| SEQ ID 2101 | TDGSPLCC |
| SEQ ID 2102 | DGSPLCCH |
| SEQ ID 2103 | GSPLCCHF |
| SEQ ID 2104 | SPLCCHFH |
| SEQ ID 2105 | PLCCHFHF |
| SEQ ID 2106 | LCCHFHFS |
| SEQ ID 2107 | CCHFHFSP |
| SEQ ID 2108 | CHFHFSPK |
| SEQ ID 2109 | HFHFSPKV |
| SEQ ID 2110 | FHFSPKVM |
| SEQ ID 2111 | HFSPKVMF |
| SEQ ID 2112 | FSPKVMFT |
| SEQ ID 2113 | SPKVMFTK |
| SEQ ID 2114 | PKVMFTKV |
| SEQ ID 2115 | KVMFTKVL |
| SEQ ID 2116 | VMFTKVLK |
| SEQ ID 2117 | MFTKVLKA |
| SEQ ID 2118 | FTKVLKAQ |
| SEQ ID 2119 | TKVLKAQL |
| SEQ ID 2120 | KVLKAQLW |
| SEQ ID 2121 | VLKAQLWV |
| SEQ ID 2122 | LKAQLWVY |
| SEQ ID 2123 | KAQLWVYL |
| SEQ ID 2124 | AQLWVYLR |
| SEQ ID 2125 | QLWVYLRP |
| SEQ ID 2126 | LWVYLRPV |
| SEQ ID 2127 | WVYLRPVP |
| SEQ ID 2128 | VYLRPVPR |
| SEQ ID 2129 | YLRPVPRP |
| SEQ ID 2130 | LRPVPRPA |
| SEQ ID 2131 | RPVPRPAT |
| SEQ ID 2132 | PVPRPATV |
| SEQ ID 2133 | VPRPATVY |
| SEQ ID 2134 | PRPATVYL |
| SEQ ID 2135 | RPATVYLQ |
| SEQ ID 2136 | PATVYLQI |
| SEQ ID 2137 | ATVYLQIL |
| SEQ ID 2138 | TVYLQILR |
| SEQ ID 2139 | VYLQILRL |
| SEQ ID 2140 | YLQILRLK |
| SEQ ID 2141 | LQILRLKP |
| SEQ ID 2142 | QILRLKPL |
| SEQ ID 2143 | ILRLKPLT |
| SEQ ID 2144 | LRLKPLTG |
| SEQ ID 2145 | RLKPLTGE |
| SEQ ID 2146 | LKPLTGEG |
| SEQ ID 2147 | KPLTGEGT |
| SEQ ID 2148 | PLTGEGTA |
| SEQ ID 2149 | LTGEGTAG |
| SEQ ID 2150 | TGEGTAGG |
| SEQ ID 2151 | GEGTAGGG |
| SEQ ID 2152 | EGTAGGGG |
| SEQ ID 2153 | GTAGGGGG |
| SEQ ID 2154 | TAGGGGGG |
| SEQ ID 2155 | AGGGGGGR |
| SEQ ID 2156 | GGGGGGRR |
| SEQ ID 2157 | GGGGGRRH |
| SEQ ID 2158 | GGGGRRHI |
| SEQ ID 2159 | GGGRRHIR |
| SEQ ID 2160 | GGRRHIRI |
| SEQ ID 2161 | GRRHIRIR |
| SEQ ID 2162 | RRHIRIRS |
| SEQ ID 2163 | RHIRIRSL |
| SEQ ID 2164 | HIRIRSLK |
| SEQ ID 2165 | IRIRSLKI |
| SEQ ID 2166 | RIRSLKIE |
| SEQ ID 2167 | IRSLKIEL |
| SEQ ID 2168 | RSLKIELH |
| SEQ ID 2169 | SLKIELHS |
| SEQ ID 2170 | LKIELHSR |
| SEQ ID 2171 | KIELHSRS |
| SEQ ID 2172 | IELHSRSG |
| SEQ ID 2173 | ELHSRSGH |
| SEQ ID 2174 | LHSRSGHW |
| SEQ ID 2175 | HSRSGHWQ |
| SEQ ID 2176 | SRSGHWQS |
| SEQ ID 2177 | RSGHWQSI |
| SEQ ID 2178 | SGHWQSID |
| SEQ ID 2179 | GHWQSIDF |
| SEQ ID 2180 | HWQSIDFK |
| SEQ ID 2181 | WQSIDFKQ |
| SEQ ID 2182 | QSIDFKQV |
| SEQ ID 2183 | SIDFKQVL |
| SEQ ID 2184 | IDFKQVLH |
| SEQ ID 2185 | DFKQVLHS |
| SEQ ID 2186 | FKQVLHSW |
| SEQ ID 2187 | KQVLHSWF |
| SEQ ID 2188 | QVLHSWFR |
| SEQ ID 2189 | VLHSWFRQ |
| SEQ ID 2190 | LHSWFRQP |
| SEQ ID 2191 | HSWFRQPQ |
| SEQ ID 2192 | SWFRQPQS |
| SEQ ID 2193 | WFRQPQSN |
| SEQ ID 2194 | FRQPQSNW |
| SEQ ID 2195 | RQPQSNWG |
| SEQ ID 2196 | QPQSNWGI |
| SEQ ID 2197 | PQSNWGIE |
| SEQ ID 2198 | QSNWGIEI |
| SEQ ID 2199 | SNWGIEIN |
| SEQ ID 2200 | NWGIEINA |
| SEQ ID 2201 | WGIEINAF |
| SEQ ID 2202 | GIEINAFD |
| SEQ ID 2203 | IEINAFDP |
| SEQ ID 2204 | EINAFDPS |
| SEQ ID 2205 | INAFDPSG |
| SEQ ID 2206 | NAFDPSGT |
| SEQ ID 2207 | AFDPSGTD |
| SEQ ID 2208 | FDPSGTDL |
| SEQ ID 2209 | DPSGTDLA |
| SEQ ID 2210 | PSGTDLAV |
| SEQ ID 2211 | SGTDLAVT |
| SEQ ID 2212 | GTDLAVTS |
| SEQ ID 2213 | TDLAVTSL |
| SEQ ID 2214 | DLAVTSLG |
| SEQ ID 2215 | LAVTSLGP |
| SEQ ID 2216 | AVTSLGPG |
| SEQ ID 2217 | VTSLGPGA |
| SEQ ID 2218 | TSLGPGAE |
| SEQ ID 2219 | SLGPGAEG |
| SEQ ID 2220 | LGPGAEGL |
| SEQ ID 2221 | GPGAEGLH |
| SEQ ID 2222 | PGAEGLHP |
| SEQ ID 2223 | GAEGLHPF |
| SEQ ID 2224 | AEGLHPFM |
| SEQ ID 2225 | EGLHPFME |
| SEQ ID 2226 | GLHPFMEL |
| SEQ ID 2227 | LHPFMELR |
| SEQ ID 2228 | HPFMELRV |
| SEQ ID 2229 | PFMELRVL |
| SEQ ID 2230 | FMELRVLE |
| SEQ ID 2231 | MELRVLEN |
| SEQ ID 2232 | ELRVLENT |
| SEQ ID 2233 | LRVLENTK |
| SEQ ID 2234 | RVLENTKR |
| SEQ ID 2235 | VLENTKRS |
| SEQ ID 2236 | LENTKRSR |
| SEQ ID 2237 | ENTKRSRR |
| SEQ ID 2238 | NTKRSRRN |
| SEQ ID 2239 | TKRSRRNL |
| SEQ ID 2240 | KRSRRNLG |
| SEQ ID 2241 | RSRRNLGL |
| SEQ ID 2242 | SRRNLGLD |
| SEQ ID 2243 | RRNLGLDC |
| SEQ ID 2244 | RNLGLDCD |
| SEQ ID 2245 | NLGLDCDE |
| SEQ ID 2246 | LGLDCDEH |
| SEQ ID 2247 | GLDCDEHS |
| SEQ ID 2248 | LDCDEHSS |
| SEQ ID 2249 | DCDEHSSE |
| SEQ ID 2250 | CDEHSSES |
| SEQ ID 2251 | DEHSSESR |
| SEQ ID 2252 | EHSSESRC |
| SEQ ID 2253 | HSSESRCC |
| SEQ ID 2254 | SSESRCCR |
| SEQ ID 2255 | SESRCCRY |
| SEQ ID 2256 | ESRCCRYP |
| SEQ ID 2257 | SRCCRYPL |
| SEQ ID 2258 | RCCRYPLT |
| SEQ ID 2259 | CCRYPLTV |
| SEQ ID 2260 | CRYPLTVD |
| SEQ ID 2261 | RYPLTVDF |
| SEQ ID 2262 | YPLTVDFE |
| SEQ ID 2263 | PLTVDFEA |
| SEQ ID 2264 | LTVDFEAF |
| SEQ ID 2265 | TVDFEAFG |
| SEQ ID 2266 | VDFEAFGW |
| SEQ ID 2267 | DFEAFGWD |
| SEQ ID 2268 | FEAFGWDW |
| SEQ ID 2269 | EAFGWDWI |
| SEQ ID 2270 | AFGWDWII |
| SEQ ID 2271 | FGWDWIIA |
| SEQ ID 2272 | GWDWIIAP |
| SEQ ID 2273 | WDWIIAPK |
| SEQ ID 2274 | DWIIAPKR |
| SEQ ID 2275 | WIIAPKRY |
| SEQ ID 2276 | IIAPKRYK |
| SEQ ID 2277 | IAPKRYKA |
| SEQ ID 2278 | APKRYKAN |
| SEQ ID 2279 | PKRYKANY |
| SEQ ID 2280 | KRYKANYC |
| SEQ ID 2281 | RYKANYCS |
| SEQ ID 2282 | YKANYCSG |
| SEQ ID 2283 | KANYCSGQ |
| SEQ ID 2284 | ANYCSGQC |
| SEQ ID 2285 | NYCSGQCE |
| SEQ ID 2286 | YCSGQCEY |
| SEQ ID 2287 | CSGQCEYM |
| SEQ ID 2288 | SGQCEYMF |
| SEQ ID 2289 | GQCEYMFM |
| SEQ ID 2290 | QCEYMFMQ |
| SEQ ID 2291 | CEYMFMQK |
| SEQ ID 2292 | EYMFMQKY |
| SEQ ID 2293 | YMFMQKYP |
| SEQ ID 2294 | MFMQKYPH |
| SEQ ID 2295 | FMQKYPHT |
| SEQ ID 2296 | MQKYPHTH |
| SEQ ID 2297 | QKYPHTHL |
| SEQ ID 2298 | KYPHTHLV |
| SEQ ID 2299 | YPHTHLVQ |
| SEQ ID 2300 | PHTHLVQQ |
| SEQ ID 2301 | HTHLVQQA |
| SEQ ID 2302 | THLVQQAN |
| SEQ ID 2303 | HLVQQANP |
| SEQ ID 2304 | LVQQANPR |
| SEQ ID 2305 | VQQANPRG |
| SEQ ID 2306 | QQANPRGS |
| SEQ ID 2307 | QANPRGSA |
| SEQ ID 2308 | ANPRGSAG |
| SEQ ID 2309 | NPRGSAGP |
| SEQ ID 2310 | PRGSAGPC |
| SEQ ID 2311 | RGSAGPCC |
| SEQ ID 2312 | GSAGPCCT |
| SEQ ID 2313 | SAGPCCTP |
| SEQ ID 2314 | AGPCCTPT |
| SEQ ID 2315 | GPCCTPTK |
| SEQ ID 2316 | PCCTPTKM |
| SEQ ID 2317 | CCTPTKMS |
| SEQ ID 2318 | CTPTKMSP |
| SEQ ID 2319 | TPTKMSPI |
| SEQ ID 2320 | PTKMSPIN |
| SEQ ID 2321 | TKMSPINM |
| SEQ ID 2322 | KMSPINML |
| SEQ ID 2323 | MSPINMLY |
| SEQ ID 2324 | SPINMLYF |
| SEQ ID 2325 | PINMLYFN |
| SEQ ID 2326 | INMLYFND |
| SEQ ID 2327 | NMLYFNDK |
| SEQ ID 2328 | MLYFNDKQ |
| SEQ ID 2329 | LYFNDKQQ |
| SEQ ID 2330 | YFNDKQQI |
| SEQ ID 2331 | FNDKQQII |
| SEQ ID 2332 | NDKQQIIY |
| SEQ ID 2333 | DKQQIIYG |
| SEQ ID 2334 | KQQIIYGK |
| SEQ ID 2335 | QQIIYGKI |
| SEQ ID 2336 | QIIYGKIP |
| SEQ ID 2337 | IIYGKIPG |
| SEQ ID 2338 | IYGKIPGM |
| SEQ ID 2339 | YGKIPGMV |
| SEQ ID 2340 | GKIPGMVV |
| SEQ ID 2341 | KIPGMVVD |
| SEQ ID 2342 | IPGMVVDR |
| SEQ ID 2343 | PGMVVDRC |
| SEQ ID 2344 | GMVVDRCG |
| SEQ ID 2345 | MVVDRCGC |
| SEQ ID 2346 | VVDRCGCS |

In some embodiments, the active agent comprises a peptide which comprises, consists essentially of, or consists of any one or more of the 9-mer amino acid sequences (SEQ ID NO: 2347-2741) listed in Table 8.

TABLE 8

| | |
|---|---|
| SEQ ID 2347 | MVLAAPLLL |
| SEQ ID 2348 | VLAAPLLLG |
| SEQ ID 2349 | LAAPLLLGF |
| SEQ ID 2350 | AAPLLLGFL |
| SEQ ID 2351 | APLLLGFLL |
| SEQ ID 2352 | PLLLGFLLL |
| SEQ ID 2353 | LLLGFLLLA |
| SEQ ID 2354 | LLGFLLLAL |
| SEQ ID 2355 | LGFLLLALE |
| SEQ ID 2356 | GFLLLALEL |
| SEQ ID 2357 | FLLLALELR |
| SEQ ID 2358 | LLLALELRP |
| SEQ ID 2359 | LLALELRPR |
| SEQ ID 2360 | LALELRPRG |
| SEQ ID 2361 | ALELRPRGE |
| SEQ ID 2362 | LELRPRGEA |
| SEQ ID 2363 | ELRPRGEAA |
| SEQ ID 2364 | LRPRGEAAE |
| SEQ ID 2365 | RPRGEAAEG |
| SEQ ID 2366 | PRGEAAEGP |
| SEQ ID 2367 | RGEAAEGPA |
| SEQ ID 2368 | GEAAEGPAA |
| SEQ ID 2369 | EAAEGPAAA |
| SEQ ID 2370 | AAEGPAAAA |
| SEQ ID 2371 | AEGPAAAAA |
| SEQ ID 2372 | EGPAAAAAA |
| SEQ ID 2373 | GPAAAAAAA |
| SEQ ID 2374 | PAAAAAAAA |
| SEQ ID 2375 | AAAAAAAAA |
| SEQ ID 2376 | AAAAAAAAG |
| SEQ ID 2377 | AAAAAAAGV |
| SEQ ID 2378 | AAAAAAGVG |
| SEQ ID 2379 | AAAAAGVGG |
| SEQ ID 2380 | AAAAGVGGE |
| SEQ ID 2381 | AAAGVGGER |
| SEQ ID 2382 | AAGVGGERS |
| SEQ ID 2383 | AGVGGERSS |
| SEQ ID 2384 | GVGGERSSR |
| SEQ ID 2385 | VGGERSSRP |
| SEQ ID 2386 | GGERSSRPA |
| SEQ ID 2387 | GERSSRPAP |
| SEQ ID 2388 | ERSSRPAPS |
| SEQ ID 2389 | RSSRPAPSV |
| SEQ ID 2390 | SSRPAPSVA |
| SEQ ID 2391 | SRPAPSVAP |
| SEQ ID 2392 | RPAPSVAPE |
| SEQ ID 2393 | PAPSVAPEP |
| SEQ ID 2394 | APSVAPEPD |
| SEQ ID 2395 | PSVAPEPDG |
| SEQ ID 2396 | SVAPEPDGC |
| SEQ ID 2397 | VAPEPDGCP |
| SEQ ID 2398 | APEPDGCPV |
| SEQ ID 2399 | PEPDGCPVC |
| SEQ ID 2400 | EPDGCPVCV |
| SEQ ID 2401 | PDGCPVCVW |
| SEQ ID 2402 | DGCPVCVWR |
| SEQ ID 2403 | GCPVCVWRQ |
| SEQ ID 2404 | CPVCVWRQH |
| SEQ ID 2405 | PVCVWRQHS |
| SEQ ID 2406 | VCVWRQHSR |
| SEQ ID 2407 | CVWRQHSRE |
| SEQ ID 2408 | VWRQHSREL |
| SEQ ID 2409 | WRQHSRELR |
| SEQ ID 2410 | RQHSRELRL |
| SEQ ID 2411 | QHSRELRLE |
| SEQ ID 2412 | HSRELRLES |
| SEQ ID 2413 | SRELRLESI |
| SEQ ID 2414 | RELRLESIK |
| SEQ ID 2415 | ELRLESIKS |
| SEQ ID 2416 | LRLESIKSQ |
| SEQ ID 2417 | RLESIKSQI |
| SEQ ID 2418 | LESIKSQIL |
| SEQ ID 2419 | ESIKSQILS |
| SEQ ID 2420 | SIKSQILSK |
| SEQ ID 2421 | IKSQILSKL |
| SEQ ID 2422 | KSQILSKLR |
| SEQ ID 2423 | SQILSKLRL |
| SEQ ID 2424 | QILSKLRLK |
| SEQ ID 2425 | ILSKLRLKE |
| SEQ ID 2426 | LSKLRLKEA |
| SEQ ID 2427 | SKLRLKEAP |
| SEQ ID 2428 | KLRLKEAPN |
| SEQ ID 2429 | LRLKEAPNI |
| SEQ ID 2430 | RLKEAPNIS |
| SEQ ID 2431 | LKEAPNISR |
| SEQ ID 2432 | KEAPNISRE |
| SEQ ID 2433 | EAPNISREV |
| SEQ ID 2434 | APNISREVV |
| SEQ ID 2435 | PNISREVVK |
| SEQ ID 2436 | NISREVVKQ |
| SEQ ID 2437 | ISREVVKQL |
| SEQ ID 2438 | SREVVKQLL |
| SEQ ID 2439 | REVVKQLLP |
| SEQ ID 2440 | EVVKQLLPK |
| SEQ ID 2441 | VVKQLLPKA |
| SEQ ID 2442 | VKQLLPKAP |
| SEQ ID 2443 | KQLLPKAPP |
| SEQ ID 2444 | QLLPKAPPL |
| SEQ ID 2445 | LLPKAPPLQ |
| SEQ ID 2446 | LPKAPPLQQ |
| SEQ ID 2447 | PKAPPLQQI |
| SEQ ID 2448 | KAPPLQQIL |
| SEQ ID 2449 | APPLQQILD |
| SEQ ID 2450 | PPLQQILDL |
| SEQ ID 2451 | PLQQILDLH |
| SEQ ID 2452 | LQQILDLHD |
| SEQ ID 2453 | QQILDLHDF |
| SEQ ID 2454 | QILDLHDFQ |
| SEQ ID 2455 | ILDLHDFQG |
| SEQ ID 2456 | LDLHDFQGD |
| SEQ ID 2457 | DLHDFQGDA |
| SEQ ID 2458 | LHDFQGDAL |
| SEQ ID 2459 | HDFQGDALQ |
| SEQ ID 2460 | DFQGDALQP |
| SEQ ID 2461 | FQGDALQPE |
| SEQ ID 2462 | QGDALQPED |
| SEQ ID 2463 | GDALQPEDF |
| SEQ ID 2464 | DALQPEDFL |
| SEQ ID 2465 | ALQPEDFLE |
| SEQ ID 2466 | LQPEDFLEE |
| SEQ ID 2467 | QPEDFLEED |
| SEQ ID 2468 | PEDFLEEDE |
| SEQ ID 2469 | EDFLEEDEY |
| SEQ ID 2470 | DFLEEDEYH |
| SEQ ID 2471 | FLEEDEYHA |
| SEQ ID 2472 | LEEDEYHAT |
| SEQ ID 2473 | EEDEYHATT |
| SEQ ID 2474 | EDEYHATTE |
| SEQ ID 2475 | DEYHATTET |
| SEQ ID 2476 | EYHATTETV |
| SEQ ID 2477 | YHATTETVI |
| SEQ ID 2478 | HATTETVIS |
| SEQ ID 2479 | ATTETVISM |
| SEQ ID 2480 | TTETVISMA |
| SEQ ID 2481 | TETVISMAQ |
| SEQ ID 2482 | ETVISMAQE |
| SEQ ID 2483 | TVISMAQET |
| SEQ ID 2484 | VISMAQETD |
| SEQ ID 2485 | ISMAQETDP |
| SEQ ID 2486 | SMAQETDPA |
| SEQ ID 2487 | MAQETDPAV |
| SEQ ID 2488 | AQETDPAVQ |
| SEQ ID 2489 | QETDPAVQT |
| SEQ ID 2490 | ETDPAVQTD |
| SEQ ID 2491 | TDPAVQTDG |
| SEQ ID 2492 | DPAVQTDGS |
| SEQ ID 2493 | PAVQTDGSP |
| SEQ ID 2494 | AVQTDGSPL |
| SEQ ID 2495 | VQTDGSPLC |
| SEQ ID 2496 | QTDGSPLCC |
| SEQ ID 2497 | TDGSPLCCH |
| SEQ ID 2498 | DGSPLCCHF |
| SEQ ID 2499 | GSPLCCHFH |
| SEQ ID 2500 | SPLCCHFHF |
| SEQ ID 2501 | PLCCHFHFS |
| SEQ ID 2502 | LCCHFHFSP |
| SEQ ID 2503 | CCHFHFSPK |
| SEQ ID 2504 | CHFHFSPKV |
| SEQ ID 2505 | HFHFSPKVM |
| SEQ ID 2506 | FHFSPKVMF |
| SEQ ID 2507 | HFSPKVMFT |
| SEQ ID 2508 | FSPKVMFTK |
| SEQ ID 2509 | SPKVMFTKV |
| SEQ ID 2510 | PKVMFTKVL |
| SEQ ID 2511 | KVMFTKVLK |
| SEQ ID 2512 | VMFTKVLKA |
| SEQ ID 2513 | MFTKVLKAQ |
| SEQ ID 2514 | FTKVLKAQL |
| SEQ ID 2515 | TKVLKAQLW |
| SEQ ID 2516 | KVLKAQLWV |
| SEQ ID 2517 | VLKAQLWVY |
| SEQ ID 2518 | LKAQLWVYL |
| SEQ ID 2519 | KAQLWVYLR |
| SEQ ID 2520 | AQLWVYLRP |
| SEQ ID 2521 | QLWVYLRPV |
| SEQ ID 2522 | LWVYLRPVP |
| SEQ ID 2523 | WVYLRPVPR |
| SEQ ID 2524 | VYLRPVPRP |
| SEQ ID 2525 | YLRPVPRPA |
| SEQ ID 2526 | LRPVPRPAT |
| SEQ ID 2527 | RPVPRPATV |
| SEQ ID 2528 | PVPRPATVY |
| SEQ ID 2529 | VPRPATVYL |
| SEQ ID 2530 | PRPATVYLQ |
| SEQ ID 2531 | RPATVYLQI |
| SEQ ID 2532 | PATVYLQIL |
| SEQ ID 2533 | ATVYLQILR |
| SEQ ID 2534 | TVYLQILRL |
| SEQ ID 2535 | VYLQILRLK |
| SEQ ID 2536 | YLQILRLKP |
| SEQ ID 2537 | LQILRLKPL |
| SEQ ID 2538 | QILRLKPLT |
| SEQ ID 2539 | ILRLKPLTG |
| SEQ ID 2540 | LRLKPLTGE |
| SEQ ID 2541 | RLKPLTGEG |
| SEQ ID 2542 | LKPLTGEGT |
| SEQ ID 2543 | KPLTGEGTA |
| SEQ ID 2544 | PLTGEGTAG |
| SEQ ID 2545 | LTGEGTAGG |
| SEQ ID 2546 | TGEGTAGGG |
| SEQ ID 2547 | GEGTAGGGG |
| SEQ ID 2548 | EGTAGGGGG |
| SEQ ID 2549 | GTAGGGGGG |
| SEQ ID 2550 | TAGGGGGGR |
| SEQ ID 2551 | AGGGGGGRR |
| SEQ ID 2552 | GGGGGGRRH |
| SEQ ID 2553 | GGGGGRRHI |
| SEQ ID 2554 | GGGGRRHIR |
| SEQ ID 2555 | GGGRRHIRI |
| SEQ ID 2556 | GGRRHIRIR |
| SEQ ID 2557 | GRRHIRIRS |
| SEQ ID 2558 | RRHIRIRSL |
| SEQ ID 2559 | RHIRIRSLK |
| SEQ ID 2560 | HIRIRSLKI |
| SEQ ID 2561 | IRIRSLKIE |
| SEQ ID 2562 | RIRSLKIEL |
| SEQ ID 2563 | IRSLKIELH |
| SEQ ID 2564 | RSLKIELHS |
| SEQ ID 2565 | SLKIELHSR |
| SEQ ID 2566 | LKIELHSRS |
| SEQ ID 2567 | KIELHSRSG |
| SEQ ID 2568 | IELHSRSGH |
| SEQ ID 2569 | ELHSRSGHW |
| SEQ ID 2570 | LHSRSGHWQ |
| SEQ ID 2571 | HSRSGHWQS |
| SEQ ID 2572 | SRSGHWQSI |
| SEQ ID 2573 | RSGHWQSID |
| SEQ ID 2574 | SGHWQSIDF |
| SEQ ID 2575 | GHWQSIDFK |
| SEQ ID 2576 | HWQSIDFKQ |
| SEQ ID 2577 | WQSIDFKQV |
| SEQ ID 2578 | QSIDFKQVL |
| SEQ ID 2579 | SIDFKQVLH |
| SEQ ID 2580 | IDFKQVLHS |
| SEQ ID 2581 | DFKQVLHSW |
| SEQ ID 2582 | FKQVLHSWF |
| SEQ ID 2583 | KQVLHSWFR |
| SEQ ID 2584 | QVLHSWFRQ |
| SEQ ID 2585 | VLHSWFRQP |
| SEQ ID 2586 | LHSWFRQPQ |
| SEQ ID 2587 | HSWFRQPQS |
| SEQ ID 2588 | SWFRQPQSN |
| SEQ ID 2589 | WFRQPQSNW |
| SEQ ID 2590 | FRQPQSNWG |
| SEQ ID 2591 | RQPQSNWGI |
| SEQ ID 2592 | QPQSNWGIE |
| SEQ ID 2593 | PQSNWGIEI |
| SEQ ID 2594 | QSNWGIEIN |
| SEQ ID 2595 | SNWGIEINA |
| SEQ ID 2596 | NWGIEINAF |
| SEQ ID 2597 | WGIEINAFD |
| SEQ ID 2598 | GIEINAFDP |
| SEQ ID 2599 | IEINAFDPS |
| SEQ ID 2600 | EINAFDPSG |
| SEQ ID 2601 | INAFDPSGT |
| SEQ ID 2602 | NAFDPSGTD |
| SEQ ID 2603 | AFDPSGTDL |
| SEQ ID 2604 | FDPSGTDLA |
| SEQ ID 2605 | DPSGTDLAV |
| SEQ ID 2606 | PSGTDLAVT |
| SEQ ID 2607 | SGTDLAVTS |
| SEQ ID 2608 | GTDLAVTSL |
| SEQ ID 2609 | TDLAVTSLG |
| SEQ ID 2610 | DLAVTSLGP |
| SEQ ID 2611 | LAVTSLGPG |
| SEQ ID 2612 | AVTSLGPGA |
| SEQ ID 2613 | VTSLGPGAE |
| SEQ ID 2614 | TSLGPGAEG |
| SEQ ID 2615 | SLGPGAEGL |
| SEQ ID 2616 | LGPGAEGLH |
| SEQ ID 2617 | GPGAEGLHP |
| SEQ ID 2618 | PGAEGLHPF |
| SEQ ID 2619 | GAEGLHPFM |
| SEQ ID 2620 | AEGLHPFME |
| SEQ ID 2621 | EGLHPFMEL |
| SEQ ID 2622 | GLHPFMELR |
| SEQ ID 2623 | LHPFMELRV |
| SEQ ID 2624 | HPFMELRVL |
| SEQ ID 2625 | PFMELRVLE |
| SEQ ID 2626 | FMELRVLEN |
| SEQ ID 2627 | MELRVLENT |
| SEQ ID 2628 | ELRVLENTK |
| SEQ ID 2629 | LRVLENTKR |
| SEQ ID 2630 | RVLENTKRS |
| SEQ ID 2631 | VLENTKRSR |
| SEQ ID 2632 | LENTKRSRR |
| SEQ ID 2633 | ENTKRSRRN |
| SEQ ID 2634 | NTKRSRRNL |
| SEQ ID 2635 | TKRSRRNLG |
| SEQ ID 2636 | KRSRRNLGL |
| SEQ ID 2637 | RSRRNLGLD |
| SEQ ID 2638 | SRRNLGLDC |
| SEQ ID 2639 | RRNLGLDCD |
| SEQ ID 2640 | RNLGLDCDE |
| SEQ ID 2641 | NLGLDCDEH |
| SEQ ID 2642 | LGLDCDEHS |
| SEQ ID 2643 | GLDCDEHSS |
| SEQ ID 2644 | LDCDEHSSE |
| SEQ ID 2645 | DCDEHSSES |
| SEQ ID 2646 | CDEHSSESR |
| SEQ ID 2647 | DEHSSESRC |
| SEQ ID 2648 | EHSSESRCC |
| SEQ ID 2649 | HSSESRCCR |
| SEQ ID 2650 | SSESRCCRY |
| SEQ ID 2651 | SESRCCRYP |
| SEQ ID 2652 | ESRCCRYPL |
| SEQ ID 2653 | SRCCRYPLT |
| SEQ ID 2654 | RCCRYPLTV |
| SEQ ID 2655 | CCRYPLTVD |
| SEQ ID 2656 | CRYPLTVDF |
| SEQ ID 2657 | RYPLTVDFE |
| SEQ ID 2658 | YPLTVDFEA |
| SEQ ID 2659 | PLTVDFEAF |
| SEQ ID 2660 | LTVDFEAFG |
| SEQ ID 2661 | TVDFEAFGW |
| SEQ ID 2662 | VDFEAFGWD |
| SEQ ID 2663 | DFEAFGWDW |
| SEQ ID 2664 | FEAFGWDWI |
| SEQ ID 2665 | EAFGWDWII |
| SEQ ID 2666 | AFGWDWIIA |
| SEQ ID 2667 | FGWDWIIAP |
| SEQ ID 2668 | GWDWIIAPK |
| SEQ ID 2669 | WDWIIAPKR |
| SEQ ID 2670 | DWIIAPKRY |
| SEQ ID 2671 | WIIAPKRYK |
| SEQ ID 2672 | IIAPKRYKA |
| SEQ ID 2673 | IAPKRYKAN |
| SEQ ID 2674 | APKRYKANY |
| SEQ ID 2675 | PKRYKANYC |
| SEQ ID 2676 | KRYKANYCS |
| SEQ ID 2677 | RYKANYCSG |
| SEQ ID 2678 | YKANYCSGQ |
| SEQ ID 2679 | KANYCSGQC |
| SEQ ID 2680 | ANYCSGQCE |
| SEQ ID 2681 | NYCSGQCEY |
| SEQ ID 2682 | YCSGQCEYM |
| SEQ ID 2683 | CSGQCEYMF |
| SEQ ID 2684 | SGQCEYMFM |
| SEQ ID 2685 | GQCEYMFMQ |
| SEQ ID 2686 | QCEYMFMQK |
| SEQ ID 2687 | CEYMFMQKY |
| SEQ ID 2688 | EYMFMQKYP |
| SEQ ID 2689 | YMFMQKYPH |
| SEQ ID 2690 | MFMQKYPHT |
| SEQ ID 2691 | FMQKYPHTH |
| SEQ ID 2692 | MQKYPHTHL |
| SEQ ID 2693 | QKYPHTHLV |
| SEQ ID 2694 | KYPHTHLVQ |
| SEQ ID 2695 | YPHTHLVQQ |
| SEQ ID 2696 | PHTHLVQQA |
| SEQ ID 2697 | HTHLVQQAN |
| SEQ ID 2698 | THLVQQANP |
| SEQ ID 2699 | HLVQQANPR |
| SEQ ID 2700 | LVQQANPRG |
| SEQ ID 2701 | VQQANPRGS |
| SEQ ID 2702 | QQANPRGSA |
| SEQ ID 2703 | QANPRGSAG |
| SEQ ID 2704 | ANPRGSAGP |
| SEQ ID 2705 | NPRGSAGPC |
| SEQ ID 2706 | PRGSAGPCC |
| SEQ ID 2707 | RGSAGPCCT |
| SEQ ID 2708 | GSAGPCCTP |
| SEQ ID 2709 | SAGPCCTPT |
| SEQ ID 2710 | AGPCCTPTK |
| SEQ ID 2711 | GPCCTPTKM |
| SEQ ID 2712 | PCCTPTKMS |
| SEQ ID 2713 | CCTPTKMSP |
| SEQ ID 2714 | CTPTKMSPI |
| SEQ ID 2715 | TPTKMSPIN |
| SEQ ID 2716 | PTKMSPINM |
| SEQ ID 2717 | TKMSPINML |
| SEQ ID 2718 | KMSPINMLY |
| SEQ ID 2719 | MSPINMLYF |
| SEQ ID 2720 | SPINMLYFN |
| SEQ ID 2721 | PINMLYFND |
| SEQ ID 2722 | INMLYFNDK |
| SEQ ID 2723 | NMLYFNDKQ |
| SEQ ID 2724 | MLYFNDKQQ |
| SEQ ID 2725 | LYFNDKQQI |
| SEQ ID 2726 | YFNDKQQII |
| SEQ ID 2727 | FNDKQQIIY |
| SEQ ID 2728 | NDKQQIIYG |
| SEQ ID 2729 | DKQQIIYGK |
| SEQ ID 2730 | KQQIIYGKI |
| SEQ ID 2731 | QQIIYGKIP |
| SEQ ID 2732 | QIIYGKIPG |
| SEQ ID 2733 | IIYGKIPGM |
| SEQ ID 2734 | IYGKIPGMV |
| SEQ ID 2735 | YGKIPGMVV |
| SEQ ID 2736 | GKIPGMVVD |
| SEQ ID 2737 | KIPGMVVDR |
| SEQ ID 2738 | IPGMVVDRC |
| SEQ ID 2739 | PGMVVDRCG |
| SEQ ID 2740 | GMVVDRCGC |
| SEQ ID 2741 | MVVDRCGCS |

In some embodiments, the active agent comprises a peptide which comprises, consists essentially of, or consists of any one or more of the 10-mer amino acid sequences (SEQ ID NO: 2742-3136) listed in Table 9.

TABLE 9

| | |
|---|---|
| SEQ ID 2742 | MVLAAPLLLG |
| SEQ ID 2743 | VLAAPLLLGF |
| SEQ ID 2744 | LAAPLLLGFL |
| SEQ ID 2745 | AAPLLLGFLL |
| SEQ ID 2746 | APLLLGFLLL |
| SEQ ID 2747 | PLLLGFLLLA |
| SEQ ID 2748 | LLLGFLLLAL |
| SEQ ID 2749 | LLGFLLLALE |
| SEQ ID 2750 | LGFLLLALEL |
| SEQ ID 2751 | GFLLLALELR |
| SEQ ID 2752 | FLLLALELRP |
| SEQ ID 2753 | LLLALELRPR |
| SEQ ID 2754 | LLALELRPRG |
| SEQ ID 2755 | LALELRPRGE |
| SEQ ID 2756 | ALELRPRGEA |
| SEQ ID 2757 | LELRPRGEAA |
| SEQ ID 2758 | ELRPRGEAAE |
| SEQ ID 2759 | LRPRGEAAEG |
| SEQ ID 2760 | RPRGEAAEGP |
| SEQ ID 2761 | PRGEAAEGPA |
| SEQ ID 2762 | RGEAAEGPAA |
| SEQ ID 2763 | GEAAEGPAAA |
| SEQ ID 2764 | EAAEGPAAAA |
| SEQ ID 2765 | AAEGPAAAAA |
| SEQ ID 2766 | AEGPAAAAAA |
| SEQ ID 2767 | EGPAAAAAAA |
| SEQ ID 2768 | GPAAAAAAAA |
| SEQ ID 2769 | PAAAAAAAAA |
| SEQ ID 2770 | AAAAAAAAAA |
| SEQ ID 2771 | AAAAAAAAAG |
| SEQ ID 2772 | AAAAAAAAGV |
| SEQ ID 2773 | AAAAAAAGVG |
| SEQ ID 2774 | AAAAAAGVGG |
| SEQ ID 2775 | AAAAAGVGGE |
| SEQ ID 2776 | AAAAGVGGER |
| SEQ ID 2777 | AAAGVGGERS |
| SEQ ID 2778 | AAGVGGERSS |
| SEQ ID 2779 | AGVGGERSSR |
| SEQ ID 2780 | GVGGERSSRP |
| SEQ ID 2781 | VGGERSSRPA |
| SEQ ID 2782 | GGERSSRPAP |
| SEQ ID 2783 | GERSSRPAPS |
| SEQ ID 2784 | ERSSRPAPSV |
| SEQ ID 2785 | RSSRPAPSVA |
| SEQ ID 2786 | SSRPAPSVAP |
| SEQ ID 2787 | SRPAPSVAPE |
| SEQ ID 2788 | RPAPSVAPEP |
| SEQ ID 2789 | PAPSVAPEPD |
| SEQ ID 2790 | APSVAPEPDG |
| SEQ ID 2791 | PSVAPEPDGC |
| SEQ ID 2792 | SVAPEPDGCP |
| SEQ ID 2793 | VAPEPDGCPV |
| SEQ ID 2794 | APEPDGCPVC |
| SEQ ID 2795 | PEPDGCPVCV |
| SEQ ID 2796 | EPDGCPVCVW |
| SEQ ID 2797 | PDGCPVCVWR |
| SEQ ID 2798 | DGCPVCVWRQ |
| SEQ ID 2799 | GCPVCVWRQH |
| SEQ ID 2800 | CPVCVWRQHS |
| SEQ ID 2801 | PVCVWRQHSR |
| SEQ ID 2802 | VCVWRQHSRE |
| SEQ ID 2803 | CVWRQHSREL |
| SEQ ID 2804 | VWRQHSRELR |
| SEQ ID 2805 | WRQHSRELRL |
| SEQ ID 2806 | RQHSRELRLE |
| SEQ ID 2807 | QHSRELRLES |
| SEQ ID 2808 | HSRELRLESI |
| SEQ ID 2809 | SRELRLESIK |
| SEQ ID 2810 | RELRLESIKS |
| SEQ ID 2811 | ELRLESIKSQ |
| SEQ ID 2812 | LRLESIKSQI |
| SEQ ID 2813 | RLESIKSQIL |
| SEQ ID 2814 | LESIKSQILS |
| SEQ ID 2815 | ESIKSQILSK |
| SEQ ID 2816 | SIKSQILSKL |
| SEQ ID 2817 | IKSQILSKLR |
| SEQ ID 2818 | KSQILSKLRL |
| SEQ ID 2819 | SQILSKLRLK |
| SEQ ID 2820 | QILSKLRLKE |
| SEQ ID 2821 | ILSKLRLKEA |
| SEQ ID 2822 | LSKLRLKEAP |
| SEQ ID 2823 | SKLRLKEAPN |
| SEQ ID 2824 | KLRLKEAPNI |
| SEQ ID 2825 | LRLKEAPNIS |
| SEQ ID 2826 | RLKEAPNISR |
| SEQ ID 2827 | LKEAPNISRE |
| SEQ ID 2828 | KEAPNISREV |
| SEQ ID 2829 | EAPNISREVV |
| SEQ ID 2830 | APNISREVVK |
| SEQ ID 2831 | PNISREVVKQ |
| SEQ ID 2832 | NISREVVKQL |
| SEQ ID 2833 | ISREVVKQLL |
| SEQ ID 2834 | SREVVKQLLP |
| SEQ ID 2835 | REVVKQLLPK |
| SEQ ID 2836 | EVVKQLLPKA |
| SEQ ID 2837 | VVKQLLPKAP |
| SEQ ID 2838 | VKQLLPKAPP |
| SEQ ID 2839 | KQLLPKAPPL |
| SEQ ID 2840 | QLLPKAPPLQ |
| SEQ ID 2841 | LLPKAPPLQQ |
| SEQ ID 2842 | LPKAPPLQQI |
| SEQ ID 2843 | PKAPPLQQIL |
| SEQ ID 2844 | KAPPLQQILD |
| SEQ ID 2845 | APPLQQILDL |
| SEQ ID 2846 | PPLQQILDLH |
| SEQ ID 2847 | PLQQILDLHD |
| SEQ ID 2848 | LQQILDLHDF |
| SEQ ID 2849 | QQILDLHDFQ |
| SEQ ID 2850 | QILDLHDFQG |
| SEQ ID 2851 | ILDLHDFQGD |
| SEQ ID 2852 | LDLHDFQGDA |
| SEQ ID 2853 | DLHDFQGDAL |
| SEQ ID 2854 | LHDFQGDALQ |
| SEQ ID 2855 | HDFQGDALQP |
| SEQ ID 2856 | DFQGDALQPE |
| SEQ ID 2857 | FQGDALQPED |
| SEQ ID 2858 | QGDALQPEDF |
| SEQ ID 2859 | GDALQPEDFL |
| SEQ ID 2860 | DALQPEDFLE |
| SEQ ID 2861 | ALQPEDFLEE |
| SEQ ID 2862 | LQPEDFLEED |
| SEQ ID 2863 | QPEDFLEEDE |
| SEQ ID 2864 | PEDFLEEDEY |
| SEQ ID 2865 | EDFLEEDEYH |
| SEQ ID 2866 | DFLEEDEYHA |
| SEQ ID 2867 | FLEEDEYHAT |
| SEQ ID 2868 | LEEDEYHATT |
| SEQ ID 2869 | EEDEYHATTE |
| SEQ ID 2870 | EDEYHATTET |
| SEQ ID 2871 | DEYHATTETV |
| SEQ ID 2872 | EYHATTETVI |
| SEQ ID 2873 | YHATTETVIS |
| SEQ ID 2874 | HATTETVISM |
| SEQ ID 2875 | ATTETVISMA |
| SEQ ID 2876 | TTETVISMAQ |
| SEQ ID 2877 | TETVISMAQE |
| SEQ ID 2878 | ETVISMAQET |
| SEQ ID 2879 | TVISMAQETD |
| SEQ ID 2880 | VISMAQETDP |
| SEQ ID 2881 | ISMAQETDPA |
| SEQ ID 2882 | SMAQETDPAV |
| SEQ ID 2883 | MAQETDPAVQ |
| SEQ ID 2884 | AQETDPAVQT |
| SEQ ID 2885 | QETDPAVQTD |
| SEQ ID 2886 | ETDPAVQTDG |
| SEQ ID 2887 | TDPAVQTDGS |
| SEQ ID 2888 | DPAVQTDGSP |
| SEQ ID 2889 | PAVQTDGSPL |
| SEQ ID 2890 | AVQTDGSPLC |
| SEQ ID 2891 | VQTDGSPLCC |
| SEQ ID 2892 | QTDGSPLCCH |
| SEQ ID 2893 | TDGSPLCCHF |
| SEQ ID 2894 | DGSPLCCHFH |
| SEQ ID 2895 | GSPLCCHFHF |
| SEQ ID 2896 | SPLCCHFHFS |
| SEQ ID 2897 | PLCCHFHFSP |
| SEQ ID 2898 | LCCHFHFSPK |
| SEQ ID 2899 | CCHFHFSPKV |
| SEQ ID 2900 | CHFHFSPKVM |
| SEQ ID 2901 | HFHFSPKVMF |
| SEQ ID 2902 | FHFSPKVMFT |
| SEQ ID 2903 | HFSPKVMFTK |
| SEQ ID 2904 | FSPKVMFTKV |
| SEQ ID 2905 | SPKVMFTKVL |
| SEQ ID 2906 | PKVMFTKVLK |
| SEQ ID 2907 | KVMFTKVLKA |
| SEQ ID 2908 | VMFTKVLKAQ |
| SEQ ID 2909 | MFTKVLKAQL |
| SEQ ID 2910 | FTKVLKAQLW |
| SEQ ID 2911 | TKVLKAQLWV |
| SEQ ID 2912 | KVLKAQLWVY |
| SEQ ID 2913 | VLKAQLWVYL |
| SEQ ID 2914 | LKAQLWVYLR |
| SEQ ID 2915 | KAQLWVYLRP |
| SEQ ID 2916 | AQLWVYLRPV |
| SEQ ID 2917 | QLWVYLRPVP |
| SEQ ID 2918 | LWVYLRPVPR |
| SEQ ID 2919 | WVYLRPVPRP |
| SEQ ID 2920 | VYLRPVPRPA |
| SEQ ID 2921 | YLRPVPRPAT |
| SEQ ID 2922 | LRPVPRPATV |
| SEQ ID 2923 | RPVPRPATVY |
| SEQ ID 2924 | PVPRPATVYL |
| SEQ ID 2925 | VPRPATVYLQ |
| SEQ ID 2926 | PRPATVYLQI |
| SEQ ID 2927 | RPATVYLQIL |
| SEQ ID 2928 | PATVYLQILR |
| SEQ ID 2929 | ATVYLQILRL |
| SEQ ID 2930 | TVYLQILRLK |
| SEQ ID 2931 | VYLQILRLKP |
| SEQ ID 2932 | YLQILRLKPL |
| SEQ ID 2933 | LQILRLKPLT |
| SEQ ID 2934 | QILRLKPLTG |
| SEQ ID 2935 | ILRLKPLTGE |
| SEQ ID 2936 | LRLKPLTGEG |
| SEQ ID 2937 | RLKPLTGEGT |
| SEQ ID 2938 | LKPLTGEGTA |
| SEQ ID 2939 | KPLTGEGTAG |
| SEQ ID 2940 | PLTGEGTAGG |
| SEQ ID 2941 | LTGEGTAGGG |
| SEQ ID 2942 | TGEGTAGGGG |
| SEQ ID 2943 | GEGTAGGGGG |
| SEQ ID 2944 | EGTAGGGGGG |
| SEQ ID 2945 | GTAGGGGGGR |
| SEQ ID 2946 | TAGGGGGGRR |
| SEQ ID 2947 | AGGGGGGRRH |
| SEQ ID 2948 | GGGGGGRRHI |
| SEQ ID 2949 | GGGGGRRHIR |
| SEQ ID 2950 | GGGGRRHIRI |
| SEQ ID 2951 | GGGRRHIRIR |
| SEQ ID 2952 | GGRRHIRIRS |
| SEQ ID 2953 | GRRHIRIRSL |
| SEQ ID 2954 | RRHIRIRSLK |
| SEQ ID 2955 | RHIRIRSLKI |
| SEQ ID 2956 | HIRIRSLKIE |
| SEQ ID 2957 | IRIRSLKIEL |
| SEQ ID 2958 | RIRSLKIELH |
| SEQ ID 2959 | IRSLKIELHS |
| SEQ ID 2960 | RSLKIELHSR |
| SEQ ID 2961 | SLKIELHSRS |
| SEQ ID 2962 | LKIELHSRSG |
| SEQ ID 2963 | KIELHSRSGH |
| SEQ ID 2964 | IELHSRSGHW |
| SEQ ID 2965 | ELHSRSGHWQ |
| SEQ ID 2966 | LHSRSGHWQS |
| SEQ ID 2967 | HSRSGHWQSI |
| SEQ ID 2968 | SRSGHWQSID |
| SEQ ID 2969 | RSGHWQSIDE |
| SEQ ID 2970 | SGHWQSIDFK |
| SEQ ID 2971 | GHWQSIDFKQ |
| SEQ ID 2972 | HWQSIDFKQV |
| SEQ ID 2973 | WQSIDFKQVL |
| SEQ ID 2974 | QSIDFKQVLH |
| SEQ ID 2975 | SIDFKQVLHS |
| SEQ ID 2976 | IDFKQVLHSW |
| SEQ ID 2977 | DFKQVLHSWF |
| SEQ ID 2978 | FKQVLHSWFR |
| SEQ ID 2979 | KQVLHSWFRQ |
| SEQ ID 2980 | QVLHSWFRQP |
| SEQ ID 2981 | VLHSWFRQPQ |
| SEQ ID 2982 | LHSWFRQPQS |
| SEQ ID 2983 | HSWFRQPQSN |
| SEQ ID 2984 | SWFRQPQSNW |
| SEQ ID 2985 | WFRQPQSNWG |
| SEQ ID 2986 | FRQPQSNWGI |
| SEQ ID 2987 | RQPQSNWGIE |
| SEQ ID 2988 | QPQSNWGIEI |
| SEQ ID 2989 | PQSNWGIEIN |
| SEQ ID 2990 | QSNWGIEINA |
| SEQ ID 2991 | SNWGIEINAF |
| SEQ ID 2992 | NWGIEINAFD |
| SEQ ID 2993 | WGIEINAFDP |
| SEQ ID 2994 | GIEINAFDPS |
| SEQ ID 2995 | IEINAFDPSG |
| SEQ ID 2996 | EINAFDPSGT |
| SEQ ID 2997 | INAFDPSGTD |
| SEQ ID 2998 | NAFDPSGTDL |
| SEQ ID 2999 | AFDPSGTDLA |
| SEQ ID 3000 | FDPSGTDLAV |
| SEQ ID 3001 | DPSGTDLAVT |
| SEQ ID 3002 | PSGTDLAVTS |
| SEQ ID 3003 | SGTDLAVTSL |
| SEQ ID 3004 | GTDLAVTSLG |
| SEQ ID 3005 | TDLAVTSLGP |
| SEQ ID 3006 | DLAVTSLGPG |
| SEQ ID 3007 | LAVTSLGPGA |
| SEQ ID 3008 | AVTSLGPGAE |
| SEQ ID 3009 | VTSLGPGAEG |
| SEQ ID 3010 | TSLGPGAEGL |
| SEQ ID 3011 | SLGPGAEGLH |
| SEQ ID 3012 | LGPGAEGLHP |
| SEQ ID 3013 | GPGAEGLHPF |
| SEQ ID 3014 | PGAEGLHPFM |
| SEQ ID 3015 | GAEGLHPFME |
| SEQ ID 3016 | AEGLHPFMEL |
| SEQ ID 3017 | EGLHPFMELR |
| SEQ ID 3018 | GLHPFMELRV |
| SEQ ID 3019 | LHPFMELRVL |
| SEQ ID 3020 | HPFMELRVLE |
| SEQ ID 3021 | PFMELRVLEN |
| SEQ ID 3022 | FMELRVLENT |
| SEQ ID 3023 | MELRVLENTK |
| SEQ ID 3024 | ELRVLENTKR |
| SEQ ID 3025 | LRVLENTKRS |
| SEQ ID 3026 | RVLENTKRSR |
| SEQ ID 3027 | VLENTKRSRR |
| SEQ ID 3028 | LENTKRSRRN |
| SEQ ID 3029 | ENTKRSRRNL |
| SEQ ID 3030 | NTKRSRRNLG |
| SEQ ID 3031 | TKRSRRNLGL |
| SEQ ID 3032 | KRSRRNLGLD |
| SEQ ID 3033 | RSRRNLGLDC |
| SEQ ID 3034 | SRRNLGLDCD |
| SEQ ID 3035 | RRNLGLDCDE |
| SEQ ID 3036 | RNLGLDCDEH |
| SEQ ID 3037 | NLGLDCDEHS |
| SEQ ID 3038 | LGLDCDEHSS |
| SEQ ID 3039 | GLDCDEHSSE |
| SEQ ID 3040 | LDCDEHSSES |
| SEQ ID 3041 | DCDEHSSESR |
| SEQ ID 3042 | CDEHSSESRC |
| SEQ ID 3043 | DEHSSESRCC |
| SEQ ID 3044 | EHSSESRCCR |
| SEQ ID 3045 | HSSESRCCRY |
| SEQ ID 3046 | SSESRCCRYP |
| SEQ ID 3047 | SESRCCRYPL |
| SEQ ID 3048 | ESRCCRYPLT |
| SEQ ID 3049 | SRCCRYPLTV |
| SEQ ID 3050 | RCCRYPLTVD |
| SEQ ID 3051 | CCRYPLTVDF |
| SEQ ID 3052 | CRYPLTVDFE |
| SEQ ID 3053 | RYPLTVDFEA |
| SEQ ID 3054 | YPLTVDFEAF |
| SEQ ID 3055 | PLTVDFEAFG |
| SEQ ID 3056 | LTVDFEAFGW |
| SEQ ID 3057 | TVDFEAFGWD |
| SEQ ID 3058 | VDFEAFGWDW |
| SEQ ID 3059 | DFEAFGWDWI |
| SEQ ID 3060 | FEAFGWDWII |
| SEQ ID 3061 | EAFGWDWIIA |
| SEQ ID 3062 | AFGWDWIIAP |
| SEQ ID 3063 | FGWDWIIAPK |
| SEQ ID 3064 | GWDWIIAPKR |
| SEQ ID 3065 | WDWIIAPKRY |
| SEQ ID 3066 | DWIIAPKRYK |
| SEQ ID 3067 | WIIAPKRYKA |
| SEQ ID 3068 | IIAPKRYKAN |
| SEQ ID 3069 | IAPKRYKANY |
| SEQ ID 3070 | APKRYKANYC |
| SEQ ID 3071 | PKRYKANYCS |
| SEQ ID 3072 | KRYKANYCSG |
| SEQ ID 3073 | RYKANYCSGQ |
| SEQ ID 3074 | YKANYCSGQC |
| SEQ ID 3075 | KANYCSGQCE |
| SEQ ID 3076 | ANYCSGQCEY |
| SEQ ID 3077 | NYCSGQCEYM |
| SEQ ID 3078 | YCSGQCEYMF |
| SEQ ID 3079 | CSGQCEYMFM |
| SEQ ID 3080 | SGQCEYMFMQ |
| SEQ ID 3081 | GQCEYMFMQK |
| SEQ ID 3082 | QCEYMFMQKY |
| SEQ ID 3083 | CEYMFMQKYP |
| SEQ ID 3084 | EYMFMQKYPH |
| SEQ ID 3085 | YMFMQKYPHT |
| SEQ ID 3086 | MFMQKYPHTH |
| SEQ ID 3087 | FMQKYPHTHL |
| SEQ ID 3088 | MQKYPHTHLV |
| SEQ ID 3089 | QKYPHTHLVQ |
| SEQ ID 3090 | KYPHTHLVQQ |
| SEQ ID 3091 | YPHTHLVQQA |
| SEQ ID 3092 | PHTHLVQQAN |
| SEQ ID 3093 | HTHLVQQANP |
| SEQ ID 3094 | THLVQQANPR |
| SEQ ID 3095 | HLVQQANPRG |
| SEQ ID 3096 | LVQQANPRGS |
| SEQ ID 3097 | VQQANPRGSA |
| SEQ ID 3098 | QQANPRGSAG |
| SEQ ID 3099 | QANPRGSAGP |
| SEQ ID 3100 | ANPRGSAGPC |
| SEQ ID 3101 | NPRGSAGPCC |
| SEQ ID 3102 | PRGSAGPCCT |
| SEQ ID 3103 | RGSAGPCCTP |
| SEQ ID 3104 | GSAGPCCTPT |
| SEQ ID 3105 | SAGPCCTPTK |
| SEQ ID 3106 | AGPCCTPTKM |
| SEQ ID 3107 | GPCCTPTKMS |
| SEQ ID 3108 | PCCTPTKMSP |
| SEQ ID 3109 | CCTPTKMSPI |
| SEQ ID 3110 | CTPTKMSPIN |
| SEQ ID 3111 | TPTKMSPINM |
| SEQ ID 3112 | PTKMSPINML |
| SEQ ID 3113 | TKMSPINMLY |
| SEQ ID 3114 | KMSPINMLYF |
| SEQ ID 3115 | MSPINMLYFN |
| SEQ ID 3116 | SPINMLYFND |
| SEQ ID 3117 | PINMLYFNDK |
| SEQ ID 3118 | INMLYFNDKQ |
| SEQ ID 3119 | NMLYFNDKQQ |
| SEQ ID 3120 | MLYFNDKQQI |
| SEQ ID 3121 | LYFNDKQQII |
| SEQ ID 3122 | YFNDKQQIIY |
| SEQ ID 3123 | FNDKQQIIYG |
| SEQ ID 3124 | NDKQQIIYGK |
| SEQ ID 3125 | DKQQIIYGKI |
| SEQ ID 3126 | KQQIIYGKIP |
| SEQ ID 3127 | QQIIYGKIPG |
| SEQ ID 3128 | QIIYGKIPGM |
| SEQ ID 3129 | IIYGKIPGMV |
| SEQ ID 3130 | IYGKIPGMVV |
| SEQ ID 3131 | YGKIPGMVVD |
| SEQ ID 3132 | GKIPGMVVDR |
| SEQ ID 3133 | KIPGMVVDRC |
| SEQ ID 3134 | IPGMVVDRCG |
| SEQ ID 3135 | PGMVVDRCGC |
| SEQ ID 3136 | GMVVDRCGCS |

In some embodiments, the active agent comprises a peptide which comprises, consists essentially of, or consists of any one or more of the 11-mer amino acid sequences (SEQ ID NO: 3137-3531) listed in Table 10.

TABLE 10

| | |
|---|---|
| SEQ ID 3137 | MVLAAPLLLGF |
| SEQ ID 3138 | VLAAPLLLGFL |
| SEQ ID 3139 | LAAPLLLGFLL |
| SEQ ID 3140 | AAPLLLGFLLL |
| SEQ ID 3141 | APLLLGFLLLA |
| SEQ ID 3142 | PLLLGFLLLAL |
| SEQ ID 3143 | LLLGFLLLALE |
| SEQ ID 3144 | LLGFLLLALEL |
| SEQ ID 3145 | LGFLLLALELR |
| SEQ ID 3146 | GFLLLALELRP |
| SEQ ID 3147 | FLLLALELRPR |
| SEQ ID 3148 | LLLALELRPRG |
| SEQ ID 3149 | LLALELRPRGE |
| SEQ ID 3150 | LALELRPRGEA |
| SEQ ID 3151 | ALELRPRGEAA |
| SEQ ID 3152 | LELRPRGEAAE |
| SEQ ID 3153 | ELRPRGEAAEG |
| SEQ ID 3154 | LRPRGEAAEGP |
| SEQ ID 3155 | RPRGEAAEGPA |
| SEQ ID 3156 | PRGEAAEGPAA |
| SEQ ID 3157 | RGEAAEGPAAA |
| SEQ ID 3158 | GEAAEGPAAAA |
| SEQ ID 3159 | EAAEGPAAAAA |
| SEQ ID 3160 | AAEGPAAAAAA |
| SEQ ID 3161 | AEGPAAAAAAA |
| SEQ ID 3162 | EGPAAAAAAAA |
| SEQ ID 3163 | GPAAAAAAAAA |
| SEQ ID 3164 | PAAAAAAAAAA |
| SEQ ID 3165 | AAAAAAAAAAA |
| SEQ ID 3166 | AAAAAAAAAAG |
| SEQ ID 3167 | AAAAAAAAAGV |
| SEQ ID 3168 | AAAAAAAAGVG |
| SEQ ID 3169 | AAAAAAAGVGG |
| SEQ ID 3170 | AAAAAAGVGGE |
| SEQ ID 3171 | AAAAAGVGGER |
| SEQ ID 3172 | AAAAGVGGERS |
| SEQ ID 3173 | AAAGVGGERSS |
| SEQ ID 3174 | AAGVGGERSSR |
| SEQ ID 3175 | AGVGGERSSRP |
| SEQ ID 3176 | GVGGERSSRPA |
| SEQ ID 3177 | VGGERSSRPAP |
| SEQ ID 3178 | GGERSSRPAPS |
| SEQ ID 3179 | GERSSRPAPSV |
| SEQ ID 3180 | ERSSRPAPSVA |
| SEQ ID 3181 | RSSRPAPSVAP |
| SEQ ID 3182 | SSRPAPSVAPE |
| SEQ ID 3183 | SRPAPSVAPEP |
| SEQ ID 3184 | RPAPSVAPEPD |
| SEQ ID 3185 | PAPSVAPEPDG |
| SEQ ID 3186 | APSVAPEPDGC |
| SEQ ID 3187 | PSVAPEPDGCP |
| SEQ ID 3188 | SVAPEPDGCPV |
| SEQ ID 3189 | VAPEPDGCPVC |
| SEQ ID 3190 | APEPDGCPVCV |
| SEQ ID 3191 | PEPDGCPVCVW |
| SEQ ID 3192 | EPDGCPVCVWR |
| SEQ ID 3193 | PDGCPVCVWRQ |
| SEQ ID 3194 | DGCPVCVWRQH |
| SEQ ID 3195 | GCPVCVWRQHS |
| SEQ ID 3196 | CPVCVWRQHSR |
| SEQ ID 3197 | PVCVWRQHSRE |
| SEQ ID 3198 | VCVWRQHSREL |
| SEQ ID 3199 | CVWRQHSRELR |
| SEQ ID 3200 | VWRQHSRELRL |
| SEQ ID 3201 | WRQHSRELRLE |
| SEQ ID 3202 | RQHSRELRLES |
| SEQ ID 3203 | QHSRELRLESI |
| SEQ ID 3204 | HSRELRLESIK |
| SEQ ID 3205 | SRELRLESIKS |
| SEQ ID 3206 | RELRLESIKSQ |
| SEQ ID 3207 | ELRLESIKSQI |
| SEQ ID 3208 | LRLESIKSQIL |
| SEQ ID 3209 | RLESIKSQILS |
| SEQ ID 3210 | LESIKSQILSK |
| SEQ ID 3211 | ESIKSQILSKL |
| SEQ ID 3212 | SIKSQILSKLR |
| SEQ ID 3213 | IKSQILSKLRL |
| SEQ ID 3214 | KSQILSKLRLK |
| SEQ ID 3215 | SQILSKLRLKE |
| SEQ ID 3216 | QILSKLRLKEA |
| SEQ ID 3217 | ILSKLRLKEAP |
| SEQ ID 3218 | LSKLRLKEAPN |
| SEQ ID 3219 | SKLRLKEAPNI |
| SEQ ID 3220 | KLRLKEAPNIS |
| SEQ ID 3221 | LRLKEAPNISR |
| SEQ ID 3222 | RLKEAPNISRE |
| SEQ ID 3223 | LKEAPNISREV |
| SEQ ID 3224 | KEAPNISREVV |
| SEQ ID 3225 | EAPNISREVVK |
| SEQ ID 3226 | APNISREVVKQ |
| SEQ ID 3227 | PNISREVVKQL |
| SEQ ID 3228 | NISREVVKQLL |
| SEQ ID 3229 | ISREVVKQLLP |
| SEQ ID 3230 | SREVVKQLLPK |
| SEQ ID 3231 | REVVKQLLPKA |
| SEQ ID 3232 | EVVKQLLPKAP |
| SEQ ID 3233 | VVKQLLPKAPP |
| SEQ ID 3234 | VKQLLPKAPPL |
| SEQ ID 3235 | KQLLPKAPPLQ |
| SEQ ID 3236 | QLLPKAPPLQQ |
| SEQ ID 3237 | LLPKAPPLQQI |
| SEQ ID 3238 | LPKAPPLQQIL |
| SEQ ID 3239 | PKAPPLQQILD |
| SEQ ID 3240 | KAPPLQQILDL |
| SEQ ID 3241 | APPLQQILDLH |
| SEQ ID 3242 | PPLQQILDLHD |
| SEQ ID 3243 | PLQQILDLHDF |
| SEQ ID 3244 | LQQILDLHDFQ |
| SEQ ID 3245 | QQILDLHDFQG |
| SEQ ID 3246 | QILDLHDFQGD |
| SEQ ID 3247 | ILDLHDFQGDA |
| SEQ ID 3248 | LDLHDFQGDAL |
| SEQ ID 3249 | DLHDFQGDALQ |
| SEQ ID 3250 | LHDFQGDALQP |
| SEQ ID 3251 | HDFQGDALQPE |
| SEQ ID 3252 | DFQGDALQPED |
| SEQ ID 3253 | FQGDALQPEDF |
| SEQ ID 3254 | QGDALQPEDFL |
| SEQ ID 3255 | GDALQPEDFLE |
| SEQ ID 3256 | DALQPEDFLEE |
| SEQ ID 3257 | ALQPEDFLEED |
| SEQ ID 3258 | LQPEDFLEEDE |
| SEQ ID 3259 | QPEDFLEEDEY |
| SEQ ID 3260 | PEDFLEEDEYH |
| SEQ ID 3261 | EDFLEEDEYHA |
| SEQ ID 3262 | DFLEEDEYHAT |
| SEQ ID 3263 | FLEEDEYHATT |
| SEQ ID 3264 | LEEDEYHATTE |
| SEQ ID 3265 | EEDEYHATTET |
| SEQ ID 3266 | EDEYHATTETV |
| SEQ ID 3267 | DEYHATTETVI |
| SEQ ID 3268 | EYHATTETVIS |
| SEQ ID 3269 | YHATTETVISM |
| SEQ ID 3270 | HATTETVISMA |
| SEQ ID 3271 | ATTETVISMAQ |
| SEQ ID 3272 | TTETVISMAQE |
| SEQ ID 3273 | TETVISMAQET |
| SEQ ID 3274 | ETVISMAQETD |
| SEQ ID 3275 | TVISMAQETDP |
| SEQ ID 3276 | VISMAQETDPA |
| SEQ ID 3277 | ISMAQETDPAV |
| SEQ ID 3278 | SMAQETDPAVQ |
| SEQ ID 3279 | MAQETDPAVQT |
| SEQ ID 3280 | AQETDPAVQTD |
| SEQ ID 3281 | QETDPAVQTDG |
| SEQ ID 3282 | ETDPAVQTDGS |
| SEQ ID 3283 | TDPAVQTDGSP |
| SEQ ID 3284 | DPAVQTDGSPL |
| SEQ ID 3285 | PAVQTDGSPLC |
| SEQ ID 3286 | AVQTDGSPLCC |
| SEQ ID 3287 | VQTDGSPLCCH |
| SEQ ID 3288 | QTDGSPLCCHF |
| SEQ ID 3289 | TDGSPLCCHFH |
| SEQ ID 3290 | DGSPLCCHFHF |
| SEQ ID 3291 | GSPLCCHFHFS |
| SEQ ID 3292 | SPLCCHFHFSP |
| SEQ ID 3293 | PLCCHFHFSPK |
| SEQ ID 3294 | LCCHFHFSPKV |
| SEQ ID 3295 | CCHFHFSPKVM |
| SEQ ID 3296 | CHFHFSPKVMF |
| SEQ ID 3297 | HFHFSPKVMFT |
| SEQ ID 3298 | FHFSPKVMFTK |
| SEQ ID 3299 | HFSPKVMFTKV |
| SEQ ID 3300 | FSPKVMFTKVL |
| SEQ ID 3301 | SPKVMFTKVLK |
| SEQ ID 3302 | PKVMFTKVLKA |
| SEQ ID 3303 | KVMFTKVLKAQ |
| SEQ ID 3304 | VMFTKVLKAQL |
| SEQ ID 3305 | MFTKVLKAQLW |
| SEQ ID 3306 | FTKVLKAQLWV |
| SEQ ID 3307 | TKVLKAQLWVY |
| SEQ ID 3308 | KVLKAQLWVYL |
| SEQ ID 3309 | VLKAQLWVYLR |
| SEQ ID 3310 | LKAQLWVYLRP |
| SEQ ID 3311 | KAQLWVYLRPV |
| SEQ ID 3312 | AQLWVYLRPVP |
| SEQ ID 3313 | QLWVYLRPVPR |
| SEQ ID 3314 | LWVYLRPVPRP |
| SEQ ID 3315 | WVYLRPVPRPA |
| SEQ ID 3316 | VYLRPVPRPAT |
| SEQ ID 3317 | YLRPVPRPATV |
| SEQ ID 3318 | LRPVPRPATVY |
| SEQ ID 3319 | RPVPRPATVYL |
| SEQ ID 3320 | PVPRPATVYLQ |
| SEQ ID 3321 | VPRPATVYLQI |
| SEQ ID 3322 | PRPATVYLQIL |
| SEQ ID 3323 | RPATVYLQILR |
| SEQ ID 3324 | PATVYLQILRL |
| SEQ ID 3325 | ATVYLQILRLK |
| SEQ ID 3326 | TVYLQILRLKP |
| SEQ ID 3327 | VYLQILRLKPL |
| SEQ ID 3328 | YLQILRLKPLT |
| SEQ ID 3329 | LQILRLKPLTG |
| SEQ ID 3330 | QILRLKPLTGE |
| SEQ ID 3331 | ILRLKPLTGEG |
| SEQ ID 3332 | LRLKPLTGEGT |
| SEQ ID 3333 | RLKPLTGEGTA |
| SEQ ID 3334 | LKPLTGEGTAG |
| SEQ ID 3335 | KPLTGEGTAGG |
| SEQ ID 3336 | PLTGEGTAGGG |
| SEQ ID 3337 | LTGEGTAGGGG |
| SEQ ID 3338 | TGEGTAGGGGG |
| SEQ ID 3339 | GEGTAGGGGGG |
| SEQ ID 3340 | EGTAGGGGGGR |
| SEQ ID 3341 | GTAGGGGGGRR |
| SEQ ID 3342 | TAGGGGGGRRH |
| SEQ ID 3343 | AGGGGGGRRHI |
| SEQ ID 3344 | GGGGGGRRHIR |
| SEQ ID 3345 | GGGGGRRHIRI |
| SEQ ID 3346 | GGGGRRHIRIR |
| SEQ ID 3347 | GGGRRHIRIRS |
| SEQ ID 3348 | GGRRHIRIRSL |
| SEQ ID 3349 | GRRHIRIRSLK |
| SEQ ID 3350 | RRHIRIRSLKI |
| SEQ ID 3351 | RHIRIRSLKIE |
| SEQ ID 3352 | HIRIRSLKIEL |
| SEQ ID 3353 | IRIRSLKIELH |
| SEQ ID 3354 | RIRSLKIELHS |
| SEQ ID 3355 | IRSLKIELHSR |
| SEQ ID 3356 | RSLKIELHSRS |
| SEQ ID 3357 | SLKIELHSRSG |
| SEQ ID 3358 | LKIELHSRSGH |
| SEQ ID 3359 | KIELHSRSGHW |
| SEQ ID 3360 | IELHSRSGHWQ |
| SEQ ID 3361 | ELHSRSGHWQS |
| SEQ ID 3362 | LHSRSGHWQSI |
| SEQ ID 3363 | HSRSGHWQSID |
| SEQ ID 3364 | SRSGHWQSIDF |
| SEQ ID 3365 | RSGHWQSIDFK |
| SEQ ID 3366 | SGHWQSIDFKQ |
| SEQ ID 3367 | GHWQSIDFKQV |
| SEQ ID 3368 | HWQSIDFKQVL |
| SEQ ID 3369 | WQSIDFKQVLH |
| SEQ ID 3370 | QSIDFKQVLHS |
| SEQ ID 3371 | SIDFKQVLHSW |
| SEQ ID 3372 | IDFKQVLHSWF |
| SEQ ID 3373 | DFKQVLHSWFR |
| SEQ ID 3374 | FKQVLHSWFRQ |
| SEQ ID 3375 | KQVLHSWFRQP |
| SEQ ID 3376 | QVLHSWFRQPQ |
| SEQ ID 3377 | VLHSWFRQPQS |
| SEQ ID 3378 | LHSWFRQPQSN |
| SEQ ID 3379 | HSWFRQPQSNW |
| SEQ ID 3380 | SWFRQPQSNWG |
| SEQ ID 3381 | WFRQPQSNWGI |
| SEQ ID 3382 | FRQPQSNWGIE |
| SEQ ID 3383 | RQPQSNWGIEI |
| SEQ ID 3384 | QPQSNWGIEIN |
| SEQ ID 3385 | PQSNWGIEINA |
| SEQ ID 3386 | QSNWGIEINAF |
| SEQ ID 3387 | SNWGIEINAFD |
| SEQ ID 3388 | NWGIEINAFDP |
| SEQ ID 3389 | WGIEINAFDPS |
| SEQ ID 3390 | GIEINAFDPSG |
| SEQ ID 3391 | IEINAFDPSGT |
| SEQ ID 3392 | EINAFDPSGTD |
| SEQ ID 3393 | INAFDPSGTDL |
| SEQ ID 3394 | NAFDPSGTDLA |
| SEQ ID 3395 | AFDPSGTDLAV |
| SEQ ID 3396 | FDPSGTDLAVT |
| SEQ ID 3397 | DPSGTDLAVTS |
| SEQ ID 3398 | PSGTDLAVTSL |
| SEQ ID 3399 | SGTDLAVTSLG |
| SEQ ID 3400 | GTDLAVTSLGP |
| SEQ ID 3401 | TDLAVTSLGPG |
| SEQ ID 3402 | DLAVTSLGPGA |
| SEQ ID 3403 | LAVTSLGPGAE |
| SEQ ID 3404 | AVTSLGPGAEG |
| SEQ ID 3405 | VTSLGPGAEGL |
| SEQ ID 3406 | TSLGPGAEGLH |
| SEQ ID 3407 | SLGPGAEGLHP |
| SEQ ID 3408 | LGPGAEGLHPF |
| SEQ ID 3409 | GPGAEGLHPFM |
| SEQ ID 3410 | PGAEGLHPFME |
| SEQ ID 3411 | GAEGLHPFMEL |
| SEQ ID 3412 | AEGLHPFMELR |
| SEQ ID 3413 | EGLHPFMELRV |
| SEQ ID 3414 | GLHPFMELRVL |
| SEQ ID 3415 | LHPFMELRVLE |
| SEQ ID 3416 | HPFMELRVLEN |
| SEQ ID 3417 | PFMELRVLENT |
| SEQ ID 3418 | FMELRVLENTK |
| SEQ ID 3419 | MELRVLENTKR |
| SEQ ID 3420 | ELRVLENTKRS |
| SEQ ID 3421 | LRVLENTKRSR |
| SEQ ID 3422 | RVLENTKRSRR |
| SEQ ID 3423 | VLENTKRSRRN |
| SEQ ID 3424 | LENTKRSRRNL |
| SEQ ID 3425 | ENTKRSRRNLG |
| SEQ ID 3426 | NTKRSRRNLGL |
| SEQ ID 3427 | TKRSRRNLGLD |
| SEQ ID 3428 | KRSRRNLGLDC |
| SEQ ID 3429 | RSRRNLGLDCD |
| SEQ ID 3430 | SRRNLGLDCDE |
| SEQ ID 3431 | RRNLGLDCDEH |
| SEQ ID 3432 | RNLGLDCDEHS |
| SEQ ID 3433 | NLGLDCDEHSS |
| SEQ ID 3434 | LGLDCDEHSSE |
| SEQ ID 3435 | GLDCDEHSSES |
| SEQ ID 3436 | LDCDEHSSESR |
| SEQ ID 3437 | DCDEHSSESRC |
| SEQ ID 3438 | CDEHSSESRCC |
| SEQ ID 3439 | DEHSSESRCCR |
| SEQ ID 3440 | EHSSESRCCRY |
| SEQ ID 3441 | HSSESRCCRYP |
| SEQ ID 3442 | SSESRCCRYPL |
| SEQ ID 3443 | SESRCCRYPLT |
| SEQ ID 3444 | ESRCCRYPLTV |
| SEQ ID 3445 | SRCCRYPLTVD |
| SEQ ID 3446 | RCCRYPLTVDF |
| SEQ ID 3447 | CCRYPLTVDFE |
| SEQ ID 3448 | CRYPLTVDFEA |
| SEQ ID 3449 | RYPLTVDFEAF |
| SEQ ID 3450 | YPLTVDFEAFG |
| SEQ ID 3451 | PLTVDFEAFGW |
| SEQ ID 3452 | LTVDFEAFGWD |
| SEQ ID 3453 | TVDFEAFGWDW |
| SEQ ID 3454 | VDFEAFGWDWI |
| SEQ ID 3455 | DFEAFGWDWII |
| SEQ ID 3456 | FEAFGWDWIIA |
| SEQ ID 3457 | EAFGWDWIIAP |
| SEQ ID 3458 | AFGWDWIIAPK |
| SEQ ID 3459 | FGWDWIIAPKR |
| SEQ ID 3460 | GWDWIIAPKRY |
| SEQ ID 3461 | WDWIIAPKRYK |
| SEQ ID 3462 | DWIIAPKRYKA |
| SEQ ID 3463 | WIIAPKRYKAN |
| SEQ ID 3464 | IIAPKRYKANY |
| SEQ ID 3465 | IAPKRYKANYC |
| SEQ ID 3466 | APKRYKANYCS |
| SEQ ID 3467 | PKRYKANYCSG |
| SEQ ID 3468 | KRYKANYCSGQ |
| SEQ ID 3469 | RYKANYCSGQC |
| SEQ ID 3470 | YKANYCSGQCE |
| SEQ ID 3471 | KANYCSGQCEY |
| SEQ ID 3472 | ANYCSGQCEYM |
| SEQ ID 3473 | NYCSGQCEYMF |
| SEQ ID 3474 | YCSGQCEYMFM |
| SEQ ID 3475 | CSGQCEYMFMQ |
| SEQ ID 3476 | SGQCEYMFMQK |
| SEQ ID 3477 | GQCEYMFMQKY |
| SEQ ID 3478 | QCEYMFMQKYP |
| SEQ ID 3479 | CEYMFMQKYPH |
| SEQ ID 3480 | EYMFMQKYPHT |
| SEQ ID 3481 | YMFMQKYPHTH |
| SEQ ID 3482 | MFMQKYPHTHL |
| SEQ ID 3483 | FMQKYPHTHLV |
| SEQ ID 3484 | MQKYPHTHLVQ |
| SEQ ID 3485 | QKYPHTHLVQQ |
| SEQ ID 3486 | KYPHTHLVQQA |
| SEQ ID 3487 | YPHTHLVQQAN |
| SEQ ID 3488 | PHTHLVQQANP |
| SEQ ID 3489 | HTHLVQQANPR |
| SEQ ID 3490 | THLVQQANPRG |
| SEQ ID 3491 | HLVQQANPRGS |
| SEQ ID 3492 | LVQQANPRGSA |
| SEQ ID 3493 | VQQANPRGSAG |
| SEQ ID 3494 | QQANPRGSAGP |
| SEQ ID 3495 | QANPRGSAGPC |
| SEQ ID 3496 | ANPRGSAGPCC |
| SEQ ID 3497 | NPRGSAGPCCT |
| SEQ ID 3498 | PRGSAGPCCTP |
| SEQ ID 3499 | RGSAGPCCTPT |
| SEQ ID 3500 | GSAGPCCTPTK |
| SEQ ID 3501 | SAGPCCTPTKM |
| SEQ ID 3502 | AGPCCTPTKMS |
| SEQ ID 3503 | GPCCTPTKMSP |
| SEQ ID 3504 | PCCTPTKMSPI |
| SEQ ID 3505 | CCTPTKMSPIN |
| SEQ ID 3506 | CTPTKMSPINM |
| SEQ ID 3507 | TPTKMSPINML |
| SEQ ID 3508 | PTKMSPINMLY |
| SEQ ID 3509 | TKMSPINMLYF |
| SEQ ID 3510 | KMSPINMLYFN |
| SEQ ID 3511 | MSPINMLYFND |
| SEQ ID 3512 | SPINMLYFNDK |
| SEQ ID 3513 | PINMLYFNDKQ |
| SEQ ID 3514 | INMLYFNDKQQ |
| SEQ ID 3515 | NMLYFNDKQQI |
| SEQ ID 3516 | MLYFNDKQQII |
| SEQ ID 3517 | LYFNDKQQIIY |
| SEQ ID 3518 | YFNDKQQIIYG |
| SEQ ID 3519 | FNDKQQIIYGK |
| SEQ ID 3520 | NDKQQIIYGKI |
| SEQ ID 3521 | DKQQIIYGKIP |
| SEQ ID 3522 | KQQIIYGKIPG |
| SEQ ID 3523 | QQIIYGKIPGM |
| SEQ ID 3524 | QIIYGKIPGMV |
| SEQ ID 3525 | IIYGKIPGMVV |
| SEQ ID 3526 | IYGKIPGMVVD |
| SEQ ID 3527 | YGKIPGMVVDR |
| SEQ ID 3528 | GKIPGMVVDRC |
| SEQ ID 3529 | KIPGMVVDRCG |
| SEQ ID 3530 | IPGMVVDRCGC |
| SEQ ID 3531 | PGMVVDRCGCS |

In some embodiments, the peptides may comprise one, two, three or more conservative substitutions of amino acids. As used herein, a “conservative substitution” is one in which substitution of one amino acid for another does not impair the function of the peptide, including substitution of an amino acid having a side chain of a certain nature (e.g., acidic, basic, aromatic, aliphatic uncharged, non-polar uncharged, hydrophilic uncharged) by another amino acid having a side chain of the same nature. Examples of conservative substitutions are shown in Table 11. In some embodiments, the conservative substitution is modified by oxidation. For example, in some embodiments, a proline residue in SEQ ID NOs 2-3531 may be replaced by an oxidized methionine (e.g., methionine sulfone, methionine sulfoxide, etc.) or an oxidized cysteine.

TABLE 11
Conservative Substitutions

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and Gln (Q) |
| Aliphatic Uncharged Residues | Gly (G), Ala (A), Val (V), Leu (L), and |
| | Ile (I) |
| Non-polar Uncharged Residues | Cys (C), Met (M), and Pro (P) |
| Aromatic Residues | Phe (F), Tyr (Y), and Trp (W) |

In some embodiments, the peptides may comprise one, two, three or more (e.g., one, two, three, etc.) non-natural and/or non-proteinogenic amino acids substituted or in place a comparable number of amino acids in SEQ ID NOs. 2-3531. In some embodiments, the peptides of the invention may comprise modified variants of SEQ ID NOs 2-3531 wherein at least one of the amino acids is replaced by the “D” (dextrorotary) analogue of the natural “L” optical isomer found in SEQ ID Nos 2-3531. In another embodiment, at least one (e.g., one, two, three, etc.) of the amino acids found in SEQ ID Nos. 2-2531 are replaced with a non-naturally occurring and/or non-proteogenic amino acid according to Formulas (III) or (IV) as detailed below.

The peptides of the invention can be modified to improve the lipophilicity, stability, or to enhance penetration through the stratum corneum. In some embodiments, the peptides are modified with a fatty acid chain (e.g., C₆₋₂₂), such as palmitoyl. In some embodiments, at least one of the nitrogen atoms in the amide bonds between adjacent amino acids may be methylated to improve metabolic stability. The peptides may also be phosphorylated, for example by forming one or more phophoserine, phosphothreonine and/or phosphotyrosine residues.

In some embodiments, the modified peptides will have the structure according to Formula (I):

R₁-Ω—R₂ (I)

where Ω represents a peptide comprising a chemically modified amino acid residue (e.g., a peptide of SEQ ID 2-3531 wherein one or more amino acid residues have been oxidized) and R₁ and R₂ are independently either absent or are selected from hydrogen or C₁₋₂₆ (C₁₋₆ or C₆₋₁₂ or C₁₂₋₁₈ or C₁₈₋₂₂) hydrocarbons, optionally substituted with a group X₁ or with 1-20 (or 1-10 or 1-6 or 1-3) heteroatoms selected from halogen (e.g., fluorine), oxygen, nitrogen, phosphorous, sulfur, silicon and combinations thereof (more typically, oxygen and nitrogen). In some embodiments, one of R₁ and R₂ is a C₁₋₂₆ hydrocarbon. In some embodiments, only one of R₁ and R₂ is a C₁₋₂₆ hydrocarbon. In some embodiments, one of R₁ and R₂ is a C₁₋₂₆ hydrocarbon selected from the group consisting of alkyl, alkenyl, alkynyl, aryl, alkyl-aryl (e.g., benzyl), and aryl-alkyl optionally substituted with halogen (e.g., fluorine), oxygen, nitrogen, phosphorous, sulfur, and combinations thereof, in various embodiments comprising heteroatoms selected from halogen (e.g., fluorine), oxygen, nitrogen, phosphorous, sulfur, and combinations thereof, in various embodiments comprising from 1-10 or 1-6 or 1-3 heteroatoms.

In some embodiments, R₁ and/or R₂ may comprise a group of the form R—(C═O)—, where R is a C₁₋₂₅ hydrocarbon as described above. In certain embodiments, the peptide derivative is N-acetylated. In one embodiment, R₁ and/or R₂ may comprise an acyl group, for example, one N-acylated or having the form CH₃—(CH₃)n-(C═O)— where “n” is an integer from 0-25 (e.g., zero or from 0-17 or 7-17). In certain embodiments, R₁ and/or R₂ may comprise an acetyl group of the form CH₃—(C═O)—. In some embodiments, the peptide derivative comprises an acetyl group at the amino terminus. In some embodiments, the peptide derivative comprises an acetyl group at the carboxylic acid terminus. In one embodiment, R₁ and/or R₂ may comprise a palmitoyl group of the form CH₃—(CH₃)₁₄—(C═O)—. R₁ and/or R₂ may be attached to a nitrogen atom on the peptide so thereby form an amide bond of the form Ω-NH—(C═O)—R, formed, for example, through the reaction of an acid of the form R—(C═O)—OH (or activated derivative of the acid) with a nitrogen atom on the N-terminal amino group of the peptide or a nitrogen atom on a side chain (e.g., lysine) of the peptide. In some embodiments, R₁ and/or R₂ may be attached to the peptide through an amide bond of the form Ω—(C═O)—NH—R, formed, for example, by reaction of an amine of the form R—NH with the carboxyl terminus of the peptide or on a carboxyl-containing side chain (e.g., aspartic acid or glutamic acid). In some embodiments, R₁ and/or R₂ may be attached to the peptide through an ester bond of the form Ω—(C═O)—O—R, formed, for example, through the reaction of an alcohol of the form R—OH with the carboxyl terminus of the peptide or carboxyl side chain (e.g., aspartic acid or glutamic acid). In some embodiments, R₁ and/or R₂ may be attached to the peptide through an ester bond of the form Ω—O—(C═O)—R, formed, for example, by the reaction of an acid of the form R—(C═O)—OH with a hydroxyl group on an amino acid side chain (e.g., serine or threonine). In any case where an acid is reacted, the acid may first be activated according to conventional practice by first converting it to an anhydride, acid halide, or activated ester, such as an N-hydroxysuccinimide ester, etc. It is also contemplated that R₁ and/or R₂ may be attached to the peptide through thioester bonds of the form Ω—S—(C═O)—R, thioether bonds of the form Ω-S—R, ether bonds of the form Ω—O—R, and amines of the form of the form Ω-NR^{N}—R, to name but a few non-limiting examples. In various embodiments, R may be branched (e.g., ethylhexyl), cyclic, or straight chained. R and R^{N} may be, without limitation methyl, ethyl, propyl, butyl, pentyl, hexyl, heptyl, octyl, nonyl, decyl, undecyl, dodecyl, or C₁₃, or C₁₄, or C₁₅, or C₁₆, or C₁₇, or C₁₈, or C₁₉, or C₂₀, or C₂₁, or C₂₂, or C₂₃, or C₂₄, or C₂₅, or C₂₆ alkyl, alkenyl, or alkynyl, etc, optionally substituted with a group X₁ or heteroatoms selected from halogen (e.g., fluorine), oxygen, nitrogen, phosphorous, sulfur, silicon, and combinations thereof, in various embodiments comprising from 1-10 or 1-6 or 1-3 heteroatoms. Any of the groups R, R₁, R₂ and R^{N} may be further substituted with from 1-3 groups X₁ where X₁ is selected independently at each occurrence from hydrogen, —F; —Cl; —Br; —I; —OH, —OR*; —NH₂; —NHR*; —N(R*)₂; —N(R*)₃⁺; —N(R*)—OH; —N(—O)(R*)₂; —O—N(R*)₂; —N(R*)—O—R*; —N(R*)—N(R*)₂; —C═N—R*; —N═C(R*)₂; —C═N—N(R*)₂; —C(═NR*)—N(R*)₂; —SH; —SR*; —CN; —NC; —(C═O)—R*; —CHO; —CO₂H; —CO₂—; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —(C═NR)—O—R*; —O—(C═NR*)—R*, —SCN; —NCS; —NSO; —SSR*; —N(R*)—C(═O)—N(R*)₂; —N(R*)—C(═S)—N(R*)₂; —SO₂—R*; —O—S(═O)₂—R*; —S(═O)₂—OR*; —N(R*)—SO₂—R*; —SO₂—N(R*)₂; —O—SO₃—R*; —O—S(═O)₂—OR*; —O—S(═O)—OR*; —O—S(═O)—R*; —S(═O)—OR*; —S(═O)—R*; —NO; —NO₂; —NO₃; —O—NO; —O—NO₂; —N₃; —N₂—R*; —N(C₂H₄); —Si(R*)₃; —CF₃; —O—CF₃; —PR*₂; —O—P(═O)(OR*)₂; —P(═O)(OR*)₂; C₁-C₈ perfluoroalkyl; an aliphatic C₁-C₈ hydrocarbon radical; a C₁-C₈ aromatic hydrocarbon radical; or a C₁-C₈ heteroaryl radical. R* is a C₁₋₁₀ hydrocarbon, such as methyl, ethyl, propyl, butyl, pentyl, hexyl, benzyl, phenyl, etc. Any two of R, R*, R^{N}, R₁, and R₂ may together form a 3-8 membered, optionally heterocyclic ring.

In some embodiments, R₁ or R₂ is attached covalently to the terminal carboxyl group. In some embodiments, R₁ and/or R₂ is attached to the terminal amino group. In some embodiments, R₁ and/or R₂ is attached to a side chain having a nitrogen, oxygen, or sulfur atom.

In some embodiments, R₁ and/or R₂ promotes adhesion to or penetration of an integument. In some embodiments, R₁ and/or R₂ comprise biotin, a beta-keto ester, or a polyarginine sequence (e.g., having 3-15 arginines).

The peptide can be pegylated to enhance water-solubility. In some embodiments, R₁ and/or R₂ have the form —(OCH₂CH₂)y-Z or —(CH₂CH₂O)y-Z, where “y” is an integer from 1-20 (or from 1-10 or from 1-6 or from 1-3) and Z is H, R₃, X₁, or R₄—X₁, where R₃ and R₄ are independently branched, straight chained, or cyclic C₁₋₆ hydrocarbons (e.g., methyl, ethyl, propyl, methylene, —(CH₂)ₙ— (n=1-6), etc.). In some embodiments, R₁ and/or R₂ comprise mini-PEG (i.e., 11-amino-3,6,9-trioxaundecanoic acid).

In some embodiments, the modified peptide may comprise a single modified amino acid residue. For example, the modified peptide may have the structure of formula (Ia):

R₁-(Ω₁)ₓ-Ωₘ-(Ω₂)_{y}—R₂ (Ia)

wherein Ω₁ and Ω₂ are independently either absent (i.e., it is a bond) or independently selected at each occurrence from amino acid residues and x and y are independently integers from 0-10 or from 1-10 (preferably from 1-3 or 1-2) with the proviso that the sum of x and y is no more than 10, no more than 6, or no more than 4, or no more than 2; and
Ωₘ is a modified amino acid residue (such as an amino acid residue having an oxidized side chain). In certain embodiments, the sum of x and y is two, three, four, five, six, seven, eight, nine, or ten. In some embodiments, Ω₁ and Ω₂ may, for example, be independently selected at each occurrence from Alanine, Cysteine, Aspartic acid, Glutamic acid, Phenylalanine, Glycine, Histidine, Isoleucine, Lysine, Leucine, Methionine, Asparagine, Pyrrolysine, Proline, Glutamine, Arginine, Serine, Threonine, Selenocysteine, Valine, Tryptophan, and Tyrosine; and Ωₘ is preferably, but not necessarily, a sulfoxide or sulfone derivative of an amino acid.

A modified amino acid residue (e.g., Ωₘ) may have the structure:

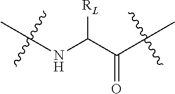

or
wherein R_{L} is a modified (e.g., oxidized) sidechain of an amino acids. The side chain is typically a C₁₋₁₂ hydrocarbon, optionally containing from 1-4 or 1-3 or 1-2 or 1 heteroatoms selected from O, N, and S. The side chain of amino acid to be oxidized may be the side chain of Alanine, Cysteine, Aspartic acid, Glutamic acid, Phenylalanine, Glycine, Histidine, Isoleucine, Lysine, Leucine, Methionine, Asparagine, Pyrrolysine, Proline, Glutamine, Arginine, Serine, Threonine, Selenocysteine, Valine, Tryptophan, or Tyrosine. For example, the side chain may be any of the side chains in Table 12.

TABLE 12

| Amino Acid | Side Chain Prior to Modification |
|---|---|
| Ala | —CH₃ |
| Ser | —CH₂OH |
| Thr | —CH(CH₃)(OH) |
| Cys | —CH₂SH |
| Val | —CH(CH₃)₂ |
| Leu | —CH₂CH(CH₃)₂ |
| Ile | 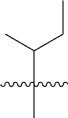 [SMILES: *C(C)CC] |
| Met | 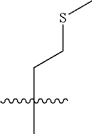 [SMILES: *CCSC] |
| Phe | 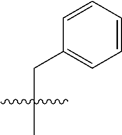 [SMILES: *Cc1ccccc1] |
| Tyr | 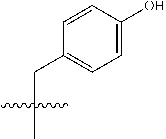 [SMILES: *Cc1ccc(O)cc1] |
| Trp | 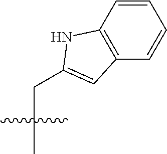 [SMILES: *Cc1cc2ccccc2[nH]1] |
| Asp | 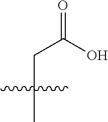 [SMILES: *CC(=O)O] |
| Glu | 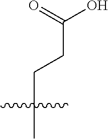 [SMILES: *CCC(=O)O] |
| Asn | 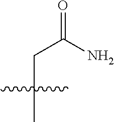 [SMILES: *CC(N)=O] |
| Gln | 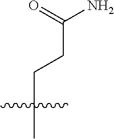 [SMILES: *CCC(N)=O] |
| His | 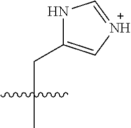 [SMILES: *Cc1c[nH+]c[nH]1] |
| Lys | 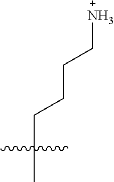 [SMILES: *CCCC[NH3+]] |
| Arg | 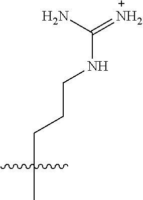 [SMILES: *CCCNC(N)=[NH2+]] |

It will be understood that in the event of any inconsistency between the depicted side chains in Table 12 and the side chains of the indicated amino acid, both will be considered within the scope of possible side chains prior to modification. In some embodiments, the side chain is the side chain of Cys, Met, Phe, Tyr, Trp, Asp, Glu, Asn, Gln, His, or Lys. The chemical modification may produce a side chain substituted one or more times substituted one or more times with a group X₁ is selected independently at each occurrence from hydrogen, —F; —Cl; —Br; —I; —OH, —OR*; —NH₂; —NHR*; —N(R*)₂; —N(R*)₃⁺; —N(R*)—OH; —N(—O)(R*)₂; —O—N(R*)₂; —N(R*)—O—R*; —N(R*)—N(R*)₂; —C═N—R*; —N═C(R*)₂; —C═N—N(R*)₂; —C(═NR*)—N(R*)₂; —SH; —SR*; —CN; —NC; —(C═O)—R*; —CHO; —CO₂H; —CO₂—; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂; —O—(C═O)—NHNH₂; —(C═S)—NH₂; —(C═S)—N(R*)₂; —N(R*)—CHO; —N(R*)—(C═O)—R*; —(C═NR)—O—R*; —O—(C═NR*)—R*, —SCN; —NCS; —NSO; —SSR*; —N(R*)—C(═O)—N(R*)₂; —N(R*)—C(═S)—N(R*)₂; —SO₂—R*; —O—S(═O)₂—R*; —S(═O)₂—OR*; —N(R*)—SO₂—R*; —SO₂—N(R*)₂; —O—SO₃—R*; —O—S(═O)₂—OR*; —O—S(═O)—OR*; —O—S(═O)—R*; —S(═O)—OR*; —S(═O)—R*; —NO; —NO₂; —NO₃; —O—NO; —O—NO₂; —N₃; —N₂—R*; —N(C₂H₄); —Si(R*)₃; —CF₃; —O—CF₃; —PR*₂; —O—P(═O)(OR*)₂; —P(═O)(OR*)₂; C₁-C₈ perfluoroalkyl; an aliphatic C₁-C₈ hydrocarbon radical; a C₁-C₈ aromatic hydrocarbon radical; or a C₁-C₈ heteroaryl radical;
wherein R* is a C₁₋₁₀ hydrocarbon. In some embodiments, the side chain is substituted with one or more oxygen atoms, for example, ═O, —OH, or →O. The resulting oxidized side chain may contain sulfone, sulfoxide, nitro, nitroso, and/or oxo group, to name a few. For example, the side chain may be an oxidized methionine side chain, wherein R_{L} may be selected from:

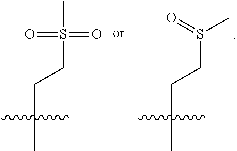

The chemically modified peptides Q may be modified to improve stability or function by incorporating one or more additional amino acids to either or both ends of SEQ ID NO: 2-3531 according to Formula (II):

Ψ₁-Φ-Ψ₂ (II)

where Φ represents a peptide derivative of the invention (e.g., a derivative of an amino acid sequence comprising any of SEQ ID NO: 2-3531) and Ψ₁ and Ψ₂ are independently either absent or are selected from hydrogen, an amino acid, a non-natural amino acid, a non-proteinogenic amino acid, a di- or tri-peptide, or combinations thereof. Suitable amino acids include without limitation, Alanine, Cysteine, Aspartic acid, Glutamic acid, Phenylalanine, Glycine, Histidine, Isoleucine, Lysine, Leucine, Methionine, Asparagine, Pyrrolysine, Proline, Glutamine, Arginine, Serine, Threonine, Selenocysteine, Valine, Tryptophan, and Tyrosine. Each of the foregoing (except glycine) may be in the “L” or “D” optical isomeric configurations. The non-natural amino acid or non-proteinogenic amino acids may be, for example, a dextrorotary “D” optical isomer of a naturally occurring L-amino acid. The non-natural amino acid or non-proteinogenic amino acids may, for example, have the structure of formula (III) or (IV):

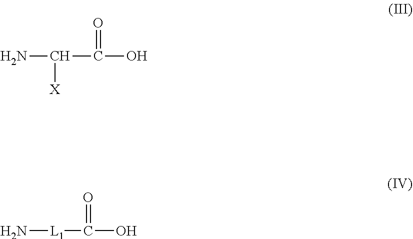

where X is selected from X₁, C₁₋₂₆ (C₁₋₆ or C₆₋₁₂ or C₁₂₋₁₈ or C₁₈₋₂₂) hydrocarbons, optionally substituted with a group X₁ or with from 1-20 (or 1-10 or 1-6 or 1-3) heteroatoms selected from halogen (e.g., fluorine, chlorine, bromine, iodine), oxygen, nitrogen, phosphorous, sulfur, silicon and combinations thereof. In some embodiments, X is ethyl, propyl, butyl, pentyl, hexyl, heptyl, octyl, nonyl, decyl, undecyl, dodecyl. In some embodiments, X is C₁₋₁₂ or C₂₆ alkyl, alkenyl, alkynyl, aryl, aryl-alkyl, alkyl-aryl, alkyl-aryl-alkyl, heteroaryl, alkyl-heteroaryl, heteroaryl-alkyl, alkyl-heteroaryl-alkyl, etc., optionally substituted with X₁, or with 1-20 (or 1-10 or 1-6 or 1-3) heteroatoms selected from halogen (e.g., fluorine), oxygen, nitrogen, phosphorous, sulfur, and combinations thereof. In some embodiments, X comprises a fused ring system having two, three, or more 5- or 6-membered rings. L₁ is a hydrocarbon spacer comprising from 1-20 carbon atoms and optionally substituted with a group X₁ or from 1-20 (or 1-10 or 1-6 or 1-3) heteroatoms selected from halogen (e.g., fluorine, chlorine, bromine, iodine), oxygen, nitrogen, phosphorous, sulfur, silicon and combinations thereof. In some embodiments, L₁ will have the form-(CH₃)p- where “p” is an integer from 1-20 or from 1-10 or from 1-6. In some embodiments, L₁ will comprise from 1-6 oxo or oxa groups. In one embodiment, the amino acid of formula (IV) is aminoethanoic acid, aminopropionic acid, aminobutyric acid, aminovaleric acid, aminocaproic acid, aminoenanthic acid, aminocaprylic acid, amino pelargonic acid, or aminocapric acid. In one embodiment, Ψ₁ and/or Ψ₂ comprises lysyl-aminovaleric acid or aminovaleric acid-lysyl. In some embodiments either terminus may be functionalized with an amino acid of the form H₂N—(CH₂)_{q}—CO₂H where “q” is an integer from 1-10, including amino valeric acid. In some embodiments, a lysine-amino valeric acid group is added at either terminus through a peptide bond. In some embodiments, Ψ₁ and/or Ψ₂ comprise oligomers having 2-16 or 2-8 or 2-6 or 2-4 amino acids, for example, naturally occurring amino acids. The peptide derivatives can also be cyclized.

The peptides of formula (II) may further be modified according to formula (I) such that they have the form of formula (V):

R₁-Ψ₁-Φ-Ψ₂-R₂ (V)

wherein, any of R₁, R₂, Ψ₁, and Ψ₂ may be absent but are otherwise defined as above.

Derivatized peptides may have one or more additional amino acids joined to the amino and/or carboxy terminus via peptide bonds. For example, polyarginine (n=2-15) may be beneficially used to enhance penetration of the peptide into skin. In some embodiments, the peptides will comprise a hydrocarbon chain on the amino and/or carboxyl terminus, including, without limitation, C₁₋₂₄ or C₆₋₁₈ or C₁₂₋₁₈ aliphatic hydrocarbons, which may be straight chained or branched or cyclic. In some embodiments, the peptides include the reaction product of a peptide with a fatty acid or fatty alcohol. A fatty acid or alcohol, as used herein, contains 6-26 carbon atoms. For example, the N-terminus may be reacted with a C₆₋₂₄ fatty acid (e.g., palmitic acid) to form an amide bond. The carboxyl terminus may be reacted with a C₆₋₂₄ fatty alcohol (e.g., cetyl alcohol) to form an ester. These fatty derivatives may improve the lipophilicity of the peptide.

Topically acceptable salts and prodrugs of the peptide derivatives are also suitable. Salts will typically be acid addition salts formed by the reaction of the peptide derivative with an inorganic or an organic acid. Inorganic acids include mineral acids such as HCl and H₂SO₄, and the like. Organic acids include citric, benzoic, tartaric, malic, maleic, succinic, acetic, and propionic acid. The peptides may exist in zwitterionic form. Prodrugs include any esters or amides that hydrolyze in vivo to yield the peptide. Examples of suitable prodrugs can be found in the book entitled “Prodrugs and Targeted Delivery: Towards Better ADME Properties,” Volume 47 (2011), published by WILEY-VCH Verlag & Co, which is herein incorporated by reference in its entirety. In one embodiment, the prodrug is formed by reacting the peptide with glyoxylic acid to produce peptidyl-α-hydroxylglycine derivatives having improved stability. In other embodiments, the prodrugs may include terminal N-acetyl derivatives, side chain N-acetyl derivatives, N-hydroxy methylation or N-phthalidation of its N-terminus and/or side chain.

The active agent may comprise a peptide having an amino acid sequence of SEQ ID NO: 2-3531 or a derivative thereof, wherein one or more amino acid residues of said sequence are oxidized. In some embodiments, the active agent may have the structure:

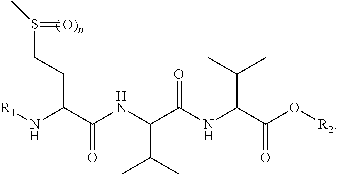

wherein n is 1 or 2;
R₁ is hydrogen or a group R*—(C═O)—;
R₂ is hydrogen or R*; and
R* is independently selected at each occurrence from C₁₋₂₀ hydrocarbons optionally containing from 1-8 heteroatoms. In some embodiments, the peptide derivative may have the structure:

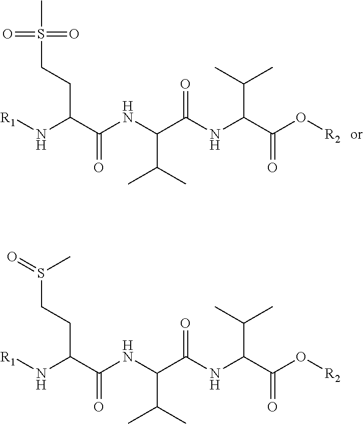

wherein R₁ and R₂ are as defined above. In some embodiments, R₂ is hydrogen. In some embodiments, R₁ is acyl (e.g., C₁-C₂₀ acyl such as acetyl, ethyl acetyl, palmitoyl, oleoyl, myristyl, etc.).

It is within the skill in the art to prepare and derivatize the peptides using, for example, conventional protection and activation chemistry. Typically, the amino functionality of a first amino acid is protected with a removable amino protecting group and the carboxyl functionality of a second amino acid is protected with a removable carboxyl protecting group. Suitable amine protecting groups include, without limitation, benzoyloxycarbonyl (Cbz), tert-butoxycarbonyl (t-Boc), and 9-flourenylmethloxycarbonyl (FMOC). The carboxyl group may be protected by forming an acid or base labile ester such as a methyl, ethyl, benzyl, or trimethylsilyl esters. After protection, the first and second amino acids are reacted in a suitable solvent such as water or DMF in the presence of an in situ activating agent such as N,N′-dicyclohexylcarbodiimide (DCCI), diisopropylcarbodiimide (DIPCDI), or 1-ethyl-3-(3′-dimethylaminopropyl)carbodiimide (EDCI) to effect peptide bond formation. Reactive moieties on the side chains of either amino acid are protected with protecting groups such as tert-butyl or benzyl for OH and SH; methyl, ethyl, tert-butyl or benzyl for carboxyl groups, and 2,2,5,7,8-pentamethylchroman-6-sulphonyl for the —NHC(NH₂)═NH functionality of Arg. Following the coupling reaction, selective deprotection of the amino group of the first amino acid is accomplished by acid hydrolysis under conditions that do not remove the carboxyl protecting group of the second amino acid. The procedure is repeated with additional amino protected amino acids. Solid phase synthesis, such as the well-known Merrifield method, is especially useful for synthesizing the peptides of the invention. Lysine-amino valeric acid (K-ava) derivatives are described in U.S. Pat. No. 8,551,956, the disclosure of which is hereby incorporated by reference.

Topical Compositions

Topical compositions of the invention may comprise an active agent comprising a peptide having an amino acid sequence of SEQ ID NO: 2-3531 or a derivative thereof, wherein one or more amino acid residues of said sequence are oxidized. In some embodiments, the active agent may have the structure:

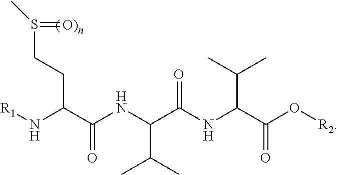

wherein n is 1 or 2;
R₁ is hydrogen or a group R*—(C═O)—;
R₂ is hydrogen or R*; and
R* is independently selected at each occurrence from C₁₋₂₀ hydrocarbons optionally containing from 1-8 heteroatoms. In some embodiments, the peptide derivative may have the structure:

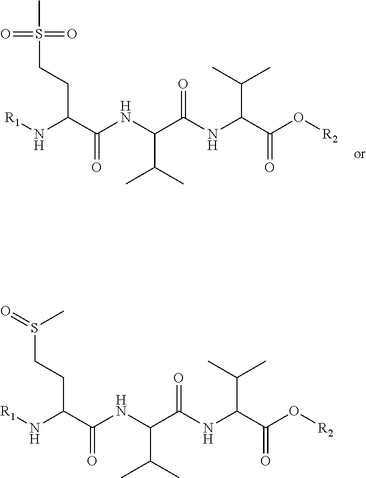

wherein R₁ and R₂ are as defined above. In some embodiments, R₂ is hydrogen. In some embodiments, R₁ is acyl (e.g., C₁-C₂₀ acyl such as acetyl, ethyl acetyl, palmitoyl, oleoyl, myristyl, etc.). The active agent may be present in an effective amount of, for example, 0.00001%-20%.

The compositions according to the invention may be formulated in a variety of forms for topical application and will typically comprise from about 0.000001% by weight to about 20% by weight of the peptide derivative. More typically, the composition will comprise from about 0.00001% peptide derivative by weight to about 10% peptide derivative by weight, and more preferably from about 0.00001% by weight to about 5% peptide derivative by weight of the composition. In one embodiment, the active peptide or a fragment or derivative thereof will comprise from about 0.0010% by weight to about 10% by weight or from about 0.0010% by weight or to about 0.1% by weight of the composition. In some embodiments, the composition will comprise between about 0.000001%-0.1% (e.g., about 0.00001%-0.1%, 0.00001%-0.01%, etc.) peptide derivative by weight of the composition. The compositions may comprise an effective amount of the peptide derivative, by which is meant an amount sufficient to stimulate production of collagen and/or hyaluronic acid in the skin (e.g., from about 0.000001% by weight to about 20% by weight of the peptide derivative). In other embodiments, the amount of peptide or derivative thereof will be sufficient to diminish the appearance of dermatological signs of aging in a given area of skin when topically applied thereto daily for a period of at least eight weeks.

The peptide derivatives (e.g., comprising any of SEQ ID NOs: 2-3531 wherein one or more amino acid residues are oxidized) may be provided in physiologically acceptable vehicles or carriers. The vehicle may be either hydrophobic or hydrophilic. Suitable, hydrophobic carriers include, for example, waxy non-ionic substances commonly used in cosmetics, such as esters and ethers of fatty alcohols and of fatty acids, with carbon chain length from C₄ to C₂₂, typically from C₈ to C₁₈, or from C₁₂ to C₁₈.

Examples of fatty hydrophobic carriers include isopropyl myristate, isopropyl palmitate, octyl palmitate, isopropyl lanolate, acetylated lanolin alcohol, the benzoate of C₁₂-C₁₅ alcohols, cetearyl octanoate, cetyl palmitate, myristyl myristate, myristyl lactate, cetyl acetate, propylene glycol dicaprylate/caprate, decyl oleate, acetylated lanolin, stearyl heptanoate, diisostearyl malate, octyl hydroxystearate, octyl hydroxystearate, isopropyl isostearate, and the like.

Suitable hydrophilic carriers may comprise, for example, water, lower alcohols (C₁₋₆) such as ethanol, mixtures of ethanol and water, glycols, and alkoxylated glycols commonly used in cosmetics, including ethylene glycol, diethylene glycol, triethylene glycol, propylene glycol, dipropylene glycol, and the like.

The topically acceptable vehicle may be in the form of an emulsion. Non-limiting examples of suitable emulsions include water-in-oil emulsions, oil-in-water emulsions, silicone-in-water emulsions, water-in-silicone emulsions, wax-in-water emulsions, water-oil-water triple emulsions or the like having the appearance of a cream, gel or microemulsions. As used herein, the term “oil” includes silicone oils unless otherwise indicated. The emulsion may include an emulsifier, such as a nonionic, anionic or amphoteric surfactant, or a gellant, typically in an amount from about 0.001% to about 5% by weight.

The topically acceptable vehicle may include water; vegetable oils; mineral oils; ester oils; ethers such as dicapryl ether and dimethyl isosorbide; alcohols such as ethanol and isopropanol; fatty alcohols such as cetyl alcohol, cetearyl alcohol, stearyl alcohol and behenyl alcohol; isoparaffins such as isooctane, isododecane (IDD) and isohexadecane; silicone oils such as cyclomethicone, dimethicone, dimethicone cross-polymer, polysiloxanes and their derivatives, including PDMS, dimethicone copolyol, dimethiconols, and amodimethiconols; hydrocarbon oils such as mineral oil, petrolatum, isoeicosane and polyolefins, e.g., (hydrogenated) polyisobutene; polyols such as propylene glycol, glycerin, butylene glycol, pentylene glycol, hexylene glycol, caprylyl glycol; waxes such as beeswax, camauba, ozokerite, microcrystalline wax, polyethylene wax, and botanical waxes; or any combinations or mixtures of the foregoing. Aqueous vehicles may include one or more solvents miscible with water, including lower alcohols, such as ethanol, isopropanol, and the like. The vehicle may comprise from about 50% to about 99% by weight of the composition. In some embodiments, the compositions are anhydrous.

In one embodiment of the invention, the compositions may include one or more additional skin actives, including but not limited to, retinoids, botanicals, keratolytic agents, desquamating agents, keratinocyte proliferation enhancers, collagenase inhibitors, elastase inhibitors, depigmenting agents, anti-inflammatory agents, steroids, anti-acne agents, antioxidants, and advanced glycation end-product (AGE) inhibitors, to name but a few. The amounts of these various ingredients are those conventionally used in the cosmetic field to achieve their intended purpose, and range individually or collectively typically from about 0.001 wt % to about 20 wt % by weight of the composition. The nature of these ingredients and their amounts must be compatible with the production and function of the compositions of the disclosure.

Exemplary anti-aging components include, without limitation, botanicals (e.g., Butea frondosa extract, Tiliacora triandra extract, Portulaca oleracea, Melicope elleryana, etc.); phytol; phytonic acid; retinoids; hydroxy acids (including alpha-hydroxy acids and beta-hydroxy acids), salicylic acid and alkyl salicylates; exfoliating agents (e.g., glycolic acid, 3,6,9-trioxaundecanedioic acid, etc.), estrogen synthetase stimulating compounds (e.g., caffeine and derivatives); compounds capable of inhibiting 5 alpha-reductase activity (e.g., linolenic acid, linoleic acid, finasteride, and mixtures thereof); and barrier function enhancing agents (e.g., ceramides, glycerides, cholesterol and its esters, alpha-hydroxy and omega-hydroxy fatty acids and esters thereof, etc.), to name a few.

Exemplary retinoids include, without limitation, retinoic acid (e.g., all-trans, or 9-cis, or 13-cis), and derivatives thereof, retinaldehyde, retinol (Vitamin A) and esters thereof, such as retinyl palmitate, retinyl acetate and retinyl propionate, and salts thereof. Particular mention may be made of retinol. When present, the retinoids will typically be included in amounts from about 0.0001% to about 5% by weight, more typically from about 0.01% to about 2.5% by weight, or from about 0.1% to about 1.0% by weight. Compositions according to this embodiment will typically include an antioxidant such as ascorbic acid and/or BHT and/or a chelating agent such as EDTA or a salt thereof (e.g., disodium EDTA) in amounts effective to stabilize the retinoid (e.g., 0.0001%-5%). The composition may include from 0.001-10% by weight phytol.

In another embodiment, the topical compositions of the present invention may also include one or more of the following: a skin penetration enhancer; an emollient, such as isopropyl myristate, petrolatum, volatile or non-volatile silicones oils (e.g., methicone, dimethicone), ester oils, mineral oils, and fatty acid esters; a humectant, such as glycerin, hexylene glycol or caprylyl glycol; a skin plumper, such as palmitoyl oligopeptide, collagen, collagen and/or glycosaminoglycan (GAG) enhancing agents; an exfoliating agent; and an antioxidant.

Suitable exfoliating agents include, for example, alpha-hydroxy acids, beta-hydroxy acids, oxa-acids, oxadiacids, and their derivatives such as esters, anhydrides and salts thereof. Suitable hydroxy acids include, for example, glycolic acid, lactic acid, malic acid, tartaric acid, citric acid, 2-hydroxyalkanoic acid, mandelic acid, salicylic acid and derivatives thereof. One exemplary exfoliating agent is glycolic acid. When present, the exfoliating agent may comprise from about 0.001% to about 20% by weight of the composition.

Examples of antioxidants that may be used in the present compositions include compounds having phenolic hydroxy functions, such as ascorbic acid and its derivatives/esters; beta-carotene; catechins; curcumin; ferulic acid derivatives (e.g., ethyl ferulate, sodium ferulate); gallic acid derivatives (e.g., propyl gallate); lycopene; reductic acid; rosmarinic acid; tannic acid; tetrahydrocurcumin; tocopherol and its derivatives, including tocopheryl acetate; uric acid; or any mixtures thereof. Other suitable antioxidants are those that have one or more thiol functions (—SH), in either reduced or non-reduced form, such as glutathione, lipoic acid, thioglycolic acid, and other sulfhydryl compounds. The antioxidant may be inorganic, such as bisulfites, metabisulfites, sulfites, or other inorganic salts and acids containing sulfur. In one embodiment, the composition comprises thiodipropionic acid or a mono- or diester thereof such as dilauryl thiodipropionic acid. Antioxidants may comprise, individually or collectively, from about 0.001% to about 10% (w/w), or from about 0.01% to about 5% (w/w) of the total weight of the composition.

Other additives include: vitamins, such as tocopherol and ascorbic acid; vitamin derivatives such as ascorbyl monopalmitate, tocopheryl acetate, and Vitamin E palmitate; thickeners such as hydroxyalkyl cellulose, carboxymethylcellulose, carbombers, and vegetable gums such as xanthan gum; gelling agents, such as ester-terminated polyester amides; structuring agents; metal chelating agents such as EDTA or salts thereof; fillers and powders, colorants, pH adjusters (citric acid, ethanolamine, sodium hydroxide, etc.); film formers, moisturizers, minerals, viscosity and/or rheology modifiers, anti-acne agents, anti-inflammatories, depigmenting agents, pharmaceutical agents, surfactants, botanicals, sunscreens, insect repellents, skin cooling compounds, skin protectants, conditioners, lubricants, fragrances, excipients, preservatives, stabilizers, emulsifiers, and mixtures thereof. The foregoing may individually or collectively comprise from about 0.0001% to about 20% by weight of the composition.

Details with respect to these and other suitable cosmetic ingredients can be found in the “International Cosmetic Ingredient Dictionary and Handbook,” 10th Edition (2004), published by the Cosmetic, Toiletry, and Fragrance Association (CTFA), at pp. 2177-2299, which is herein incorporated by reference in its entirety. The amounts of these various substances are those that are conventionally used in the cosmetic or pharmaceutical fields, for example, they can constitute individually or in the aggregate, from about 0.01% to about 20% of the total weight of the composition.

A sunscreen may be included to protect the skin from damaging ultraviolet rays. The sunscreen may provide both UVA and UVB protection, by using either a single sunscreen or a combination of sunscreens. Among the sunscreens that can be employed in the present compositions are avobenzone, cinnamic acid derivatives (such as octylmethoxy cinnamate), octyl salicylate, homosalate, oxybenzone, octocrylene, titanium dioxide, zinc oxide, or any mixtures thereof. The sunscreen may be present from about 1 wt % to about 30 wt % of the total weight of the composition.

In one embodiment, the topical composition will have a pH range from 1 to 13, with a pH in the range of from 2 to 12 being typical. In some embodiment, the composition will have a pH in the range of from 3.5 to 7 or from 7-10.5. In some embodiments, the pH will be in the range of 3-4, or 4-5, or 5-6, or 6-7, or 7-8, or 8-9, or 9-10, or 10-11, or 11-12. Suitable pH adjusters such as sodium hydroxide, citric acid and triethanolamine may be added to bring the pH within the desired range.

Another embodiment of the present disclosure is directed to the delivery of the described compositions by the use of targeted delivery systems, for example, liposomes, microspheres (see, e.g., U.S. Pat. No. 5,770,222 to Unger et al.), and the like, so that the components and/or active constituents can more readily reach and affect the subcutaneous layer of the area of application, e.g., face or neck, or the other area of the skin.

The compositions may be formulated in a variety of product forms, such as, for example, a lotion, cream, serum, spray, aerosol, cake, ointment, essence, gel, paste, patch, pencil, towelette, mask, stick, foam, elixir, concentrate, and the like, particularly for topical administration. The composition is typically formulated as a lotion, cream, ointment, serum, or gel.

Methods of Treatment

The invention also provides a method for ameliorating and/or preventing signs of human skin photo- and intrinsic aging comprising topically applying the compositions of the invention. The compositions of the invention are preferably applied to affected skin areas once or twice daily for as long as is necessary to achieve desired anti-aging results. In one embodiment, the compositions of the invention will be applied to the skin in an amount from about 0.001 to about 100 mg/cm², more typically from about 0.01 to about 20 mg/cm², or from about 0.1 to about 10 mg/cm².

In some embodiments, methods for enhancing the production of pro-collagen, collagen and/or HA in human skin comprise topically applying to an area of the skin in need thereof (e.g., sagging skin, thinning skin, skin suffering from wrinkles and fine lines, etc.) a topical composition comprising a topically acceptable vehicle, and an effective amount of a peptide derivative (e.g., a peptide comprising any of SEQ ID NOs: 2-3531 wherein in one or more amino acid residues of SEQ ID NOs 2-3531 are chemically modified such as by oxidation), for a time sufficient to enhance the levels of pro-collagen, collagen, and/or HA in the dermis. The treatment may be at least once or twice daily and may last for a period of at least four weeks, typically at least eight weeks, twelve weeks, or longer.

In another aspect of the invention, the compositions are applied topically to improve the aesthetic appearance of human skin. The method comprises topically applying to an area of the skin in need thereof a composition comprising an effective amount of a peptide derivative (e.g., a peptide comprising any of SEQ ID NOs: 2-3531 wherein in one or more amino acid residues of SEQ ID NOs 2-3531 are chemically modified such as by oxidation) for a time sufficient to improve the aesthetic appearance of said human skin. The composition may optionally further comprise a retinoid (e.g., from 0.0001-5%) and/or an alpha-hydroxy acid (e.g., glycolic acid) (e.g., from 0.0001-25%) and/or a beta-hydroxy acid (e.g., salicylic acid or a derivative) (e.g., from 0.0001-15%).

The improvement in aesthetic appearance of human skin may be an improvement of any attribute or characteristic of skin, including without limitation:
(a) treatment, reduction, and/or prevention of fine lines or wrinkles;
(b) reduction of skin pore size;
(c) improvement in skin thickness, plumpness, and/or tautness;
(d) improvement in skin smoothness, suppleness and/or softness;
(e) improvement in skin tone, radiance, and/or clarity;
(f) improvement in procollagen, and/or collagen production;
(g) improvement in maintenance and remodeling of elastin;
(h) improvement in skin texture and/or promotion of retexturization;
(i) improvement in skin barrier repair and/or function;
(j) improvement in appearance of skin contours;
(k) restoration of skin luster and/or brightness;
(l) replenishment of essential nutrients and/or constituents in the skin;
(m) improvement of skin appearance decreased by aging and/or menopause;
(n) improvement in skin moisturization;
(o) increase in skin elasticity and/or resiliency;
(p) treatment, reduction, and/or prevention of skin sagging;
(q) improvement in skin firmness; and
(r) reduction of pigment spots and/or mottled skin; and
(s) improvement of optical properties of skin by light diffraction or reflection.

As used herein, “aesthetic improvement” may be measured by evaluation of before and after pictures by panels of dermatologists, or by other objective measures known in the art.

In a related implementation, a method is provided for the treatment of wrinkles and/or fine lines on the skin human skin (typically, skin of the face) comprising topically applying to an area of the skin in need thereof (e.g., applying to a wrinkle or fine line) a composition comprising a peptide derivative (e.g., a peptide comprising any of SEQ ID NOs: 2-3531 wherein in one or more amino acid residues of SEQ ID NOs 2-3531 are chemically modified such as by oxidation), for a time sufficient to reduce the visibility, number, or depth of said wrinkles and/or fine lines. The treatment may be a least once or twice daily and may last for a period of at least four weeks, typically at least eight weeks, twelve weeks, or longer. The composition may optionally further comprise a retinoid (e.g., retinol or retinyl palmitate) and/or an alpha-hydroxy acid (e.g., glycolic acid) and/or a beta-hydroxy acid (e.g., salicylic acid or derivative) in amounts effective to improve the appearance of skin. In some embodiments, methods reduce the severity of, reduce the number of, or prevent or forestall the onset of, wrinkles or fine lines on human skin. The composition may be topically applied to an area of the skin in need thereof (e.g., directly to wrinkled skin), an effective amount (e.g., 0.000001%-1% by weight, w/w) of a peptide derivative (e.g., a peptide comprising any of SEQ ID NOs: 2-3531 wherein in one or more amino acid residues of SEQ ID NOs 2-3531 are chemically modified such as by oxidation) in combination with an effective amount (e.g., 0.01%-5% by weight, w/w) of retinol and/or an effective amount (e.g., 0.001%-20% by weight, w/w) of an alpha-hydroxy acid (e.g., glycolic acid) and/or a beta-hydroxy acid (e.g., salicylic acid). The effect of a composition on the formation or appearance of fine lines and wrinkles can be evaluated qualitatively, e.g., by visual inspection, or quantitatively, e.g., by microscopic or computer assisted measurements of wrinkle morphology (e.g., the number, depth, length, area, volume and/or width of wrinkles per unit area of skin).

Topical application of a composition comprising a peptide derivative (e.g., a peptide comprising any of SEQ ID NOs: 2-3531 wherein in one or more amino acid residues of SEQ ID NOs 2-3531 are chemically modified such as by oxidation), typically in a physiologically acceptable vehicle, over an affected area of skin may remediate, reverse, reduce, ameliorate, or prevent dermatological signs of aging. Generally, the improvement in the condition and/or appearance of skin is selected from the group consisting of reducing dermatological signs of chronological aging, photo-aging, hormonal aging, and/or actinic aging; preventing and/or reducing the appearance of lines and/or wrinkles; reducing the noticeability of facial lines and wrinkles, facial wrinkles on the cheeks, forehead, perpendicular wrinkles between the eyes, horizontal wrinkles above the eyes, and around the mouth, marionette lines, and particularly deep wrinkles or creases; improving the appearance of suborbital lines and/or periorbital lines; reducing the appearance of crow's feet; rejuvenating and/or revitalizing skin, particularly aging skin; reducing skin fragility; preventing and/or reversing of loss of glycosaminoglycans and/or collagen; ameliorating the effects of estrogen imbalance; preventing skin atrophy; preventing, reducing, and/or treating hyperpigmentation or hypopigmentation; minimizing skin discoloration; improving skin tone, radiance, clarity and/or tautness; preventing, reducing, and/or ameliorating skin sagging; improving skin firmness, plumpness, suppleness and/or softness; improving procollagen and/or collagen production; improving skin texture and/or promoting retexturization; improving skin barrier repair and/or function; improving the appearance of skin contours; restoring skin luster and/or brightness; minimizing dermatological signs of fatigue and/or stress; resisting environmental stress; replenishing ingredients in the skin decreased by aging and/or menopause; improving communication among skin cells; increasing cell proliferation and/or multiplication; increasing skin cell metabolism decreased by aging and/or menopause; retarding cellular aging; improving skin moisturization; enhancing skin thickness; slowing or halting skin thinning; increasing skin elasticity and/or resiliency; enhancing exfoliation; improving microcirculation; decreasing and/or preventing cellulite formation; and any combinations thereof. In some embodiments, each of the forgoing is associated with female skin.

It is also contemplated that the compositions of the invention will be useful for treating thin skin by topically applying the composition comprising the active peptides derivatives (e.g., a peptide comprising any of SEQ ID NOs: 2-3531 wherein in one or more amino acid residues of SEQ ID NOs 2-3531 are chemically modified such as by oxidation) to thin skin of an individual in need thereof. “Thin skin” is intended to include skin that is thinned due to chronological aging, menopause, or photo-damage and skin that is thinning prematurely. In some embodiments, the treatment is for thin skin in men, whereas other embodiments treat thin skin in women, pre-menopausal or post-menopausal, as it is believed that skin thins differently with age in men and women, and in particular in women at different stages of life.

The method of the invention may be employed prophylactically to forestall aging including in individuals that have not manifested signs of skin aging, most commonly in individuals under 25 years of age. The method may also reverse or treat signs of aging once manifested as is common in individuals over 25 years of age, or to slow the progression of dermatological aging in such individuals.

In one embodiment, the compositions of the invention comprising active peptide derivatives (e.g., a peptide comprising any of SEQ ID NOs: 2-3531 wherein in one or more amino acid residues of SEQ ID NOs 2-3531 are chemically modified such as by oxidation) are applied to human skin to reduce sebum production or improve the appearance of skin affected by cellulite, and/or reduce unwanted lipogenesis or increase lipolysis. In this embodiment, the peptides of the invention can be formulated in topically acceptable vehicles (as described herein) and may include one or more additional agents such as anti-acne ingredients (e.g., salicylic acid, benzoyl peroxide and other peroxides, sulfur, retinoids, etc.) in the case of a facial composition, or, in the case of a cellulite treatment, the formulation may comprise any ingredients suitable for treatment of cellulite, including without limitation, perilla oil and other unsaturated fatty oils and omega-3 fatty acids such as alpha-linolenic acid; caffeine; theophylline; xanthines; retinoids (e.g., retinol); and the like. A cellulite treatment according to the invention will typically be applied topically to skin suffering from cellulite, including skin of the buttocks and thighs for a period of time sufficient to improve the appearance thereof, including for example, daily treatment for at least four weeks, at least eight weeks, at least twelve weeks, or longer. In one embodiment, the compositions are topically applied to treat acne.

In certain embodiments, the compositions described herein comprising active peptide derivatives (e.g., a peptide comprising any of SEQ ID NOs: 2-3531 wherein in one or more amino acid residues of SEQ ID NOs 2-3531 are chemically modified such as by oxidation) can be used to treat and/or prevent hyper-pigmentation of skin and/or of the hair, for example, to lighten skin or hair. In some embodiments, the compositions are topically applied to the skin or hair, for example to an area of hyper-pigmented skin or hair. Hyper-pigmentation includes any coloration of an individual's skin or hair that is darker than desired by the individual and that is caused by melanocytes. Hyper-pigmented areas of the skin include areas of discrete or mottled hyper-pigmentation. Areas of discrete hyper-pigmentation can be distinct, uniform areas of darker color and may appear as brown spots or freckles on the skin, including marks commonly called pigment spots or “age spots.” Areas of mottled hyper-pigmentation of the skin can be dark blotches that are larger and more irregular in size and shape than areas of discrete pigmentation. Areas of hyper-pigmentation also include areas of tanned skin, for example, skin tanned due to UV exposure. Hyper-pigmented hair includes any shade of hair that is darker than desired.

Treating hyper-pigmentation or hyper-pigmented skin/hair refers to eradicating, reducing, ameliorating, or reversing one or more of the unwanted features associated with hyper-pigmentation, such as producing a perceptible lightening of the skin or hair in the affected area. Lightening hyper-pigmented areas of the skin may be effective in diminishing age spots; lightening a suntan; evening or optimizing skin tones, e.g., in areas of mottled hyper-pigmentation; in treating melasmic and chloasmic patches, freckles, after-burn scars, and post-injury hyper-pigmentation. Preventing hyper-pigmentation or hyper-pigmented skin refers to affording skin, not yet affected by hyper-pigmentation, a benefit that serves to avoid, delay, forestall, or minimize one or more unwanted features associated with skin hyper-pigmentation, such as reducing the darkness or size of hyper-pigmented areas that eventually develop.

In some embodiments, the compositions of the invention are used in a rotational, alternating, or sequential treatment regimen comprising topical application of the compositions of the invention for a first period of time (e.g., at least once daily for at least one day), followed by a second period of time in which at least one additional treatment modality is administered for at least one additional day following said first period of time. The second treatment modality may comprise topical application of any skin benefit agent, such as a retinoid (e.g., retinol), phytol, antioxidants (e.g., ascorbic acid or TDPA or esters thereof), botanicals, such as Tiliacora triandra, niacinamide, vitamins such as Vitamin E and Vitamin E acetate, salicylic acid, salicylates and derivatives thereof, moisturizers, emollients, etc.

In another embodiment, the peptide derivative (e.g., a peptide comprising any of SEQ ID NOs: 2-3531 wherein in one or more amino acid residues of SEQ ID NOs 2-3531 are chemically modified such as by oxidation) may be intended for oral use, including for pharmaceutical use. Pharmaceutical formulations will include pharmaceutically acceptable carriers (i.e., diluents and excipients). The pharmaceutical compositions may be included in solid dosage forms, including compressed tablets and capsules, or in liquid or powder forms (including lyophilized powders of the peptide suitable for reconstitution with water). Pharmaceutical compositions may also be in the form of creams, serums, etc., or formulated for injection. Pharmaceutical dosage forms will typically include from about 0.1 mg to about 200 mg, or from about 1 mg to about 100 mg of the peptides of the invention. Solid dosage forms may be immediate release, in which case they will typically comprise a water-soluble or dispersible carrier such as microcrystalline cellulose, mannitol, hydroxypropyl methyl cellulose, PVP or the like, or may be delayed, sustained, or modified release, in which case they may comprise water-insoluble polymers such as cellulose ethers (e.g., ethylcellulose), alone or in combination with water soluble or dispersible polymers, to regulate the rate of dissolution of the dosage form in the stomach.

In one embodiment, the composition is intended for use as a non-therapeutic treatment. In another embodiment, the composition is an article intended to be rubbed, poured, sprinkled, or sprayed on, introduced into, or otherwise applied to the human body for cleansing, beautifying, promoting attractiveness, or altering the appearance, in accordance with the US FD&C Act, § 201(i).

### EXAMPLES

The following example illustrates a specific aspect of the instant description. The example should not be construed as limiting, as the example merely provides specific understanding and practice of the embodiments and its various aspects.

### Example 1: In Vitro Measurements on Human Dermal Fibroblasts

The peptides of the invention were synthesized by GenScript (Piscataway, N.J.).

Human dermal fibroblast cells were grown in a 96 well plate in DMEM media (available from Corning, N.Y.) supplemented with 10% Fetal Bovine Serum (FBS) and L-glutamine (0.07×10⁵ cells/plate). After reaching about 75% confluence, cells were transferred into DMEM media without FBS and incubated for 4-6 hours. Next, cells were treated with a peptide or prodrug thereof (MVV, Ac-MVV) or peptide derivative or prodrug thereof (M(O)₂VV, Ac-M(O)₂VV, Ac-M(O)VV) at 0.00001%, 0.0001%, 0.0005%, or 0.001% final concentration in DMEM media without FBS for 48 h. After treatment, the media were collected and cell viability was measured using MTT. The amount of collagen or hyaluronic acid (HA) secreted was tested in the media using an HTRF human pro-collagen I kit or an HTRF HABP kit, respectively (each available from Cisbio Inc., Bedford, Mass.).

The results for HA production following administration to the fibroblasts are summarized in Table 13 as percent change of HA production for each active relative to vehicle control. p values are given with respect to the change in HA on the fibroblasts with vehicle control administration or with respect to MWV administration.

TABLE 13

| | Concentration | HA % | p value | p value |
|---|---|---|---|---|
| Active | (wt %) | change | (vehicle) | (MW) |
| MW | 0.001% | 9.00% | 0.000518 | |
| | 0.0005% | 3.66% | 0.111352 | |
| | 0.0001% | 1.63% | 0.442766 | |
| | 0.00001% | 5.79% | 0.05904 | |
| Ac-MVV | 0.001% | 12.14% | 0.000223 | 0.0176 |
| | 0.0005% | 6.62% | 0.014093 | 0.0149 |
| | 0.0001% | 14.49% | 2.8E−05 | 4.34E−05 |
| | 0.00001% | 5.47% | 0.206767 | 0.0365 |
| M(O)₂VV | 0.001% | 19.43% | 1.88E−05 | 0.281 |
| | 0.0005% | 10.25% | 0.000195 | 0.316 |
| | 0.0001% | 17.49% | 9.83E−07 | 0.000784 |
| | 0.00001% | 14.01% | 1.1E−05 | 0.957 |
| Ac-M(O)₂VV | 0.001% | 11.04% | 0.005651 | 0.409 |
| | 0.0005% | 7.90% | 0.069938 | 0.253 |
| | 0.0001% | 7.31% | 0.058824 | 0.111 |
| | 0.00001% | 10.33% | 0.014505 | 0.316 |
| Ac-M(O)VV | 0.001% | 21.87% | 0.000145 | 0.0114 |
| | 0.0005% | 17.69% | 0.001447 | 0.0205 |
| | 0.0001% | 16.95% | 0.000128 | 0.000145 |
| | 0.00001% | 17.50% | 6.2E−05 | 0.00658 |

The results for collagen production following administration to the fibroblasts are summarized in Table 14 as percent change of collagen production for each active relative to vehicle control. p values are given with respect to the change in collagen on the fibroblasts with vehicle control administration or MVV administration.

TABLE 14

| | Concentration | Collagen % | p value | p value |
|---|---|---|---|---|
| Active | (wt %) | change | (vehicle) | (MW) |
| MW | 0.001% | 6.22% | 0.034145 | |
| | 0.0005% | 31.22% | 0.164416 | |
| | 0.0001% | 10.56% | 0.0167 | |
| | 0.00001% | 7.22% | 0.011684 | |
| Ac-MVV | 0.001% | 2.21% | 0.366131 | 0.200 |
| | 0.0005% | −2.64% | 0.400509 | 0.231 |
| | 0.0001% | 10.60% | 0.028688 | 0.996 |
| | 0.00001% | −8.24% | 0.004029 | 0.000196 |
| M(O)₂VV | 0.001% | 6.07% | 0.010456 | 0.951 |
| | 0.0005% | 1.56% | 0.474114 | 0.287 |
| | 0.0001% | 12.51% | 0.000636 | 0.702 |
| | 0.00001% | 5.74% | 0.051757 | 0.645 |
| Ac-M(O)₂VV | 0.001% | 13.65% | 0.000235 | 0.0947 |
| | 0.0005% | −0.56% | 0.785196 | 0.257 |
| | 0.0001% | 11.29% | 7.69E−06 | 0.874 |
| | 0.00001% | 12.29% | 2.74E−05 | 0.111 |
| Ac-M(O)VV | 0.001% | 13.49% | 6E−06 | 0.0364 |
| | 0.0005% | 14.02% | 0.000116 | 0.532 |
| | 0.0001% | 4.59% | 0.026163 | 0.220 |
| | 0.00001% | 7.20% | 0.008935 | 0.994 |

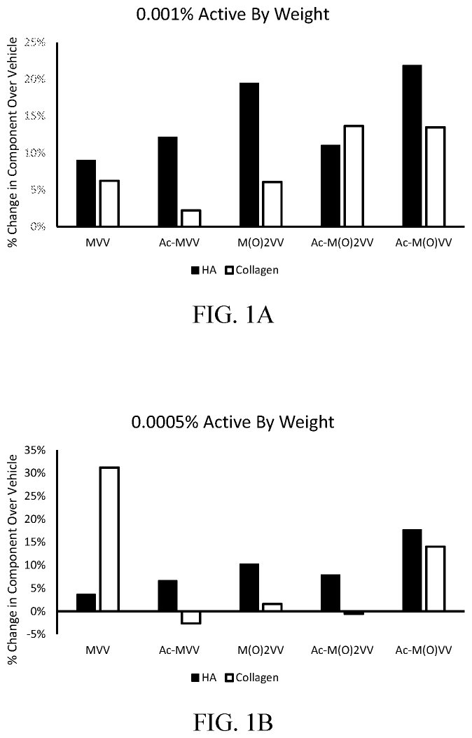

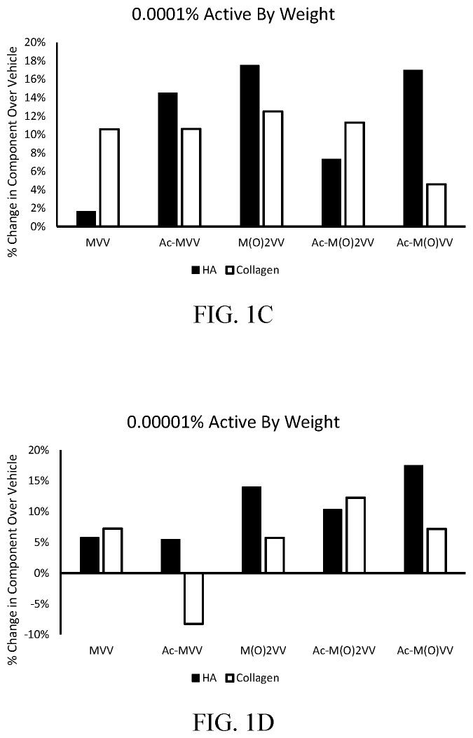

As shown in Table 14, peptides derivatives of the invention effectively increase pro-collagen I acid production in human dermal fibroblast cells greater than the underivatized version. FIGS. 1A-ID graphically illustrate the percent increases in HA and collagen at each tested derivative concentration shown in Tables 13 and 14.

### Example 2: Full Thickness Skin Model Assays

A series of experiments was conducted to assess the effects of peptide derivatives on the production of hyaluronic acid (HA) on full thickness 3D skin cultures. Five different treatment regimens were assessed: (1) 0.01% by weight M(O)₂VV in vehicle; (2) 0.002% by weight M(O)₂VV in vehicle; (3) 0.01% by weight Ac-M(O)₂VV in vehicle; (4) 0.002% by weight Ac-M(O)₂VV in vehicle; and (5) vehicle alone (DMSO). Compositions at the indicated weight percentages in DMSO were applied to 3D skin equivalents daily for 3 consecutive days.

Human 3D skin EFT400FT tissues (MatTek, MA) were cultured following the manufacturer's instructions. Each treatment regimen was topically applied to six 3D skin samples. Different 3D skin samples were assigned to one of treatment regimens (1)-(5) as described above, with six skin tissue models allocated to each treatment regimen (i.e., n=6). Following treatment, media were collected and the amount of hyaluronic acid secreted from the cells was measured using a HTRF HABP kit (available from Cisbio Inc., Bedford, Mass.).

The results for HA production are shown in Table 15. p values are given in relation to administration with vehicle control alone or in relation to MVV.

TABLE 15

| Peptide | Concentration | | % HA | |
|---|---|---|---|---|
| Derivative | (wt % (ppm)) | | change | p value |
| M(O)₂VV | 0.01% | (100 ppm) | 10.27567 | 6.11E−05 |
| M(O)₂VV | 0.002% | (20 ppm) | 6.236025 | |
| Ac-M(O)₂VV | 0.01% | (100 ppm) | 12.46642 | 7.17E−05 |
| Ac-M(O)₂VV | 0.002% | (20 ppm) | 1.386264 | |

### Example 3: Ex Vivo Assays

Samples of explanted skin tissue were obtained from an abdominal plasty of a47 year old Caucasian woman. Samples (33 in total) were maintained in tissue culture medium at 37° C. in a humid, 5% CO₂ atmosphere for the duration of the study.

The skin tissue explants were then distributed into appropriate controls or treated samples in triplicates. The control received only a vehicle (DMSO), and the treatment samples received vehicle comprising either M(O)₂VV or Ac-M(O)₂VV at 0.0005%, 0.001%, or 0.01% by weight of the composition. Administration of vehicle or vehicle with the peptide occurred on days 0, 1, 2, 5, and 7.

Stained tissue sections (in triplicates) were examined for qualitative and semi-qualitative differences between treated and control samples and low and high power using Zeiss Mimax and Midi BF microscope and software system. For HA evaluation, samples were washed and processed with Methylthiazolyldiphenyl-tetrazolium bromide (MTT). MTT is converted to water-insoluble dark blue colored MTT-formazan by mitochondrial dehydrogenases. Subsequently the blue crystals have been solubilized and the color intensity, that is directly proportional to skin vitality, has been measured with a plate reader at a wavelength of 570 nm. For each analyzed skin sample the obtained absorbance value has been normalized upon the weight in grams. Twelve skin suctions were stained with an acian blue stain that dyes acid mucins such as hyaluronan to develop the HA score. Dermal collagen was also measured using a picrosirius red histochemical stain that dyes collagen fibers in purple-red. The papillary dermis was selected for the analysis as thi sis the portion of the dermis more subjected to collagen variation in response to treatment.

8-bit grey-scale photomicrographs were captured and transformed from RGB to the standard CIE L*a*b* color space. The L* value for each pixel in the ROI was evaluated using ImageJ software (National Institutes of Health, Bethesda, Md.) with the Color Inspector 3D plug-in. The amount of hyaluron present was evaluated based on the intensity and distribution of blue color in the dermis using an imaging method analogous to that used for procollagen I. Scores reflecting the amount of hyaluronan detected were assigned using an image analysis algorithm proprietary to Cutech Srl. Higher HA scores is indicative of a higher HA content. Similarly, the amount of procollagen I was evaluated based on the intensity and distribution of immunostained procollagen I in the area corresponding to the papillary dermis, just below the epidermis. As above, 8-bit grey-scale photomicrographs were captured and transformed from RGB to the standard CIE L*a*b* color space. The L* value for each pixel in the ROI was evaluated using ImageJ software (National Institutes of Health, Bethesda, Md.) with the Color Inspector 3D plug-in. Scores reflecting the amount of procollagen I detected were assigned using an image analysis algorithm proprietary to Cutech Srl.

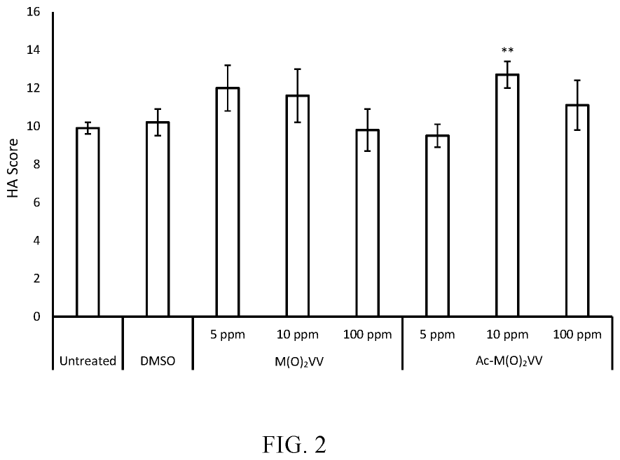

Table 16 shows the measured HA scores in various samples and FIG. 2 graphically represents the results of the explant measurements for HA measured. Columns marked with “**” were highly significant (p<0.05) with respect to untreated samples and with respect to samples administered vehicle alone (DMSO).

TABLE 16

| | | | | | % Change | % Change |
|---|---|---|---|---|---|---|
| | | | | | Over | Over |
| | | | Mean | SEM | Untreated | DMSO |
| Untreated | | | 9.9 | 0.3 | | |
| DMSO | | | 10.2 | 0.7 | 3% | |
| M(O)₂VV | 5 | ppm | 12 | 1.2 | 21% | 18% |
| | 10 | ppm | 11.6 | 1.4 | 17% | 14% |
| | 100 | ppm | 9.8 | 1.1 | −1% | −4% |
| Ac- | 5 | ppm | 9.5 | 0.6 | −4% | −7% |
| M(O)₂VV | 10 | ppm | 12.7 | 0.7 | 28% | 25% |
| | 100 | ppm | 11.1 | 1.3 | 12% | 9% |

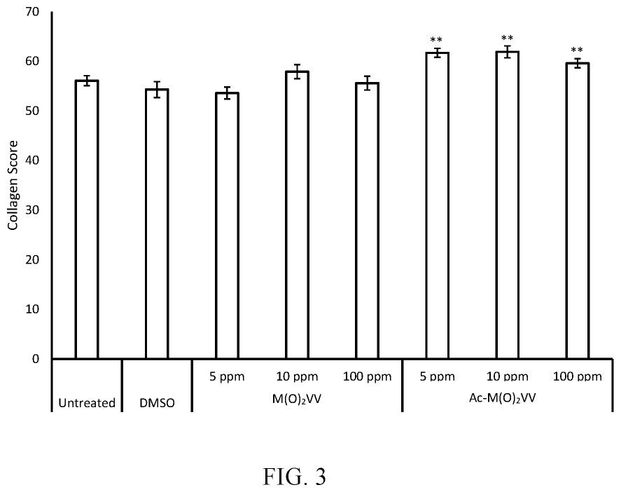

Table 17 shows the measured collagen scores in various samples and FIG. 3 graphically represents the results of the explant measurements for collagen measured. Columns marked with “**” were highly significant (p<0.05) with respect to untreated samples and with respect to samples administered vehicle alone (DMSO.

TABLE 17

| | | | | | % Change | % Change |
|---|---|---|---|---|---|---|
| | | | | | Over | Over |
| | | | Mean | SEM | Untreated | DMSO |
| Untreated | | | 56.1 | 1 | | |
| DMSO | | | 54.3 | 1.6 | −18% | |
| M(O)₂VV | 5 | ppm | 53.6 | 1.2 | −25% | −7% |
| | 10 | ppm | 57.9 | 1.4 | 18% | 35% |
| | 100 | ppm | 55.6 | 1.4 | −5% | 13% |
| Ac- | 5 | ppm | 61.7 | 0.9 | 57% | 73% |
| M(O)₂VV | 10 | ppm | 61.9 | 1.2 | 59% | 75% |
| | 100 | ppm | 59.6 | 0.9 | 35% | 52% |

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings. Accordingly, the present description is intended to embrace all such alternatives, modifications, and variances which fall within the scope of the appended claims.

1. A topical composition comprising an active agent comprising a peptide having an amino acid sequence comprising or consisting of M(O)₂VV or M(O)VV, wherein the methionine amino acid residue of said sequence is oxidized.

2. The topical composition of claim 1, wherein the active agent is present in an amount from about 0.0001% to about 10% by weight of the composition.

3. The topical composition of claim 1, wherein the topically acceptable vehicle of said topical composition comprises a water-in-oil, oil-in-water, silicone-in-water, or water-in-silicone emulsion, further comprising an emulsifier.

4. The topical composition of claim 1, further comprising an additional active ingredient selected from the group consisting of alpha-hydroxy acids, thiodipropionic acid or esters thereof, salicylic acid, niacinamide, hexyl resorcinol, phytol, and retinoids.

5. The topical composition of claim 1, wherein said sequence consists of from 3 to 10 amino acids.

6. The topical composition of claim 1, wherein said active agent is N-acetylated.

7. The topical composition of claim 1, wherein said active agent has the structure: 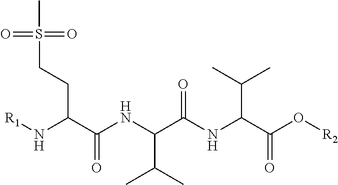
wherein R₁ is hydrogen or a group R*—(C═O)—;
R₂ is hydrogen or R*; and
R* is independently selected at each occurrence from C₁₋₂₀ hydrocarbons optionally containing from 1-8 heteroatoms.

8. The topical composition according to claim 7, wherein R₁ is R*—(C═O)— and R* is C₁₋₂₀ alkyl.

9. The topical composition of claim 1, wherein said active agent has the structure:

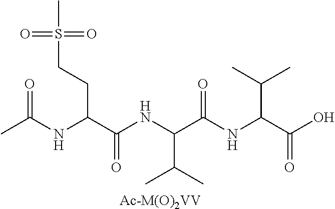

10. The topical composition of claim 1, wherein said active agent has the structure:

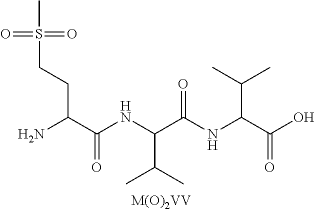

11. The topical composition of claim 1, wherein said active agent has the structure: 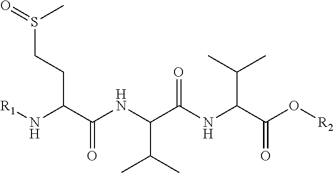
wherein R₁ is hydrogen or a group R*—(C═O)—;
R₂ is hydrogen or R*; and
R* is independently selected at each occurrence from C₁₋₂₀ hydrocarbons optionally containing from 1-8 heteroatoms.

12. The topical composition of claim 1, wherein said peptide derivative has the structure:

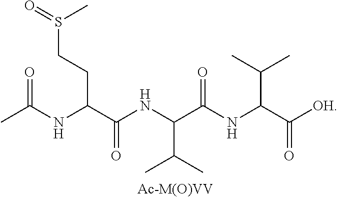

13. The topical composition of claim 1, wherein said peptide derivative has the structure:

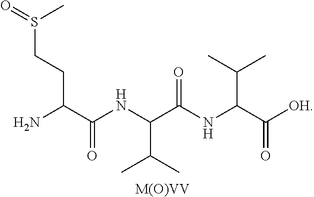

14. A topical composition comprising an effective amount of a compound having the structure: 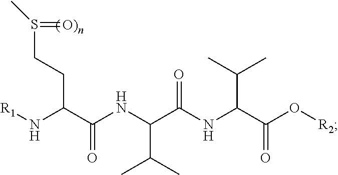
wherein n is 1 or 2;
R₁ is hydrogen or a group R*—(C═O)—;
R₂ is hydrogen or R*; and
R* is independently selected at each occurrence from C₁₋₂₀ hydrocarbons optionally containing from 1-8 heteroatoms.

15. The topical composition of claim 14, wherein said compound has the structure:

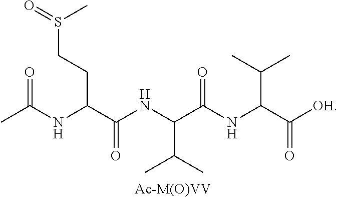

16. The topical composition of claim 14, wherein said compound has the structure:

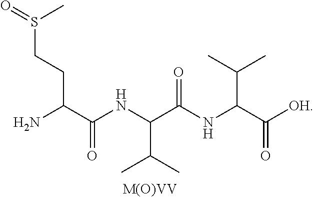

17. The topical composition of claim 14, wherein said compound has the structure:

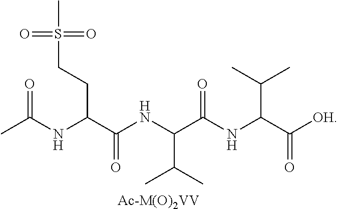

18. The topical composition of claim 14, wherein said compound has the structure:

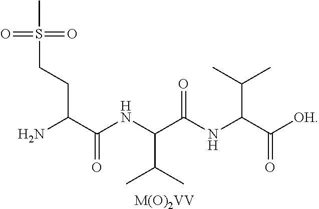

19. A method for diminishing the appearance of dermatological signs of aging comprising topically applying to the skin in need thereof a topical composition according to claim 1.

20. The method according to claim 19, wherein said dermatological signs of aging include fine lines and/or wrinkles.

21. The method according to claim 19, wherein said peptide derivative increases collagen production in skin.

22. The method according to claim 19, wherein said peptide derivative increases hyaluronic acid production in skin.

23. The method according to claim 19, wherein said composition is applied at least once daily for at least eight weeks.

24. (canceled)

25. (canceled)

26. (canceled)

27. (canceled)

28. (canceled)

29. (canceled)
